SEQUENCE LISTING (135 sequences)

SEQ ID NO: 1 - DNA, 21 bp, synthetic construct
  source 1..21: mol_type = other DNA
ggttgaggctcttatagctga

SEQ ID NO: 2 - DNA, 19 bp, synthetic construct
  source 1..19: mol_type = other DNA
ttgaggctcttatagctga

SEQ ID NO: 3 - DNA, 20 bp, synthetic construct
  source 1..20: mol_type = other DNA
gttgaggctcttatagctga

SEQ ID NO: 4 - DNA, 22 bp, synthetic construct
  source 1..22: mol_type = other DNA
tggttgaggctcttatagctga

SEQ ID NO: 5 - DNA, 96 bp, synthetic construct
  source 1..96: mol_type = other DNA
ggttgaggctcttatagctgagtttagtactctggaaacagaatctactaaaacaaggcaaaatgccgtgtttatctcgtcaacttgttggcgaga

SEQ ID NO: 6 - DNA, 94 bp, synthetic construct
  source 1..94: mol_type = other DNA
ttgaggctcttatagctgagtttagtactctggaaacagaatctactaaaacaaggcaaaatgccgtgtttatctcgtcaacttgttggcgaga

SEQ ID NO: 7 - DNA, 95 bp, synthetic construct
  source 1..95: mol_type = other DNA
gttgaggctcttatagctgagtttagtactctggaaacagaatctactaaaacaaggcaaaatgccgtgtttatctcgtcaacttgttggcgaga

SEQ ID NO: 8 - DNA, 97 bp, synthetic construct
  source 1..97: mol_type = other DNA
tggttgaggctcttatagctgagtttagtactctggaaacagaatctactaaaacaaggcaaaatgccgtgtttatctcgtcaacttgttggcgaga

SEQ ID NO: 9 - DNA, 76 bp, synthetic construct
  source 1..76: mol_type = other DNA
gttttagtactctggaaacagaatctactaaaacaaggcaaaatgccgtgtttatctcgtcaacttgttggcgaga

SEQ ID NO: 10 - DNA, 3258 bp, synthetic construct
  source 1..3258: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaa

SEQ ID NO: 11 - protein, 1085 aa, synthetic construct
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 12 - DNA, 600 bp, synthetic construct
  source 1..600: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 13 - DNA, 500 bp, synthetic construct
  source 1..500: mol_type = other DNA
gatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 14 - DNA, 400 bp, synthetic construct
  source 1..400: mol_type = other DNA
gttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 15 - DNA, 300 bp, synthetic construct
  source 1..300: mol_type = other DNA
gttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 16 - DNA, 1497 bp, synthetic construct
  source 1..1497: mol_type = other DNA
agaggaccctttcaaggacattagtggtggaggcagcatagtagctcccaaggcagagggattgagagaagagtttgaggactgggaaggcgggacacatgattgggtgatgggagaagggggcagagaatagcgagattgctttctttgcccacggagaaacagaggagtgtggatcatgaatgggcaagatctttaagtgccagggggggtcatggaggaggggagggcctgctccagaggaggaccattcctgcttcagagccaagcaggacctaggctgtgaagattcggagaaagagatggaggggagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctctggggaagaggagagcagagaatggacggggagatgtgagggtcttgggccctggcatatttacccagagtctgcctgtgtccgcagaagtccatggcccctcctggtggaggccacacttcagaggacaggttgccaggtctgggctccaagattggtacaatagagcagagaga

SEQ ID NO: 17 - DNA, 413 bp, synthetic construct
  source 1..413: mol_type = other DNA
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 18 - DNA, 21 bp, synthetic construct
  source 1..21: mol_type = other DNA
agtggaggccctgatagccga

SEQ ID NO: 19 - DNA, 90 bp, synthetic construct
  source 1..90: mol_type = other DNA
tatttgcatgtcgctatgtgttctgggaaatcaccataaacgtgaaatgtctttggatttgggaatcttataagttctgtatgagaccac

SEQ ID NO: 20 - DNA, 215 bp, synthetic construct
  source 1..215: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 21 - DNA, 95 bp, synthetic construct
  source 1..95: mol_type = other DNA
atatttgcatgtcgctatgtgttctgggaaatcaccataaacgtgaaatccctatcagtgatagagacttataagttccctatcagtgatagaga

SEQ ID NO: 22 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 23 - DNA, 314 bp, synthetic construct
  source 1..314: mol_type = other DNA
gatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtt

SEQ ID NO: 24 - DNA, 500 bp, synthetic construct
  source 1..500: mol_type = other DNA
ctccaagaactcgtacaaaaatgctctgaagtaggtctcgagatcaatactgggaagacgaaagtcttgcgaaaccgattcgctgaccccagtaaagtctacttcggtagcccttcccccaccacccagctcgacgacgtcgacgagtacatctacctcggtcgtcaaatcaacgcccaaaacaacttgatgccggaaatccaccgaagacgtcgagcagcctgggctgcattcaatggaatcaagaatgccaccgactccatcaccgacaagaagattcgtgcgaatctgttcgactcaattgtccttccagcgctcacctacgtttcagaagcctggacattcaccaaagctctatccgaacgagtacgaatcacacatgcctcccccacaaattattgatctacatccccatttggtatagtgtcttacaggactctcaatatgtcagctgcatgataaatggtccctatataattgaattgcaaatctaaatgttt

SEQ ID NO: 25 - DNA, 350 bp, synthetic construct
  source 1..350: mol_type = other DNA
caaaaaaaactagcaataaaggaataaaaaactgtacaccttaaaggcgcacactctgttttgcaaattttatttttagttgtgaattttctgctgagacctgaaaatagcaactttagtactactataatttgtcaaccttttcaaaaaaagcatgcaatttttgagaaactcttataaaagctattattaaaaaaacaccttttttccaaaattattccacaaaaaatatgttatgaaatgcctacaccctctcacacacactctttatactactctgtcaaactcacgagatgtctgccgcctcttgtgttgcccctatataaacacctcctattgcgagatgtctt

SEQ ID NO: 26 - DNA, 797 bp, synthetic construct
  source 1..797: mol_type = other DNA
attttcaacgtcctcgatagtatagtggttagtatccccgcctgtcacgcgggagaccggggttcaattccccgtcggggagaatctgtgattctttttttttttcttttactttgttatataaacaatttttgttttaattgaatctaatttgccattgcttttaggaatctcaggcatccagcaagcgtttgtccgccgaatcgcccatcagtgaagaagatcctgtggcggctacgaaaatctccccggccatgtcggcctccacctccagcgaaaaacccatcagcgagctggccacctctgtgctgacccaccgctttccagactccacctcctcacccggcgaacatggccttggacgaatgcagttgtcgatccgctacagcgcccagcgtcaaaaactagacgtgaccatacacaaaatccagaagataccacttcgcgatcccagcaatatccccgatccgtatgttaagctgtatctgttgcctggacgcaccaaggagtcgaaacgcaagacgagcgtgatcaaggacaactgcaaccccgtctacgatgcatcctttgagtacctgatttccattgccgaactcaggcagacggaactggaggtgacggtgtgcacccaaaagggattcctatccggcggtagtcccatcattggcatggtaggtacccgaaagcaaccccttagttacagacacagcgcgtacgtccttcgcatccttatgattcccaagtacatattctgcaagagtacagtatatataggaaagatatccgggtgaacttcg

SEQ ID NO: 27 - DNA, 400 bp, synthetic construct
  source 1..400: mol_type = other DNA
gttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaataggacactttgattctaaaggaaattttgaaaatcttaagcagagggttcttaagaccatttgccaattcttataattctcaactgctctttcctgatgttgatcatttatataggtatgttttcctcaatacttc

SEQ ID NO: 28 - DNA, 421 bp, synthetic construct
  source 1..421: mol_type = other DNA
ttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcg

SEQ ID NO: 29 - DNA, 215 bp, synthetic construct
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 30 - DNA, 134 bp, synthetic construct
  source 1..134: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagt

SEQ ID NO: 31 - DNA, 159 bp, Homo sapiens
  source 1..159: mol_type = genomic DNA
atggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctga

SEQ ID NO: 32 - protein, 52 aa, Homo sapiens
MEKVQYLTRSAIRRASTIEMPQQARQKLQNLFINFCLILICLLLICIIVMLL

SEQ ID NO: 33 - DNA, 156 bp, synthetic construct
  source 1..156: mol_type = other DNA
atggagaaagtccaatacctcactcgctcagctataagagcctcaaccattgaaatgcctcaacaagcacgtcaaaagctacagaatctatttatcaatttctgtctcatcttaatatgtctcttgctgatctgtatcatcgtgatgcttctctga

SEQ ID NO: 34 - protein, 51 aa, synthetic construct
MEKVQYLTRSAIRASTIEMPQQARQKLQNLFINFCLILICLLLICIIVMLL

SEQ ID NO: 35 - DNA, 4610 bp, synthetic construct
  source 1..4610: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggttgaggctcttatagctgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 36 - DNA, 4610 bp, synthetic construct
  source 1..4610: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtggaggccctgatagccgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 37 - DNA, 5079 bp, synthetic construct
  source 1..5079: mol_type = other DNA
aagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatccccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtggaggccctgatagccgagttttagtactctggaaacagaatctactaaaacaaggcaaaatgccgtgtttatctcgtcaacttgttggcgaga

SEQ ID NO: 38 - DNA, 10 bp, synthetic construct
  source 1..10: mol_type = other DNA
gccaccatgg

SEQ ID NO: 39 - DNA, 21 bp, synthetic construct
  source 1..21: mol_type = other DNA
ccaaagaagaagcggaaggtc

SEQ ID NO: 40 - DNA, 48 bp, synthetic construct
  source 1..48: mol_type = other DNA
aaaaggccggcggccacgaaaaaggccggccaggcaaaaaagaaaaag

SEQ ID NO: 41 - DNA, 358 bp, synthetic construct
  source 1..358: mol_type = other DNA
aagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctg

SEQ ID NO: 42 - DNA, 417 bp, synthetic construct
  source 1..417: mol_type = other DNA
ggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcc

SEQ ID NO: 43 - DNA, 375 bp, synthetic construct
  source 1..375: mol_type = other DNA
ccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 44 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 45 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 46 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 599 aa, synthetic construct
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 534 aa, synthetic construct
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 49 - protein, 7 aa, synthetic construct
NGSGQNQ

SEQ ID NO: 50 - protein, 6 aa, synthetic construct
NNSEFA

SEQ ID NO: 51 - protein, 5 aa, synthetic construct
GRDNV

SEQ ID NO: 52 - protein, 14 aa, synthetic construct
ATNHQSAQAQAQTG

SEQ ID NO: 53 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 54 - protein, 588 aa, synthetic construct
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 55 - protein, 532 aa, synthetic construct
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 56 - protein, 14 aa, synthetic construct
  VARIANT 1: note = X is any amino acid
  VARIANT 5..6: note = X is any amino acid
  VARIANT 10..14: note = X is any amino acid
XDVQXXPGFXXXXX

SEQ ID NO: 57 - protein, 14 aa, synthetic construct
  VARIANT 6: note = X is any amino acid
  VARIANT 10..12: note = X is any amino acid
  VARIANT 14: note = X is any amino acid
ADVQQXPGFXXXTX

SEQ ID NO: 58 - protein, 14 aa, synthetic construct
  VARIANT 1: note = X is any amino acid
  VARIANT 5..6: note = X is any amino acid
  VARIANT 10: note = X is any amino acid
  VARIANT 13..14: note = X is any amino acid
XDVQXXPGFXAQXX

SEQ ID NO: 59 - protein, 14 aa, synthetic construct
  VARIANT 1: note = X is any amino acid
  VARIANT 5..6: note = X is any amino acid
  VARIANT 10: note = X is any amino acid
  VARIANT 13..14: note = X is any amino acid
XDVQXXPGFXAEXX

SEQ ID NO: 60 - protein, 14 aa, synthetic construct
  VARIANT 1: note = X is any amino acid
  VARIANT 5..6: note = X is any amino acid
  VARIANT 10: note = X is any amino acid
  VARIANT 13..14: note = X is any amino acid
XDVQXXPGFXPQXX

SEQ ID NO: 61 - protein, 14 aa, synthetic construct
  VARIANT 1: note = X is any amino acid
  VARIANT 5..6: note = X is any amino acid
  VARIANT 10: note = X is any amino acid
  VARIANT 13..14: note = X is any amino acid
XDVQXXPGFXPEXX

SEQ ID NO: 62 - protein, 14 aa, synthetic construct
  VARIANT 6: note = X is any amino acid
  VARIANT 10: note = X is any amino acid
  VARIANT 14: note = X is any amino acid
ADVQQXPGFXAQTX

SEQ ID NO: 63 - protein, 14 aa, synthetic construct
  VARIANT 6: note = X is any amino acid
  VARIANT 10: note = X is any amino acid
  VARIANT 14: note = X is any amino acid
ADVQQXPGFXAETX

SEQ ID NO: 64 - protein, 14 aa, synthetic construct
  VARIANT 6: note = X is any amino acid
  VARIANT 10: note = X is any amino acid
  VARIANT 14: note = X is any amino acid
ADVQQXPGFXPQTX

SEQ ID NO: 65 - protein, 14 aa, synthetic construct
  VARIANT 6: note = X is any amino acid
  VARIANT 10: note = X is any amino acid
  VARIANT 14: note = X is any amino acid
ADVQQXPGFXPETX

SEQ ID NO: 66 - protein, 14 aa, synthetic construct
  VARIANT 1: note = X is any amino acid
  VARIANT 5..6: note = X is any amino acid
  VARIANT 13..14: note = X is any amino acid
XDVQXXPGFQAQXX

SEQ ID NO: 67 - protein, 14 aa, synthetic construct
  VARIANT 6: note = X is any amino acid
  VARIANT 14: note = X is any amino acid
ADVQQXPGFQAQTX

SEQ ID NO: 68 - protein, 14 aa, synthetic construct
  VARIANT 1: note = X is any amino acid
  VARIANT 5: note = X is any amino acid
  VARIANT 10..13: note = X is any amino acid
XDVQXLPGFXXXXE

SEQ ID NO: 69 - protein, 14 aa, synthetic construct
  VARIANT 10..12: note = X is any amino acid
ADVQQLPGFXXXTE

SEQ ID NO: 70 - protein, 14 aa, synthetic construct
  VARIANT 1: note = X is any amino acid
  VARIANT 5..6: note = X is any amino acid
  VARIANT 11..14: note = X is any amino acid
XDVQXXPGFQXXXX

SEQ ID NO: 71 - protein, 14 aa, synthetic construct
  VARIANT 6: note = X is any amino acid
  VARIANT 11..12: note = X is any amino acid
  VARIANT 14: note = X is any amino acid
ADVQQXPGFQXXTX

SEQ ID NO: 72 - protein, 14 aa, synthetic construct
ADVQQLPGFQAQTE

SEQ ID NO: 73 - protein, 14 aa, synthetic construct
ADVQQHPGFQAQTE

SEQ ID NO: 74 - protein, 14 aa, synthetic construct
ADVQQVPGFQAQTM

SEQ ID NO: 75 - protein, 14 aa, synthetic construct
ADVQQVPGFQAQTQ

SEQ ID NO: 76 - protein, 14 aa, synthetic construct
ADVQQLPGFGAQTE

SEQ ID NO: 77 - protein, 14 aa, synthetic construct
ADVQQLPGFRPETE

SEQ ID NO: 78 - protein, 14 aa, synthetic construct
ADVQQLPGFNAQTE

SEQ ID NO: 79 - protein, 7 aa, synthetic construct
RGDAARL

SEQ ID NO: 80 - protein, 7 aa, synthetic construct
SHVRGDL

SEQ ID NO: 81 - protein, 7 aa, synthetic construct
VVSSGAR

SEQ ID NO: 82 - protein, 4 aa, synthetic construct
VRGD

SEQ ID NO: 83 - protein, 7 aa, synthetic construct
RTDLKGL

SEQ ID NO: 84 - protein, 7 aa, synthetic construct
YPSTGSG

SEQ ID NO: 85 - protein, 8 aa, synthetic construct
FAGSLTRA

SEQ ID NO: 86 - protein, 7 aa, synthetic construct
DRTLTTR

SEQ ID NO: 87 - protein, 7 aa, synthetic construct
RIAGRDV

SEQ ID NO: 88 - protein, 7 aa, synthetic construct
SLGSGVR

SEQ ID NO: 89 - protein, 7 aa, synthetic construct
PQYGRGG

SEQ ID NO: 90 - protein, 7 aa, synthetic construct
LQVSRVS

SEQ ID NO: 91 - protein, 7 aa, synthetic construct
VRSYSSN

SEQ ID NO: 92 - protein, 7 aa, synthetic construct
TMRVGSL

SEQ ID NO: 93 - protein, 7 aa, synthetic construct
GAYSRGV

SEQ ID NO: 94 - protein, 7 aa, synthetic construct
LRGGSLG

SEQ ID NO: 95 - protein, 7 aa, synthetic construct
VYGTGVR

SEQ ID NO: 96 - protein, 7 aa, synthetic construct
DKLIIVS

SEQ ID NO: 97 - protein, 7 aa, synthetic construct
AEDRTKL

SEQ ID NO: 98 - protein, 7 aa, synthetic construct
LSASASL

SEQ ID NO: 99 - protein, 7 aa, synthetic construct
LADQTKL

SEQ ID NO: 100 - protein, 7 aa, synthetic construct
LLLKLQE

SEQ ID NO: 101 - protein, 7 aa, synthetic construct
ELPVKTG

SEQ ID NO: 102 - protein, 7 aa, synthetic construct
LDLKVVG

SEQ ID NO: 103 - protein, 5 aa, synthetic construct
RDAVL

SEQ ID NO: 104 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFQAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 105 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQHPGFQAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 106 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHVRGDLSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 107 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGVVSSGARSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 108 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDAARLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 109 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTMWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 110 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTQWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 111 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFGAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 112 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFRPETEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 113 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFNAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 114 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTISQVNGRPRDLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 115 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLGLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 116 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGASTNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLLLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 117 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLLLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 118 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGASTNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 119 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 120 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 121 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDFNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 122 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDHASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 123 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 124 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDHGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 125 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANKTTNKDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 126 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANKIQRTDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 127 - DNA, 10 bp, synthetic construct
  source 1..10: mol_type = other DNA
ttatagctga

SEQ ID NO: 128 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatgactcgctcagctataagagcctcaaccgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggttgaggctcttatagctgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 129 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctcgctcagctataagagcctcaaccgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggttgaggctcttatagctgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 130 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatgtctcgctcagctataagagcctcaaccgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggttgaggctcttatagctgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 131 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatgcctcgctcagctataagagcctcaaccgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggttgaggctcttatagctgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 132 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatgactcgctcggctatcagggcctccactgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtggaggccctgatagccgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 133 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctcgctcggctatcagggcctccactgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtggaggccctgatagccgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 134 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatgtctcgctcggctatcagggcctccactgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtggaggccctgatagccgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

SEQ ID NO: 135 - DNA, 4637 bp, synthetic construct
  source 1..4637: mol_type = other DNA
tgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatgcctcgctcggctatcagggcctccactgccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtggaggccctgatagccgagttttagtactctggaaacagaatctactaaaacaaggcaaaatgccgtgtttatctcgtcaacttgttggcgagatttttgctcgagttaagggcgaattcccgattaggatcttcctagagcatggctacgtagataagtagcatggcgggttaatcattaactaca

(19) United States
(12) Patent Application Publication
(10) Pub. No.: US 2026/0027236 A1
(43) Pub. Date: Jan. 29, 2026

(54) GUIDE RNAS, VECTORS, AND VIRIONS FOR TARGETING MUTATIONS IN THE PLN GENE

(71) Applicant: Tenaya Therapeutics, Inc., South San Francisco, CA (US)

(72) Inventor: Huanyu ZHOU, San Mateo, CA (US)

(21) Appl. No.: 19/348,088

(22) Filed: Oct. 2, 2025

Related U.S. Application Data
(63) Continuation of application No. PCT/US2024/023697, filed on Apr. 9, 2024.
(60) Provisional application No. 63/495,292, filed on Apr. 10, 2023.
(60) Provisional application No. 63/594,946, filed on Oct. 31, 2023.
(60) Provisional application No. 63/565,988, filed on Mar. 15, 2024.

(51) Int. Cl. A61K 48/00; A61K 31/7088; A61K 35/76; C12N 9/22; C12N 15/113; C12N 15/864; C12N 15/867

(52) U.S. Cl. CPC A61K 48/005; A61K 31/7088; A61K 35/76; C12N 9/226; C12N 15/113; C12N 15/8645; C12N 15/867; C12N 2310/20; C12N 2740/15043; C12N 2750/14143; C12N 2830/001

(57) ABSTRACT

The present disclosure provides gRNAs targeting the phospholamban (PLN) gene, expression cassettes, vectors, virions and compositions comprising the same, as well as methods useful for the treatment or prevention of heart disease. In some embodiments, the present disclosure provides expression cassettes and vectors comprising a polynucleotide encoding a Cas endonuclease protein operably linked to a protein expression-driving promoter (e.g., a human troponin T promoter) and/or a polynucleotide encoding a gRNA targeting a sequence of the PLN gene comprising a mutation or deletion operably linked to an RNA expression-driving promoter. The present disclosure also provides guide RNAs, expression cassettes, vectors, virions and compositions for specifically targeting PLN gene comprising a deletion of Arg14.

### CROSS-REFERENCE TO RELATED APPLICATIONS

This application is a Continuation of International Application No. PCT/US2024/023697, filed Apr. 9, 2024, which claims the benefit of U.S. Provisional Patent Application No. 63/495,292 filed Apr. 10, 2023, U.S. Provisional Patent Application No. 63/594,946 filed Oct. 31, 2023, and U.S. Provisional Patent Application No. 63/565,988 filed Mar. 15, 2024, the entire disclosures of each of which are incorporated herein by reference in their entireties.

### REFERENCE TO SEQUENCE LISTING

The present application is being filed electronically via USPTO Patent Center and includes an electronically submitted Sequence Listing in XML format. The contents of the electronic sequence listing (TENA_043_03US_SeqList_ST26.xml; Size: 222,493 bytes; and Date of Creation: Oct. 2, 2025) are herein incorporated by reference in its entirety.

### FIELD OF THE INVENTION

The present disclosure relates generally to guide RNAs targeting a mutant phospholamban (PLN) gene, expression cassettes, vectors, virions, and compositions comprising the same, as well as methods useful for editing the PLN gene and for the treatment or prevention of heart diseases associated with mutations in the PLN gene.

### BACKGROUND

Phospholamban (PLN) plays an important role in the regulation of sarcoendoplasmic reticulum (SR) calcium transport ATPase (SERCA), which uptakes Ca²⁺ to SR to maintain intracellular calcium homeostasis of cardiomyocytes. See Jiang et al., 2020, Scientific Reports 10: 16478. Disruption of PLN disrupts the calcium homeostasis in cardiomyocytes and myocardial contraction. Id. Mutations on PLN result in intracellular calcium disorder, myocardial contraction defect, heart failure, and/or malignant ventricular arrhythmia. Id. Several PLN mutations have been identified in dilated cardiomyopathy (DCM) and arrhythmogenic cardiomyopathy (ACM) patients, including PN-R14Del mutation, R9C (Arg to Cys), R9L, R9H, Leu-39stop, and R25C. Id. The PLN-R14Del mutation, involving a non-frameshift deletion of R at position 14 at the loci 6q22, exerts a dominant negative effect on normal PLNs, disrupting cardiomyocyte calcium homeostasis. Id. PLN-R14 del patients exhibit DCM or ACM characteristics, as well as female dominant morbidity, late onset heart failure, symptoms, early onset sudden death risks, a low voltage of QRS, and/or abnormal T wave. Id. R14del is a well-known mutation in Dutch, causing 10-15% of DCM and ACM. Id.

Gene therapy approaches are promising for the treatment of mutations that cause heart disease; however, this approach is not effective for all genetic causes of heart disease.

Several challenges exist to effectively target deleterious mutations. One challenge is to identify guide RNAs and vectors that achieve high editing efficiency. Another challenge is to successfully deliver polynucleotides encoding large proteins, such as Cas9 enzymes, due in part to the packaging limit of viral vectors. Additionally, using CRISPR/Cas9 methodology to target a specific, pathogenic mutation, such as the PLN-R14Del mutation, while not targeting the wild-type allele is an additional obstacle to overcome to ensure effective treatment. Given these challenges, there remains a need in the art for improved guide RNAs, vectors, virions, and methods for prevention and treatment of heart diseases, such as those associated with PLN mutations.

### SUMMARY

In some aspects, the present disclosure provides guide RNAs (gRNAs) for targeting phospholamban (PLN) gene mutations.

In some aspects, the present disclosure provides expression cassettes and/or vectors for targeting PLN gene mutations. In some embodiments, the expression cassettes and vectors comprise a first polynucleotide encoding a Cas endonuclease protein (e.g., Cas9 such as saCas9) operably linked to a protein-expression driving promoter (e.g., a human TNNT2 promoter), and/or a second polynucleotide encoding a guide RNA (gRNA) complementary to a sequence of the PLN gene comprising a mutation operably linked to an RNA expression-driving promoter (e.g., a U6 promoter). In some embodiments, the first polynucleotide and the second polynucleotide have a head-to-tail orientation in the same expression cassette or vector. In some embodiments, the gRNA is any gRNA targeting a mutant PLN (such as a deleterious mutant of PLN). In some embodiments, the gRNA targets a deletion of Arg14 in a PLN gene (“PLN-R14Del” mutation). In some embodiments, the gRNA is complementary to a sequence of the PLN gene comprising a deletion of Arg14. In some embodiments, the gRNA is any gRNA that can target a mutant PLN, e.g., PLN-R14Del mutation. In some embodiments, gRNAs provided herein can target a mutant PLN with high editing efficiency of indel formation (e.g., equal to or more than 30%, 40% or 50%). In some embodiments, the vector is a viral vector, such as any viral vector, e.g., AAV (e.g., AAV9).

In some aspects, the present disclosure provides virions (e.g., recombinant AAV or rAAV) comprising any gRNA, expression cassette and/or vector described herein. In some embodiments, provided herein are AAV virions, comprising any gRNA, expression cassette and/or vector described herein and a capsid protein. In some embodiments, AAV is AAV9 or a variant thereof. In some embodiments, the capsid protein is wild-type AAV9 capsid protein or a variant thereof (such as any variant capsid protein described herein).

In some aspects, the present disclosure provides pharmaceutical compositions comprising any gRNA, expression cassette, vector and/or virion described herein, and a pharmaceutically acceptable carrier.

In some aspects, the disclosure provides cells compositions comprising any gRNA, expression cassette, vector and/or virion described herein. In some aspects, the disclosure provides a method of transducing an iPSC cell or a cardiac cell (e.g., a cardiomyocyte) with any gRNA, expression cassette, vector and/or virion described herein, wherein the cell expresses the polynucleotide transgene(s).

In some aspects, the disclosure provides methods of treating or preventing a disease or condition (e.g., a cardiac disease) in a subject (e.g., a mammal such as a human) with a mutation in a PLN gene comprising administering to the subject a gRNA targeting the mutation (e.g., a deletion) in the PLN gene (e.g., a deleterious mutation). In some embodiments, the gRNA targets PLN-R14Del mutation in the PLN gene. In some embodiments, the gRNA is any gRNA described herein. In some embodiments, the method further comprises administering a Cas endonuclease, for example a polynucleotide encoding a Cas endonuclease (e.g., Cas9 such as saCas9). In some embodiments, the gRNA is administered in a vector, virion or composition. In some embodiments, the Cas endonuclease is administered in the same vector, virion or composition as the gRNA. In some embodiments, a vector is administered which comprises a polynucleotide encoding PLN mutant-targeting gRNA operably linked to an RNA expression-driving promoter (e.g., U6 or any other such promoter described herein). In some embodiments, a vector is administered which comprises a polynucleotide encoding a Cas endonuclease (e.g., Cas9 such as saCas9), which optionally further comprises a polynucleotide encoding PLN mutant-targeting gRNA as described herein. In some embodiments, the polynucleotide encoding Cas9 and the polynucleotide encoding gRNA have head-to-tail orientation in the same expression cassette or vector. The disease or condition can be any disease or condition associated with one or more PLN mutations (e.g., PLN-R14Del mutation). In some embodiments, the disease is cardiomyopathy (e.g., DCM, HCM, ACM, or ARVC), e.g., a cardiomyopathy associated with a PLN mutation. In some embodiments, the disease is arrhythmia, e.g., an arrhythmia associated with a PLN mutation. In some embodiments, the disease or condition is heart failure, e.g., heart failure associated with a PLN mutation. In some embodiments, the subject has been diagnosed with a mutation in the PLN gene (e.g., PLN-R14Del mutation).

In some embodiments, provided herein are guide RNAs for targeting a PLN gene comprising a deletion of Arg14, expression cassettes, vectors, virions compositions and kits comprising the same, and/or methods of treatment of a human with such a PLN-R14 deletion.

In some embodiments, the present invention provides a vector for specifically targeting a phospholamban (PLN) gene comprising a deletion of Arg14 comprising: (i) a first polynucleotide encoding a Cas endonuclease protein operably linked to a human troponin T promoter, (ii) a second polynucleotide encoding a guide RNA (gRNA) operably linked to an RNA expression-driving promoter, wherein the gRNA is complementary to a sequence of the PLN gene comprising a deletion of Arg14, and wherein the first polynucleotide and the second polynucleotide have head-to-tail orientation.

In some embodiments, the guide RNA comprises at least 16 nucleotides with up to 3 mismatches of the nucleotide sequence ggttgaggctcttatagctga (SEQ ID NO: 1), the first polynucleotide and the second polynucleotide have head-to-tail orientation, and optionally the mismatch(es) is not in the sequence of ttatagctga (SEQ ID NO: 127) within SEQ ID NO: 1. In some embodiments, the gRNA comprises at least 16, 17, 18 or 19 nucleotides of the nucleotide sequence ggttgaggctcttatagctga (SEQ ID NO: 1). In some embodiments, the gRNA comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), and tggttgaggctcttatagctga (SEQ ID NO: 4). In some embodiments, the gRNA comprises the nucleotide sequence of ggttgaggctcttatagctga (SEQ ID NO: 1). In some embodiments, the gRNA comprises the nucleotide sequence of SEQ ID NO: 5, SEQ ID NO: 6, SEQ ID NO: 7, or SEQ ID NO: 8.

In some embodiments, the RNA expression-driving promoter is a U6 promoter. In some embodiments, the U6 promoter is the human U6 promoter having the nucleotide sequence of SEQ ID NO: 22 or a variant thereof (e.g., having at last 90% or 95% identity thereto).

In some embodiments, the Cas endonuclease protein is saCas9. In some embodiments, the saCas9 comprises a nucleotide sequence having at least 70%, 80%, 85%, 90%, 95%, 98% or 100% sequence identity to the nucleotide sequence of SEQ ID NO: 10 and/or an amino acid sequence having at least 70%, 80% 85%, 90%, 95%, 98% or 100% sequence identity to the amino acid sequence of SEQ ID NO: 11. In some embodiments, the saCas9 comprises the nucleotide sequence of SEQ ID NO: 10 and/or the amino acid sequence of SEQ ID NO: 11.

In some embodiments, the human troponin T promoter has a nucleotide sequence having at least 70%, 80%, 85%, 90%, 95%, 98% or 100% sequence identity to the nucleotide sequence of SEQ ID NO: 12. In some embodiments, the human troponin T promoter has the nucleotide sequence of SEQ ID NO: 12. In some embodiments, the human troponin T promoter has a nucleotide sequence having at least 70%, 80%, 85%, 90%, 95%, 98% or 100% sequence identity to the nucleotide sequence of SEQ ID NO: 13. In some embodiments, the human troponin T promoter has the nucleotide sequence of SEQ ID NO: 13. In some embodiments, the human troponin T promoter has a nucleotide sequence having at least 70%, 80%, 85%, 90%, 95%, 98% or 100% sequence identity to the nucleotide sequence of SEQ ID NO: 14.

In some embodiments, the vector of the present invention comprises the following 5′ to 3′ arrangement of elements: 5′-the human troponin T promoter-the polynucleotide encoding the Cas endonuclease protein-the RNA expression-driving promoter-the polynucleotide encoding gRNA-3′. In some embodiments, the vector comprises the following 5′ to 3′ arrangement of elements: 5′-the human troponin T promoter-polynucleotide encoding saCas9-human U6 promoter-the polynucleotide encoding gRNA-3′.

In some embodiments, the vector of the present invention comprises a polyadenylation sequence. In some embodiments, the polyadenylation sequence is selected from a BGH polyadenylation sequence and a SV40 polyadenylation sequence. In some embodiments, the polyadenylation sequence has at least 80%, 85%, 90%, 95%, 98% or 100% identity to SEQ ID NO: 29 or SEQ ID NO: 30.

In some embodiments, the polyadenylation sequence is comprised in the vector between the polynucleotide encoding the Cas endonuclease protein and the sequence of the RNA expression-driving promoter, or wherein the vector comprises the following 5′ to 3′ arrangement of elements: 5′-the human troponin T promoter-the polynucleotide encoding the Cas endonuclease protein-the polyadenylation sequence-the RNA expression-driving promoter-the polynucleotide encoding gRNA-3′.

In some embodiments, the vector of the present invention is a DNA-based vector, an mRNA-based vector, an adeno-associated virus-based vector, a retrovirus-based vector, or a lentivirus-based vector. In some embodiments, the vector is an adeno-associated virus vector, a retroviral vector, or a lentiviral vector. In some embodiments, the vector is an AAV9 vector. In some embodiments, an rAAV virion comprises a wild-type AAV capsid protein (e.g., wild-type AAV9 capsid protein). In some embodiments, the vector is a modified adeno-associated virus vector. In some embodiments, an rAAV virion comprises a modified capsid protein (e.g., a modified AAV9 capsid protein, such as any of the modified capsid proteins described herein.

In some embodiments, the vector of the present invention, when introduced into iPSC-derived cardiomyocytes, has editing efficiency of, or efficiency of indel formation in, the PLN gene comprising a deletion of Arg14 of at least 20%, 25%, 30%, 35%, 40%, 45%, 50%, 55%, 60% or 70%. In some embodiments, the vector has editing efficiency of, or efficiency of indel formation in, the PLN gene comprising a deletion of Arg14 of at least 50% or at least 70%. In some embodiments, the vector does not, or substantially does not, edit or form indels in the wild-type PLN gene.

In some embodiments, the present invention provides a guide RNA for specifically targeting a phospholamban (PLN) gene comprising a deletion of Arg14 comprising the nucleotide sequence of SEQ ID NO: 5, the nucleotide sequence of SEQ ID NO: 6, the nucleotide sequence of SEQ ID NO: 7, or the nucleotide sequence of SEQ ID NO: 8.

In some embodiments, the present invention provides a gene editing composition or kit comprising the guide RNA of the previous paragraph and saCas9, optionally wherein the saCas9 comprises the nucleotide sequence of SEQ ID NO: 10 and/or the amino acid sequence of SEQ ID NO: 11.

In some embodiments, the present invention provides a vector for specifically targeting a phospholamban (PLN) gene comprising a deletion of Arg14 comprising: (i) a polynucleotide encoding a Cas endonuclease protein, operably linked to a protein expression-driving promoter, and (ii) a polynucleotide encoding the guide RNA (such as any gRNA described herein), operably linked to an RNA expression-driving promoter. In some embodiments, the Cas endonuclease protein is saCas9, the protein expression-driving promoter is human troponin T promoter, and/or the RNA expression-driving promoter is human U6 promoter. In some embodiments, the vector, when introduced into iPSC-derived cardiomyocytes, has editing efficiency of, or efficiency of indel formation in, the PLN gene comprising a deletion of Arg14 of at least 20%, 25%, 30%, 35%, 40%, 45%, 50%, 55%, 60% or 70%. In some embodiments, the vector has editing efficiency of, or efficiency of indel formation in, the PLN gene comprising a deletion of Arg14 of at least 50% or at least 70%. In some embodiments, the vector does not, or substantially does not, edit or form indels in the wild-type PLN gene.

In some embodiments, the present invention provides a recombinant adeno-associated virus (AAV) virion comprising any of the vectors described herein and a capsid protein, wherein the capsid protein is any of the capsid proteins described herein or known in the art (such as wild-type capsid proteins and variants thereof). In some embodiments, the capsid protein is a wild-type AAV9 capsid protein. In some embodiments, the capsid protein is an engineered or modified AAV9 capsid protein, such as any of the capsid proteins described herein.

In some embodiments, the present invention provides a pharmaceutical composition comprising any one of the vectors described herein. In some embodiments, the present invention provides a pharmaceutical composition comprising any one of the virions (e.g., rAAV) described herein.

In some embodiments, the present invention provides a method of treating or preventing a disease or condition in a human with deletion of Arg14 in a phospholamban (PLN) gene comprising administering to the human a therapeutically effective amount of a composition, vector or virion comprising a guide RNA (gRNA), wherein the gRNA comprises at least 16 nucleotides with up to 3 mismatches of the nucleotide sequence ggttgaggctcttatagctga (SEQ ID NO: 1), optionally wherein the mismatch(es) is not in the sequence of ttatagctga (SEQ ID NO: 127) within SEQ ID NO: 1. In some embodiments, the gRNA comprises at least 16, 17, 18 or 19 nucleotides of the nucleotide sequence ggttgaggctcttatagctga (SEQ ID NO: 1). In some embodiments, the gRNA comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), and tggttgaggctcttatagctga (SEQ ID NO: 4). In some embodiments, the gRNA comprises the nucleotide sequence of ggttgaggctcttatagctga (SEQ ID NO: 1). In some embodiments, the gRNA comprises the nucleotide sequence of SEQ ID NO: 5, SEQ ID NO: 6, SEQ ID NO: 7, or SEQ ID NO: 8. In some embodiments, the gRNA comprises the nucleotide sequence of SEQ ID NO: 5.

In some embodiments, the method comprises administering the gRNA in the vector, and the vector comprises: (i) a first polynucleotide encoding a Cas endonuclease protein, operably linked to a protein expression-driving promoter, and (ii) a second polynucleotide encoding the gRNA, operably linked to an RNA expression-driving promoter. In some embodiments, the Cas endonuclease protein is saCas9. In some embodiments, the saCas9 comprises a nucleotide sequence having at least 70%, 85%, 90%, 95% or 98% sequence identity to the nucleotide sequence of SEQ ID NO: 10 and/or an amino acid sequence having at least 70%, 85%, 90%, 95% or 98% sequence identity to the amino acid sequence of SEQ ID NO: 11. In some embodiments, the saCas9 comprises the nucleotide sequence of SEQ ID NO: 10 and/or the amino acid sequence of SEQ ID NO: 11.

In some embodiments, the protein expression-driving promoter in the provided method is a cardiac cell-specific or cardiomyocyte-specific promoter. In some embodiments, the protein expression-driving promoter is a human troponin T promoter. In some embodiments, the human troponin T promoter has a nucleotide sequence having at least 70%, 80%, 85%, 90%, 95%, 98% or 100% sequence identity to the nucleotide sequence of SEQ ID NO: 12. In some embodiments, the human troponin T promoter has the nucleotide sequence of SEQ ID NO: 12. In some embodiments, the human troponin T promoter has a nucleotide sequence having at least 70%, 80%, 85%, 90%, 95%, 98% or 100% sequence identity to the nucleotide sequence of SEQ ID NO: 13. In some embodiments, the human troponin T promoter has the nucleotide sequence of SEQ ID NO: 13. In some embodiments, the human troponin T promoter has a nucleotide sequence having at least 70%, 80%, 85%, 90%, 95%, 98% or 100% sequence identity to the nucleotide sequence of SEQ ID NO: 14.

In some embodiments, the RNA expression-driving promoter is a U6 promoter. In some embodiments, the U6 promoter is a human U6 promoter having the nucleotide sequence of SEQ ID NO: 22 or a variant thereof (e.g., having at last 90% or 95% identity thereto).

In some embodiments of the provided method, the Cas endonuclease protein is saCas9, the protein expression-driving promoter is human troponin T promoter, and the RNA expression-driving promoter is human U6 promoter.

In some embodiments of the provided method, the vector is a DNA-based vector, an mRNA-based vector, an adeno-associated virus-based vector, a retrovirus-based vector, or a lentivirus-based vector. In some embodiments, the vector is an adeno-associated virus vector, a retroviral vector, or a lentiviral vector. In some embodiments, the vector is an AAV9 vector. In some embodiments, an rAAV virion comprises a wild-type AAV capsid protein (e.g., wild-type AAV9 capsid protein). In some embodiments, the vector is a modified adeno-associated virus vector. In some embodiments, an rAAV virion comprises a modified capsid protein (e.g., a modified AAV9 capsid protein, such as any of the modified capsid proteins described herein).

In some embodiments, the present invention provides a method of treating or preventing a disease or condition in a subject (e.g., a human) with deletion of Arg14 in a phospholamban (PLN) gene comprising administering to the human a vector, a virion or a composition described herein. In some embodiments, the disease or condition is a cardiac disease or condition. In some embodiments, the cardiac disease or condition is cardiomyopathy (e.g., DCM, HCM, ACM or ARVC). In some embodiments, the cardiac disease or condition is heart failure. In some embodiments, the cardiac disease or condition is malignant ventricular tachycardia. In some embodiments, the cardiac disease or condition is arrhythmia.

In some embodiments, the provided method improves one or more measures of cardiac function. In some embodiments, the method increases ejection fraction. In some embodiments, the method reduces left ventricular internal dimension and/or left ventricular mass.

In some embodiments, the subject (e.g., a human) has a heterozygous deletion of Arg14 in a phospholamban (PLN) gene.

In some embodiments, the administering is systemic administration or local administration to the heart. In some embodiments, the systemic administration is intravenous administration. In some embodiments, the local administration is by direct injection into the heart or cardiac tissue, intracoronary administration or retrograde coronary sinus infusion.

In some embodiments of the provided method, the administering results in editing efficiency of, or efficiency of indel formation in, the PLN gene comprising a deletion of Arg14 of at least 20%, 25%, 30%, 35%, 40%, 45%, 50%, 55%, 60% or 70%. In some embodiments, the administering results in editing efficiency of, or efficiency of indel formation in, the PLN gene comprising a deletion of Arg14 of at least 50% or at least 70%. In some embodiments, the method does not, or substantially does not, edit or form indels in the wild-type PLN gene.

In some embodiments of the provided method, the vector is administered at a dose in the range of about 1×10¹³ vector genomes (vg) per kg to about 1×10¹⁵ vector genomes (vg) per kg. In some embodiments of the provided method, the vector is administered at a dose in the range of about 1×10¹² vector genomes (vg) per kg to about 1×10¹⁴ vector genomes (vg) per kg. In some embodiments of the provided method, the vector is administered at a dose of less than about 1×10¹⁴ vector genomes (vg) per kg, or less than about 1×10¹³ vector genomes (vg) per kg.

In some aspects, the present disclosure provides self-inactivating expression cassettes and/or vectors for targeting a genomic locus, such as a gene, e.g., a PLN gene or a PLN gene comprising a Arg14 deletion. In some embodiments, the expression cassette or vector comprises (i) a first polynucleotide encoding a Cas endonuclease protein operably linked to a human troponin T promoter; and (ii) a second polynucleotide encoding a guide RNA (gRNA) operably linked to an RNA expression-driving promoter, wherein the gRNA is complementary to a sequence of the PLN gene comprising a deletion of Arg14; and (iii) a self-inactivation site, wherein the first polynucleotide and the second polynucleotide have a head-to-tail orientation.

In some embodiments, the Cas endonuclease protein is saCas9. In some embodiments, the saCas9 comprises a nucleotide sequence having at least 70%, 85%, 90%, 95% or 98% identity to SEQ ID NO: 10 and/or an amino acid sequence having at least 70%, 85%, 90%, 95% or 98% identity to SEQ ID NO: 11. In some embodiments, the saCas9 comprises the nucleotide sequence of SEQ ID NO: 10 and/or the amino acid sequence of SEQ ID NO: 11.

In some embodiments, the human troponin T promoter has a nucleotide sequence having at least 70%, 80%, 85%, 90%, 95% or 98% identity to SEQ ID NO: 12, SEQ ID NO: 13, or SEQ ID NO: 14. In some embodiments, the human troponin T promoter has the nucleotide sequence of SEQ ID NO: 12, SEQ ID NO: 13, or SEQ ID NO: 14.

In some embodiments, the gRNA comprises at least 16 nucleotides with up to 3 mismatches of the nucleotide sequence ggttgaggctcttatagctga (SEQ ID NO: 1), optionally wherein the mismatch(es) is not in the sequence of ttatagctga (SEQ ID NO: 127) within SEQ ID NO: 1. In some embodiments, the gRNA comprises at least 16, 17, 18 or 19 nucleotides of the nucleotide sequence ggttgaggctcttatagctga (SEQ ID NO: 1). In some embodiments, the gRNA comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), and tggttgaggctcttatagctga (SEQ ID NO: 4). In some embodiments, the gRNA comprises the nucleotide sequence of ggttgaggctcttatagctga (SEQ ID NO: 1). In some embodiments, the gRNA comprises the nucleotide sequence of SEQ ID NO: 5, SEQ ID NO: 6, SEQ ID NO: 7, or SEQ ID NO: 8.

In some embodiments, the RNA expression-driving promoter is a U6 promoter. In some embodiments, the U6 promoter is a human U6 promoter having the nucleotide sequence of SEQ ID NO: 22.

In some embodiments, the self-inactivation site is 5′ to the human troponin T promoter, within the human troponin T promoter, between the human troponin T promoter and the first polynucleotide encoding the Cas endonuclease protein, within the first polynucleotide encoding the Cas endonuclease protein, or 3′ to the first polynucleotide encoding the Cas endonuclease protein. In some embodiments, the self-inactivation site is within the first polynucleotide encoding the Cas endonuclease protein, optionally near the 5′ end of the first polynucleotide encoding the Cas endonuclease protein, further optionally after the start codon “ATG”.

In some embodiments, the self-inactivation site comprises a gRNA target region. In some embodiments, the gRNA target region is recognized by the gRNA encoded by the second polynucleotide. In some embodiments, the gRNA target region comprises at least 16 nucleotides with up to 3 mismatches of the nucleotide sequence ggttgaggctcttatagctga (SEQ ID NO: 1), or a nucleotide sequence reverse complement to at least 16 nucleotides with up to 3 mismatches of the nucleotide sequence ggttgaggctcttatagctga (SEQ ID NO: 1), optionally wherein the mismatch(es) is not in the sequence of ttatagctga (SEQ ID NO: 127) within SEQ ID NO: 1. In some embodiments, the gRNA target region comprises at least 16, 17, 18 or 19 nucleotides of the nucleotide sequence ggttgaggctcttatagctga (SEQ ID NO: 1), or a nucleotide sequence reverse complement to at least 16, 17, 18 or 19 nucleotides of the nucleotide sequence ggttgaggctcttatagctga (SEQ ID NO: 1). In some embodiments, the gRNA target region comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), tggttgaggctcttatagctga (SEQ ID NO: 4), and a nucleotide sequence reverse complement to any one of the foregoing. In some embodiments, the gRNA target region comprises the nucleotide sequence of ggttgaggctcttatagctga (SEQ ID NO: 1), or a nucleotide sequence reverse complement to ggttgaggctcttatagctga (SEQ ID NO: 1).

In some embodiments, the self-inactivation site further comprises a less optimal PAM sequence of the Cas endonuclease protein encoded by the first polynucleotide. In some embodiments, when the Cas endonuclease protein is saCas9, the less optimal PAM sequence is NNGRRC, NNGRRG, or NNGRRA, wherein N is any nucleotide, and R is guanine or adenine. In some embodiments, the less optimal PAM sequence is gcgagc, gcgaga, or gcgagg.

In some embodiments, the vector comprises the following 5′ to 3′ arrangement of elements: 5′-the human troponin T promoter-the first polynucleotide encoding the Cas endonuclease protein-the RNA expression-driving promoter-the second polynucleotide encoding the gRNA-3′, wherein the self-inactivation site is within the first polynucleotide encoding the Cas endonuclease protein, optionally near the 5′ end of the first polynucleotide encoding the Cas endonuclease protein, further optionally after the start codon “ATG”.

In some embodiments, the vector further comprises a polyadenylation sequence. In some embodiments, the polyadenylation sequence is selected from a BGH polyadenylation sequence and a SV40 polyadenylation sequence. In some embodiments, the BGH polyadenylation sequence has at least 90%, 95%, 98% or 100% identity to SEQ ID NO: 29, and/or the SV40 polyadenylation sequence has at least 90%, 95%, 98% or 100% identity to SEQ ID NO: 30.

In some embodiments, the polyadenylation sequence is between the first polynucleotide encoding the Cas endonuclease protein and the RNA expression-driving promoter, or wherein the vector comprises the following 5′ to 3′ arrangement of elements: 5′-the human troponin T promoter-the first polynucleotide encoding the Cas endonuclease protein-the polyadenylation sequence-the RNA expression-driving promoter-the second polynucleotide encoding the gRNA-3′.

In some embodiments, the vector is a DNA-based vector, an mRNA-based vector, an adeno-associated virus-based vector, a retrovirus-based vector, or a lentivirus-based vector. In some embodiments, the vector is an adeno-associated virus vector, a retroviral vector, or a lentiviral vector. In some embodiments, the vector is an AAV9 vector. In some embodiments, the vector is a modified adeno-associated virus vector. In some embodiments, the modified adeno-associated virus comprises any capsid protein described herein, optionally wherein the capsid protein is wild-type AAV9 capsid protein or a variant thereof.

In some embodiments, the expression cassette or vector, when introduced into iPSC-derived cardiomyocytes, has an at least 20%, 25%, 30%, 35%, 40%, 45%, 50%, 55%, 60% or 70% editing efficiency of, or efficiency of indel formation in, the PLN gene comprising a deletion of Arg14. In some embodiments, the expression cassette or vector, when introduced into iPSC-derived cardiomyocytes, has an at least 50% or at least 70% editing efficiency of, or efficiency of indel formation in, the PLN gene comprising a deletion of Arg14. In some embodiments, the expression cassette or vector, when introduced into iPSC-derived cardiomyocytes, does not, or substantially does not, edit or form indels in the wild-type PLN gene. In some embodiments, expression of the Cas endonuclease protein is terminated within about 4-8 weeks after introduced into iPSC-derived cardiomyocytes.

In some embodiments, the expression cassette and/or vector according to various embodiments disclosed herein is in a pharmaceutical composition.

In some aspects, the present disclosure provides methods of treating or preventing a disease or condition in a human associated with a genetic mutation, e.g., a PLN gene with a Arg14 deletion, the methods comprising administering to the human an expression cassette, vector, and/or pharmaceutical composition containing the same according to various embodiments disclosed herein. In some embodiments, the disease or condition is a cardiac disease or condition, for example, cardiomyopathy (e.g., dilated cardiomyopathy, hypertrophic cardiomyopathy, arrhythmogenic cardiomyopathy, and/or arrhythmogenic right ventricular cardiomyopathy), heart failure, malignant ventricular tachycardia, or arrhythmia. In some embodiments, the method improves one or more measures of cardiac function, for example, increases ejection fraction, and/or reduces left ventricular internal dimension and/or left ventricular mass.

In some embodiments, the administering is systemic administration or local administration to the heart, for example, intravenous administration or by direct injection into the heart or cardiac tissue, intracoronary administration or retrograde coronary sinus infusion. In some embodiments, the administering results in an at least 20%, 25%, 30%, 35%, 40%, 45%, 50%, 55%, 60% or 70% editing efficiency of, or efficiency of indel formation in, the PLN gene comprising a deletion of Arg14. In some embodiments, the administering results in an at least 50% or at least 70% editing efficiency of, or efficiency of indel formation in, the PLN gene comprising a deletion of Arg14.

In some embodiments, the expression cassette, vector, and/or pharmaceutical composition containing the same does not, or substantially does not, edit or form indels in the wild-type PLN gene. In some embodiments, expression of the Cas endonuclease protein is terminated within about 4-8 weeks after the administering to the human.

In some embodiments, the vector is administered at a dose in the range of about 1×10¹² vector genomes (vg) per kg to about 1×10¹⁴ vector genomes (vg) per kg, less than about 1×10¹⁴ vector genomes (vg) per kg, or less than about 1×10¹³ vector genomes (vg) per kg.

In some aspects, the present disclosure provides self-inactivating expression cassettes and/or vectors for specifically targeting a gene of interest, wherein the self-inactivating expression cassette or vector comprises (i) a first polynucleotide encoding a Cas endonuclease protein operably linked to a protein expression-driving promoter; (ii) a second polynucleotide encoding a guide RNA (gRNA) operably linked to an RNA expression-driving promoter, wherein the gRNA is complementary to a sequence of the gene of interest; and (iii) a self-inactivation site, optionally wherein the first polynucleotide and the second polynucleotide have a head-to-tail orientation.

In some embodiments, the Cas endonuclease protein is saCas9. In some embodiments, the saCas9 comprises a nucleotide sequence having at least 70%, 85%, 90%, 95% or 98% identity to SEQ ID NO: 10 and/or an amino acid sequence having at least 70%, 85%, 90%, 95% or 98% identity to SEQ ID NO: 11. In some embodiments, the saCas9 comprises the nucleotide sequence of SEQ ID NO: 10 and/or the amino acid sequence of SEQ ID NO: 11.

In some embodiments, the RNA expression-driving promoter is a U6 promoter. In some embodiments, the U6 promoter is a human U6 promoter having the nucleotide sequence of SEQ ID NO: 22.

In some embodiments, the self-inactivation site is 5′ to the protein expression-driving promoter, within the protein expression-driving promoter, between the protein expression-driving promoter and the first polynucleotide encoding the Cas endonuclease protein, within the first polynucleotide encoding the Cas endonuclease protein, or 3′ to the first polynucleotide encoding the Cas endonuclease protein. In some embodiments, the self-inactivation site is within the first polynucleotide encoding the Cas endonuclease protein, optionally near the 5′ end of the first polynucleotide encoding the Cas endonuclease protein, further optionally after the start codon “ATG”.

In some embodiments, the self-inactivation site comprises a gRNA target region. In some embodiments, the gRNA target region is recognized by the gRNA encoded by the second polynucleotide. In some embodiments, the gRNA target region comprises the gRNA sequence or a portion thereof, or a nucleotide sequence reverse complement to the gRNA sequence or a portion thereof.

In some embodiments, the self-inactivation site further comprises a less optimal PAM sequence of the Cas endonuclease protein encoded by the first polynucleotide. In some embodiments, when the Cas endonuclease protein is saCas9, the less optimal PAM sequence is NNGRRC, NNGRRG, or NNGRRA, wherein N is any nucleotide, and R is guanine or adenine. In some embodiments, the less optimal PAM sequence is gcgagc, gcgaga, or gcgagg.

In some embodiments, the vector comprises the following 5′ to 3′ arrangement of elements: 5′-the protein expression-driving promoter-the first polynucleotide encoding the Cas endonuclease protein-the RNA expression-driving promoter-the second polynucleotide encoding the gRNA-3′, wherein the self-inactivation site is within the first polynucleotide encoding the Cas endonuclease protein, optionally near the 5′ end of the first polynucleotide encoding the Cas endonuclease protein, further optionally after the start codon “ATG”.

In some embodiments, the vector is a DNA-based vector, an mRNA-based vector, an adeno-associated virus-based vector, a retrovirus-based vector, or a lentivirus-based vector. In some embodiments, the vector is an adeno-associated virus vector, a retroviral vector, or a lentiviral vector. In some embodiments, the vector is an AAV9 vector. In some embodiments, the vector is a modified adeno-associated virus vector. In some embodiments, the modified adeno-associated virus comprises any capsid protein described herein, optionally wherein the capsid protein is wild-type AAV9 capsid protein or a variant thereof.

In some embodiments, expression of the Cas endonuclease protein is terminated within about 4-8 weeks after the vector is introduced into a cell.

In some embodiments, the gene of interest is selected from the group consisting of phospholamban (PLN), cardiac troponin T (TNNT2), BAG family molecular chaperone regulator 3 (BAG3), myosin heavy chain (MYH7), tropomyosin 1 (TPM1), myosin binding protein C (MYBPC3), 5′-AMP-activated protein kinase subunit gamma-2 (PRKAG2), troponin I type 3 (TNNI3), titin (TTN), myosin light chain 2 (MYL2), actin, alpha cardiac muscle 1 (ACTC1), potassium voltage-gated channel, KQT-like subfamily, member 1 (KCNQ1), myocyte enhancer factor 2c (MEF2C), cardiac LIM protein (CSRP3), DWORF, junctophilin (JPH2), alpha-crystallin B chain (CRYAB), LMNA (Lamin A and Lamin C isoforms), troponin I type 3 (TNNI3), lysosomal-associated membrane protein 2 (LAMP2, including LAMP2a, LAMP2b and LAMP2c isoforms), desmoplakin (DSP, including DPI and DPII isoforms), desmoglein 2 (DSG2), junction plakoglobin (JUP), plakophilin-2 (PKP2), matrix metallopeptidase 11 (MMP11), synaptopodin 2 like (SYNPO2L, including SYNPO2LA and SYNPO2LB), RNA binding motif protein 20 (RBM20), metastasis suppressor protein 1 (MTSS1), proprotein convertase subtilisin/kexin type 9 (PCSK9), acid alpha-glucosidase (GAA), and frataxin (FXN).

In some embodiments, the expression cassette and/or vector according to various embodiments disclosed herein is in a pharmaceutical composition.

In some aspects, the present disclosure provides methods of editing a gene of interest in a cell, the method comprising introducing to the cell an expression cassette or vector according to various embodiments disclosed herein.

In some embodiments, the expression cassette and/or vector according to various embodiments disclosed herein is in a pharmaceutical composition. In some aspects, the present disclosure provides methods of expression of the Cas endonuclease protein is terminated within about 4-8 weeks after introducing the vector to the cell.

### BRIEF DESCRIPTION OF THE FIGURES

FIG. 1A and FIG. 1B show diagrams of PCR amplification of either a pathogenic PLN allele (FIG. 1A) or a WT PLN allele (FIG. 1B). FIG. 1C shows results of an agarose gel electrophoresis of the resulting PCR products from FIG. 1A and FIG. 1B.

FIG. 2A is schematic showing the design of a PLN R14del specific gRNA. FIG. 2B is a schematic of a retroviral saCas9 construct (RV-pHZ132) that encodes the gRNA of FIG. 2A. The construct is 6,381 bp measured from 5′ long-terminal repeat (LTR) to 3′ LTR. FIG. 2C is a schematic of an experiment in which the retroviral saCas9/gRNA vector (RV-pHZ132) or retroviral dsRed vector (RV-dsRed) was used to edit mouse embryonic fibroblasts (MEFs) from PLN R14del homozygous mice with the pathogenic allele or PLN WT mice. FIG. 2D is an agarose gel showing the results of a T7 endonuclease I (T7E1) assay, showing that RV-pHZ132 edited MEFs with the PLN R14del allele but not MEFs with the PLN WT allele. FIG. 2E is a table showing that RV-pHZ132 efficiently edited the PLN R14del allele but not the PLN WT allele. FIG. 2F shows the sequencing results of clones after treating the PLN R14del homozygotes MEFs with RV-pHZ132.

FIG. 3A is a schematic of the experimental design and gene editing outcomes of retroviral saCas9-sgRNA in PLN WT/R14del heterozygote MEFs. FIG. 3B is a table showing that RV-pHZ132 efficiently edited the PLN R14del allele but not the PLN WT allele in PLN WT/R14del heterozygote MEFs.

FIG. 4A is a diagram of AAV cassette pHZ131, which contains both saCas9 and a mouse PLN R14del-specific gRNA. FIG. 4B is a schematic of an in vivo PLN editing experiment in which PLN WT/R14del heterozygote mice were administered three different doses of AAV9-pHZ131 (1E13 vg/kg, 3E13 vg/kg, and 1E14 vg/kg). FIG. 4C is a table showing AAV9-pHZ132 selectively edited the PLN R14del allele in a dose-dependent manner while leaving the PLN WT allele unedited.

FIG. 5A is a graph showing disease progression through fractional shortening from weeks 2 to 7 in WT, PLN-R14^{WT/Δ} heterozygotes, and PLN-R14^{Δ/Δ} homozygotes. FIG. 5B is a table showing the experimental configuration and dosage of AAV9-pHZ131 used for gene editing in a PLN-R14^{Δ/Δ} (PLN-R14del homozygous) mouse model. HBSS or AAV9-pHZ131 was administered via retro-orbital injection in PLN-R14^{Δ/Δ} homozygous mice at three weeks of age, before disease onset.

FIG. 6A to FIG. 6C present the results of the experiment described in FIGS. 5A-5B.

FIGS. 6A-6C are graphs showing increased ejection fraction in a PLN-R14^{Δ/Δ} mouse model following treatment with AAV9-pHZ131, as compared to vehicle control (HBSS).

FIGS. 7A-7E are graphs showing improved LV internal dimension systole (LVIDs, FIG. 7A and FIG. 7B), LV internal dimension diastole (LVIDd, FIG. 7C), left ventricular mass (LV Mass, FIG. 7D), and stroke volume (SV, FIG. 7E) respectively, in a PLN-R14^{Δ/Δ} mouse model after treatment with AAV9-pHZ131 (PLNR14del pHZ131), as compared to vehicle control (PLNR14del HBSS).

FIGS. 8A-8D are graphs showing improved R amplitude (FIGS. 8A-8C) and QRS interval (FIG. 8D) in a PLN-R14^{Δ/Δ} mouse model following treatment with AAV9-pHZ131 (PLNR14del pHZ131), as compared to vehicle control (PLNR14del HBSS)

FIG. 9A is a graph showing decreased mortality in a PLN-R14^{Δ/Δ} mouse model following treatment with AAV9-pHZ131 (PLNR14Del-pHZ131), as compared to vehicle control (PLNR14-Del HBSS). PLNR14Del-pHZ131 line is superimposed upon WT HBSS line.

FIG. 9B is a graph of body weight from 4 to 7 weeks of age in a PLN-R14^{Δ/Δ} mouse model, following treatment with AAV9-pHZ131 (PLNR14Del pHZ131), as compared to vehicle control (PLNR14Del HBSS)

FIG. 9C is a graph of the change in body weight between weeks 6.5 and 7 in a PLN-R14^{Δ/Δ} mouse model, following treatment with AAV9-pHZ131 (PLNR14Del pHZ131), as compared to vehicle control (PLNR14Del HBSS).

FIG. 10A is a table showing the experimental configuration and three dosages of with AAV9-pHZ131 used for gene editing in a PLN-R14^{Δ/Δ} (PLN-R14del homozygous) mouse model. HBSS or AAV9-pHZ131 was administered via retro-orbital injection in PLN-R14^{Δ/Δ} homozygous mice at three weeks of age, before disease onset.

FIGS. 10B-10E are graphs showing increased ejection fraction (FIG. 10B), improved LV internal dimension systole (LVIDs, FIG. 10C), improved R amplitude (FIG. 10D), and decreased mortality (FIG. 10E) in a PLN-R14^{Δ/Δ} mouse model following treatment with various doses of AAV9-pHZ131(PLN R14Del 1E14 pHZ131, PLN R14Del 1E13 pHZ131, and PLN R14Del 3E13 pHZ131), as compared to vehicle control (PLNR14Del vehicle or PLNR14Del HBSS). In FIG. 10E, the lines for PLN R14Del 1E14 pHZ131, PLN R14Del 1E13 pHZ131, and PLN R14Del 3E13 pHZ131 are superimposed upon the WT HBSS line.

FIG. 10F and FIG. 10G show trichrome staining (FIG. 10F) and DAP staining (FIG. 10G) of heart tissue from 2-month-old mice (5 weeks after administration of AAV9-pHZ131) showing reduced cardiac fibrosis (FIG. 10F) and reduced PLN protein aggregation (FIG. 10G) following treatment with various doses of AAV9-pHZ131, as compared to vehicle control (HBSS).

FIG. 11A is a schematic that shows the gRNA design for human PLN R14del compared to mouse PLN R14del.

FIG. 11B is a schematic of the protocol for generation of human PLN R14del/R14del iPSCs and human PLN −/− iPSCs over time (bottom row of table indicates day).

FIG. 11C is a schematic of the protocol for generating cardiomyocytes from human iPSCs.

FIG. 11D is a diagram of AAV cassette pHZ128, which contains both saCas9 and a human PLN R14del-specific gRNA from FIG. 11A.

FIG. 11E and FIG. 11F show a schematic of the experimental design and results of gene editing by AAVCR9-1-pHZ128 in PLN WT/WT and R14del/R14del human iPSC-CMs. AAVCR9-1-pHZ1128 selectively edited the PLN R14del allele while leaving the PLN WT allele unedited.

FIG. 12 is a plasmid map of pHZ128_cTnT-saCas9-U6-hPLNR14del SagRNA21nt, which comprises the human hPLNR14del SagRNA 21nt and SaCas9 in an AAV2 expression cassette.

FIG. 13 is a plasmid map of pHZ131_cTnT-saCas9-U6-hPLNR14del SagRNA21nt which comprises the mouse hPLNR14del SagRNA 21nt and SaCas9 in an AAV2 expression cassette.

FIG. 14 is a plasmid map of pHZ132_cTnT-saCas9-U6-hPLNR14del SagRNA21nt which comprises the mouse hPLNR14del SagRNA 21nt and SaCas9 in an MMLV expression cassette

FIG. 15A is a schematic showing the design of a self-inactivation site for a self-inactivating Cas construct. The self-inactivation site comprises a gRNA target region and a less optimal PAM sequence. As a result, gene editing of the genome target site, which is usually designed to be near an optimal PAM sequence, is more efficient; gene editing of the AAV gnome, which leads to self-inactivation of the Cas construct, is less efficient. This is reflected in the first and second waves of editing shown in the right panel.

FIG. 15B is a diagram of a self-inactivating Cas cassette, which contains saCas9 and a gRNA, and the saCas9 encoding sequence contains a self-inactivation site. The gRNA directs Cas-dependent cutting of the target human genome site first and the expression cassette at the self-inactivation site second.

FIG. 16 is a diagram showing an exemplary design of a self-inactivating Cas cassette. The self-inactivation site located at the 5′ end of the Cas9 coding sequence, after the start codon “ATG,” and comprises a gRNA target site that is identical to the human PLN 14del gRNA target site within the human genome. Thus, the gRNA encoded by the expression cassette can recognize both the genome target site and the gRNA target site within the self-inactivation site. The difference is that the genome target site is next to an optimal PAM sequence, making it more efficient for the Cas protein to cut, and the gRNA target site is next to a less optimal PAM sequence within the self-inactivation site, making it less efficient for the Cas protein to cut.

FIG. 17A and FIG. 17B show the design and sequences of a constitutive Cas construct (TNGE101), a self-inactivating Cas construct with an optimal PAM sequence (TNGE102), and self-inactivating Cas constructs with less optimal PAM sequences (TNGE103-TNGE105) targeting human (h, FIG. 17A) and mouse (m, FIG. 17B) PLN 14del.

FIG. 18A is a schematic of an experiment where mice were injected via retro-orbital (RO) injection with either a vehicle control (HBSS), the constitutive Cas construct (TNGE101), or each of the self-inactivating Cas constructs (TNGE102-TNGE105) at a dose of 3E13 vg/kg.

Both human and mouse constructs were tested. Four weeks post injection, heart tissues were harvested for Western blot analysis to examine the level of Cas9 expression. FIG. 18B shows Western blot results for the experiment in FIG. 18A, in which Cas9 expression was only detected in the constitutive Cas construct (TNGE101) treatment.

FIG. 19A is a schematic of an in vivo PLN editing experiment where three-week-old PLN R14del/R14del homozygous mice were injected via RO injection with either a vehicle control (HBSS), the constitutive Cas construct (mTNGE101), or each of the self-inactivating Cas constructs (mTNGE102-mTNGE105) at a dose of 3E13 vg/kg. Four weeks post injection, cardiac function was assessed by echocardiography and electrocardiogram. FIGS. 19B-19E show cardiac function measurement results for the experiment in FIG. 19A, including ejection fraction (FIG. 19B), LVIDs (FIG. 19C), R amplitude (FIG. 19D), and QTc interval (FIG. 19E). FIG. 19F shows a measurement of survival rate of the mice from the experiment in FIG. 19A.

### DETAILED DESCRIPTION

In some aspects, the present disclosure provides guide RNAs (gRNAs) for targeting phospholamban (PLN) gene mutations, for example, for use in a gene editing system (e.g., the CRISPR/Cas system) to edit the PLN gene.

In some aspects, the present disclosure provides expression cassettes and/or vectors for targeting PLN gene mutations. In some embodiments, the expression cassettes and vectors comprise a first polynucleotide encoding a Cas endonuclease protein (e.g., Cas9 such as saCAs9) operably linked to a protein-expression driving promoter (e.g., a human TNNT2 promoter), and/or a second polynucleotide encoding a guide RNA (gRNA) complementary to a sequence of the PLN gene comprising a mutation operably linked to an RNA expression-driving promoter (e.g., a U6 promoter). In some embodiments, the first polynucleotide and the second polynucleotide have a head-to-head orientation in the same expression cassette or vector. In some embodiments, the first polynucleotide and the second polynucleotide have a head-to-tail orientation in the same expression cassette or vector. In some embodiments, the first polynucleotide and the second polynucleotide have a tail-to-tail orientation in the same expression cassette or vector. In some embodiments, the first polynucleotide and the second polynucleotide are in separate expression cassettes or vectors. In some embodiments, the gRNA is any gRNA targeting a mutant PLN (such as a deleterious mutant of PLN). In some embodiments, the gRNA targets a deletion of Arg14 in a PLN gene (“PLN-R14Del” mutation). In some embodiments, the gRNA is complementary to a sequence of the PLN gene comprising a deletion of Arg14. In some embodiments, the gRNA is any gRNA that can target a mutant PLN, e.g., PLN-R14Del mutation. In some embodiments, gRNAs provided herein can target a mutant PLN with high editing efficiency of indel formation (e.g., equal to or more than 30%, 40% or 50%). In some embodiments, the vector is a viral vector, such as any viral vector, e.g., an AAV vector (e.g., AAV9).

In some aspects, the present disclosure provides virions (e.g., recombinant AAV or rAAV) comprising any gRNA, expression cassette, and/or vector described herein. In some embodiments, provided herein are AAV virions, comprising any gRNA, expression cassette, and/or vector described herein and a capsid protein. In some embodiments, AAV is AAV9 or a variant thereof. In some embodiments, the capsid protein is wild-type AAV9 capsid protein or a variant thereof (such as any capsid protein described herein).

In some aspects, the present disclosure provides pharmaceutical compositions comprising any gRNA, expression cassette, vector, and/or virion described herein, and a pharmaceutically acceptable carrier.

In some aspects, the disclosure provides cells compositions comprising any gRNA, expression cassette, vector and/or virion described herein. In some aspects, the disclosure provides a method of transducing an iPSC cell or a cardiac cell (e.g., a cardiomyocyte) with any gRNA, expression cassette, vector, and/or virion described herein, wherein the cell expresses the polynucleotide transgene(s).

In some aspects, the disclosure provides methods of treating or preventing a disease or condition (e.g., a cardiac disease) in a subject (e.g., a mammal such as a human) with a mutation in a PLN gene comprising administering to the subject a gRNA targeting the mutation (e.g., a deletion) in the PLN gene (e.g., a deleterious mutation). In some embodiments, the gRNA targets PLN-R14Del mutation in the PLN gene. In some embodiments, the gRNA is any gRNA described herein. In some embodiments, the method further comprises administering a Cas endonuclease, for example a polynucleotide encoding a Cas endonuclease (e.g., Cas9 such as saCas9). In some embodiments, the gRNA is administered in a vector, virion or composition.

In some embodiments, the Cas endonuclease is administered in the same vector, virion or composition as the gRNA. In some embodiments, a vector is administered which comprises a polynucleotide encoding PLN mutant-targeting gRNA operably linked to an RNA expression-driving promoter (e.g., U6 or any other such promoter described herein). In some embodiments, a vector is administered which comprises a polynucleotide encoding a Cas endonuclease (e.g., Cas9 such as saCas9), which optionally further comprises a polynucleotide encoding PLN mutant-targeting gRNA as described herein. In some embodiments, the polynucleotide encoding Cas9 and the polynucleotide encoding gRNA have head-to-tail orientation in the same expression cassette or vector. The disease or condition can be any disease or condition associated with one or more PLN mutations (e.g., PLN-R14Del mutation). In some aspects, the disclosed gRNAs, polynucleotides, expression cassettes, vectors, virions and compositions may be used in treatment or prevention of: heart disease, such as cardiomyopathy, e.g., arrhythmogenic cardiomyopathy (ACM), arrhythmogenic right ventricular cardiomyopathy (ARVC), dilated cardiomyopathy (DCM), hypertrophic cardiomyopathy (HCM), or restrictive cardiomyopathy (RC)). In some embodiments, the disease is cardiomyopathy (e.g., DCM, HCM, ACM, or ARVC), e.g., a cardiomyopathy associated with a PLN mutation. In some embodiments, the disease is arrhythmia, e.g., an arrhythmia associated with a PLN mutation. In some embodiments, the disease or condition is heart failure, e.g., heart failure associated with a PLN mutation. In some embodiments, the subject has been diagnosed with a mutation in the PLN gene (e.g., PLN-R14Del mutation).

In some embodiments, provided herein are guide RNAs for targeting a PLN gene comprising a deletion of Arg14, expression cassettes, vectors, virions compositions, and kits comprising the same, and methods of treatment of a human with such a PLN-R14 deletion.

Other embodiments, features, and advantages of the disclosure will be apparent from and encompassed by the following detailed description and claims.

### Terminology

Unless the context indicates otherwise, the features of the invention can be used in any combination. Any feature or combination of features set forth can be excluded or omitted. Certain features of the invention, which are described in separate embodiments may also be provided in combination in a single embodiment. Features of the invention, which are described in a single embodiment may also be provided separately or in any suitable sub-combination.

Generally, technical and scientific terms used herein have the same meaning as commonly understood by one of ordinary skill in the art to which this invention belongs. The detailed description is divided into sections only for the reader's convenience and disclosure found in any section may be combined with that in another section.

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3rd edition; Ausubel et al. eds. (2007) Current Protocols in Molecular Biology; Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (2002) Cold Spring Harbor Laboratory Press; Sohail (2004) Gene Silencing by RNA Interference: Technology and Application (CRC Press); and Sell (2013) Stem Cells Handbook.

As used in this specification and the appended claims, the singular forms “a,” “an” and “the” include plural references unless the content clearly dictates otherwise.

As used in this specification, the term “and/or” is used in this disclosure to mean either “and” or “or” unless indicated otherwise.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

“AAV” is an abbreviation for adeno-associated virus. The term covers all subtypes of AAV, except where a subtype is indicated, and to both naturally occurring and recombinant forms. The abbreviation “rAAV” refers to recombinant adeno-associated virus. “AAV” includes AAV or any subtype. “AAV5” refers to AAV subtype 5. “AAV9” refers to AAV subtype 9. The genomic sequences of various serotypes of AAV, as well as the sequences of the native inverted terminal repeats (ITRs), Rep proteins, and capsid subunits may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001829 (AAV4), U89790 (AAV4), NC_006152 (AAV5), AF513851 (AAV7), AF513852 (AAV8), NC_006261 (AAV8), and AY530579 (AAV9). Publications describing AAV include Srivistava et al. (1983) J. Virol. 45:555; Chiorini et al. (1998) J. Virol. 71:6823; Chiorini et al. (1999) J. Virol. 73:1309; Bantel-Schaal et al. (1999) J. Virol. 73:939; Xiao et al. (1999) J. Virol. 73:3994; Muramatsu et al. (1996) Virol. 221:208; Shade et al. (1986) J. Virol. 58:921; Gao et al. (2002) Proc. Nat. Acad. Sci. USA 99: 11854; Moris et al. (2004) Virology 33:375-383; Int'l Pat. Publ Nos. WO2018/222503A1, WO2012/145601A2, WO2000/028061A2, WO1999/61601A2, and WO1998/11244A2; U.S. Pat. Appl. Nos. 15/782,980 and 15/433,322; and U.S. Pat. Nos. 10,036,016, 9,790,472, 9,737,618, 9,434,928, 9,233,131, 8,906,675, 7,790,449, 7,906,111, 7,718,424, 7,259,151, 7,198,951, 7,105,345, 6,962,815, 6,984,517, and 6,156,303.

An “AAV vector” or “rAAV vector” as used in the art to refer either to the DNA packaged into in the rAAV virion or to the rAAV virion itself, depending on context. As used herein, unless otherwise apparent from context, rAAV vector refers to a nucleic acid (typically a plasmid) comprising a polynucleotide sequence capable of being packaged into an rAAV virion, but with the capsid or other proteins of the rAAV virion. Generally an rAAV vector comprises a heterologous polynucleotide sequence (i.e., a polynucleotide not of AAV origin) and one or two AAV inverted terminal repeat sequences (ITRs) flanking the heterologous polynucleotide sequence. Only one of the two ITRs may be packaged into the rAAV and yet infectivity of the resulting rAAV virion may be maintained. See Wu et al. (2010) Mol Ther. 18:80. An rAAV vector may be designed to generate either single-stranded (ssAAV) or self-complementary (scAAV). See McCarty D. (2008) Mo. Ther. 16:1648-1656; WO2001/11034; WO2001/92551; WO2010/129021.

An “AAV particle” refers to an extracellular viral particle including at least one viral capsid protein (e.g. VP1) and an encapsidated AAV vector (or fragment thereof), including the capsid proteins.

For brevity and clarity, the disclosure refers to “capsid protein” or “capsid proteins” of AAV. Those skilled in the art understand that such references refer to VP1, VP2, or VP3, or combinations of VP1, VP2, and VP3. As in wild-type AAV and most recombinant expression systems VP1, VP2, and VP3 are expressed from the same open reading frame, engineering of the sequence that encodes VP3 inevitably alters the sequences of the C-terminal domain of VP1 and VP2. One may also express the capsid proteins from different open reading frames, in which case the capsid of the resulting rAAV virion could contain a mixture of wild-type and engineered capsid proteins, and mixtures of different engineered capsid proteins.

The term “promoter” as used herein refers a polynucleotide sequence that has one or more recognition site(s) to which an RNA polymerase binds, such that in a host or target cell, an RNA polymerase may initiate and transcribe a polynucleotide sequence “downstream” of the promoter into an RNA. Similarly stated, a “promoter” is operably linked or operatively linked to a polynucleotide sequence if in a host or target cell in which the promoter is active, an RNA polymerase initiates transcription of the polynucleotide at a transcription state site. Promoters operative in mammalian cells generally comprise an AT-rich region located approximately 25 to 30 bases upstream from the site where transcription is initiated and/or another sequence found 70 to 80 bases upstream from the start of transcription, a CNCAAT region where N may be any nucleotide.

The terms “upstream” and “upstream end” refer to a portion of a polynucleotide that is, with reference to a transcription start site (TSS), 5′ to the TSS on the sense strand (or coding strand) of the polynucleotide; and 3′ to the TSS on the antisense strand of the polynucleotide. The terms “downstream” and “downstream end” refer to a portion of a polynucleotide that is, with reference to a TSS, 3′ to TSS on the sense strand (or coding strand) of the polynucleotide; and 5′ to the TSS on the antisense strand of the polynucleotide. Thus, a deletion from the upstream end of a promoter is a deletion of one or more base pairs in the non-transcribed region of the polynucleotide, 5′ to the TSS on the sense strand (or equivalently, 3′ to the TSS on the antisense strand). A deletion from the downstream end of a promoter is a deletion of one or more base pairs in the transcribed region of the polynucleotide, 3′ to the TSS on the sense strand (or equivalently, 5′ to the TSS on the antisense strand).

The term “transgene” refers to a nucleic acid sequence encoding a protein or RNA (e.g., a therapeutic protein), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

Methods of sequence alignment for comparison and determination of percent sequence identity is well known in the art. Optimal alignment of sequences for comparison can be conducted, e.g., by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al, Current Protocols in Molecular Biology (2003)), by use of algorithms know in the art including the BLAST and BLAST 2.0 algorithms, which are described in Altschul et al., Nuc. Acids Res. 25:3389-3402 (1977); and Altschul et al., J. Mol. Biol. 215:403-410 (1990), respectively. Software for performing BLAST analyses is publicly available through the National Center for Biotechnology Information.

In some embodiments, the determination of the percentage of sequence identity may take place after a local alignment. Such alignments are well known in the art, for instance the service EMBOSS Matcher identifies local similarities between two sequences using an algorithm based on the LALIGN application, version 2.0u4. In an example, the identity between two nucleic acid sequences may be calculated using the service Matcher (EMBOSS) set to the default parameters, e.g., matrix (DNAfull), gap open (16), gap extend (4), alternative matches (1).

An “expression cassette” or “expression construct” refers to a DNA polynucleotide sequence operably linked to a promoter. “Operably linked” or “operatively linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

“Recombinant,” as applied to a polynucleotide means that the polynucleotide is the product of various combinations of cloning, restriction or ligation steps, and other procedures that result in a construct that is distinct from a polynucleotide found in nature, or that the polynucleotide is assembled from synthetic oligonucleotides. A “recombinant” protein is a protein produced from a recombinant polypeptide. A recombinant virion is a virion that comprises a recombinant polynucleotide and/or a recombinant protein, e.g. a recombinant capsid protein.

The term “delivery”, which is used interchangeably with “transduction,” refers to the process by which exogenous nucleic acid molecules are transferred into a cell such that they are located inside the cell. Delivery of nucleic acids is a distinct process from expression of nucleic acids.

The term “modified” refers to a substance or compound (e.g., a cell, a polynucleotide sequence, and/or a polypeptide sequence) that has been altered or changed as compared to the corresponding unmodified substance or compound.

The term “sample” refers to abiological composition (e.g., a cell or a portion of a tissue) that is subjected to analysis and/or genetic modification. In some embodiments, a sample is a “primary sample” in that it is obtained directly from a subject; in some embodiments, a “sample” is the result of processing of a primary sample, for example to remove certain components and/or to isolate or purify certain components of interest.

The term “transfection” refers to the uptake of foreign DNA by a cell. A cell has been “transfected” when exogenous DNA has been introduced inside the cell membrane. A number of transfection techniques are generally known in the art. See, e.g., Graham et ah, Virology 52:456 (1973); Sambrook et al., Molecular Cloning: A Laboratory Manual (1989); Davis et ah, Basic Methods in Molecular Biology (1986); Chu et al., Gene 13:197 (1981). Such techniques can be used to introduce one or more exogenous DNA moieties, such as a nucleotide integration vector and other nucleic acid molecules, into suitable host cells. The term captures chemical and electrical transfection procedures.

The term “expression” refers to the process by which a nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

The term “gene therapy” involves the transfer of heterologous DNA to cells of a mammal, particularly a human, with a disorder or conditions for which therapy or diagnosis is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product; it may encode a product, such as a peptide or RNA that in some manner mediates, directly or indirectly, expression of a therapeutic product. Gene therapy may also be used to deliver nucleic acid encoding a gene product to replace a defective gene or supplement a gene product produced by the mammal or the cell in which it is introduced. The introduced nucleic acid may encode a therapeutic gene product that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, a “heterologous” polynucleotide or nucleic acid refers to a polynucleotide or portion of a polynucleotide derived from a source other than the host organism or, for a viral vector, the native, non-recombinant virus. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies.

The term “wild-type” refers to the naturally-occurring polynucleotide sequence encoding a protein, or a portion thereof, or protein sequence, or portion thereof, respectively, as it normally exists in vivo in a normal or healthy subject.

The term “variant” refers to a protein or nucleic acid having one or more genetic changes (e.g., insertions, deletions, substitutions, or the like) that returns all or substantially all of the functions of the reference protein or nucleic acid. For example, a variant of a therapeutic protein retains the same or substantially the same activity and/or provides the same or substantially the same therapeutic benefit to a subject in need thereof. A variant of a promoter sequence retains the ability to initiate transcription at the same or substantially the same level as the reference promoter and retains the same or substantially the same cell type specificity. In particular embodiments, polynucleotides variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence. In particular embodiments, protein variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

The term “subject” includes animals, such as e.g., mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

The term “administering” to a subject is a procedure by which one or more delivery agents, together or separately, are introduced into or applied onto a subject such that target cells which are present in the subject are eventually contacted with the agent.

The term “gene product” refers to a protein or nucleic acid produced by the transcription of a polynucleotide and, in the case of a protein gene product, the subsequent translation of transcript into a protein. A “therapeutic gene product” refers to a gene product that provides a therapeutic physiological effect or benefit to a subject in need when expressed in a therapeutic amount in a subject.

The term “cardiomyopathy” refers to the deterioration of the function of the myocardium (i.e., the actual heart muscle) for any reason. Subjects with cardiomyopathy are often at risk of arrhythmia or sudden cardiac death or both.

The term “hypertrophic cardiomyopathy” refers to a disease of the heart and myocardium in which a portion of the myocardium is hypertrophied.

The term “familial hypertrophic cardiomyopathy” refers to a genetic disorder characterized by increased growth (i.e., hypertrophy) in thickness of the wall of the left ventricle.

The term “dilated cardiomyopathy” refers to a disease that occurs as a result of restricted blood flow to the heart muscles, resulting in weakening and/or thinning of the heart chamber walls.

The term “arrhythmogenic right ventricular cardiomyopathy” refers to a disease in which right ventricular muscle is replaced by fat or scar tissue, interfering with normal heartbeat rhythm.

As used herein, the term “left ventricular internal diameter at diastole” or “LVIDd” refers to left ventricular size at diastole.

As used herein, the term “left ventricular internal diameter at systole” or “LVIDs” refers to left ventricular size at systole.

As used herein, the term “left ventricular mass” refers to the weight of the left ventricle.

As used herein, the term “ejection fraction” refers to the amount of blood being bumped out of the left ventricle each time it contracts, expressed as a percentage to the total amount of blood in left ventricle.

The term “effective amount” refers to the minimum amount of an agent or composition required to result in a particular physiological effect. The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (ECso), which refers to the concentration of an agent that results in a magnitude of a particular physiological response that is half-way between a reference level and a maximum response level.

### Mutant Phospholamban as a Therapeutic Target

Phospholamban (PLN) plays an important role in cardiomyocyte calcium homeostasis by inhibiting sarco/endoplasmic reticulum Ca2+-ATPase (SERCA).

Several PLN mutations have been identified in dilated cardiomyopathy patients, including PLN-R14Del mutation, R9C (Arg to Cys), R9L, R9H, Leu-39stop, and R25C.

The PLN-R14Del pathogenic mutation is associated with both idiopathic dilated cardiomyopathy (DCM) and arrhythmogenic right ventricular cardiomyopathy (ARVC). In one study, the PLN-R14Del pathogenic mutation was identified in 15% of DCM patients and 12% of ARVC patients. See van der Zwaag et al. European Journal of Heart Failure (14)1199-1207 (2012).

Other PLN mutations include a T116G point mutation, substituting a termination codon for Leu-39 (L39stop) and a C25T point mutation, substituting a cysteine for Arg-9 (R9C). See Haghighi et al., JCI (111)869-876 (2003) and Schmitt et al., Science (299)1410-1413 (2003), respectively.

In some embodiments, the gRNAs, vectors, virions, compositions, and methods of the disclosure target any mutant PLN, such as a deleterious mutant PLN associated with a disease or condition. In some embodiments, the vectors, virions, compositions, and methods of the disclosure target any mutant PLN by introducing a specific alteration in the mutation site but not in a wildtype PLN gene. In some embodiments, the gRNAs are complementary to a sequence of the PLN gene comprising a mutation. In some embodiments, provided herein are polynucleotides encoding any guide RNA complementary to a sequence of the PLN gene comprising a mutation, as well as vectors, virions and compositions comprising such guide RNA. The mutant PLN can be any mutant PLN described herein or known in the art.

In some embodiments, the guide RNA, vectors, virions, compositions, and methods of the disclosure target PLN-R14Del mutation. In some embodiments, the vectors, virions, compositions, and methods of the disclosure target PLN-R14Del mutation by introducing a specific alteration in the mutation site but not in a wildtype PLN gene. In some embodiments, the gRNAs are complementary to a sequence of the PLN gene comprising the PLN-R14Del mutation. In some embodiments, provided herein are polynucleotides encoding any guide RNA complementary to a sequence of the PLN gene comprising the PLN-R14Del mutation, as well as vectors, virions and compositions comprising such guide RNA.

In some embodiments, the alteration has a specificity for the PLN-R14Del mutation with an off-target alteration of less than 10%, less than 9%, less than 8%, less than 7%, less than 6%, less than 5%, less than 4%, less than 3%, less than 2%, less than 1%, or 0%.

The PLN and PLN-R14Del sequences are provided in Table 1 below. In some embodiments, the gRNAs, vectors, virions, compositions, and methods of the disclosure target any mutant PLN, such as a variant of SEQ ID NO: 31, or target PLN-R14Del mutant of SEQ ID NO: 33.

TABLE 1
Exemplary PLN sequences

| | | SEQ |
|---|---|---|
| | | ID |
| Name | Sequence | NO: |
| WT | atggagaaagtccaatacctcactcgctcagct | 31 |
| human | ataagaagagcctcaaccattgaaatgcctcaa | |
| PLN | caagcacgtcaaaagctacagaatctatttatc | |
| DNA | aatttctgtctcatcttaatatgtctcttgctg | |
| | atctgtatcatcgtgatgcttctctga | |
| WT | MEKVQYLTRSAIRRASTIEMPQQARQKLQNLFI | 32 |
| human | NFCLILICLLLICIIVMLL | |
| PLN | | |
| protein | | |
| PLN | atggagaaagtccaatacctcactcgctcagcta | 33 |
| R14Del | taagagcctcaaccattgaaatgcctcaacaagc | |
| DNA | acgtcaaaagctacagaatctatttatcaatttc | |
| | tgtctcatcttaatatgtctcttgctgatctgta | |
| | tcatcgtgatgcttctctga | |
| PLN | MEKVQYLTRSAIRASTIEMPQQARQKLQNLFINF | 34 |
| R14Del | | |
| protein | CLILICLLLICIIVMLL | |

### CRISPR/Cas Technology

Components of any known CRISPR/Cas technology may be used in the aspects and embodiments of the invention described herein.

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracrRNA (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide RNA sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes or Staphylococcus aureus.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 13, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The components of a CRISPR system can be implemented in any suitable manner, meaning that the components of such systems including the RNA-guided nuclease (e.g., a Cas enzyme) and gRNA can be delivered, formulated, or administered in any suitable form to the cells. For example, the RNA-guided nuclease may be delivered to a cell complexed with a gRNA (e.g., as a ribonucleoprotein (RNP) complex), the RNA-guided nuclease may be delivered to a cell separate (e.g., uncomplexed) to a gRNA, the RNA-guided nuclease may be delivered to a cell as a polynucleotide (e.g., DNA or RNA) encoding the nuclease that is separate from a gRNA, or both the RNA-guided nuclease and the gRNA molecule may be delivered as polynucleotides encoding each component.

One or more vectors driving expression of one or more elements of the CRISPR system can be introduced into the cell such that expression of the elements of the CRISPR system direct formation of the CRISPR complex at one or more target sites. Components can also be delivered to cells as ribonucleoprotein complexes, proteins, DNA, and/or RNA. For example, a Cas enzyme, an RNA guide sequence linked to a tracr-mate sequence, and a tracrRNA sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. In addition, a nucleic acid encoding the endonuclease (e.g., a Cas enzyme such as Cas8 or Cas9) may be delivered with gRNAs. In some embodiments, multiple gRNA sequences are used.

In some embodiments, formation of the CRISPR complex (comprising a guide RNA hybridized to the target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. The CRISPR system can induce double stranded breaks (DSBs) at the target site, followed by disruptions or alterations, such as insertions and/or deletions (indels), as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The tracrRNA sequence, which may comprise or consist of all or a portion of the tracrRNA sequence according to SEQ ID NO: 9 (e.g., about or more than about 60%, about 65%, about 70%, about 75%, about 80%, about 85%, about 90%, about 95%, 97%, 98% or 100% sequence identity to SEQ ID NO: 9), may also form part of the CRISPR complex, such as by hybridization to all or a portion of a tracr-mate sequence that is operably linked to the guide RNA sequence. The tracrRNA sequence has sufficient complementarity to a tracr-mate sequence to hybridize and participate in formation of the CRISPR complex, such as at least 50%, 60%, 70%, 80%, 90%, 95%, 99% or 100% of sequence complementarity along the length of the tracr-mate sequence when optimally aligned.

### Guide RNAs for Targeting PLN

In some embodiments, a guide RNA (gRNA) sequence used to target any PLN mutation is any gRNA sequence described herein or known in the art. The gRNAs provided herein can be used for site-directed editing of a genomic DNA target (e.g., a PLN gene), for example, in association with the CRISPR system.

In some embodiments, a guide RNA sequence is used to target the PLN-R14Del mutation. In some embodiments, any guide RNA sequences described herein can be used for this purpose.

In some embodiments, at least 1, at least 2, at least 3, or at least 4 different guide RNA sequences are used to target the PLN-R14Del mutation.

In some embodiments, provided herein are polynucleotides encoding any guide RNA described herein. In some embodiments, provided herein are polynucleotides encoding any guide RNA complementary to a sequence of the PLN gene comprising PLN-R14Del mutation. In some embodiments, provided herein are polynucleotides encoding any guide RNA described herein linked to an RNA expression-driving promoter, such as any Pol III promoter, e.g., a U6 promoter.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies), ELAND (Illumina, San Diego, Calif.), SOAP (available at soap.genomics.org.cn), and Maq (available at maq.sourceforge.net).

In some embodiments, the gRNA sequence comprises a crRNA sequence, which in turn comprises a complementary region (also can be referred to as a spacer) that recognizes and binds a complementary target DNA of interest. The length of the spacer or complementary region is generally between 15 and 30 nucleotides, usually about 20 nucleotides in length, although will vary based on the requirements of the specific CRISPR system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In some embodiments, the gRNA sequence comprises a polynucleotide sequence comprising at least 16, 17, 18, or 19 nucleotides with up to 3 mismatches of any one of SEQ ID NOs: 1-4, optionally wherein the polynucleotide sequence comprises ttatagctga (SEQ ID NO: 127).

In some embodiments, the gRNA sequence comprises a polynucleotide sequence comprising at least 16, 17, 18, or 19 nucleotides with up to 1 or 2 mismatches of any one of SEQ ID NOs: 1-4, optionally wherein the polynucleotide sequence comprises ttatagctga (SEQ ID NO: 127).

In some embodiments, the gRNA sequence comprises a polynucleotide sequence comprising at least 16, 17, 18, or 19 nucleotides with no mismatches of any one of SEQ ID NOs: 1-4, optionally wherein the polynucleotide sequence comprises ttatagctga (SEQ ID NO: 127).

In some embodiments, the gRNA sequence comprises a polynucleotide sequence of any one of SEQ ID NOs: 1-4, with up to 3 mismatches, optionally wherein the polynucleotide sequence comprises ttatagctga (SEQ ID NO: 127).

In some embodiments, the gRNA sequence comprises a polynucleotide sequence of any one of SEQ ID NOs: 1-4, with up to 1 or 2 mismatches, optionally wherein the polynucleotide sequence comprises ttatagctga (SEQ ID NO: 127).

In some embodiments, the gRNA sequence comprises a polynucleotide sequence of any one of SEQ ID NOs: 1-4. In some embodiments, the gRNA crispr sequence consists of a polynucleotide sequence of any one of SEQ ID NOs: 1-4.

In some embodiments, the gRNA sequence comprises a polynucleotide sequence comprising at least 16, 17, 18, or 19 nucleotides with 1, 2 or 3 mismatches of SEQ ID NO: 18. In some embodiments, the gRNA sequence comprises a polynucleotide sequence comprising at least 16, 17, 18, or 19 nucleotides with no mismatches of SEQ ID NO: 18. In some embodiments, the gRNA sequence comprises a polynucleotide sequence of SEQ ID NO: 18. In some embodiments, the gRNA crispr sequence consists of a polynucleotide sequence of SEQ ID NO: 18.

In some embodiments, the gRNA sequence comprises less than 5 nucleotide substitutions, less than 4 nucleotides substitutions, less than 3 nucleotides substitutions, less than 2 nucleotides substitutions, less than 1 nucleotides substitutions, or no nucleotide substitutions in SEQ ID NO: 1.

In some embodiments, the gRNA sequence comprises less than 5 nucleotide substitutions, less than 4 nucleotides substitutions, less than 3 nucleotides substitutions, less than 2 nucleotides substitutions, less than 1 nucleotides substitutions, or no nucleotide substitutions in SEQ ID NO: 2.

In some embodiments, the gRNA sequence comprises less than 5 nucleotide substitutions, less than 4 nucleotides substitutions, less than 3 nucleotides substitutions, less than 2 nucleotides substitutions, less than 1 nucleotides substitutions, or no nucleotide substitutions in SEQ ID NO: 3.

In some embodiments, the gRNA sequence comprises less than 5 nucleotide substitutions, less than 4 nucleotides substitutions, less than 3 nucleotides substitutions, less than 2 nucleotides substitutions, less than 1 nucleotides substitutions, or no nucleotide substitutions in SEQ ID NO: 4.

In some embodiments, the gRNA sequence comprises less than 5 nucleotides substitutions, less than 4 nucleotides substitutions, less than 3 nucleotides substitutions, less than 2 nucleotides substitutions, less than 1 nucleotides substitutions, or no nucleotide substitutions in SEQ ID NO: 18.

TABLE 2A
Exemplary gRNA complementary region sequences

| Name | Sequence | SEQ ID NO: |
|---|---|---|
| Human sgRNA1 complementary | ggttgaggctc | 1 |
| region (21 nt) | ttatagctga | |
| Human sgRNA2 complementary | ttgaggctctt | 2 |
| region (19 nt) | atagctga | |
| Human sgRNA3 complementary | gttgaggctct | 3 |
| region (20 nt) | tatagctga | |
| Human sgRNA4 complementary | tggttgaggct | 4 |
| region (22 nt) | cttatagctga | |
| Mouse sgRNA complementary | agtggaggccc | 18 |
| region | tgatagccga | |

In some embodiments, the gRNA sequence further comprises a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the tracrRNA sequence comprises a polynucleotide sequence of SEQ ID NO: 9.

In some embodiments, the tracrRNA sequence comprises the polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 9. In some embodiments, the tracrRNA sequence comprises the polynucleotide sequence that shares at least 85%, at least 90%, at least 95%, at least 96%, at least 97%, at least 98%, at least 99%, or 100% identity to SEQ ID NO: 9.

TABLE 2B
Exemplary gRNA tracrRNA sequences

| Name | Sequence | SEQ ID NO: |
|---|---|---|
| tracrRNA | gttttagtactctggaaacagaat | 9 |
| | ctactaaaacaaggcaaaatgccgt | |
| | gtttatctcgtcaacttgttggcga | |
| | ga | |

In some embodiments, the gRNA sequence comprises two or more gRNA molecules, for example, a crRNA and a tracrRNA, as two separate molecules. In other embodiments, the gRNA sequence is a single guide RNA (sgRNA), including a sgRNA comprising a crRNA and a tracrRNA on a single RNA molecule. In certain of these embodiments, the crRNA and tracrRNA are linked by an intervening tetraloop.

In some embodiments, the gRNA sequence comprises, essentially consists of or consists of a polynucleotide sequence of SEQ ID NO: 5. In some embodiments, the gRNA sequence comprises, essentially consists of or consists of the polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 5. In some embodiments, the gRNA sequence comprises, essentially consists of or consists of a polynucleotide sequence comprising at least 16, 17, 18, or 19 nucleotides with 0, 1, 2 or 3 mismatches of SEQ ID NO: 1, optionally wherein the polynucleotide sequence comprises ttatagctga (SEQ ID NO: 127), and wherein the polynucleotide sequence shares at least 75%, at least 80%, at least 85%, at least 90%, at least 95%, at least 96%, at least 97%, at least 98%, at least 99%, or 100% identity to SEQ ID NO: 5.

In some embodiments, the gRNA sequence comprises, essentially consists of or consists of a polynucleotide sequence of SEQ ID NO: 6. In some embodiments, the gRNA sequence comprises, essentially consists of or consists of the polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 6. In some embodiments, the gRNA sequence comprises, essentially consists of or consists of a polynucleotide sequence comprising at least 16, 17, 18, or 19 nucleotides with 0, 1, 2 or 3 mismatches of SEQ ID NO: 2, optionally wherein the polynucleotide sequence comprises ttatagctga (SEQ ID NO: 127), and wherein the polynucleotide shares at least 75%, at least 80%, at least 85%, at least 90%, at least 95%, at least 96%, at least 97%, at least 98%, at least 99%, or 100% identity to SEQ ID NO: 6.

In some embodiments, the gRNA sequence comprises, essentially consists of or consists of a polynucleotide sequence of SEQ ID NO: 7. In some embodiments, the gRNA sequence comprises, essentially consists of or consists of the polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 7. In some embodiments, the gRNA sequence comprises, essentially consists of or consists of a polynucleotide sequence comprising at least 16, 17, 18, or 19 nucleotides with 0, 1, 2 or 3 mismatches of SEQ ID NO: 3, optionally wherein the polynucleotide sequence comprises ttatagctga (SEQ ID NO: 127), and wherein the polynucleotide shares at least 75%, at least 80%, at least 85%, at least 90%, at least 95%, at least 96%, at least 97%, at least 98%, at least 99%, or 100% identity to SEQ ID NO: 7.

In some embodiments, the gRNA sequence comprises, essentially consists of or consists of a polynucleotide sequence of SEQ ID NO: 8. In some embodiments, the gRNA sequence comprises, essentially consists of or consists of the polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 8. In some embodiments, the gRNA sequence comprises, essentially consists of or consists of a polynucleotide sequence comprising at least 16, 17, 18, or 19 nucleotides with 0, 1, 2 or 3 mismatches of SEQ ID NO: 4, optionally wherein the polynucleotide sequence comprises TTATAGCTGA, and wherein the polynucleotide shares at least 75%, at least 80%, at least 85%, at least 90%, at least 95%, at least 96%, at least 97%, at least 98%, at least 99%, or 100% identity to SEQ ID NO: 8.

TABLE 2C
Exemplary sgRNA sequences

| Name | Sequence | SEQ ID NO: |
|---|---|---|
| Human sgRNA1 (with | ggttgaggctcttatagctgagtttagtactc | 5 |
| complementary region | tggaaacagaatctactaaaacaaggcaaaat | |
| highlighted) | gccgtgtttatctcgtcaacttgttggcgaga | |
| Human sgRNA2 (with | ttgaggctcttatagctgagtttagtactctg | 6 |
| complementary region | gaaacagaatctactaaaacaaggcaaaatgc | |
| highlighted) | cgtgtttatctcgtcaacttgttggcgaga | |
| Human sgRNA3 (with | gttgaggctcttatagctgagtttagtactct | 7 |
| complementary region | ggaaacagaatctactaaaacaaggcaaaatg | |
| highlighted) | ccgtgtttatctcgtcaacttgttggcgaga | |
| Human sgRNA4 (with | tggttgaggctcttatagctgagtttagtactc | 8 |
| complementary region | tggaaacagaatctactaaaacaaggcaaaat | |
| highlighted) | gccgtgtttatctcgtcaacttgttggcgaga | |

### Cas9s for Targeting PLN

In some embodiments, any Cas endonuclease is used in the present invention. The Cas endonucleases provided herein can be used for site-directed editing of a genomic DNA target (e.g., a PLN gene), for example, in association with the CRISPR system and the gRNAs described herein.

In some embodiments, provided herein are polynucleotides encoding any Cas endonuclease. In some embodiments, a Cas endonuclease is a Cas9 endonuclease. In some embodiments, provided herein are polynucleotides encoding any Cas endonuclease described herein operably linked to a protein expression-driving promoter (such as any protein expression-driving promoter suitable for expression in mammalian cells), for example a muscle cell-specific promoter or a cardiac cell-specific promoter. In some embodiments, provided herein are polynucleotides encoding any Cas endonuclease described herein operably linked to a TNNT2 promoter described herein, such as a human TNNT2 promoter.

In some embodiments, a Cas endonuclease is any Cas endonuclease (e.g., any Cas9 endonuclease) which is encoded by a gene equal to or less than 3.3 kb in size, equal to or less than 3.2 kb in size, equal to or less than 3.1 kb in size, equal to or less than 3 kb in size, equal to or less than 2.9 kb in size, or equal to or less than 2.8 kb in size. In some embodiments, a Cas endonuclease is any Cas endonuclease (e.g., any Cas9 endonuclease) which has the protein size of equal to or less than 1,100 amino acids, equal to or less than 1,075 amino acids, equal to or less than 1,060 amino acids, equal to or less than 1,050 amino acids, equal to or less than 1,000 amino acids, equal to or less than 950 amino acids, or equal to or less than 900 amino acids.

Non-limiting examples of Cas endonucleases that can be used as described here include Cas1, Cas1B, Cas2, Cas3, Cas4, Cas5, Cas6, Cas7, Cas8, Cas9 (also known as Csn1 and Csx12), Cas10, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known, for example, the amino acid sequence of S. pyogenes Cas9 protein may be found in the SwissProt database under accession number Q99ZW2.

In some embodiments, Cas9 endonuclease is a S. aureus Cas9 (SaCas9) or a variant thereof. In some embodiments, Cas9 endonuclease is a S. pyogenes Cas9 (SpCas9) or a variant thereof.

In order for the Cas nuclease to function, there must be a protospacer adjacent motif (PAM) immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene, and there may be an optimal PAM sequence for a particular Cas protein and less optimal PAM sequences that differ in one or more nucleotides from the optimal sequence. For example, the SpCas recognizes a PAM sequence of 5′-NGG-3′ or, at less efficient rates, 5′-NAG-3′, where N can be any nucleotide. For another example, the SaCas9 PAM sequence is NNGRR or for optimal on-target cutting is NNGRRT, wherein N can be any nucleotide, and R can be guanine or adenine. Other Cas nuclease variants with alternative PAMs have also been characterized and successfully used for genome editing. Therefore, the PAM sequence adjacent to the target sequence is an essential targeting component for the design of CRISPR/Cas9-mediated gene editing, and when designing gRNAs targeting a specific genomic locus, one skilled in the art needs to consider the availability and/or location of PAM sequences optimal for the Cas nuclease of choice at the target locus and, if necessary, select a different Cas nuclease/PAM sequence pair based on the DNA sequence at the target locus. PAMs for use with different Cas endonucleases are known in the art. Illustrative examples of Cas enzymes that can be used as described herein and PAMs for use with their respective Cas endonucleases are shown in Table 3A below.

TABLE 3A
Exemplary Cas proteins and their corresponding PAM sequences

| | | PAM | Size |
|---|---|---|---|
| Name | Species | Sequence | (amino acids) |
| SpCas9 | Streptococcus | NGG | 1,368 |
| | pyogenes | | |
| SaCas9 | Staphylococcus | NNGRRT | 1,053 |
| | aureus | | |
| FnCas9 | Francisella | NGG | 1,244 |
| | novicida | | |
| NmCas9 | Neisseria | NNNNGATT | 1,055 |
| | meningitidis | | |
| StCas9 | Streptococcus. | NGG | 1,364 |
| | thermophilus | | |
| AaCas9 | Acidaminococcus sp | NNNNRYAC | 984 |
| GstCas9 | Geobacillus | NGG | 1,245 |
| | stearothermophilus | | |
| NmuCas9 | Neisseria | NNAGAAW | 1,054 |
| | mucosa | | |
| ScCas9 | Streptococcus | NGG | 1,342 |
| | canis | | |
| LbCas9 | Lachnospiraceae | TTTA | 1,305 |
| | bacterium | | |
| SsCas9 | Streptococcus | NNGRRT | 1,365 |
| | sanguinis | | |
| NsuCas9 | Neisseria | NNNNGMTT | 1,056 |
| | subflava | | |
| SeCas9 | Staphylococcus | NNGS | 1,054 |
| | epidermidis | | |
| SagalCas9 | Streptococcus | NNGRRT | 1,363 |
| | agalactiae | | |
| LmCas9 | Listeria | NGG | 1,359 |
| | monocytogenes | | |
| AspCas9 | Actinomyces sp. | NNAGAAW | 1,045 |
| SangCas9 | Streptococcus | NNNNGATT | 1,056 |
| | anginosus | | |
| SdCas9 | Streptococcus | NNGRRT | 1,341 |
| | dysgalactiae | | |
| CjCa9 | Campylobacter | NNNNRYAC | 1,235 |
| | jejuni | | |
| BtCas9 | Bacillus | NGG | 1,243 |
| | thuringiensis | | |
| CdCas9 | Clostridium | NGG | 1,406 |
| | difficile | | |
| ScristCas9 | Streptococcus | NNNNGATT | 1,056 |
| | cristatus | | |
| SmCas9 | Streptococcus | NNGG | 1,333 |
| | mutans | | |
| AsCas12a | Acidaminococcus sp. | TTTV | 1,278 |
| LbCas12b | Lachnospiraceae | TYCV | 1,187 |
| | bacterium | | |
| MboCas12a | Moraxella | TTTV | 1,286 |
| | bovoculi | | |
| RspCas12b | Ruminococcus sp | TYCV | 1,185 |
| AccCas12a | Acidothermus | TTTV | 1,285 |
| | cellulolyticus | | |
| PspCas12a | Prevotella sp. | TTTV | 1,264 |
| PgiCas12b | Porphyromonas | TYCV | 1,167 |
| | gingivalis | | |
| SmaCas12a | Streptococcus | TTTV | 1,259 |
| | mutans | | |
| PinCas12b | Prevotella | TYCV | 1,188 |
| | intermedia | | |
| AspCas12a | Acidovorax sp. | TTTV | 1,284 |
| EspCas12b | Enterobacter sp. | TYCV | 1,296 |
| SpyCas12b | Streptococcus | TYCV | 1,157 |
| | pneumoniae | | |
| AacCas12a | Alicyclobacillus | TTTV | 1,285 |
| | acidocaldarius | | |
| AcbCas12b | Acidobacterium sp. | TYCV | 1,174 |
| PaeCas12a | Pseudomonas | TTTV | 1,284 |
| | aeruginosa | | |
| BfrCas12b | Bacteroides | TYCV | 1,181 |
| | fragilis | | |
| LcrCas12a | Lactobacillus | TTTV | 1,259 |
| | crispatus | | |
| SmaCas12b | Serratia | TYCV | 1,188 |
| | marcescens | | |
| MolCas12a | Moraxella | TTTV | 1,284 |
| | osloensis | | |
| BunCas12b | Bacteroides | TYCV | 1,197 |
| | uniformis | | |
| KoxCas12a | Klebsiella | TTTV | 1,292 |
| | oxytoca | | |
| BthCas12b | Bacillus | TYCV | 1,182 |
| | thuringiensis | | |
| RspCasX | Ruminococcus sp. | TTN | 926 |
| PspCasX | Pseudomonas sp. | TTN | 918 |
| SepCasX | Staphylococcus | TTN | 927 |
| | epidermidis | | |

In some embodiments, the CRISPR enzyme is Cas9. In some embodiments, the Cas9 is derived from S. aureus, S. pyogenes, F. novicida, N. meningitidis, S. thermophilus, Acidaminococcus sp., G. stearothermophilus, N. mucosa, S. canis, Lachnospiraceae bacterium., S. sanguinis, N. subflava, S. epidermidis, S. agalactiae, L. monocytogenes, Actinomyces sp., S. anginosus, S. dysgalactiae, C. jejuni, B. thuringiensis, C. difficile, S. cristatus, S. mutans, or S. pneumonia.

In some embodiments, the CRISPR enzyme is Cas12a. In some embodiments, the Cas12a is derived from Acidaminococcus sp., M. bovoculi, A. cellulolyticus, Prevotella sp., S. mutans, Acidovorax sp., A. acidocaldarius, P. aeruginosa, L. crispatus, M. osloensis, or K oxytoca.

In some embodiments, the CRISPR enzyme is Cas12b. In some embodiments, the Cas12b is derived from Lachnospiraceae bacterium, Ruminococcus sp., P. gingivalis, P. intermedia, Enterobacter sp., S. pneumoniae, Acidobacterium sp., B. fragilis, S. marcescens, B. uniformis, or B. thuringiensis.

In some embodiments, the CRISPR enzyme is CasX. In some embodiments, the CasX is derived from Ruminococcus sp., Pseudomonas sp., or S. epidermidis.

The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding Cas endonuclease is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In some embodiments, a polynucleotide encoding a Cas9 endonuclease comprises or consists of a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 10.

In some embodiments, the Cas9 endonuclease used as described herein or encoded by the polynucleotides described herein comprises an amino acid sequence that shares at least 70%, at least 75%, at least 80%, at least 85%, at least 90%, at least 95%, at least 96%, at least 97%, at least 98%, at least 99%, or 100% identity to SEQ ID NO: 11.

TABLE 3B
Example Cas protein sequences

| | | SEQ ID |
|---|---|---|
| Name | Sequence | NO: |
| saCas9 | atggccccaaagaagaagcggaaggtcggtatcca | 10 |
| | cggagtcccagcagccaagcggaactacatcctgg | |
| | gcctggacatcggcatcaccagcgtgggctacggc | |
| | atcatcgactacgagacacgggacgtgatcgatgc | |
| | cggcgtgcggctgttcaaagaggccaacgtggaaa | |
| | acaacgagggcaggcggagcaagagaggcgccaga | |
| | aggctgaagcggcggaggcggcatagaatccagag | |
| | agtgaagaagctgctgttcgactacaacctgctga | |
| | ccgaccacagcgagctgagggcatcaacccctacg | |
| | aggccagagtgaagggcctgagccagaagctgagc | |
| | gaggaagagttctctgccgccctgctgcacctggc | |
| | caagagaagaggcgtgcacaacgtgaacgaggtgg | |
| | aagaggacaccggcaacgagctgtccaccaaagag | |
| | cagatcagccggaacagcaaggccctggaagagaa | |
| | atacgtggccgaactgcagctggaacggctgaaga | |
| | aagacggcgaagtgcggggcagcatcaacagattc | |
| | aagaccagcgactacgtgaaagaagccaaacagct | |
| | gctgaaggtgcagaaggcctaccaccagctggacc | |
| | agagcttcatcgacacctacatcgacctgctggaa | |
| | acccggcggacctactatgagggacctggcgaggg | |
| | cagccccttcggctggaaggacatcaaagaatggt | |
| | acgagatgctgatgggccactgcacctacttcccc | |
| | gaggaactgcggagcgtgaagtacgcctacaacgc | |
| | cgacctgtacaacgccctgaacgacctgaacaatc | |
| | tcgtgatcaccagggacgagaacgagaagctggaa | |
| | tattacgagaagttccagatcatcgagaacgtgtt | |
| | caagcagaagaagaagcccaccctgaagcagatcg | |
| | ccaaagaaatcctcgtgaacgaagaggatattaag | |
| | ggctacagagtgaccagcaccggcaagcccgagtt | |
| | caccaacctgaaggtgtaccacgacatcaaggaca | |
| | ttaccgcccggaaagagattattgagaacgccgag | |
| | ctgctggatcagattgccaagatcctgaccatcta | |
| | ccagagcagcgaggacatccaggaagaactgacca | |
| | atctgaactccgagctgacccaggaagagatcgag | |
| | cagatctctaatctgaagggctataccggcaccca | |
| | caacctgagcctgaaggccatcaacctgatcctgg | |
| | acgagctgtggcacaccaacgacaaccagatcgct | |
| | atcttcaaccggctgaagctggtgcccaagaaggt | |
| | ggacctgtcccagcagaaagagatccccaccaccc | |
| | tggtggacgacttcatcctgagccccgtcgtgaag | |
| | agaagcttcatccagagcatcaaagtgatcaacgc | |
| | catcatcaagaagtacggcctgcccaacgacatca | |
| | ttatcgagctggcccgcgagaagaactccaaggac | |
| | gcccagaaaatgatcaacgagatgcagaagcggaa | |
| | ccggcagaccaacgagcggatcgaggaaatcatcc | |
| | ggaccaccggcaaagagaacgccaagtacctgatc | |
| | gagaagatcaagctgcacgacatgcaggaaggcaa | |
| | gtgcctgtacagcctggaagccatccctctggaag | |
| | atctgctgaacaaccccttcaactatgaggtggac | |
| | cacatcatccccagaagcgtgtccttcgacaacag | |
| | cttcaacaacaaggtgctcgtgaagcaggaagaaa | |
| | acagcaagaagggcaaccggaccccattccagtac | |
| | ctgagcagcagcgacagcaagatcagctacgaaac | |
| | cttcaagaagcacatcctgaatctggccaagggca | |
| | agggcagaatcagcaagaccaagaaagagtatctg | |
| | ctggaagaacgggacatcaacaggttctccgtgca | |
| | gaaagacttcatcaaccggaacctggtggatacca | |
| | gatacgccaccagaggcctgatgaacctgctgcgg | |
| | agctacttcagagtgaacaacctggacgtgaaagt | |
| | gaagtccatcaatggcggcttcaccagctttctgc | |
| | ggcggaagtggaagtttaagaaagagcggaacaag | |
| | gggtacaagcaccacgccgaggacgccctgatcat | |
| | tgccaacgccgatttcatcttcaaagagtggaaga | |
| | aactggacaaggccaaaaaagtgatggaaaaccag | |
| | atgttcgaggaaaagcaggccgagagcatgcccga | |
| | gatcgaaaccgagcaggagtacaaagagatcttca | |
| | tcaccccccaccagatcaagcacattaaggacttc | |
| | aaggactacaagtacagccaccgggtggacaagaa | |
| | gcctaatagagagctgattaacgacaccctgtact | |
| | ccacccggaaggacgacaagggcaacaccctgatc | |
| | gtgaacaatctgaacggcctgtacgacaaggacaa | |
| | tgacaagctgaaaaagctgatcaacaagagccccg | |
| | aaaagctgctgatgtaccaccacgacccccagacc | |
| | taccagaaactgaagctgattatggaacagtacgg | |
| | cgacgagaagaatcccctgtacaagtactacgagg | |
| | aaaccgggaactacctgaccaagtactccaaaaag | |
| | gacaacggccccgtgatcaagaagattaagtatta | |
| | cggcaacaaactgaacgcccatctggacatcaccg | |
| | acgactaccccaacagcagaaacaaggtcgtgaag | |
| | ctgtccctgaagccctacagattcgacgtgtacct | |
| | ggacaatggcgtgtacaagttcgtgaccgtgaaga | |
| | atctggatgtgatcaaaaaagaaaactactacgaa | |
| | gtgaatagcaagtgctatgaggaagctaagaagct | |
| | gaagaagatcagcaaccaggccgagtttatcgcct | |
| | ccttctacaacaacgatctgatcaagatcaacggc | |
| | gagctgtatagagtgatcggcgtgaacaacgacct | |
| | gctgaaccggatcgaagtgaacatgatcgacatca | |
| | cctaccgcgagtacctggaaaacatgaacgacaag | |
| | aggccccccaggatcattaagacaatcgcctccaa | |
| | gacccagagcattaagaagtacagcacagacattc | |
| | tgggcaacctgtatgaagtgaaatctaagaagcac | |
| | cctcagatcatcaaaaagggcaaaaggccggcggc | |
| | cacgaaaaaggccggccaggcaaaaaagaaaaagt | |
| | aa | |
| saCas9 | MAPKKKRKVGIHGVPAAKRNYILGLDIGITSVGYG | 11 |
| | IIDYETRDVIDAGVRLFKEANVENNEGRRSKRGAR | |
| | RLKRRRRHRIQRVKKLLFDYNLLTDHSELSGINPY | |
| | EARVKGLSQKLSEEEFSAALLHLAKRRGVHNVNEV | |
| | EEDTGNELSTKEQISRNSKALEEKYVAELQLERLK | |
| | KDGEVRGSINRFKTSDYVKEAKQLLKVQKAYHQLD | |
| | QSFIDTYIDLLETRRTYYEGPGEGSPFGWKDIKEW | |
| | YEMLMGHCTYFPEELRSVKYAYNADLYNALNDLNN | |
| | LVITRDENEKLEYYEKFQIIENVFKQKKKPTLKQI | |
| | AKEILVNEEDIKGYRVTSTGKPEFTNLKVYHDIKD | |
| | ITARKEIIENAELLDQIAKILTIYQSSEDIQEELT | |
| | NLNSELTQEEIEQISNLKGYTGTHNLSLKAINLIL | |
| | DELWHTNDNQIAIFNRLKLVPKKVDLSQQKEIPTT | |
| | LVDDFILSPVVKRSFIQSIKVINAIIKKYGLPNDI | |
| | IIELAREKNSKDAQKMINEMQKRNRQTNERIEEII | |
| | RTTGKENAKYLIEKIKLHDMQEGKCLYSLEAIPLE | |
| | DLLNNPFNYEVDHIIPRSVSFDNSFNNKVLVKQEE | |
| | NSKKGNRTPFQYLSSSDSKISYETFKKHILNLAKG | |
| | KGRISKTKKEYLLEERDINRFSVQKDFINRNLVDT | |
| | RYATRGLMNLLRSYFRVNNLDVKVKSINGGFTSFL | |
| | RRKWKFKKERNKGYKHHAEDALIIANADFIFKEWK | |
| | KLDKAKKVMENQMFEEKQAESMPEIETEQEYKEIF | |
| | ITPHQIKHIKDFKDYKYSHRVDKKPNRELINDTLY | |
| | STRKDDKGNTLIVNNLNGLYDKDNDKLKKLINKSP | |
| | EKLLMYHHDPQTYQKLKLIMEQYGDEKNPLYKYYE | |
| | ETGNYLTKYSKKDNGPVIKKIKYYGNKLNAHLDIT | |
| | DDYPNSRNKVVKLSLKPYRFDVYLDNGVYKFVTVK | |
| | NLDVIKKENYYEVNSKCYEEAKKLKKISNQAEFIA | |
| | SFYNNDLIKINGELYRVIGVNNDLLNRIEVNMIDI | |
| | TYREYLENMNDKRPPRIIKTIASKTQSIKKYSTDI | |
| | LGNLYEVKSKKHPQIIKKGKRPAATKKAGQAKKKK | |

### Expression Cassettes for Targeting PLN

In some embodiments, the present disclosure provides expression cassettes comprising a first polynucleotide encoding a Cas9 protein operably linked to a first promoter and a second polynucleotide encoding a gRNA operably linked to a second promoter. In some embodiments, the first promoter is any promoter suitable for protein expression described herein. In some embodiments, the first promoter is a muscle-specific promoter or a cardiac-specific promoter described herein or known in the art. In some embodiments, the first promoter is a TNNT2 promoter, such as any TNNT2 promoter described herein. In some embodiments, the first promoter is a human TNNT2 promoter. In some embodiments, the first promoter comprises, essentially consists of, or consists of any one of SEQ ID NOs: 12-15. In some embodiments, the second promoter is any promoter suitable for expression of RNA. In some embodiments, the second promoter is a Pol III promoter (e.g., human Pol III promoter) described herein or known in the art. In some embodiments, the second promoter is a U6 promoter. In some embodiments, the second promoter is a human U6 promoter. In some embodiments, the second promoter comprises, essentially consists of, or consists of SEQ ID NO: 22.

The polynucleotides and expression cassettes contemplated herein, regardless of the length of the coding sequence itself, may be combined with other sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art.

In some embodiments, the first polynucleotide sequence further comprises a polyadenylation (poly(A)) signal, and optionally a transcription termination signal. In some embodiments, the poly(A) signal or sequence comprises, essentially consists of or consists of bGHpA pol(A) sequence.

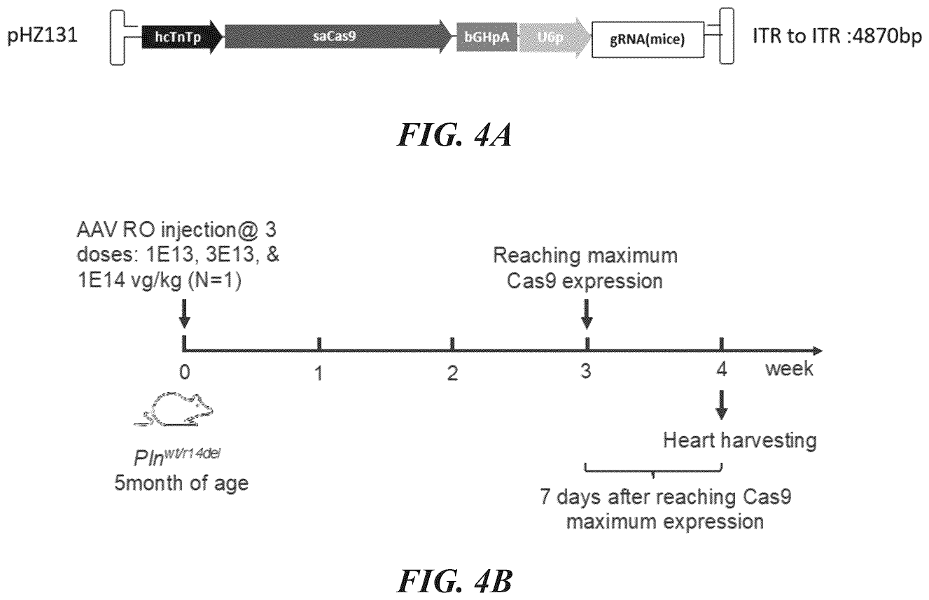

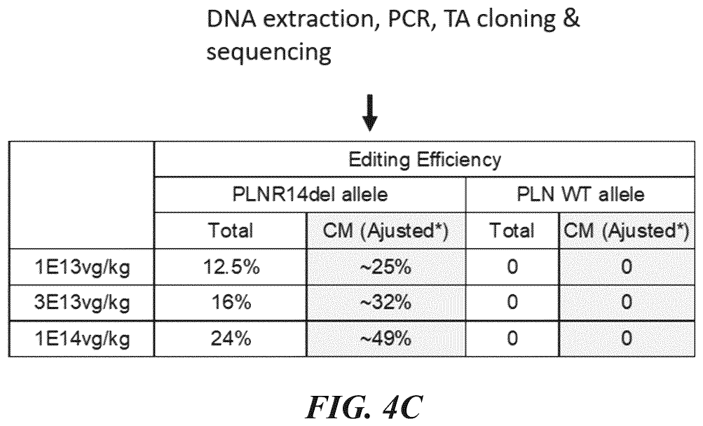

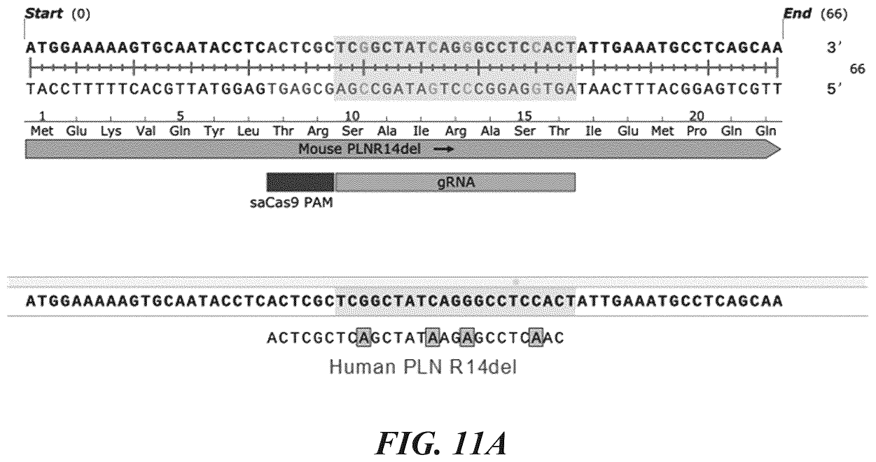

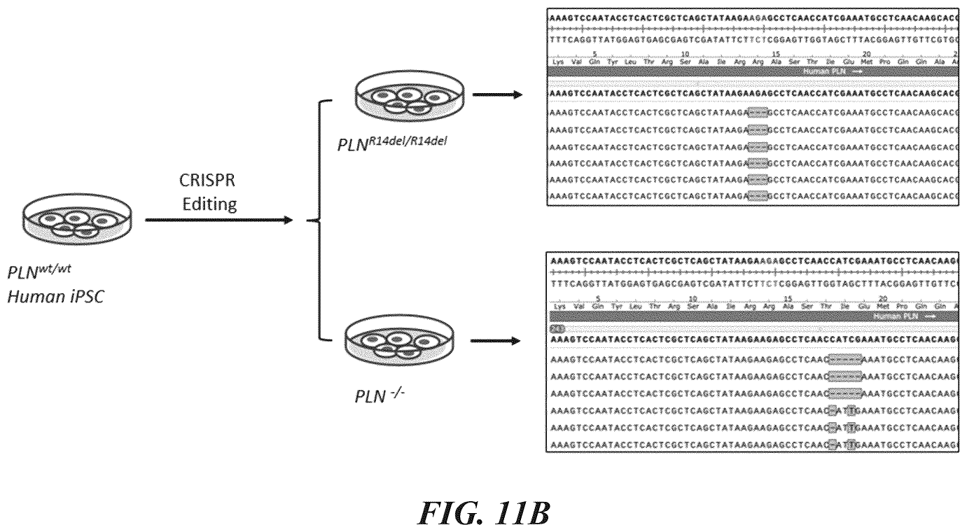

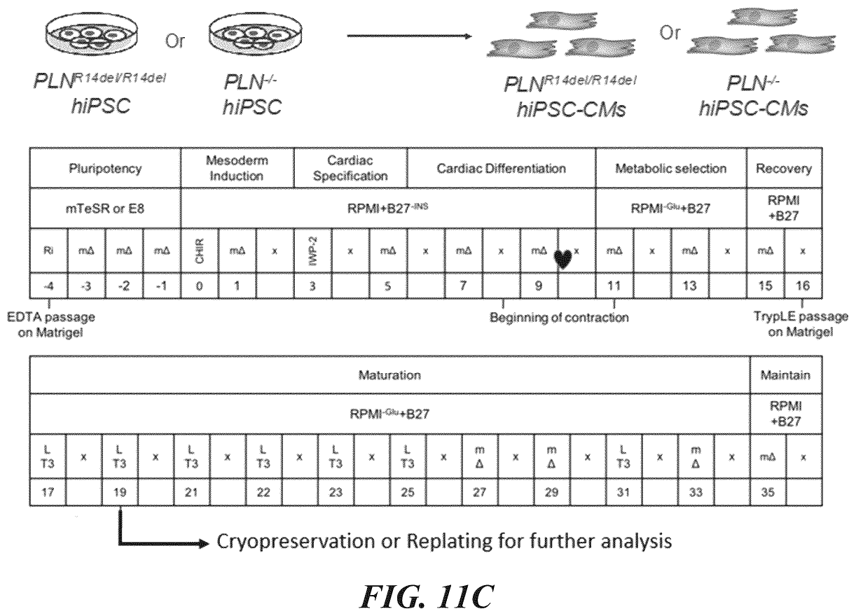

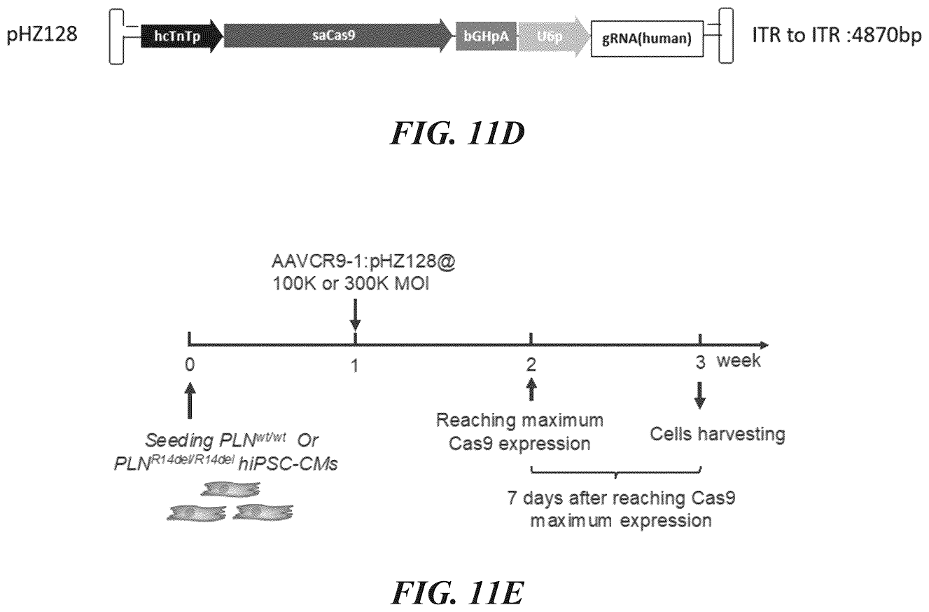

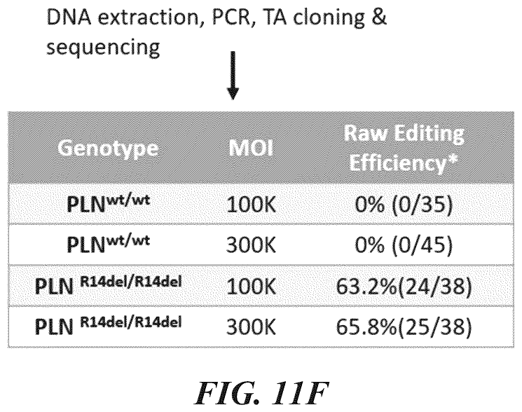

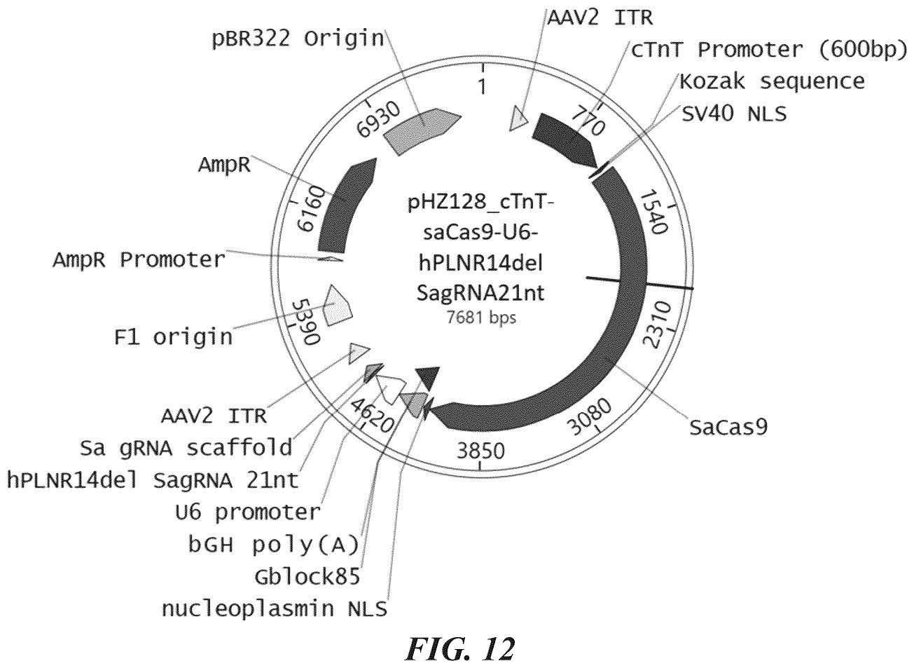

In some embodiments, the expression cassettes described herein comprises, essentially consists of or consists of (i) a first polynucleotide sequence encoding a Cas protein (e.g., saCAs9) operably linked to a first promoter (e.g., human TNNT2) and further comprising a poly(A) sequence, and (ii) a second polynucleotide encoding a gRNA (such as targeting human PLN-R14Del, e.g., gRNA described herein) operably linked to a second promoter (e.g., human U6). In some embodiments, the first polynucleotide and the second polynucleotide are in a head-to-tail orientation. In some embodiments, the expression cassette provided herein is the same or similar to that depicted in FIG. 4A and/or FIG. 11D, including the relative location and/or orientation of elements as presented therein. In some embodiments, the expression cassette provided herein is the same or similar in some or all respects to that depicted in the maps of any one of FIGS. 12-14.

The expression cassette may be flanked by one or more inverted terminal repeats (ITRs). An expression cassette flanked by one or more ITRs is herein referred to as a “viral genome.” The ITRs in an expression cassette serve as markers used for viral packaging of the expression cassette (Clark et al. Hum Gene Ther. 6:1329-41 (1995)). The expression cassette can be integrated into the host cell genome by, for example, infecting the host cell with an rAAV virion comprising a capsid protein and a viral genome comprising the expression cassette, thereby expressing the transgene within a host cell.

The polynucleotide, expression cassette or vector described herein may also contain a ribosome binding site for translation initiation, a transcription terminator, and/or polynucleotide sequences for amplifying expression.

### Illustrative Expression Cassettes

Illustrative, non-limiting arrangements and orientations of the elements in the expression cassettes described herein are shown in FIG. 4A and FIG. 11D. In some embodiments, the 5′ to 3′ arrangement of elements is selected from:
5′-promoter-Cas protein-p(A)-promoter-gRNA-3′;
5′-muscle cell-specific or cardiac cell-specific (e.g., cardiomyocyte) promoter-Cas protein-p(A)-promoter (e.g., pol III promoter such as U6)-gRNA-3′;
5′-Constitutive promoter-Cas protein-p(A)-promoter (e.g., pol III promoter such as U6)-gRNA-3′;
5′-human TNNT2 promoter-Cas protein-p(A)-promoter (e.g., pol III promoter such as U6)-gRNA-3′;
5′-human TNNT2 promoter-Cas protein-p(A)-promoter (e.g., pol III promoter such as U6)-gRNA-3′.

In some embodiments, the polynucleotide encoding the Cas protein is oriented in head-to tail orientation relative to the polynucleotide encoding the guide RNA. In some embodiments, such expression cassettes exhibit improved performance (e.g., as measured by gRNA editing efficiency) relative to expression cassettes wherein the polynucleotide encoding the Cas protein is oriented in head-to head orientation relative to the polynucleotide encoding the guide RNA. In some embodiments, the polynucleotide encoding the Cas protein is not oriented in head-to head orientation relative to the polynucleotide encoding the guide RNA.

In some embodiments, different expression cassette arrangements are also contemplated herein, including different arrangement and orientation of elements.

In some embodiments, the expression cassettes described herein further comprise one or more enhancers. In some embodiments, the expression cassettes described herein do not comprise an enhancer.

In some embodiments, the expression cassettes provided herein comprise any regulatory element known in the art. In some embodiments, the expression cassettes provided herein comprise any post-transcriptional regulatory element known in the art. In some embodiments, the expression cassettes provided herein do not comprise a post-transcriptional regulatory element (e.g., do not comprise the WPRE element).

In some embodiments, the expression cassettes provided herein comprise an intron. In some embodiments, the expression cassettes provided herein do not comprise an intron.

It should be understood that the illustrative orientations of the expression cassette can include flanking inverted terminal repeat (ITR) sequences on the 5′ and 3′ ends of the expression cassette.

### Regulatory Elements

As used herein, the term “regulatory element” refers those non-translated regions of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interact with host cellular proteins to carry out transcription and translation. Such elements may vary in their strength and specificity. The transcriptional regulatory element may be functional in either a eukaryotic cell (e.g., a mammalian cell) or a prokaryotic cell (e.g., bacterial or archaeal cell). In some embodiments, a polynucleotide sequence described herein is operably linked to multiple control elements that allow expression of the polynucleotide in both prokaryotic and eukaryotic cells.

As used herein, the term “transcription start site” or “TSS” refers to the first base pair transcribed by an RNA polymerase when the RNA polymerase initiates transcription. A TSS is different from the start codon (canonically, ATG), which must be downstream of the TSS in the transcribed region of the polynucleotide. The location of a transcription start site can be determined experimentally or by prediction using any of various prediction algorithms. Annotated TSSs are available from the Eukaryotic Promoter Database and the UCSC Genome Browser. Multiple TSSs for TNNT2 are identified in the UCSC Genome Browser.

As used herein, the TSS for TNNT2 is defined to be the sequence identified by the C at the 5′ end of the motif identified by dbTSS: CTCCATC.

### Promoters for Cas Endonuclease

The term “promoter” as used herein refers to a DNA sequence that directs the binding of RNA polymerase and thereby promotes RNA synthesis. Promoters and corresponding protein or polypeptide expression may be ubiquitous, meaning strongly active in a wide range of cells, tissues and species or cell-type specific, tissue-specific, or species specific. Examples of ubiquitous promoters include the CAG promoter and CMB promoter (Yue et al. BioTechniques 33:672-678 (2002)). Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

In some embodiments, the expression cassette comprises a single promoter. In some embodiments, the expression cassette comprises at least one promoter. In some embodiments, the expression cassette comprises two promoters. In some embodiments, the expression cassette comprises a ubiquitous promoter. In some embodiments, the expression cassette comprises an inducible promoter. In some embodiments, the expression cassette comprises a cell-type specific promoter. In some embodiments, the promoter specifically promotes expression of the polynucleotide encoding a polypeptide, or functional variant thereof, in a cardiac cell (e.g., a cardiomyocyte).

### Constitutive Promoters

In some embodiments, the promoter is a constitutive promoter. As used herein, a “constitutive promoter” is one wherein the level of expression does not vary from one cell type compared to a different cell type. Suitable constitutive promoters include a human elongation factor 1 a subunit (EFlα) promoter, a P-actin promoter, an a-actin promoter, a p-glucuronidase promoter, CAG promoter, super core promoter, and a ubiquitin promoter.

In some embodiments, the Cas endonuclease is operably linked to a constitutive promoter.

### Cell Specific Promoters

In some embodiments, the promoter is a muscle cell-specific promoter. In some embodiments, the promoter is a cardiac-specific promoter. In some embodiments, the promoter is a cardiomyocyte-specific promoter.

A “cardiomyocyte-specific promoter”, as used herein, specifies a promoter whose activity in cardiomyocytes is at least 2-fold higher than in any other non-cardiac cell type or cardiac cell which is not a cardiomyocyte. Preferably, a cardiomyocyte-specific promoter suitable for being used in the vector of the present disclosure has an activity in cardiomyocytes which is at least 5-fold, at least 10-fold, at least 15-fold, at least 20-fold, at least 25-fold, or at least 50-fold higher compared to its activity in a non-cardiac cell type or a cardiac cell type which is not a cardiomyocyte.

In some embodiments, the cardiac-specific or cardiomyocyte-specific promoter is a human promoter. Examples of cardiac-specific or cardiomyocyte-specific promoter include, but are not limited to, the alpha myosin heavy chain promoter, the myosin light chain 2v promoter, the alpha myosin heavy chain promoter, the alpha-cardiac actin promoter, the alpha-tropomyosin promoter, the cardiac troponin C promoter, the cardiac troponin I promoter, the cardiac myosin-binding protein C promoter, and the sarco/endoplasmic reticulum Ca²⁺ ATPase (SERCA) promoter (e.g. isoform 2 of SERCA2).

In some embodiments, the cardiac-specific promoter is the cardiac troponin T promoter (TNNT2) promoter. In some embodiments, the polynucleotides, expression cassettes and vectors described herein comprise a TNNT2 promoter, such as any of the TNNT2 promoters described herein. In some embodiments, the TNNT2 promoter is a human TNNT2 promoter. In some embodiments, the TNNT2 promoter is a chicken TNNT2 promoter. In some embodiments, the cardiac TNNT2 promoter is modified, e.g., by the deletion, insertion, or substitution of polynucleotides. In some embodiments, TNNT2 promoter is a modified human TNNT2 promoter (e.g., a truncated human TNNT2 promoter). Illustrative polynucleotide sequences of the cardiac TNNT2 promoter are shown in Table 4 below. The transcription start site (TSS) of the TNNT2 promoters are bolded and underlined.

TABLE 4
Exemplary troponin T (TNNT2) promoter sequences

| | | SEQ |
|---|---|---|
| Name | DNA Sequence | ID NO: |
| TNNT2p-600 | gtcatggagaagacccaccttgcagatgtcctcactggggctggc | 12 |
| | agagccggcaacctgcctaaggctgctcagtccattaggagccag | |
| | tagcctggaagatgtctttacccccagcatcagttcaagtggagc | |
| | agcacataactcttgccctctgccttccaagattctggtgctgag | |
| | acttatggagtgtcttggaggttgccttctgccccccaaccctgc | |
| | tcccagctggccctcccaggcctgggttgctggcctctgctttat | |
| | caggattctcaagagggacagctggtttatgttgcatgactgttc | |
| | cctgcatatctgctctggttttaaatagcttatctgagcagctgg | |
| | aggaccacatgggcttatatggcgtggggtacatgttcctgtagc | |
| | cttgtccctggcacctgccaaaatagcagccaacaccccccaccc | |
| | ccaccgccatccccctgccccacccgtcccctgtegcacattcct | |
| | ccctccgcagggctggctcaccaggccccagcccacatgcctgct | |
| | taaagccctctccatcctctgcctcacccagtccccgctgagact | |
| | gagcagacgcctcca | |
| TNNT2p-500 | gatgtctttacccccagcatcagttcaagtggagcagcacataac | 13 |
| | tcttgccctctgccttccaagattctggtgctgagacttatggag | |
| | tgtcttggaggttgccttctgccccccaaccctgctcccagctgg | |
| | ccctcccaggcctgggttgctggcctctgctttatcaggattctc | |
| | aagagggacagctggtttatgttgcatgactgttccctgcatatc | |
| | tgctctggttttaaatagcttatctgagcagctggaggaccacat | |
| | gggcttatatggcgtggggtacatgttcctgtagccttgtccctg | |
| | gcacctgccaaaatagcagccaacaccccccacccccaccgccat | |
| | ccccctgccccaccegtcccctgtegcacattcctccctccgcag | |
| | ggctggctcaccaggccccagcccacatgcctgettaaagccctc | |
| | tccatcctctgcctcacccagtccccgctgagactgagcagacgc | |
| | ctcca | |
| TNNT2p-400 | gttgccttctgccccccaaccctgctcccagctggccctcccagg | |
| | cctgggttgctggcctctgctttatcaggattctcaagagggaca | |
| | gctggtttatgttgcatgactgttccctgcatatctgctctggtt | |
| | ttaaatagcttatctgagcagctggaggaccacatgggcttatat | |
| | ggcgtggggtacatgttcctgtagccttgtccctggcacctgcca | |
| | aaatagcagccaacaccccccacccccaccgccatccccctgccc | |
| | cacccgtcccctgtcgcacattcctccctccgcagggctggctca | |
| | ccaggccccagcccacatgcctgcttaaagccctctccatcctct | |
| | gcctcacccagtccccgctgagactgagcagacgcctcca | 14 |
| TNNT2p-300 | gttgcatgactgttccctgcatatctgctctggttttaaatagct | 15 |
| | tatctgagcagctggaggaccacatgggcttatatggcgtggggt | |
| | acatgttcctgtagccttgtccctggcacctgccaaaatagcagc | |
| | caacaccccccacccccacegccatccccctgccccaccegtccc | |
| | ctgtegcacattcctccctccgcagggctggctcaccaggcccca | |
| | gcccacatgcctgettaaagccctctccatcctctgcctcaccca | |
| | gtccccgctgagactgagcagacgcctcca | |
| Long TNNT | agaggaccctttcaaggacattagtggtggaggcagcatagtagc | 16 |
| | tcccaaggcagagggattgagagaagagtttgaggactgggaagg | |
| | cgggacacatgattgggtgatgggagaagggggcagagaatagcg | |
| | agattgctttctttgcccacggagaaacagaggagtgtggatcat | |
| | gaatgggcaagatctttaagtgccagggggggtcatggaggaggg | |
| | gagggcctgctccagaggaggaccattcctgcttcagagccaagc | |
| | aggacctaggctgtgaagattcggagaaagagatggaggggagag | |
| | tcagctcagctgcttactggcttgctttcctcctgtctctttcat | |
| | tttcataatctaccaaaccctgcaatgggccagccttgaacatac | |
| | aagtgcatgtgcatggtcagacacaggcaagcaagcaagacccct | |
| | aggcctgacctatgcatctgcaatctagtaggtttagcagatcat | |
| | agccccgcactgcttgattttaaagccgttaggggatgacctttg | |
| | acagtccgcatcacccctctcacacaacgagcgcctgttcaaggt | |
| | tcttgactggaagttctaccttgtatctggcctcctgtagcagtt | |
| | tcagtccattccctgtgaggagggtgtgccacatggctttggggg | |
| | tcatggagaagacccaccttgcagatgtcctcactggggctggca | |
| | gagccggcaacctgcctaaggctgctcagtccattaggagccagt | |
| | agcctggaagatgtctttacccccagcatcagttcaagtggagca | |
| | gcacataactcttgccctctgccttccaagattctggtgctgaga | |
| | cttatggagtgtcttggaggttgccttctgccccccaaccctgct | |
| | cccagctggccccccaggcctgggttgctggcctctgctttatca | |
| | ggattctcaagagggacagctggtttatgttgcatgactgttccc | |
| | tgcatatctgctctggttttaaatagcttatctgagcagctggag | |
| | gaccacatgggcttatatggcgtggggtacatgttcctgtagcct | |
| | tgtccctggcacctgccaaaatagcagccaacaccccccaccccc | |
| | accgccatccccctgccccaccegtcccctgtcgcacattcctcc | |
| | ctccgcagggctggctcaccaggccccagcccacatgcctgctta | |
| | aagccctctccatcctctgcctcacccagtccccgctgagactga | |
| | gcagacgcctccaggatctgtcggcagctgctgttctgaggtaag | |
| | gctcgggcagggctctggggaagaggagagcagagaatggacggg | |
| | gagatgtgagggtcttgggccctggcatatttacccagagtctgc | |
| | ctgtgtccgcagaagtccatggcccctcctggtggaggccacact | |
| | tcagaggacaggttgccaggtctgggctccaagattggtacaata | |
| | gagcagagaga | |
| Chicken TnT | gggataaaagcagtctgggctttcacatgacagcatctggggctg | 17 |
| promoter | cggcagagggtcgggtccgaagcgctgccttatcagcgtccccag | |
| | ccctgggaggtgacagctggctggcttgtgtcagcccctcgggca | |
| | ctcacgtatctccgtccgacgggtttaaaatagcaaaactctgag | |
| | gccacacaatagcttgggcttatatgggctcctgtgggggaaggg | |
| | ggagcacggagggggccggggccgctgctgccaaaatagcagctc | |
| | acaagtgttgcattcctctctgggcgccgggcacattcctgctgg | |
| | ctctgcccgccccggggtgggcgccggggggaccttaaagcctct | |
| | gccccccaaggagcccttcccagacagccgccggcacccaccgct | |
| | ccgtggga | |

In some embodiments, the promoter is any promoter, e.g., any TNNT2 promoter, described in WO2021/163357A2 and/or U.S. Pat. No. 11,129,908, both of which are incorporated by reference herein in their entirety.

In some embodiments, the promoter is any promoter, e.g., any TNNT2 promoter, described in WO2023/283649, which is incorporated by reference herein in its entirety.

In some embodiments, the promoter comprises a sequence that shares at least 80%, at least 90%, or at least 100% identity to any one of SEQ ID NOs: 12-15. In some embodiments, the promoter comprises a sequence that shares at least 80%, at least 90%, or at least 100% identity to SEQ ID NO: 12. In some embodiments, the promoter comprises a sequence that shares at least 80%, at least 90%, or at least 100% identity to SEQ ID NO: 13. In some embodiments, the promoter comprises a sequence that shares at least 80%, at least 90%, or at least 100% identity to SEQ ID NO: 14. In some embodiments, the promoter comprises a sequence that shares at least 80%, at least 90%, or at least 100% identity to SEQ ID NO: 15.

In some embodiments, the promoter to which Cas endonuclease is operably linked comprises, essentially consists of or consists of a sequence of SEQ ID NO: 12. In some embodiments, the promoter to which Cas endonuclease is operably linked comprises, essentially consists of or consists of a sequence of SEQ ID NO: 13. In some embodiments, the promoter to which Cas endonuclease is operably linked comprises, essentially consists of or consists of a sequence of SEQ ID NO: 14. In some embodiments, the promoter to which Cas endonuclease is operably linked comprises, essentially consists of or consists of a sequence of SEQ ID NO: 15.

In some embodiments, the promoter to which Cas endonuclease is operably linked comprises, essentially consists of or consists of a sequence having at least 70%, 80%, 85%, 90%, 95%, 97%, 98%, 99% or 100% sequence identity to SEQ ID NO: 12. In some embodiments, the promoter to which Cas endonuclease is operably linked comprises, essentially consists of or consists of a sequence having at least 70%, 80%, 85%, 90%, 95%, 97%, 98%, 99% or 100% sequence identity to SEQ ID NO: 13. In some embodiments, the promoter to which Cas endonuclease is operably linked comprises, essentially consists of or consists of a sequence having at least 70%, 80%, 85%, 90%, 95%, 97%, 98%, 99% or 100% sequence identity to SEQ ID NO: 14. In some embodiments, the promoter to which Cas endonuclease is operably linked comprises, essentially consists of or consists of a sequence having at least 70%, 80%, 85%, 90%, 95%, 97%, 98%, 99% or 100% sequence identity to SEQ ID NO: 15.

The term “modified cardiac TNNT2 promoter” as used herein refers to a promoter that comprises a polynucleotide sequence of at least 200 base pairs that comprises one or more continuous or discontinuous polynucleotide segments each sharing 70%, 80%, 90%, 95%, 96%, 97%, 98%, 99%, or 100% identity to a corresponding segment of the TNNT2p-600 segment provided in Table 4 as SEQ ID NO: 12. As it is a “promoter,” a modified cardiac TNNT2 promoter must be capable of promoting initiation of transcription by an RNA polymerase in a host or target cell at or near a TSS within the promoter (i.e., at or near the TTS of TNNT2 as defined herein) or, if the endogenous TSS of TNNT2 is not present in the modified cardiac TNNT2 promoter then at a heterologous TSS at most 100 base pairs downstream (3′ on the sense strand) to the downstream (3′) end of the modified cardiac TNNT2 promoter. Similarly stated, a modified cardiac TNNT2 promoter may comprise only sequences upstream of the TSS of TNNT2 or more comprise the TSS of TNNT2.

In some embodiments, the promoter is a cardiac TNNT2 promoter modified to comprise a polynucleotide sequence of between about 300 to 500 base pairs, between about 350 to 500 base pairs, between about 350 to 450 bp, between about 400 to 450 base pairs, or between about 375 to 425 base pairs in length. In some embodiments, the modified cardiac TNNT2 promoter comprises a polynucleotide sequence of between about 350 base pairs to about 450 base pairs, between about 375 base pairs to about 425 base pairs, between about 375 base pairs to about 400 base pairs, between about 375 base pairs to about 425 base pairs, between about 400 base pairs to about 425 base pairs, or between about 400 base pairs to about 450 base pairs.

In some embodiments, the cardiac TNNT2 promoter comprises a polynucleotide sequence of about 400 base pairs.

In some embodiments, the promoter has the same cell-type specificity as a native troponin T promoter of about 600 bp. In some embodiments, the promoter described herein has the same cell-type specificity as a reference promoter comprising SEQ ID NO: 12.

In some embodiments, the modified cardiac troponin T promoter comprises between 300 bp and 500 bp of SEQ ID NO: 12. For instance, the modified cardiac troponin T promoter may comprise SEQ ID NO: 13. In some examples, the 300 bp-500 bp sequence may be linked to further polynucleotide sequences but may not be linked to additional sequences derived from SEQ ID NO: 12. For example, in an embodiment, the modified cardiac troponin T promoter may include no more than 500 bp of SEQ ID NO: 12 but may include additional unrelated polynucleotide sequences. In another example, the modified cardiac troponin T promoter may include SEQ ID NO: 13, no additional sequences derived from SEQ ID NO: 12, but may include additional unrelated polynucleotide sequences.

In some embodiments, the cardiac TNNT2 promoter is modified by the deletion of polynucleotides. A modification may include one, two, three or more internal deletions. Each deletion may be a deletion of 1 base pair, 2 base pairs, 3 base pairs, 4 base pairs, 5 base pairs, 10 base pairs, 15 base pairs, 20 base pairs, 25 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs with respect to a reference cardiac TNNT2 promoter (SEQ ID NO: 12) having about 600 base pairs.

In some embodiments, the TNNT2 promoter is modified by the deletion of polynucleotides from the upstream end of the promoter with respect to a reference cardiac TNNT2 promoter (SEQ ID NO: 12) having about 600 base pairs. A modification may include the deletion of 1 base pair, 2 base pairs, 3 base pairs, 4 base pairs, 5 base pairs, 10 base pairs, 15 base pairs, 20 base pairs, 25 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs from the upstream end of the promoter with respect to a reference cardiac TNNT2 promoter (SEQ ID NO: 12) having about 600 base pairs. In some embodiments, the modification is a 200 base pair deletion from the upstream end of the promoter with respect to a reference cardiac TNNT2 promoter (SEQ ID NO: 12) having about 600 base pairs.

In some embodiments, the cardiac TNNT2 promoter is modified by the deletion of polynucleotides from the downstream end of the promoter with respect to a reference cardiac TNNT2 promoter (SEQ ID NO: 12) having about 600 base pairs. A modification may include the deletion of 1 base pair, 2 base pairs, 3 base pairs, 4 base pairs, 5 base pairs, 10 base pairs, 15 base pairs, 20 base pairs, 25 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs from the downstream end of the promoter with respect to a reference cardiac TNNT2 promoter (SEQ ID NO: 12) having about 600 base pairs.

In some embodiments, the cardiac TNNT2 promoter is modified by an internal deletion of polynucleotides. A modification may include the internal deletion of 1 base pair, 2 base pairs, 3 base pairs, 4 base pairs, 5 base pairs, 10 base pairs, 15 base pairs, 20 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs with respect to a reference cardiac TNNT2 promoter (SEQ ID NO: 12).

In some embodiments, the cardiac TNNT2 promoter is modified by the insertion of polynucleotides. A modification may include the insertion of 1 base pair, 2 base pairs, 3 base pairs, 4 base pairs, 5 base pairs, 10 base pairs, 15 base pairs, 20 base pairs, 25 base pairs, 30 base pairs, 35 base pairs, 40 base pairs, 45 base pairs, 50 base pairs, 55 base pairs, 60 base pairs, 65 base pairs, 70 base pairs, 75 base pairs, 80, base pairs, 85 base pairs, 90 base pairs, 100 base pairs, 125 base pairs, 150 base pairs, 175 base pairs, 200 base pairs, 225 base pairs, 250 base pairs, 275 base pairs, or 300 base pairs with respect to a reference cardiac TNNT2 promoter (SEQ ID NO: 12).

In some embodiments, the cardiac TNNT2 promoter is modified by the substitution of polynucleotides. A modification may include the substitution of 1 base pair, 2 base pairs, 3 base pairs, 4 base pairs, 5 base pairs, 6 base pairs, 7 base pairs, 8 base pairs, 9 base pairs, or 10 base pairs with respect to a reference cardiac TNNT2 promoter (SEQ ID NO: 12).

In some embodiments, the cardiac TNNT2 promoter comprises a polynucleotide comprising a sequence that shares at least 65%, 70%, 75%, 80%, 85%, 90%, 95%, 96%, 97%, 98%, 99%, or and 100% identity to any one of SEQ ID NOs: 12-17. In some embodiments, the polynucleotide comprises a sequence that shares at least 80% identity to any one of SEQ ID NOS: 12-17. In some embodiments, the polynucleotide comprises a sequence that shares 100% identity to any one of SEQ ID NOS: 12-17. In some embodiments, the polynucleotide comprises a sequence that shares at least 80% identity to SEQ ID NO: 12. In some embodiments, the polynucleotide comprises a sequence that shares at least 90% identity to SEQ ID NO: 12. In some embodiments, the polynucleotide comprises a sequence that shares at least 100% identity to SEQ ID NO: 12. In some embodiments, the polynucleotide comprises a sequence that shares at least 80% identity to SEQ ID NO: 13. In some embodiments, the polynucleotide comprises a sequence that shares at least 90% identity to SEQ ID NO: 13. In some embodiments, the polynucleotide comprises a sequence that shares at least 100% identity to SEQ ID NO: 13.

In some embodiments, the promoter is TNNT2p-600, as described herein.

In some embodiments, the promoter is TNNT2p-500, as described herein.

In some embodiments, the promoter is TNNT2p-400, as described herein.

In some embodiments, the promoter is TNNT2p-300, as described herein.

In some embodiments, the promoter is a Chicken TnT promoter, as described herein.

In some embodiments, the promoter is a long TNT promoter, as described herein.

RNA Expression-Driving Promoters for gRNA

In some embodiments, the expression cassette comprises an RNA expression-driving promoter operably linked to gRNA. In some embodiments, the expression cassette comprises a promoter that expresses a downstream gRNA sequence.

In some embodiments, the RNA expression-driving promoter is a Pol III promoter, such as any Pol III promoters known in the art or described herein. In some embodiments, the RNA expression-driving promoter is any promoter suitable for expression in mammalian cells. In some embodiments, the RNA expression-driving promoter is a human Pol III promoter.

In some embodiments, the RNA expression-driving promoter is Human H1 promoter 1. In some embodiments, the promoter is Human H1 promoter 1 of SEQ ID NO: 19.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 19.

In some embodiments, the RNA expression-driving promoter is Human H1 promoter 2. In some embodiments, the promoter is Human H1 promoter 2 of SEQ ID NO: 20.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 20.

In some embodiments, the RNA expression-driving promoter is Doxycycline-inducible variant of the human H1 RNA promoter. In some embodiments, the promoter is Doxycycline-inducible variant of the human H1 RNA promoter of SEQ ID NO: 21.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 21.

In some embodiments, the RNA expression-driving promoter is Human U6 promoter. In some embodiments, the promoter is Human U6 promoter of SEQ ID NO: 22.

In some embodiments, the promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 22.

In some embodiments, the RNA expression-driving promoter is Mouse U6 promoter. In some embodiments, the promoter is Mouse U6 promoter of SEQ ID NO: 23.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 23.

In some embodiments, the RNA expression-driving promoter is C. elegans U6 promoter 1. In some embodiments, the RNA expression-driving promoter is C. elegans U6 promoter 1 of SEQ ID NO: 24.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 24.

In some embodiments, the RNA expression-driving promoter is C. elegans U6 promoter 2. In some embodiments, the RNA expression-driving promoter is C. elegans U6 promoter 2 of SEQ ID NO: 25

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 25.

In some embodiments, the RNA expression-driving promoter is Drosophila U6 promoter 1. In some embodiments, the RNA expression-driving promoter is Drosophila U6 promoter 1 of SEQ ID NO: 26.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 26.

In some embodiments, the RNA expression-driving promoter is Drosophila U6 promoter 2. In some embodiments, the RNA expression-driving promoter is Drosophila U6 promoter 2 of SEQ ID NO: 27.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 27.

In some embodiments, the RNA expression-driving promoter is Drosophila U6 promoter 3. In some embodiments, the promoter is Drosophila U6 promoter 3 of SEQ ID NO: 28.

In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 28.

TABLE 5
Exemplary RNA expression-driving promoter sequences

| | | SEQ |
|---|---|---|
| Name | Sequence | ID NO: |
| Human H1 | tatttgcatgtcgctatgtgttctgggaaatcaccataaacgtgaaatgtctttggatttgggaa | 19 |
| promoter 1 | tcttataagttctgtatgagaccac | |
| Human H1 | gaacgctgacgtcatcaacccgctccaaggaatcgcgggcccagtgtcactaggcgggaac | 20 |
| promoter 2 | acccagcgcgcgtgcgccctggcaggaagatggctgtgagggacaggggagtggcgccctg | |
| | caatatttgcatgtcgctatgtgttctgggaaatcaccataaacgtgaaatgtctttggat | |
| | ttgggaatcttataagttctgtatgagaccac | |
| Doxycycline- | atatttgcatgtcgctatgtgttctgggaaatcaccataaacgtgaaatccctatcagtgatagag | 21 |
| inducible | acttataagttccctatcagtgatagaga | |
| variant human | | |
| H1 promoter | | |
| Human U6 | gagggcctatttcccatgattccttcatatttgcatatacgatacaaggctgttagagagataatt | 22 |
| promoter | ggaattaatttgactgtaaacacaaagatattagtacaaaatacgtgacgtagaaagtaataattt | |
| | cttgggtagtttgcagttttaaaattatgttttaaaatggactatcatatgcttaccgtaacttga | |
| | aagtatttcgatttcttggctttatatatcttgtggaaaggac | |
| Mouse U6 | gatccgacgccgccatctctaggcccgcgccggccccctcgcacagacttgtgggagaagctcg | 23 |
| promoter | gctactcccctgccccggttaatttgcatataatatttcctagtaactatagaggcttaatgtg | |
| | cgataaaagacagataatctgttctttttaatactagctacattttacatgataggcttggatt | |
| | tctataagagatacaaatactaaattattattttaaaaaacagcacaaaaggaaactcacccta | |
| | actgtaaagtaattgtgtgttttgagactataaatatcccttggagaaaagccttgtt | |
| C. elegans U6 | ctccaagaactcgtacaaaaatgctctgaagtaggtctcgagatcaatactgggaagacgaaa | 24 |
| promoter 1 | gtcttgcgaaaccgattcgctgaccccagtaaagtctactteggtagcccttcccccaccaccc | |
| | agctcgacgacgtcgacgagtacatctacctcggtcgtcaaatcaacgcccaaaacaacttga | |
| | tgccggaaatccaccgaagacgtcgagcagcctgggctgcattcaatggaatcaagaatgcc | |
| | accgactccatcaccgacaagaagattcgtgcgaatctgttcgactcaattgtccttccagcgc | |
| | tcacctacgtttcagaagcctggacattcaccaaagctctatccgaacgagtacgaatcacaca | |
| | tgcctcccccacaaattattgatctacatccccatttggtatagtgtcttacaggactctcaat | |
| | atgtcagctgcatgataaatggtccctatataattgaattgcaaatctaaatgttt | |
| C. elegans U6 | caaaaaaaactagcaataaaggaataaaaaactgtacaccttaaaggcgcacactctgttttgcaa | 25 |
| promoter 2 | attttatttttagttgtgaattttctgctgagacctgaaaatagcaactttagtactactataatt | |
| | tgtcaaccttttcaaaaaaagcatgcaatttttgagaaactcttataaaagctattattaaaaaaa | |
| | caccttttttccaaaattattccacaaaaaatatgttatgaaatgcctacaccctctcacacacac | |
| | tctttatactactctgtcaaactcacgagatgtctgccgcctcttgtgttgcccctatataaacac | |
| | ctcctattgcgagatgtctt | |
| Drosophila U6 | attttcaacgtcctcgatagtatagtggttagtatccccgcctgtcacgcgggagaccggggttcaa | 26 |
| promoter 1 | ttccccgtcggggagaatctgtgattctttttttttttcttttactttgttatataaacaatttttg | |
| | ttttaattgaatctaatttgccattgcttttaggaatctcaggcatccagcaagcgtttgtccgccg | |
| | aatcgcccatcagtgaagaagatcctgtggcggctacgaaaatctccccggccatgteggcctcc | |
| | acctccagcgaaaaacccatcagcgagctggccacctctgtgctgacccaccgctttccaga | |
| | ctccacctcctcacccggcgaacatggccttggacgaatgcagttgtcgatccgctacagcgc | |
| | ccagcgtcaaaaactagacgtgaccatacacaaaatccagaagataccacttegcgatccca | |
| | gcaatatccccgatccgtatgttaagctgtatctgttgcctggacgcaccaaggagtcgaaacg | |
| | caagacgagcgtgatcaaggacaactgcaaccccgtctacgatgcatcctttgagtacctgat | |
| | ttccattgccgaactcaggcagacggaactggaggtgacggtgtgcacccaaaagggattcc | |
| | tatccggcggtagtcccatcattggcatggtaggtacccgaaagcaaccccttagttacagac | |
| | acagcgcgtacgtccttcgcatccttatgattcccaagtacatattctgcaagagtacagtatata | |
| | taggaaagatatccgggtgaacttcg | |
| Drosophila U6 | gttcgacttgcagcctgaaatacggcacgagtaggaaaagccgagtcaaatgccgaatgcagagt | 27 |
| promoter 2 | ctcattacagcacaatcaactcaagaaaaactcgacacttttttaccatttgcacttaaatcctt | |
| | ttttattcgttatgtatactttttttggtccctaaccaaaacaaaaccaaactctcttagtcgtg | |
| | cctctatatttaaaactatcaatttattatagtcaataaatcgaactgtgttttcaacaaacgaa | |
| | caataggacactttgattctaaaggaaattttgaaaatcttaagcagagggttcttaagaccatt | |
| | tgccaattcttataattctcaactgctctttcctgatgttgatcatttatataggtatgttttcct | |
| | caatacttc | |
| Drosophila U6 | ttttttgctcacctgtgattgctcctactcaaatacaaaaacatcaaattttctgtcaataaagca | 28 |
| promoter 3 | tatttatttatatttattttacaggaaagaattccttttaaagtgtattttaacctataatgaaaa | |
| | acgattaaaaaaaatacataaaataattcgaaaatttttgaatagcccaggttgataaaaattcat | |
| | ttcatacgttttataacttatgcccctaagtattttttgaccatagtgtttcaattctacattaat | |
| | tttacagagtagaatgaaacgccacctactcagccaagaggcgaaaaggttagctcgccaagcaga | |
| | gagggcgccagtgctcactactttttataattctcaacttctttttccagactcagttcgtatata | |
| | tagacctattttcaatttaacgtcg | |

### Poly(A) Tail

In some embodiments, the expression cassette comprises a poly(A) signal sequence. The poly(A) sequence can be any poly(A) sequence known in the art or described herein.

In some embodiments, expression cassettes comprise a polyadenylation sequence 3′ of a polynucleotide encoding a polypeptide to be expressed. The term “poly(A) site” or “poly(A) sequence” as used herein denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal poly(A) sequence (e.g., AATAAA, ATTAAA, AGTAAA). In particular embodiments, the poly(A) sequence is an SV40 poly(A) sequence, a bovine growth hormone poly(A) sequence (BGHpA), a rabbit β-globin poly(A) sequence (rogpA), variants thereof, or another suitable heterologous or endogenous poly(A) sequence known in the art.

In some embodiments, the poly(A) signal is a BGH poly(A) sequence.

In some embodiments, the BGH poly(A) signal sequence comprises the polynucleotide sequence that shares at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 29.

In some embodiments, the poly(A) signal is an SV40 poly(A) sequence.

In some embodiments, the SV40 poly(A) signal sequence comprises the polynucleotide sequence that shares at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 30.

TABLE 6
Exemplary poly(A) sequences

| | | SEQ ID |
|---|---|---|
| Name | Sequence | NO: |
| BGH poly(A) | gcctcgactgtgccttctagttgccagccatctgttgtttgcccc | 29 |
| sequence | tcccccgtgccttccttgaccctggaaggtgccactcccactgtc | |
| | ctttcctaataaaatgaggaaattgcatcgcattgtctgagtagg | |
| | tgtcattctattctggggggtggggggggcaggacagcaaggggg | |
| | aggattgggaagacaatagcaggcatgctgggga | |
| SV40 poly(A) | gatccagacatgataagatacattgatgagtttggacaaaccaca | 30 |
| sequence | actagaatgcagtgaaaaaaatgctttatttgtgaaatttgtgat | |
| | gctattgctttatttgtaaccattataagctgcaataaacaagt | |

### Additional Elements

In some embodiments of the expression cassette, the Cas protein coding sequence is preceded by a Kozak sequence. Suitable Kozak sequences are known in the art and include, but are not limited to, the Kozak sequence of SEQ ID NO: 38.

In some embodiments, the coding sequence of the Cas protein is linked to one or more nuclear localization sequences (NLS). Suitable NLS sequences are known in the art and include, but are not limited to, the sequence encoded by the SV40 NLS (SEQ ID NO: 39) and the nucleoplasmin NLS (SEQ ID NO: 40). In some embodiments, a Cas protein coding sequence comprises an NLS at 5′ end (or an N-terminal end of Cas) and/or 3′ end (or a C-terminal end of Cas9).

TABLE 7
Exemplary additional element sequences

| Name | Sequence | SEQ ID NO: |
|---|---|---|
| Kozak | gccaccatgg | 38 |
| SV40 NLS | ccaaagaagaagcggaaggtc | 39 |
| Nucleoplasmin | aaaaggccggcggccacgaaa | 40 |
| NLS | aaggccggccaggcaaaaaag | |
| | aaaaag | |

In some embodiments, the expression cassettes described herein comprise a transcription termination signal. Elements directing the efficient termination and polyadenylation of the heterologous nucleic acid transcripts increases heterologous gene expression. Transcription termination signals are generally found downstream of the polyadenylation signal.

In some embodiments, the expression cassette described herein comprises a termination signal for producing the guide RNA. In some embodiments, the polynucleotide encoding the guide RNA described herein, and/or the expression cassette encoding the guide RNA described herein, comprise a pol III termination signal. In some embodiments, the pol III termination signal is TTTTTG.

### Examples of Expression Cassettes

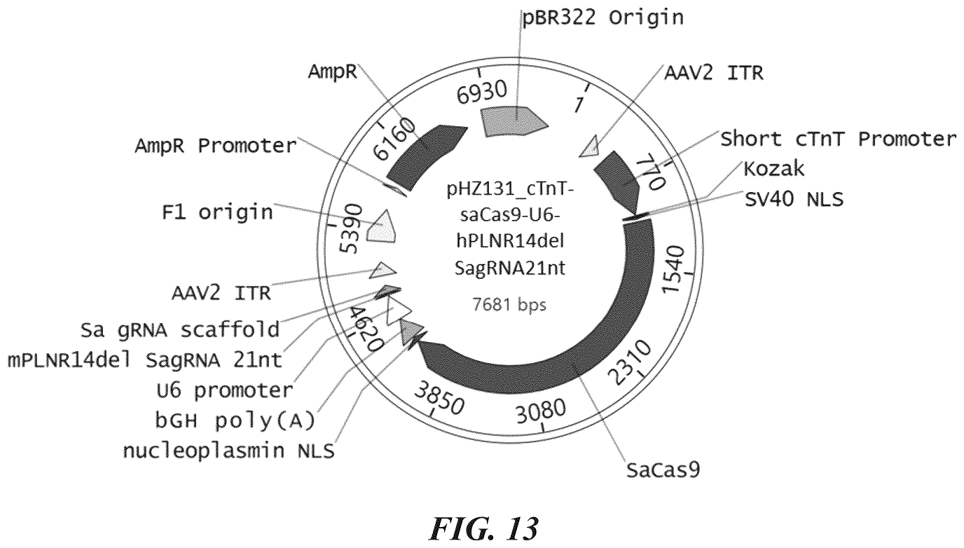

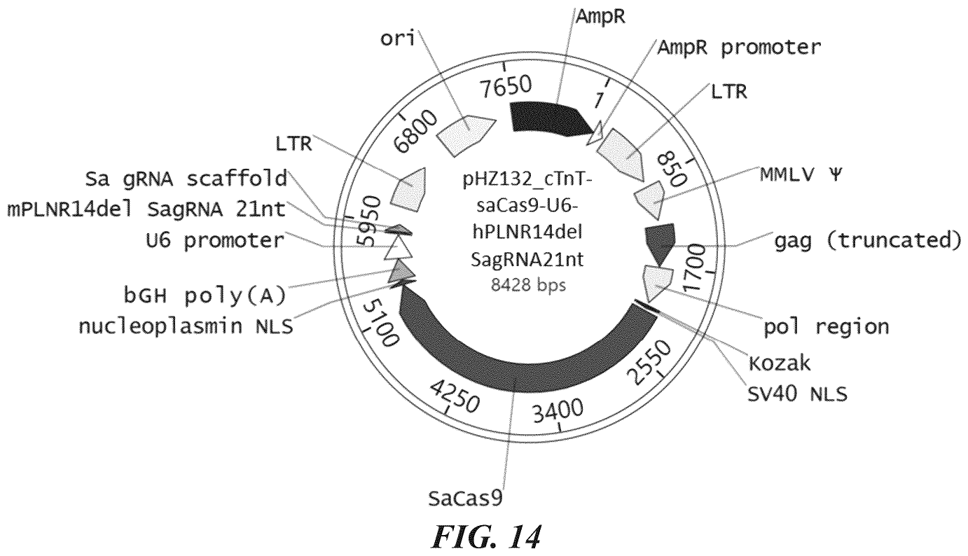

In some embodiments, the expression cassette described herein comprises any one of SEQ ID NOs: 35-37. SEQ ID NO: 35 is the sequence of the expression cassette pHZ128 described herein, the map of which is provided in FIG. 12. SEQ ID NO: 36 is the sequence of the expression cassette pHZ131 described herein, the map of which is provided in FIG. 13. SEQ ID NO: 37 is the sequence of the expression cassette pHZ132 described herein, the map of which is provided in FIG. 14.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 35.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 36.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 37.

TABLE 8
Exemplary expression vector sequences

| | | SEQ ID |
|---|---|---|
| Name | Sequence (5′ ITR to 3′ ITR, exclusive of ITRs) | NO: |
| pHZ128 | tgtagttaatgattaacccgccatgctacttatctacgtagccatgctctaggaagatcggaattc | 35 |
| (hTNGE101) | gcccttaaggtcatggagaagacccaccttgcagatgtcctcactggggctggcagagccgg | |
| | caacctgcctaaggctgctcagtccattaggagccagtagcctggaagatgtctttaccccca | |
| | gcatcagttcaagtggagcagcacataactcttgccctctgccttccaagattctggtgctgag | |
| | acttatggagtgtcttggaggttgccttctgccccccaaccctgctcccagctggccctcccag | |
| | gcctgggttgctggcctctgctttatcaggattctcaagagggacagctggtttatgttgcatga | |
| | ctgttccctgcatatctgctctggttttaaatagcttatctgagcagctggaggaccacatgggct | |
| | tatatggcgtggggtacatgttcctgtagccttgtccctggcacctgccaaaatagcagccaac | |
| | accccccacccccaccgccatccccctgccccacccgtcccctgtcgcacattcctccctccg | |
| | cagggctggctcaccaggccccagcccacatgcctgcttaaagccctctccatcctctgcctc | |
| | acccagtccccgctgagactgagcagacgcctccagcggccgcccgccaccatggcccca | |
| | aagaagaagcggaaggtcggtatccacggagtcccagcagccaagcggaactacatcctg | |
| | ggcctggacatcggcatcaccagcgtgggctacggcatcatcgactacgagacacgggac | |
| | gtgatcgatgccggcgtgcggctgttcaaagaggccaacgtggaaaacaacgagggcagg | |
| | cggagcaagagaggcgccagaaggctgaagcggcggaggcggcatagaatccagagag | |
| | tgaagaagctgctgttcgactacaacctgctgaccgaccacagcgagctgagcggcatcaac | |
| | ccctacgaggccagagtgaagggcctgagccagaagctgagcgaggaagagttctctgcc | |
| | gccctgctgcacctggccaagagaagaggcgtgcacaacgtgaacgaggtggaagagga | |
| | caccggcaacgagctgtccaccaaagagcagatcagccggaacagcaaggccctggaag | |
| | agaaatacgtggccgaactgcagctggaacggctgaagaaagacggcgaagtgcggggc | |
| | agcatcaacagattcaagaccagcgactacgtgaaagaagccaaacagctgctgaaggtgc | |
| | agaaggcctaccaccagctggaccagagcttcatcgacacctacatcgacctgctggaaacc | |
| | cggcggacctactatgagggacctggcgagggcagccccttcggctggaaggacatcaaa | |
| | gaatggtacgagatgctgatgggccactgcacctacttccccgaggaactgcggagcgtgaa | |
| | gtacgcctacaacgccgacctgtacaacgccctgaacgacctgaacaatctcgtgatcacca | |
| | gggacgagaacgagaagctggaatattacgagaagttccagatcatcgagaacgtgttcaag | |
| | cagaagaagaagcccaccctgaagcagatcgccaaagaaatcctcgtgaacgaagaggat | |
| | attaagggctacagagtgaccagcaccggcaagcccgagttcaccaacctgaaggtgtacc | |
| | acgacatcaaggacattaccgcccggaaagagattattgagaacgccgagctgctggatcag | |
| | attgccaagatcctgaccatctaccagagcagcgaggacatccaggaagaactgaccaatct | |
| | gaactccgagctgacccaggaagagatcgagcagatctctaatctgaagggctataccggca | |
| | cccacaacctgagcctgaaggccatcaacctgatcctggacgagctgtggcacaccaacga | |
| | caaccagatcgctatcttcaaccggctgaagctggtgcccaagaaggtggacctgtcccagc | |
| | agaaagagatccccaccaccctggtggacgacttcatcctgagccccgtcgtgaagagaag | |
| | cttcatccagagcatcaaagtgatcaacgccatcatcaagaagtacggcctgcccaacgacat | |
| | cattatcgagctggcccgcgagaagaactccaaggacgcccagaaaatgatcaacgagatg | |
| | cagaagcggaaccggcagaccaacgagcggatcgaggaaatcatccggaccaccggcaa | |
| | agagaacgccaagtacctgatcgagaagatcaagctgcacgacatgcaggaaggcaagtg | |
| | cctgtacagcctggaagccatccctctggaagatctgctgaacaaccccttcaactatgaggt | |
| | ggaccacatcatccccagaagcgtgtccttcgacaacagcttcaacaacaaggtgctcgtga | |
| | agcaggaagaaaacagcaagaagggcaaccggaccccattccagtacctgagcagcagc | |
| | gacagcaagatcagctacgaaaccttcaagaagcacatcctgaatctggccaagggcaagg | |
| | gcagaatcagcaagaccaagaaagagtatctgctggaagaacgggacatcaacaggttctc | |
| | cgtgcagaaagacttcatcaaccggaacctggtggataccagatacgccaccagaggcctg | |
| | atgaacctgctgcggagctacttcagagtgaacaacctggacgtgaaagtgaagtccatcaat | |
| | ggcggcttcaccagctttctgcggcggaagtggaagtttaagaaagagcggaacaaggggt | |
| | acaagcaccacgccgaggacgccctgatcattgccaacgccgatttcatcttcaaagagtgg | |
| | aagaaactggacaaggccaaaaaagtgatggaaaaccagatgttcgaggaaaagcaggcc | |
| | gagagcatgcccgagatcgaaaccgagcaggagtacaaagagatcttcatcaccccccacc | |
| | agatcaagcacattaaggacttcaaggactacaagtacagccaccgggtggacaagaagcc | |
| | taatagagagctgattaacgacaccctgtactccacccggaaggacgacaagggcaacacc | |
| | ctgatcgtgaacaatctgaacggcctgtacgacaaggacaatgacaagctgaaaaagctgat | |
| | caacaagagccccgaaaagctgctgatgtaccaccacgacccccagacctaccagaaactg | |
| | aagctgattatggaacagtacggcgacgagaagaatcccctgtacaagtactacgaggaaac | |
| | cgggaactacctgaccaagtactccaaaaaggacaacggccccgtgatcaagaagattaagt | |
| | attacggcaacaaactgaacgcccatctggacatcaccgacgactaccccaacagcagaaa | |
| | caaggtcgtgaagctgtccctgaagccctacagattcgacgtgtacctggacaatggcgtgta | |
| | caagttcgtgaccgtgaagaatctggatgtgatcaaaaaagaaaactactacgaagtgaatag | |
| | caagtgctatgaggaagctaagaagctgaagaagatcagcaaccaggccgagtttatcgcct | |
| | ccttctacaacaacgatctgatcaagatcaacggcgagctgtatagagtgatcggcgtgaaca | |
| | acgacctgctgaaccggatcgaagtgaacatgatcgacatcacctaccgcgagtacctggaa | |
| | aacatgaacgacaagaggccccccaggatcattaagacaatcgcctccaagacccagagca | |
| | ttaagaagtacagcacagacattctgggcaacctgtatgaagtgaaatctaagaagcaccctc | |
| | agatcatcaaaaagggcaaaaggccggcggccacgaaaaaggccggccaggcaaaaaa | |
| | gaaaaagtaaagatctgcctcgactgtgccttctagttgccagccatctgttgtttgcccctccc | |
| | ccgtgccttccttgaccctggaaggtgccactcccactgtcctttcctaataaaatgaggaaatt | |
| | gcatcgcattgtctgagtaggtgtcattctattctggggggggggtggggcaggacagcaag | |
| | ggggaggattgggaagacaatagcaggcatgctggggagagggcctatttcccatgattcct | |
| | tcatatttgcatatacgatacaaggctgttagagagataattggaattaatttgactgtaaacaca | |
| | aagatattagtacaaaatacgtgacgtagaaagtaataatttcttgggtagtttgcagttttaaaat | |
| | tatgttttaaaatggactatcatatgcttaccgtaacttgaaagtatttcgatttcttggctttatatat | |
| | cttgtggaaaggacgaaacaccgggttgaggctcttatagctgagttttagtactctggaaaca | |
| | gaatctactaaaacaaggcaaaatgccgtgtttatctcgtcaacttgttggcgagatttttgctcg | |
| | agttaagggcgaattcccgattaggatcttcctagagcatggctacgtagataagtagcatggc | |
| | gggttaatcattaactaca | |
| pHZ131 | tgtagttaatgattaacccgccatgctacttatctacgtagccatgctctaggaagatcggaattc | 36 |
| (mTNGE101) | gcccttaaggtcatggagaagacccaccttgcagatgtcctcactggggctggcagagccgg | |
| | caacctgcctaaggctgctcagtccattaggagccagtagcctggaagatgtctttaccccca | |
| | gcatcagttcaagtggagcagcacataactcttgccctctgccttccaagattctggtgctgag | |
| | acttatggagtgtcttggaggttgccttctgccccccaaccctgctcccagctggccctcccag | |
| | gcctgggttgctggcctctgctttatcaggattctcaagagggacagctggtttatgttgcatga | |
| | ctgttccctgcatatctgctctggttttaaatagcttatctgagcagctggaggaccacatgggct | |
| | tatatggcgtggggtacatgttcctgtagccttgtccctggcacctgccaaaatagcagccaac | |
| | accccccacccccaccgccatccccctgccccacccgtcccctgtcgcacattcctccctccg | |
| | cagggctggctcaccaggccccagcccacatgcctgcttaaagccctctccatcctctgcctc | |
| | acccagtccccgctgagactgagcagacgcctccagcggccgcccgccaccatggcccca | |
| | aagaagaagcggaaggtcggtatccacggagtcccagcagccaagcggaactacatcctg | |
| | ggcctggacatcggcatcaccagcgtgggctacggcatcatcgactacgagacacgggac | |
| | gtgatcgatgccggcgtgcggctgttcaaagaggccaacgtggaaaacaacgagggcagg | |
| | cggagcaagagaggcgccagaaggctgaagcggcggaggcggcatagaatccagagag | |
| | tgaagaagctgctgttcgactacaacctgctgaccgaccacagcgagctgagcggcatcaac | |
| | ccctacgaggccagagtgaagggcctgagccagaagctgagcgaggaagagttctctgcc | |
| | gccctgctgcacctggccaagagaagaggcgtgcacaacgtgaacgaggtggaagagga | |
| | caccggcaacgagctgtccaccaaagagcagatcagccggaacagcaaggccctggaag | |
| | agaaatacgtggccgaactgcagctggaacggctgaagaaagacggcgaagtgcggggc | |
| | agcatcaacagattcaagaccagcgactacgtgaaagaagccaaacagctgctgaaggtgc | |
| | agaaggcctaccaccagctggaccagagcttcatcgacacctacatcgacctgctggaaacc | |
| | cggcggacctactatgagggacctggcgagggcagccccttcggctggaaggacatcaaa | |
| | gaatggtacgagatgctgatgggccactgcacctacttccccgaggaactgcggagcgtgaa | |
| | gtacgcctacaacgccgacctgtacaacgccctgaacgacctgaacaatctcgtgatcacca | |
| | gggacgagaacgagaagctggaatattacgagaagttccagatcatcgagaacgtgttcaag | |
| | cagaagaagaagcccaccctgaagcagatcgccaaagaaatcctcgtgaacgaagaggat | |
| | attaagggctacagagtgaccagcaccggcaagcccgagttcaccaacctgaaggtgtacc | |
| | acgacatcaaggacattaccgcccggaaagagattattgagaacgccgagctgctggatcag | |
| | attgccaagatcctgaccatctaccagagcagcgaggacatccaggaagaactgaccaatct | |
| | gaactccgagctgacccaggaagagatcgagcagatctctaatctgaagggctataccggca | |
| | cccacaacctgagcctgaaggccatcaacctgatcctggacgagctgtggcacaccaacga | |
| | caaccagatcgctatcttcaaccggctgaagctggtgcccaagaaggtggacctgtcccagc | |
| | agaaagagatccccaccaccctggtggacgacttcatcctgagccccgtcgtgaagagaag | |
| | cttcatccagagcatcaaagtgatcaacgccatcatcaagaagtacggcctgcccaacgacat | |
| | cattatcgagctggcccgcgagaagaactccaaggacgcccagaaaatgatcaacgagatg | |
| | cagaagcggaaccggcagaccaacgagcggatcgaggaaatcatccggaccaccggcaa | |
| | agagaacgccaagtacctgatcgagaagatcaagctgcacgacatgcaggaaggcaagtg | |
| | cctgtacagcctggaagccatccctctggaagatctgctgaacaaccccttcaactatgaggt | |
| | ggaccacatcatccccagaagcgtgtccttcgacaacagcttcaacaacaaggtgctcgtga | |
| | agcaggaagaaaacagcaagaagggcaaccggaccccattccagtacctgagcagcagc | |
| | gacagcaagatcagctacgaaaccttcaagaagcacatcctgaatctggccaagggcaagg | |
| | gcagaatcagcaagaccaagaaagagtatctgctggaagaacgggacatcaacaggttctc | |
| | cgtgcagaaagacttcatcaaccggaacctggtggataccagatacgccaccagaggcctg | |
| | atgaacctgctgcggagctacttcagagtgaacaacctggacgtgaaagtgaagtccatcaat | |
| | ggcggcttcaccagctttctgcggcggaagtggaagtttaagaaagagcggaacaaggggt | |
| | acaagcaccacgccgaggacgccctgatcattgccaacgccgatttcatcttcaaagagtgg | |
| | aagaaactggacaaggccaaaaaagtgatggaaaaccagatgttcgaggaaaagcaggcc | |
| | gagagcatgcccgagatcgaaaccgagcaggagtacaaagagatcttcatcaccccccacc | |
| | agatcaagcacattaaggacttcaaggactacaagtacagccaccgggtggacaagaagcc | |
| | taatagagagctgattaacgacaccctgtactccacccggaaggacgacaagggcaacacc | |
| | ctgatcgtgaacaatctgaacggcctgtacgacaaggacaatgacaagctgaaaaagctgat | |
| | caacaagagccccgaaaagctgctgatgtaccaccacgacccccagacctaccagaaactg | |
| | aagctgattatggaacagtacggcgacgagaagaatcccctgtacaagtactacgaggaaac | |
| | cgggaactacctgaccaagtactccaaaaaggacaacggccccgtgatcaagaagattaagt | |
| | attacggcaacaaactgaacgcccatctggacatcaccgacgactaccccaacagcagaaa | |
| | caaggtcgtgaagctgtccctgaagccctacagattcgacgtgtacctggacaatggcgtgta | |
| | caagttcgtgaccgtgaagaatctggatgtgatcaaaaaagaaaactactacgaagtgaatag | |
| | caagtgctatgaggaagctaagaagctgaagaagatcagcaaccaggccgagtttatcgcct | |
| | ccttctacaacaacgatctgatcaagatcaacggcgagctgtatagagtgatcggcgtgaaca | |
| | acgacctgctgaaccggatcgaagtgaacatgatcgacatcacctaccgcgagtacctggaa | |
| | aacatgaacgacaagaggccccccaggatcattaagacaatcgcctccaagacccagagca | |
| | ttaagaagtacagcacagacattctgggcaacctgtatgaagtgaaatctaagaagcaccctc | |
| | agatcatcaaaaagggcaaaaggccggcggccacgaaaaaggccggccaggcaaaaaa | |
| | gaaaaagtaaagatctgcctcgactgtgccttctagttgccagccatctgttgtttgcccctccc | |
| | ccgtgccttccttgaccctggaaggtgccactcccactgtcctttcctaataaaatgaggaaatt | |
| | gcatcgcattgtctgagtaggtgtcattctattctggggggtggggggggcaggacagcaag | |
| | ggggaggattgggaagacaatagcaggcatgctggggagagggcctatttcccatgattcct | |
| | tcatatttgcatatacgatacaaggctgttagagagataattggaattaatttgactgtaaacaca | |
| | aagatattagtacaaaatacgtgacgtagaaagtaataatttcttgggtagtttgcagttttaaaat | |
| | tatgttttaaaatggactatcatatgcttaccgtaacttgaaagtatttcgatttcttggctttatatat | |
| | cttgtggaaaggacgaaacaccgagtggaggccctgatagccgagttttagtactctggaaa | |
| | cagaatctactaaaacaaggcaaaatgccgtgtttatctcgtcaacttgttggcgagatttttgct | |
| | cgagttaagggcgaattcccgattaggatcttcctagagcatggctacgtagataagtagcatg | |
| | gcgggttaatcattaactaca | |
| pHZ132 | aagctggccagcaacttatctgtgtctgtccgattgtctagtgtctatgactgattttatgcgcctg | 37 |
| | cgtcggtactagttagctaactagctctgtatctggcggacccgtggtggaactgacgagttcg | |
| | gaacacccggccgcaaccctgggagacgtcccagggacttcgggggccgtttttgtggccc | |
| | gacctgagtccaaaaatcccgatcgttttggactctttggtgcacccccctaataggagggata | |
| | tgtggttctggtaggagacgagaacctaaaacagttcccgcctccgtctgaatttttgctttcggt | |
| | ttgggaccgaagccgcgccgcgcgtcttgtctgctgcagcatcgttctgtgttgtctctgtctga | |
| | ctgtgtttctgtatttgtctgaaaattagggccagactgttaccactcccttaagtttgaccttaggt | |
| | cactggaaagatgtcgagcggatcgctcacaaccagtcggtagatgtcaagaagagacgttg | |
| | ggttaccttctgctctgcagaatggccaacctttaacgtcggatggccgcgagacggcaccttt | |
| | aaccgagacctcatcacccaggttaagatcaaggtcttttcacctggcccgcatggacaccca | |
| | gaccaggtcccctacatcgtgacctgggaagccttggcttttgacccccctccctgggtcaag | |
| | ccctttgtacaccctaagcctccgcctcctcttcctccatccgccccgtctctcccccttgaacct | |
| | cctcgttcgaccccgcctcgatcctccctttatccagccctcactccttctctaggcgcccccat | |
| | atggccatatgagatcttatatggggcacccccgccccttgtaaacttccctgaccctgacatg | |
| | acaagagttactaacagcccctctctccaagctcacttacaggctctctacttagtccagcacg | |
| | aagtctggagacctctggcggcagcctaccaagaacaactggaccgaccggtggtacctca | |
| | cccttaccgagtcggcgacacagtgtgggtccgccgacaccagactaagaacctagaacct | |
| | cgctggaaaggaccttacacagtcctgctgaccacccccaccgccctcaaagtagacggcat | |
| | cgcagcttggatacacgccgcccacgtgaaggctgccgaccccgggggtggaccatcctct | |
| | agactgccggatctagctagttaattaaggatccccgccaccatggccccaaagaagaagcg | |
| | gaaggtcggtatccacggagtcccagcagccaagcggaactacatcctgggcctggacatc | |
| | ggcatcaccagcgtgggctacggcatcatcgactacgagacacgggacgtgatcgatgccg | |
| | gcgtgcggctgttcaaagaggccaacgtggaaaacaacgagggcaggcggagcaagaga | |
| | ggcgccagaaggctgaagcggcggaggcggcatagaatccagagagtgaagaagctgct | |
| | gttcgactacaacctgctgaccgaccacagcgagctgagcggcatcaacccctacgaggcc | |
| | agagtgaagggcctgagccagaagctgagcgaggaagagttctctgccgccctgctgcacc | |
| | tggccaagagaagaggcgtgcacaacgtgaacgaggtggaagaggacaccggcaacgag | |
| | ctgtccaccaaagagcagatcagccggaacagcaaggccctggaagagaaatacgtggcc | |
| | gaactgcagctggaacggctgaagaaagacggcgaagtgcggggcagcatcaacagattc | |
| | aagaccagcgactacgtgaaagaagccaaacagctgctgaaggtgcagaaggcctaccac | |
| | cagctggaccagagcttcatcgacacctacatcgacctgctggaaacccggcggacctacta | |
| | tgagggacctggcgagggcagccccttcggctggaaggacatcaaagaatggtacgagat | |
| | gctgatgggccactgcacctacttccccgaggaactgcggagcgtgaagtacgcctacaac | |
| | gccgacctgtacaacgccctgaacgacctgaacaatctcgtgatcaccagggacgagaacg | |
| | agaagctggaatattacgagaagttccagatcatcgagaacgtgttcaagcagaagaagaag | |
| | cccaccctgaagcagatcgccaaagaaatcctcgtgaacgaagaggatattaagggctaca | |
| | gagtgaccagcaccggcaagcccgagttcaccaacctgaaggtgtaccacgacatcaagga | |
| | cattaccgcccggaaagagattattgagaacgccgagctgctggatcagattgccaagatcct | |
| | gaccatctaccagagcagcgaggacatccaggaagaactgaccaatctgaactccgagctg | |
| | acccaggaagagatcgagcagatctctaatctgaagggctataccggcacccacaacctga | |
| | gcctgaaggccatcaacctgatcctggacgagctgtggcacaccaacgacaaccagatcgc | |
| | tatcttcaaccggctgaagctggtgcccaagaaggggacctgtcccagcagaaagagatcc | |
| | ccaccaccctggtggacgacttcatcctgagccccgtcgtgaagagaagcttcatccagagc | |
| | atcaaagtgatcaacgccatcatcaagaagtacggcctgcccaacgacatcattatcgagctg | |
| | gcccgcgagaagaactccaaggacgcccagaaaatgatcaacgagatgcagaagcggaa | |
| | ccggcagaccaacgagcggatcgaggaaatcatccggaccaccggcaaagagaacgcca | |
| | agtacctgatcgagaagatcaagctgcacgacatgcaggaaggcaagtgcctgtacagcct | |
| | ggaagccatccctctggaagatctgctgaacaaccccttcaactatgaggtggaccacatcat | |
| | ccccagaagcgtgtccttcgacaacagcttcaacaacaaggtgctcgtgaagcaggaagaa | |
| | aacagcaagaagggcaaccggaccccattccagtacctgagcagcagcgacagcaagatc | |
| | agctacgaaaccttcaagaagcacatcctgaatctggccaagggcaagggcagaatcagca | |
| | agaccaagaaagagtatctgctggaagaacgggacatcaacaggttctccgtgcagaaaga | |
| | cttcatcaaccggaacctggtggataccagatacgccaccagaggcctgatgaacctgctgc | |
| | ggagctacttcagagtgaacaacctggacgtgaaagtgaagtccatcaatggggcttcacc | |
| | agctttctgcggcggaagtggaagtttaagaaagagcggaacaaggggtacaagcaccacg | |
| | ccgaggacgccctgatcattgccaacgccgatttcatcttcaaagagtggaagaaactggaca | |
| | aggccaaaaaagtgatggaaaaccagatgttcgaggaaaagcaggccgagagcatgcccg | |
| | agatcgaaaccgagcaggagtacaaagagatcttcatcaccccccaccagatcaagcacatt | |
| | aaggacttcaaggactacaagtacagccaccgggtggacaagaagcctaatagagagctga | |
| | ttaacgacaccctgtactccacccggaaggacgacaagggcaacaccctgatcgtgaacaat | |
| | ctgaacggcctgtacgacaaggacaatgacaagctgaaaaagctgatcaacaagagccccg | |
| | aaaagctgctgatgtaccaccacgacccccagacctaccagaaactgaagctgattatggaa | |
| | cagtacggcgacgagaagaatcccctgtacaagtactacgaggaaaccgggaactacctga | |
| | ccaagtactccaaaaaggacaacggccccgtgatcaagaagattaagtattacggcaacaaa | |
| | ctgaacgcccatctggacatcaccgacgactaccccaacagcagaaacaaggtcgtgaagc | |
| | tgtccctgaagccctacagattcgacgtgtacctggacaatggcgtgtacaagttcgtgaccgt | |
| | gaagaatctggatgtgatcaaaaaagaaaactactacgaagtgaatagcaagtgctatgagg | |
| | aagctaagaagctgaagaagatcagcaaccaggccgagtttatcgcctccttctacaacaac | |
| | gatctgatcaagatcaacggcgagctgtatagagtgatcggcgtgaacaacgacctgctgaa | |
| | ccggatcgaagtgaacatgatcgacatcacctaccgcgagtacctggaaaacatgaacgaca | |
| | agaggccccccaggatcattaagacaatcgcctccaagacccagagcattaagaagtacag | |
| | cacagacattctgggcaacctgtatgaagtgaaatctaagaagcaccctcagatcatcaaaaa | |
| | gggcaaaaggccggcggccacgaaaaaggccggccaggcaaaaaagaaaaagtaaaga | |
| | tctgcctcgactgtgccttctagttgccagccatctgttgtttgcccctcccccgtgccttccttga | |
| | ccctggaaggtgccactcccactgtcctttcctaataaaatgaggaaattgcatcgcattgtctg | |
| | agtaggtgtcattctattctggggggtggggggggcaggacagcaagggggaggattggg | |
| | aagacaatagcaggcatgctggggagagggcctatttcccatgattccttcatatttgcatatac | |
| | gatacaaggctgttagagagataattggaattaatttgactgtaaacacaaagatattagtacaa | |
| | aatacgtgacgtagaaagtaataatttcttgggtagtttgcagttttaaaattatgttttaaaatgga | |
| | ctatcatatgcttaccgtaacttgaaagtatttcgatttcttggctttatatatcttgtggaaaggac | |
| | gaaacaccgagtggaggccctgatagccgagttttagtactctggaaacagaatctactaaaa | |
| | caaggcaaaatgccgtgtttatctcgtcaacttgttggcgaga | |

### Inverted Terminal Repeat Sequences

In some embodiments, the expression cassette is flanked by AAV inverted terminal repeats (ITRs). In some embodiments, the expression cassette is flanked by AAV ITRs at the 5′ and 3′ ends.

Any suitable ITRs described herein or known in the art may be used. The ITRs function as recognition sites for replication. ITRs serve as markers used for viral packaging of the expression cassette (Clark et al. Hum Gene Ther. 6:1329-41 (1995)). ITRs form T-shaped secondary structures by two adjacent inverted repeats separated by a sing unpaired nucleotide. ITRs are required for packaging the expression cassette into an rAAV virion, which provide the function of expressing the transgene after a host cell is targeted by the rAAV virion. The ITRs contain tetranucleotide repeat motifs called Rep-binding elements (RBE) that act as contact points for the Rep68/78 proteins encoded by the rep gene. The ITRs also contain a packaging signal for genome encapsidation, which directs 3′ genomic transport into preassembled capsids by Rep proteins (Wilmott et al. Hum Gene Ther Methods. 30:206-213 (2019)). Any naturally occurring or synthetically derived ITRs described herein or known in the art can be used.

In some embodiments, the ITRs flanking the transgene expression cassette are ITRs of the same AAV serotype as the Rep protein used in making the virions described herein. For example, where a Rep protein from AAV9 is used, the transgene expression cassette used in the expression system comprises ITRs from AAV9 as well. In another example, where a Rep protein from AAV2 is used, the transgene expression cassette used in the expression system comprises ITRs from AAV2 as well. In another example, where a Rep protein from AAV5 is used, the transgene expression cassette used in the expression system comprises ITRs from AAV5 as well. The ITRs may be of the same or different serotype as the capsid protein used in packaging the virion described herein.

In some embodiments, the ITRs comprise the polynucleotide sequence that shares at least 80%, 85%, 90%, 95%, 96%, 97%, 98%, 99%, or 100% identity to the ITR sequences presented below.

TABLE 9
Exemplary ITR sequences

| | | SEQ ID |
|---|---|---|
| Name | Sequences | NO: |
| 5′ ITR | ctgcgcgctcgctcgctcactgaggccgcccgggca | 44 |
| | aagcccgggcgtcgggcgacctttggtcgcccggcc | |
| | tcagtgagcgagcgagcgcgcagagagggagtggcc | |
| | aactccatcactaggggttcct | |
| 3′ ITR | aggaacccctagtgatggagttggccactccctctc | 45 |
| | tgcgcgctcgctcgctcactgaggccgggcgaccaa | |
| | aggtcgcccgacgcccgggctttgcccgggcggcct | |
| | cagtgagcgagcgagcgcgcag | |

In some embodiments, rAAV genome comprises an expression cassette that is flanked by one or both of a 5′ inverted terminal repeat (ITR) and a 3′ ITR. In some embodiments, the 5′ ITR comprises a sequence that shares at least 90%, 95%, 98% or 100% identity to SEQ ID NO: 44. In some embodiments, the 3′ ITR comprises a sequence that shares at least 90%, 95%, 98% or 100% identity to SEQ ID NO: 45.

### Self-Inactivating Cas Constructs

In some embodiments, the present disclosure provides self-inactivating expression cassettes comprising a first polynucleotide encoding a Cas9 protein operably linked to a first promoter and a second polynucleotide encoding a gRNA operably linked to a second promoter, wherein the expression cassette is self-inactivating and Cas9 expression can be self-terminated after expression of the Cas9 protein, resulting in transient expression of Cas9. Self-inactivating Cas constructs can be useful where long-term or constitutive expression of Cas proteins (e.g., when introduced in a gene therapy) is undesirable due to potential off-target effects, immune responses, and unintended genomic modifications. Because Cas9 is a prokaryotic protein, it will be highly immunogenic in eukaryotic organisms such as human. Thus, and especially in gene therapies for human subjects, limiting Cas expression after the intended genomic editing of target genes is complete may offer several advantages, including increased safety profile, reduced immunogenicity by Cas proteins, and/or reduced off-target effects.

In some embodiments, the self-inactivating expression cassette comprises a self-inactivation site which, upon recognition and cleavage by the Cas protein expressed by the expression cassette, can terminate transcription/translation of the first polynucleotide encoding the Cas9 protein, e.g., through a negative feedback loop. The self-inactivation site may be placed anywhere in the expression cassette, e.g., within the first promoter driving the expression of the Cas9 protein, within the first polynucleotide encoding the Cas9 protein, in between the first polynucleotide and the first promoter, or after the first polynucleotide encoding the Cas9 protein (e.g., before or within a poly(A) sequence). If the self-inactivation site is placed within the first promoter driving the expression of the Cas9 protein, it can be near the 5′ end of the first polynucleotide (e.g., after the start codon “ATG”), near the 3′ end, or somewhere in between.

In some embodiments, the self-inactivation site comprises a gRNA target region. Upon recognition by a gRNA, the gRNA target region can be cleaved by a Cas protein (e.g., the Cas9 protein encoded by the first polynucleotide of the expression cassette), resulting in termination of the Cas transcript. The gRNA that recognizes and/or directs Cas-dependent cleaving of this target sequence can be different from the gRNA encoded by the second polynucleotide of the expression cassette or can be the same as the gRNA encoded by the second polynucleotide of the expression cassette. If the former, then the expression cassette may comprise a third polynucleotide encoding a second gRNA operably linked to a third promoter, and the second gRNA may upon expression direct the Cas protein to cut at the gRNA target region, thereby terminating the Cas expression.

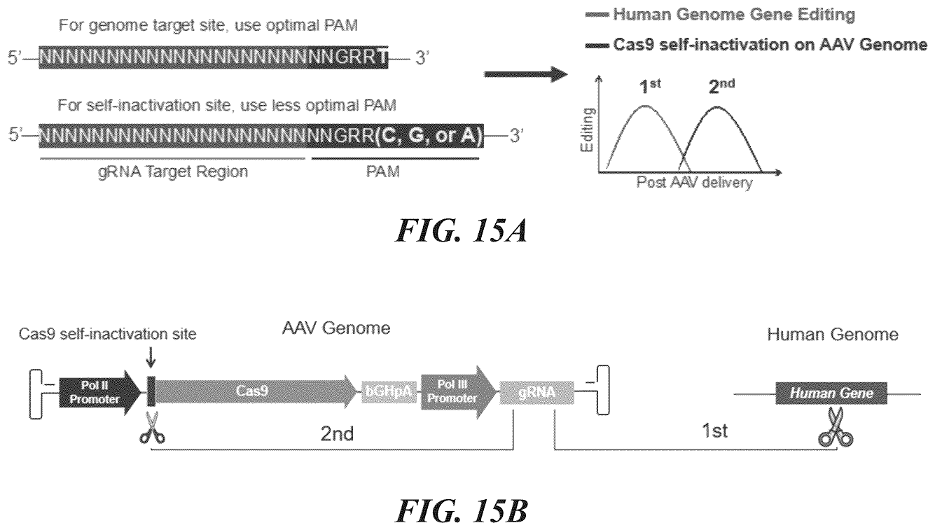

In some embodiments, the self-inactivation site comprises a gRNA target region recognized by the same gRNA encoded by the second polynucleotide of the expression cassette (see FIG. 15A). For example, the gRNA target region can comprise the same sequence as the gRNA encoded by the second polynucleotide of the expression cassette or a portion thereof (e.g., the complementary region or spacer portion), or a sequence that is reverse complement to the gRNA encoded by the second polynucleotide of the expression cassette or a portion thereof (e.g., the complementary region or spacer portion). In some embodiments, the gRNA target region comprises a nucleotide sequence corresponding to any gRNA sequence described herein or a portion thereof (e.g., the complementary region or spacer portion). In some embodiments, the gRNA target region comprises at least 16, 17, 18, or 19 nucleotides of the nucleotide sequence ggttgaggctcttatagctga (SEQ ID NO: 1) or a sequence reverse complement to at least 16, 17, 18, or 19 nucleotides of SEQ ID NO: 1, with up to 3 mismatches, optionally wherein the mismatch(es) is not in the sequence of ttatagctga (SEQ ID NO: 127) within SEQ ID NO: 1. In some embodiments, the gRNA target region comprises a nucleotide sequence selected from the group consisting of ggttgaggctcttatagctga (SEQ ID NO: 1), ttgaggctcttatagctga (SEQ ID NO: 2), gttgaggctcttatagctga (SEQ ID NO: 3), tggttgaggctcttatagctga (SEQ ID NO: 4), and a sequence reverse complement to any of the foregoing. In some embodiments, the gRNA target region comprises the nucleotide sequence of ggttgaggctcttatagctga (SEQ ID NO: 1) or a sequence reverse complement to SEQ ID NO: 1.

In certain of these embodiments, the self-inactivation site further comprises a less optimal PAM sequence of the particular Cas9 protein encoded by the first polynucleotide of the expression cassette (see FIG. 15A). The PAM sequence can be 5′ or 3′ to the gRNA target region. For a non-limiting example, if the Cas9 protein is saCas9, the less optimal PAM sequence is placed 3′ to the gRNA target region and can be NNGRRC, NNGRRG, or NNGRRA, wherein N is any nucleotide, and R is guanine or adenine (see FIG. 15A). By incorporating a less optimal PAM sequence adjacent to the gRNA garget region, Cas-dependent cleaving at the self-inactivation site is less efficient compared to cutting near an optimal PAM sequence. Because the genomic target site intended by the expression cassette is usually adjacent to an optimal PAM sequence (e.g., in the case of saCas9, it is NNGRRT), upon expression of the Cas9 protein and gRNA encoded by the expression cassette, the cleavage at the intended genomic target site would be more effective and would thus take place more efficiently. The cleavage directed by the same Cas9 and gRNA at the self-inactivation site would be less effective and take place less efficiently due to the less optimal PAM sequence (FIGS. 15A-15B). Accordingly, the self-inactivating expression cassette would achieve the sequential cutting of (1) its intended genomic target site, thereby to complete the genomic gene editing; and (2) the self-inactivation site of the expression cassette, thereby to terminate the expression of the Cas protein after the genomic gene editing is complete.

In any of the above-described embodiments, as with the constitutive expression cassette, the first promoter driving Cas9 expression can be any promoter suitable for protein expression described herein, for example, a muscle-specific promoter or a cardiac-specific promoter described herein or known in the art. In some embodiments, the first promoter is a TNNT2 promoter, such as any TNNT2 promoter described herein. In some embodiments, the first promoter is a human TNNT2 promoter. In some embodiments, the first promoter comprises, essentially consists of, or consists of any one of SEQ ID NOs: 12-17. Similarly, the second promoter driving gRNA expression can be any promoter suitable for expression of RNA. In some embodiments, the second promoter is a Pol III promoter (e.g., a human Pol III promoter) described herein or known in the art. In some embodiments, the second promoter is a U6 promoter. In some embodiments, the second promoter is a human U6 promoter. In some embodiments, the second promoter comprises, essentially consists of, or consists of SEQ ID NO: 22.

The self-inactivating expression cassettes contemplated herein may be combined with other sequences described herein, such as promoters, enhancers, UTRs, signal sequences, Kozak sequences, polyadenylation signals, additional restriction enzyme sites, multiple cloning sites, IRES, recombinase recognition sites (e.g., LoxP, FRT, and Att sites), termination codons, transcriptional termination signals, and polynucleotides encoding self-cleaving polypeptides, epitope tags.

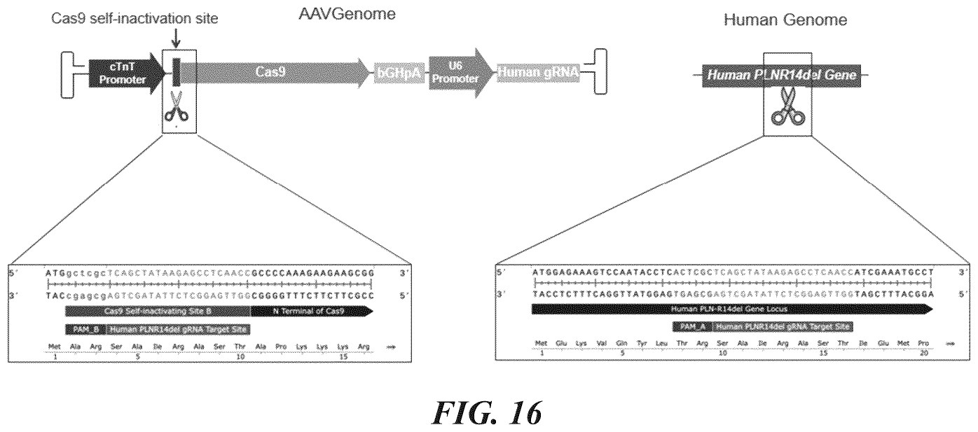

In some embodiments, the self-inactivating expression cassettes described herein comprises, essentially consists of, or consists of (i) a first polynucleotide sequence encoding a Cas protein (e.g., saCAs9) operably linked to a first promoter (e.g., human TNNT2), a self-inactivation site, and a poly(A) sequence, and (ii) a second polynucleotide encoding a gRNA operably linked to a second promoter (e.g., human U6). In some embodiments, the first polynucleotide and the second polynucleotide are in the same vector in a head-to-tail orientation. In some embodiments, the expression cassette provided herein is the same or similar to that depicted in FIG. 15B and/or FIG. 16, including the relative location and/or orientation of elements as presented therein.

It should be noted that self-inactivating expression cassettes for editing of the PLN gene are discussed for illustrative purposes only, and the same concept of self-inactivating Cas constructs can be applied to the editing of any other gene, upon selection of appropriate gRNA sequences and/or PAM sequences of the self-inactivation site that are dependent on the particular Cas9 protein of choice and the target gene of interest. Non-limiting examples of genes that can be edited through a self-inactivating Cas construct include cardiac troponin T (TNNT2), BAG family molecular chaperone regulator 3 (BAG3), myosin heavy chain (MYH7), tropomyosin 1 (TPM1), myosin binding protein C (MYBPC3), 5′-AMP-activated protein kinase subunit gamma-2 (PRKAG2), troponin I type 3 (TNNI3), titin (TTN), myosin, light chain 2 (MYL2), actin, alpha cardiac muscle 1 (ACTC1), potassium voltage-gated channel, KQT-like subfamily, member 1 (KCNQ1), myocyte enhancer factor 2c (MEF2C), cardiac LIM protein (CSRP3), DWORF, junctophilin (e.g., JPH2), alpha-crystallin B chain (CRYAB), LMNA (such as Lamin A and Lamin C isoforms), troponin I type 3 (TNNI3), lysosomal-associated membrane protein 2 (LAMP2, such as LAMP2a, LAMP2b and LAMP2c isoforms), desmoplakin (DSP, such as DPI and DPII isoforms), desmoglein 2 (DSG2), junction plakoglobin (JUP), plakophilin-2 (PKP2), matrix metallopeptidase 11 (MMP11), synaptopodin 2 like (SYNPO2L, such as SYNPO2LA and SYNPO2LB), RNA binding motif protein 20 (RBM20), metastasis suppressor protein 1 (MTSS1), proprotein convertase subtilisin/kexin type 9 (PCSK9), acid alpha-glucosidase (GAA), and frataxin (FXN).

Moreover, the self-inactivating expression cassettes may be flanked by one or more inverted terminal repeats (ITRs). The expression cassettes can be integrated into the host cell genome by, for example, infecting the host cell with an rAAV virion comprising a capsid protein and a viral genome comprising the expression cassette.

### Illustrative Self-Inactivating Expression Cassettes

Illustrative, non-limiting arrangements and orientations of the elements in the self-inactivating expression cassettes described herein are shown in FIG. 15B and FIG. 16. In some embodiments, the 5′ to 3′ arrangement of elements is selected from:
5′-promoter-Cas protein coding sequence (inclusive of a self-inactivation site near the 5′ end)-poly(A)-promoter-gRNA coding sequence-3′;
5′-muscle cell-specific or cardiac cell-specific (e.g., cardiomyocyte) promoter-Cas protein coding sequence (inclusive of a self-inactivation site near the 5′ end)-poly(A)-promoter (e.g., pol III promoter such as U6)-gRNA coding sequence-3′;
5′-human TNNT2 promoter-Cas protein coding sequence (inclusive of a self-inactivation site near the 5′ end)-poly(A)-promoter (e.g., pol III promoter such as U6)-gRNA coding sequence-3′.

In some embodiments, the first polynucleotide encoding the Cas protein is oriented in head-to tail orientation relative to the second polynucleotide encoding the guide RNA.

### Examples of Self-inactivating Expression Cassettes

In some embodiments, the expression cassette described herein comprises any one of SEQ ID NOs: 128-135. SEQ ID NO: 128 is the sequence of the expression cassette of hTNGE102 described herein. SEQ ID NO: 129 is the sequence of the expression cassette of hTNGE103 described herein. SEQ ID NO: 130 is the sequence of the expression cassette hTNGE104 described herein. SEQ ID NO: 131 is the sequence of the expression cassette hTNGE105 described herein. SEQ ID NO: 132 is the sequence of the expression cassette of mTNGE102 described herein. SEQ ID NO: 133 is the sequence of the expression cassette of mTNGE103 described herein. SEQ ID NO: 134 is the sequence of the expression cassette mTNGE104 described herein. SEQ ID NO: 135 is the sequence of the expression cassette mTNGE105 described herein.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 128.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 129.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 130.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 131.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 132.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 133.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 134.

In some embodiments, the expression cassette comprises a polynucleotide sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 135.

The sequences of SEQ ID NOs: 128-135 are provided in Table 10 below, with the start codon of the Cas coding sequence highlighted in capital letters, the self-inactivation site highlighted by underline, and the PAM sequence highlighted in bold.

TABLE 10
Exemplary expression vector sequences

| | | SEQ ID |
|---|---|---|
| Name | Sequence (5′ ITR to 3′ ITR, exclusive of ITRs) | NO: |
| hTNGE102 | tgtagttaatgattaacccgccatgctacttatctacgtagccatgctctaggaagatcggaattcg | 128 |
| | cccttaaggtcatggagaagacccaccttgcagatgtcctcactggggctggcagagccggca | |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGactcgctcagctataagagcctc | |
| | aaccgccccaaagaagaagcggaaggtcggtatccacggagtcccagcagccaagcggaac | |
| | tacatcctgggcctggacatcggcatcaccagcgtgggctacggcatcatcgactacgagacac | |
| | gggacgtgatcgatgccggcgtgcggctgttcaaagaggccaacgtggaaaacaacgagggc | |
| | aggcggagcaagagaggcgccagaaggctgaagcggcggaggcggcatagaatccagaga | |
| | gtgaagaagctgctgttcgactacaacctgctgaccgaccacagcgagctgagcggcatcaac | |
| | ccctacgaggccagagtgaagggcctgagccagaagctgagcgaggaagagttctctgccgc | |
| | cctgctgcacctggccaagagaagaggcgtgcacaacgtgaacgaggtggaagaggacacc | |
| | ggcaacgagctgtccaccaaagagcagatcagccggaacagcaaggccctggaagagaaat | |
| | acgtggccgaactgcagctggaacggctgaagaaagacggcgaagtgcggggcagcatcaa | |
| | cagattcaagaccagcgactacgtgaaagaagccaaacagctgctgaaggtgcagaaggccta | |
| | ccaccagctggaccagagcttcatcgacacctacatcgacctgctggaaacccggcggaccta | |
| | ctatgagggacctggcgagggcagccccttcggctggaaggacatcaaagaatggtacgagat | |
| | gctgatgggccactgcacctacttccccgaggaactgcggagcgtgaagtacgcctacaacgc | |
| | cgacctgtacaacgccctgaacgacctgaacaatctcgtgatcaccagggacgagaacgagaa | |
| | gctggaatattacgagaagttccagatcatcgagaacgtgttcaagcagaagaagaagcccacc | |
| | ctgaagcagatcgccaaagaaatcctcgtgaacgaagaggatattaagggctacagagtgacc | |
| | agcaccggcaagcccgagttcaccaacctgaaggtgtaccacgacatcaaggacattaccgcc | |
| | cggaaagagattattgagaacgccgagctgctggatcagattgccaagatcctgaccatctacca | |
| | gagcagcgaggacatccaggaagaactgaccaatctgaactccgagctgacccaggaagaga | |
| | tcgagcagatctctaatctgaagggctataccggcacccacaacctgagcctgaaggccatcaa | |
| | cctgatcctggacgagctgtggcacaccaacgacaaccagatcgctatcttcaaccggctgaag | |
| | ctggtgcccaagaaggtggacctgtcccagcagaaagagatccccaccaccctggtggacga | |
| | cttcatcctgagccccgtcgtgaagagaagcttcatccagagcatcaaagtgatcaacgccatca | |
| | tcaagaagtacggcctgcccaacgacatcattatcgagctggcccgcgagaagaactccaagg | |
| | acgcccagaaaatgatcaacgagatgcagaagcggaaccggcagaccaacgagcggatcga | |
| | ggaaatcatccggaccaccggcaaagagaacgccaagtacctgatcgagaagatcaagctgc | |
| | acgacatgcaggaaggcaagtgcctgtacagcctggaagccatccctctggaagatctgctgaa | |
| | caaccccttcaactatgaggtggaccacatcatccccagaagcgtgtccttcgacaacagcttca | |
| | acaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgggttgaggctcttat | |
| | agctgagttttagtactctggaaacagaatctactaaaacaaggcaaaatgccgtgtttatctcgtc | |
| | aacttgttggcgagatttttgctcgagttaagggcgaattcccgattaggatcttcctagagcatgg | |
| | ctacgtagataagtagcatggcgggttaatcattaactaca | |
| hTNGE103 | tgtagttaatgattaacccgccatgctacttatctacgtagccatgctctaggaagatcggaattcg | 129 |
| | cccttaaggtcatggagaagacccaccttgcagatgtcctcactggggctggcagagccggca | |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGgctcgctcagctataagagcctc | |
| | aaccgccccaaagaagaagcggaaggtcggtatccacggagtcccagcagccaagcggaac | |
| | tacatcctgggcctggacatcggcatcaccagcgtgggctacggcatcatcgactacgagacac | |
| | gggacgtgatcgatgccggcgtgcggctgttcaaagaggccaacgtggaaaacaacgagggc | |
| | aggcggagcaagagaggcgccagaaggctgaagcggcggaggcggcatagaatccagaga | |
| | gtgaagaagctgctgttcgactacaacctgctgaccgaccacagcgagctgagcggcatcaac | |
| | ccctacgaggccagagtgaagggcctgagccagaagctgagcgaggaagagttctctgccgc | |
| | cctgctgcacctggccaagagaagaggcgtgcacaacgtgaacgaggtggaagaggacacc | |
| | ggcaacgagctgtccaccaaagagcagatcagccggaacagcaaggccctggaagagaaat | |
| | acgtggccgaactgcagctggaacggctgaagaaagacggcgaagtgcggggcagcatcaa | |
| | cagattcaagaccagcgactacgtgaaagaagccaaacagctgctgaaggtgcagaaggccta | |
| | ccaccagctggaccagagcttcatcgacacctacatcgacctgctggaaacccggggaccta | |
| | ctatgagggacctggcgagggcagccccttcggctggaaggacatcaaagaatggtacgagat | |
| | gctgatgggccactgcacctacttccccgaggaactgcggagcgtgaagtacgcctacaacgc | |
| | cgacctgtacaacgccctgaacgacctgaacaatctcgtgatcaccagggacgagaacgagaa | |
| | gctggaatattacgagaagttccagatcatcgagaacgtgttcaagcagaagaagaagcccacc | |
| | ctgaagcagatcgccaaagaaatcctcgtgaacgaagaggatattaagggctacagagtgacc | |
| | agcaccggcaagcccgagttcaccaacctgaaggtgtaccacgacatcaaggacattaccgcc | |
| | cggaaagagattattgagaacgccgagctgctggatcagattgccaagatcctgaccatctacca | |
| | gagcagcgaggacatccaggaagaactgaccaatctgaactccgagctgacccaggaagaga | |
| | tcgagcagatctctaatctgaagggctataccggcacccacaacctgagcctgaaggccatcaa | |
| | cctgatcctggacgagctgtggcacaccaacgacaaccagatcgctatcttcaaccggctgaag | |
| | ctggtgcccaagaaggtggacctgtcccagcagaaagagatccccaccaccctggtggacga | |
| | cttcatcctgagccccgtcgtgaagagaagcttcatccagagcatcaaagtgatcaacgccatca | |
| | tcaagaagtacggcctgcccaacgacatcattatcgagctggcccgcgagaagaactccaagg | |
| | acgcccagaaaatgatcaacgagatgcagaagcggaaccggcagaccaacgagcggatcga | |
| | ggaaatcatccggaccaccggcaaagagaacgccaagtacctgatcgagaagatcaagctgc | |
| | acgacatgcaggaaggcaagtgcctgtacagcctggaagccatccctctggaagatctgctgaa | |
| | caaccccttcaactatgaggtggaccacatcatccccagaagcgtgtccttcgacaacagcttca | |
| | acaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgggttgaggctcttat | |
| | agctgagttttagtactctggaaacagaatctactaaaacaaggcaaaatgccgtgtttatctcgtc | |
| | aacttgttggcgagatttttgctcgagttaagggcgaattcccgattaggatcttcctagagcatgg | |
| | ctacgtagataagtagcatggcgggttaatcattaactaca | |
| hTNGE104 | tgtagttaatgattaacccgccatgctacttatctacgtagccatgctctaggaagatcggaattcg | 130 |
| | cccttaaggtcatggagaagacccaccttgcagatgtcctcactggggctggcagagccggca | |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGtctcgctcagctataagagcctc | |
| | aaccgccccaaagaagaagcggaaggtcggtatccacggagtcccagcagccaagcggaac | |
| | tacatcctgggcctggacatcggcatcaccagcgtgggctacggcatcatcgactacgagacac | |
| | gggacgtgatcgatgccggcgtgcggctgttcaaagaggccaacgtggaaaacaacgagggc | |
| | aggcggagcaagagaggcgccagaaggctgaagcggcggaggcggcatagaatccagaga | |
| | gtgaagaagctgctgttcgactacaacctgctgaccgaccacagcgagctgagcggcatcaac | |
| | ccctacgaggccagagtgaagggcctgagccagaagctgagcgaggaagagttctctgccgc | |
| | cctgctgcacctggccaagagaagaggcgtgcacaacgtgaacgaggtggaagaggacacc | |
| | ggcaacgagctgtccaccaaagagcagatcagccggaacagcaaggccctggaagagaaat | |
| | acgtggccgaactgcagctggaacggctgaagaaagacggcgaagtgcggggcagcatcaa | |
| | cagattcaagaccagcgactacgtgaaagaagccaaacagctgctgaaggtgcagaaggccta | |
| | ccaccagctggaccagagcttcatcgacacctacatcgacctgctggaaacccggcggaccta | |
| | ctatgagggacctggcgagggcagccccttcggctggaaggacatcaaagaatggtacgagat | |
| | gctgatgggccactgcacctacttccccgaggaactgcggagcgtgaagtacgcctacaacgc | |
| | cgacctgtacaacgccctgaacgacctgaacaatctcgtgatcaccagggacgagaacgagaa | |
| | gctggaatattacgagaagttccagatcatcgagaacgtgttcaagcagaagaagaagcccacc | |
| | ctgaagcagatcgccaaagaaatcctcgtgaacgaagaggatattaagggctacagagtgacc | |
| | agcaccggcaagcccgagttcaccaacctgaaggtgtaccacgacatcaaggacattaccgcc | |
| | cggaaagagattattgagaacgccgagctgctggatcagattgccaagatcctgaccatctacca | |
| | gagcagcgaggacatccaggaagaactgaccaatctgaactccgagctgacccaggaagaga | |
| | tcgagcagatctctaatctgaagggctataccggcacccacaacctgagcctgaaggccatcaa | |
| | cctgatcctggacgagctgtggcacaccaacgacaaccagatcgctatcttcaaccggctgaag | |
| | ctggtgcccaagaaggtggacctgtcccagcagaaagagatccccaccaccctggtggacga | |
| | cttcatcctgagccccgtcgtgaagagaagcttcatccagagcatcaaagtgatcaacgccatca | |
| | tcaagaagtacggcctgcccaacgacatcattatcgagctggcccgcgagaagaactccaagg | |
| | acgcccagaaaatgatcaacgagatgcagaagcggaaccggcagaccaacgagcggatcga | |
| | ggaaatcatccggaccaccggcaaagagaacgccaagtacctgatcgagaagatcaagctgc | |
| | acgacatgcaggaaggcaagtgcctgtacagcctggaagccatccctctggaagatctgctgaa | |
| | caaccccttcaactatgaggtggaccacatcatccccagaagcgtgtccttcgacaacagcttca | |
| | acaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgggttgaggctcttat | |
| | agctgagttttagtactctggaaacagaatctactaaaacaaggcaaaatgccgtgtttatctcgtc | |
| | aacttgttggcgagatttttgctcgagttaagggcgaattcccgattaggatcttcctagagcatgg | |
| | ctacgtagataagtagcatggcgggttaatcattaactaca | |
| hTNGE105 | tgtagttaatgattaacccgccatgctacttatctacgtagccatgctctaggaagatcggaattcg | 131 |
| | cccttaaggtcatggagaagacccaccttgcagatgtcctcactggggctggcagagccggca | |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGcctcgctcagctataagagcctc | |
| | aaccgccccaaagaagaagcggaaggtcggtatccacggagtcccagcagccaagcggaac | |
| | tacatcctgggcctggacatcggcatcaccagcgtgggctacggcatcatcgactacgagacac | |
| | gggacgtgatcgatgccggcgtgcggctgttcaaagaggccaacgtggaaaacaacgagggc | |
| | aggcggagcaagagaggcgccagaaggctgaagcggcggaggcggcatagaatccagaga | |
| | gtgaagaagctgctgttcgactacaacctgctgaccgaccacagcgagctgagcggcatcaac | |
| | ccctacgaggccagagtgaagggcctgagccagaagctgagcgaggaagagttctctgccgc | |
| | cctgctgcacctggccaagagaagaggcgtgcacaacgtgaacgaggtggaagaggacacc | |
| | ggcaacgagctgtccaccaaagagcagatcagccggaacagcaaggccctggaagagaaat | |
| | acgtggccgaactgcagctggaacggctgaagaaagacggcgaagtgcggggcagcatcaa | |
| | cagattcaagaccagcgactacgtgaaagaagccaaacagctgctgaaggtgcagaaggccta | |
| | ccaccagctggaccagagcttcatcgacacctacatcgacctgctggaaacccggcggaccta | |
| | ctatgagggacctggcgagggcagccccttcggctggaaggacatcaaagaatggtacgagat | |
| | gctgatgggccactgcacctacttccccgaggaactgcggagcgtgaagtacgcctacaacgc | |
| | cgacctgtacaacgccctgaacgacctgaacaatctcgtgatcaccagggacgagaacgagaa | |
| | gctggaatattacgagaagttccagatcatcgagaacgtgttcaagcagaagaagaagcccacc | |
| | ctgaagcagatcgccaaagaaatcctcgtgaacgaagaggatattaagggctacagagtgacc | |
| | agcaccggcaagcccgagttcaccaacctgaaggtgtaccacgacatcaaggacattaccgcc | |
| | cggaaagagattattgagaacgccgagctgctggatcagattgccaagatcctgaccatctacca | |
| | gagcagcgaggacatccaggaagaactgaccaatctgaactccgagctgacccaggaagaga | |
| | tcgagcagatctctaatctgaagggctataccggcacccacaacctgagcctgaaggccatcaa | |
| | cctgatcctggacgagctgtggcacaccaacgacaaccagatcgctatcttcaaccggctgaag | |
| | ctggtgcccaagaaggtggacctgtcccagcagaaagagatccccaccaccctggtggacga | |
| | cttcatcctgagccccgtcgtgaagagaagcttcatccagagcatcaaagtgatcaacgccatca | |
| | tcaagaagtacggcctgcccaacgacatcattatcgagctggcccgcgagaagaactccaagg | |
| | acgcccagaaaatgatcaacgagatgcagaagcggaaccggcagaccaacgagcggatcga | |
| | ggaaatcatccggaccaccggcaaagagaacgccaagtacctgatcgagaagatcaagctgc | |
| | acgacatgcaggaaggcaagtgcctgtacagcctggaagccatccctctggaagatctgctgaa | |
| | caaccccttcaactatgaggtggaccacatcatccccagaagcgtgtccttcgacaacagcttca | |
| | acaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgggttgaggctcttat | |
| | agctgagttttagtactctggaaacagaatctactaaaacaaggcaaaatgccgtgtttatctcgtc | |
| | aacttgttggcgagatttttgctcgagttaagggcgaattcccgattaggatcttcctagagcatgg | |
| | ctacgtagataagtagcatggcgggttaatcattaactaca | |
| mTNGE102 | tgtagttaatgattaacccgccatgctacttatctacgtagccatgctctaggaagatcggaattcg | |
| | cccttaaggtcatggagaagacccaccttgcagatgtcctcactggggctggcagagccggca | 132 |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGactcgctcggctatcagggcct | |
| | ccactgccccaaagaagaagcggaaggtcggtatccacggagtcccagcagccaagcggaa | |
| | ctacatcctgggcctggacatcggcatcaccagcgtgggctacggcatcatcgactacgagaca | |
| | cgggacgtgatcgatgccggcgtgcggctgttcaaagaggccaacgtggaaaacaacgaggg | |
| | caggcggagcaagagaggcgccagaaggctgaagcggcggaggcggcatagaatccagag | |
| | agtgaagaagctgctgttcgactacaacctgctgaccgaccacagcgagctgagcggcatcaa | |
| | cccctacgaggccagagtgaagggcctgagccagaagctgagcgaggaagagttctctgccg | |
| | ccctgctgcacctggccaagagaagaggcgtgcacaacgtgaacgaggtggaagaggacac | |
| | cggcaacgagctgtccaccaaagagcagatcagccggaacagcaaggccctggaagagaaa | |
| | tacgtggccgaactgcagctggaacggctgaagaaagacggcgaagtgcggggcagcatca | |
| | acagattcaagaccagcgactacgtgaaagaagccaaacagctgctgaaggtgcagaaggcct | |
| | accaccagctggaccagagcttcatcgacacctacatcgacctgctggaaacccggcggacct | |
| | actatgagggacctggcgagggcagccccttcggctggaaggacatcaaagaatggtacgag | |
| | atgctgatgggccactgcacctacttccccgaggaactgcggagcgtgaagtacgcctacaacg | |
| | ccgacctgtacaacgccctgaacgacctgaacaatctcgtgatcaccagggacgagaacgaga | |
| | agctggaatattacgagaagttccagatcatcgagaacgtgttcaagcagaagaagaagcccac | |
| | cctgaagcagatcgccaaagaaatcctcgtgaacgaagaggatattaagggctacagagtgac | |
| | cagcaccggcaagcccgagttcaccaacctgaaggtgtaccacgacatcaaggacattaccgc | |
| | ccggaaagagattattgagaacgccgagctgctggatcagattgccaagatcctgaccatctacc | |
| | agagcagcgaggacatccaggaagaactgaccaatctgaactccgagctgacccaggaagag | |
| | atcgagcagatctctaatctgaagggctataccggcacccacaacctgagcctgaaggccatca | |
| | acctgatcctggacgagctgtggcacaccaacgacaaccagatcgctatcttcaaccggctgaa | |
| | gctggtgcccaagaaggtggacctgtcccagcagaaagagatccccaccaccctggtggacg | |
| | acttcatcctgagccccgtcgtgaagagaagcttcatccagagcatcaaagtgatcaacgccatc | |
| | atcaagaagtacggcctgcccaacgacatcattatcgagctggcccgcgagaagaactccaag | |
| | gacgcccagaaaatgatcaacgagatgcagaagcggaaccggcagaccaacgagcggatcg | |
| | aggaaatcatccggaccaccggcaaagagaacgccaagtacctgatcgagaagatcaagctg | |
| | cacgacatgcaggaaggcaagtgcctgtacagcctggaagccatccctctggaagatctgctga | |
| | acaaccccttcaactatgaggtggaccacatcatccccagaagcgtgtccttcgacaacagcttc | |
| | aacaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgagtggaggccctg | |
| | atagccgagttttagtactctggaaacagaatctactaaaacaaggcaaaatgccgtgtttatctcg | |
| | tcaacttgttggcgagatttttgctcgagttaagggcgaattcccgattaggatcttcctagagcatg | |
| | gctacgtagataagtagcatggcgggttaatcattaactaca | |
| mTNGE103 | tgtagttaatgattaacccgccatgctacttatctacgtagccatgctctaggaagatcggaattcg | 133 |
| | cccttaaggtcatggagaagacccaccttgcagatgtcctcactggggctggcagagccggca | |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGgctcgctcggctatcagggcct | |
| | ccactgccccaaagaagaagcggaaggtcggtatccacggagtcccagcagccaagcggaa | |
| | ctacatcctgggcctggacatcggcatcaccagcgtgggctacggcatcatcgactacgagaca | |
| | cgggacgtgatcgatgccggcgtgcggctgttcaaagaggccaacgtggaaaacaacgaggg | |
| | caggcggagcaagagaggcgccagaaggctgaagcggcggaggcggcatagaatccagag | |
| | agtgaagaagctgctgttcgactacaacctgctgaccgaccacagcgagctgagcggcatcaa | |
| | cccctacgaggccagagtgaagggcctgagccagaagctgagcgaggaagagttctctgccg | |
| | ccctgctgcacctggccaagagaagaggcgtgcacaacgtgaacgaggtggaagaggacac | |
| | cggcaacgagctgtccaccaaagagcagatcagccggaacagcaaggccctggaagagaaa | |
| | tacgtggccgaactgcagctggaacggctgaagaaagacggcgaagtgcggggcagcatca | |
| | acagattcaagaccagcgactacgtgaaagaagccaaacagctgctgaaggtgcagaaggcct | |
| | accaccagctggaccagagcttcatcgacacctacatcgacctgctggaaacccggcggacct | |
| | actatgagggacctggcgagggcagccccttcggctggaaggacatcaaagaatggtacgag | |
| | atgctgatgggccactgcacctacttccccgaggaactgcggagcgtgaagtacgcctacaacg | |
| | ccgacctgtacaacgccctgaacgacctgaacaatctcgtgatcaccagggacgagaacgaga | |
| | agctggaatattacgagaagttccagatcatcgagaacgtgttcaagcagaagaagaagcccac | |
| | cctgaagcagatcgccaaagaaatcctcgtgaacgaagaggatattaagggctacagagtgac | |
| | cagcaccggcaagcccgagttcaccaacctgaaggtgtaccacgacatcaaggacattaccgc | |
| | ccggaaagagattattgagaacgccgagctgctggatcagattgccaagatcctgaccatctacc | |
| | agagcagcgaggacatccaggaagaactgaccaatctgaactccgagctgacccaggaagag | |
| | atcgagcagatctctaatctgaagggctataccggcacccacaacctgagcctgaaggccatca | |
| | acctgatcctggacgagctgtggcacaccaacgacaaccagatcgctatcttcaaccggctgaa | |
| | gctggtgcccaagaaggtggacctgtcccagcagaaagagatccccaccaccctggtggacg | |
| | acttcatcctgagccccgtcgtgaagagaagcttcatccagagcatcaaagtgatcaacgccatc | |
| | atcaagaagtacggcctgcccaacgacatcattatcgagctggcccgcgagaagaactccaag | |
| | gacgcccagaaaatgatcaacgagatgcagaagcggaaccggcagaccaacgagcggatcg | |
| | aggaaatcatccggaccaccggcaaagagaacgccaagtacctgatcgagaagatcaagctg | |
| | cacgacatgcaggaaggcaagtgcctgtacagcctggaagccatccctctggaagatctgctga | |
| | acaaccccttcaactatgaggtggaccacatcatccccagaagcgtgtccttcgacaacagcttc | |
| | aacaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgagtggaggccctg | |
| | atagccgagttttagtactctggaaacagaatctactaaaacaaggcaaaatgccgtgtttatctcg | |
| | tcaacttgttggcgagatttttgctcgagttaagggcgaattcccgattaggatcttcctagagcatg | |
| | gctacgtagataagtagcatgggggttaatcattaactaca | |
| mTNGE104 | tgtagttaatgattaacccgccatgctacttatctacgtagccatgctctaggaagatcggaattcg | 134 |
| | cccttaaggtcatggagaagacccaccttgcagatgtcctcactggggctggcagagccggca | |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGtctcgctcggctatcagggcctc | |
| | cactgccccaaagaagaagcggaaggtcggtatccacggagtcccagcagccaagcggaact | |
| | acatcctgggcctggacatcggcatcaccagcgtgggctacggcatcatcgactacgagacac | |
| | gggacgtgatcgatgccggcgtgcggctgttcaaagaggccaacgtggaaaacaacgagggc | |
| | aggcggagcaagagaggcgccagaaggctgaagcggcggaggcggcatagaatccagaga | |
| | gtgaagaagctgctgttcgactacaacctgctgaccgaccacagcgagctgagcggcatcaac | |
| | ccctacgaggccagagtgaagggcctgagccagaagctgagcgaggaagagttctctgccgc | |
| | cctgctgcacctggccaagagaagaggcgtgcacaacgtgaacgaggtggaagaggacacc | |
| | ggcaacgagctgtccaccaaagagcagatcagccggaacagcaaggccctggaagagaaat | |
| | acgtggccgaactgcagctggaacggctgaagaaagacggcgaagtgcggggcagcatcaa | |
| | cagattcaagaccagcgactacgtgaaagaagccaaacagctgctgaaggtgcagaaggccta | |
| | ccaccagctggaccagagcttcatcgacacctacatcgacctgctggaaacccggcggaccta | |
| | ctatgagggacctggcgagggcagccccttcggctggaaggacatcaaagaatggtacgagat | |
| | gctgatgggccactgcacctacttccccgaggaactgcggagcgtgaagtacgcctacaacgc | |
| | cgacctgtacaacgccctgaacgacctgaacaatctcgtgatcaccagggacgagaacgagaa | |
| | gctggaatattacgagaagttccagatcatcgagaacgtgttcaagcagaagaagaagcccacc | |
| | ctgaagcagatcgccaaagaaatcctcgtgaacgaagaggatattaagggctacagagtgacc | |
| | agcaccggcaagcccgagttcaccaacctgaaggtgtaccacgacatcaaggacattaccgcc | |
| | cggaaagagattattgagaacgccgagctgctggatcagattgccaagatcctgaccatctacca | |
| | gagcagcgaggacatccaggaagaactgaccaatctgaactccgagctgacccaggaagaga | |
| | tcgagcagatctctaatctgaagggctataccggcacccacaacctgagcctgaaggccatcaa | |
| | cctgatcctggacgagctgtggcacaccaacgacaaccagatcgctatcttcaaccggctgaag | |
| | ctggtgcccaagaaggtggacctgtcccagcagaaagagatccccaccaccctggtggacga | |
| | cttcatcctgagccccgtcgtgaagagaagcttcatccagagcatcaaagtgatcaacgccatca | |
| | tcaagaagtacggcctgcccaacgacatcattatcgagctggcccgcgagaagaactccaagg | |
| | acgcccagaaaatgatcaacgagatgcagaagcggaaccggcagaccaacgagcggatcga | |
| | ggaaatcatccggaccaccggcaaagagaacgccaagtacctgatcgagaagatcaagctgc | |
| | acgacatgcaggaaggcaagtgcctgtacagcctggaagccatccctctggaagatctgctgaa | |
| | caaccccttcaactatgaggtggaccacatcatccccagaagcgtgtccttcgacaacagcttca | |
| | acaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgagtggaggccctg | |
| | atagccgagttttagtactctggaaacagaatctactaaaacaaggcaaaatgccgtgtttatctcg | |
| | tcaacttgttggcgagatttttgctcgagttaagggcgaattcccgattaggatcttcctagagcatg | |
| | gctacgtagataagtagcatgggggttaatcattaactaca | |
| mTNGE105 | tgtagttaatgattaacccgccatgctacttatctacgtagccatgctctaggaagatcggaattcg | 135 |
| | cccttaaggtcatggagaagacccaccttgcagatgtcctcactggggctggcagagccggca | |
| | acctgcctaaggctgctcagtccattaggagccagtagcctggaagatgtctttacccccagcat | |
| | cagttcaagtggagcagcacataactcttgccctctgccttccaagattctggtgctgagacttatg | |
| | gagtgtcttggaggttgccttctgccccccaaccctgctcccagctggccctcccaggcctgggt | |
| | tgctggcctctgctttatcaggattctcaagagggacagctggtttatgttgcatgactgttccctgc | |
| | atatctgctctggttttaaatagcttatctgagcagctggaggaccacatgggcttatatggcgtgg | |
| | ggtacatgttcctgtagccttgtccctggcacctgccaaaatagcagccaacaccccccaccccc | |
| | accgccatccccctgccccacccgtcccctgtcgcacattcctccctccgcagggctggctcac | |
| | caggccccagcccacatgcctgcttaaagccctctccatcctctgcctcacccagtccccgctga | |
| | gactgagcagacgcctccagcggccgcccgccaccATGcctcgctcggctatcagggcctc | |
| | cactgccccaaagaagaagcggaaggtcggtatccacggagtcccagcagccaagcggaact | |
| | acatcctgggcctggacatcggcatcaccagcgtgggctacggcatcatcgactacgagacac | |
| | gggacgtgatcgatgccggcgtgcggctgttcaaagaggccaacgtggaaaacaacgagggc | |
| | aggcggagcaagagaggcgccagaaggctgaagcggcggaggcggcatagaatccagaga | |
| | gtgaagaagctgctgttcgactacaacctgctgaccgaccacagcgagctgagcggcatcaac | |
| | ccctacgaggccagagtgaagggcctgagccagaagctgagcgaggaagagttctctgccgc | |
| | cctgctgcacctggccaagagaagaggcgtgcacaacgtgaacgaggtggaagaggacacc | |
| | ggcaacgagctgtccaccaaagagcagatcagccggaacagcaaggccctggaagagaaat | |
| | acgtggccgaactgcagctggaacggctgaagaaagacggcgaagtgcggggcagcatcaa | |
| | cagattcaagaccagcgactacgtgaaagaagccaaacagctgctgaaggtgcagaaggccta | |
| | ccaccagctggaccagagcttcatcgacacctacatcgacctgctggaaacccggcggaccta | |
| | ctatgagggacctggcgagggcagccccttcggctggaaggacatcaaagaatggtacgagat | |
| | gctgatgggccactgcacctacttccccgaggaactgcggagcgtgaagtacgcctacaacgc | |
| | cgacctgtacaacgccctgaacgacctgaacaatctcgtgatcaccagggacgagaacgagaa | |
| | gctggaatattacgagaagttccagatcatcgagaacgtgttcaagcagaagaagaagcccacc | |
| | ctgaagcagatcgccaaagaaatcctcgtgaacgaagaggatattaagggctacagagtgacc | |
| | agcaccggcaagcccgagttcaccaacctgaaggtgtaccacgacatcaaggacattaccgcc | |
| | cggaaagagattattgagaacgccgagctgctggatcagattgccaagatcctgaccatctacca | |
| | gagcagcgaggacatccaggaagaactgaccaatctgaactccgagctgacccaggaagaga | |
| | tcgagcagatctctaatctgaagggctataccggcacccacaacctgagcctgaaggccatcaa | |
| | cctgatcctggacgagctgtggcacaccaacgacaaccagatcgctatcttcaaccggctgaag | |
| | ctggtgcccaagaaggtggacctgtcccagcagaaagagatccccaccaccctggtggacga | |
| | cttcatcctgagccccgtcgtgaagagaagcttcatccagagcatcaaagtgatcaacgccatca | |
| | tcaagaagtacggcctgcccaacgacatcattatcgagctggcccgcgagaagaactccaagg | |
| | acgcccagaaaatgatcaacgagatgcagaagcggaaccggcagaccaacgagcggatcga | |
| | ggaaatcatccggaccaccggcaaagagaacgccaagtacctgatcgagaagatcaagctgc | |
| | acgacatgcaggaaggcaagtgcctgtacagcctggaagccatccctctggaagatctgctgaa | |
| | caaccccttcaactatgaggtggaccacatcatccccagaagcgtgtccttcgacaacagcttca | |
| | acaacaaggtgctcgtgaagcaggaagaaaacagcaagaagggcaaccggaccccattcca | |
| | gtacctgagcagcagcgacagcaagatcagctacgaaaccttcaagaagcacatcctgaatctg | |
| | gccaagggcaagggcagaatcagcaagaccaagaaagagtatctgctggaagaacgggaca | |
| | tcaacaggttctccgtgcagaaagacttcatcaaccggaacctggtggataccagatacgccac | |
| | cagaggcctgatgaacctgctgcggagctacttcagagtgaacaacctggacgtgaaagtgaa | |
| | gtccatcaatggcggcttcaccagctttctgcggcggaagtggaagtttaagaaagagcggaac | |
| | aaggggtacaagcaccacgccgaggacgccctgatcattgccaacgccgatttcatcttcaaag | |
| | agtggaagaaactggacaaggccaaaaaagtgatggaaaaccagatgttcgaggaaaagcag | |
| | gccgagagcatgcccgagatcgaaaccgagcaggagtacaaagagatcttcatcacccccca | |
| | ccagatcaagcacattaaggacttcaaggactacaagtacagccaccgggtggacaagaagcc | |
| | taatagagagctgattaacgacaccctgtactccacccggaaggacgacaagggcaacaccct | |
| | gatcgtgaacaatctgaacggcctgtacgacaaggacaatgacaagctgaaaaagctgatcaac | |
| | aagagccccgaaaagctgctgatgtaccaccacgacccccagacctaccagaaactgaagctg | |
| | attatggaacagtacggcgacgagaagaatcccctgtacaagtactacgaggaaaccgggaac | |
| | tacctgaccaagtactccaaaaaggacaacggccccgtgatcaagaagattaagtattacggca | |
| | acaaactgaacgcccatctggacatcaccgacgactaccccaacagcagaaacaaggtcgtga | |
| | agctgtccctgaagccctacagattcgacgtgtacctggacaatggcgtgtacaagttcgtgacc | |
| | gtgaagaatctggatgtgatcaaaaaagaaaactactacgaagtgaatagcaagtgctatgagg | |
| | aagctaagaagctgaagaagatcagcaaccaggccgagtttatcgcctccttctacaacaacgat | |
| | ctgatcaagatcaacggcgagctgtatagagtgatcggcgtgaacaacgacctgctgaaccgg | |
| | atcgaagtgaacatgatcgacatcacctaccgcgagtacctggaaaacatgaacgacaagagg | |
| | ccccccaggatcattaagacaatcgcctccaagacccagagcattaagaagtacagcacagac | |
| | attctgggcaacctgtatgaagtgaaatctaagaagcaccctcagatcatcaaaaagggcaaaa | |
| | ggccggcggccacgaaaaaggccggccaggcaaaaaagaaaaagtaaagatctgcctcgac | |
| | tgtgccttctagttgccagccatctgttgtttgcccctcccccgtgccttccttgaccctggaaggtg | |
| | ccactcccactgtcctttcctaataaaatgaggaaattgcatcgcattgtctgagtaggtgtcattct | |
| | attctggggggtggggggggcaggacagcaagggggaggattgggaagacaatagcaggc | |
| | atgctggggagagggcctatttcccatgattccttcatatttgcatatacgatacaaggctgttaga | |
| | gagataattggaattaatttgactgtaaacacaaagatattagtacaaaatacgtgacgtagaaagtaa | |
| | taatttcttgggtagtttgcagttttaaaattatgttttaaaatggactatcatatgcttaccgtaactt | |
| | gaaagtatttcgatttcttggctttatatatcttgtggaaaggacgaaacaccgagtggaggccctg | |
| | atagccgagttttagtactctggaaacagaatctactaaaacaaggcaaaatgccgtgtttatctcg | |
| | tcaacttgttggcgagatttttgctcgagttaagggcgaattcccgattaggatcttcctagagcatg | |
| | gctacgtagataagtagcatggcgggttaatcattaactaca | |

### Vectors

In some aspects, the disclosure provides vectors comprising the expression cassettes provided herein. The vector can be any viral vector or any non-viral vector known in the art or described herein.

In some embodiments, the vector is a viral vector. In some embodiments the viral vector is an adeno-associated virus vector (AAV), an adenoviral vector (AV), a lentiviral vector (LV), a retroviral vector (RV), a herpes simplex virus vector (HSV), or a poxvirus vector.

In some embodiments, provided herein is an AAV comprising any expression cassette described herein. In some embodiments, provided herein is an AV comprising any expression cassette described herein. In some embodiments, provided herein is an LV comprising any expression cassette described herein. In some embodiments, provided herein is an RV comprising any expression cassette described herein. In some embodiments, provided herein is an HSV comprising any expression cassette described herein. In some embodiments, provided herein is a poxvirus-based vector comprising any expression cassette described herein.

In some embodiments, the viral vector is a retroviral vector, e.g., a lentiviral vector. As used herein, the term “retrovirus” or “retroviral” refers an RNA virus that reverse transcribes its genomic RNA into a linear double-stranded DNA copy and subsequently covalently integrates its genomic DNA into a host genome. Retrovirus vectors are a common tool for gene delivery (Miller, Nature. 357: 455-460 (2000)). Once the virus is integrated into the host genome, it is referred to as a “provirus.” The provirus serves as a template for RNA polymerase II and directs the expression of RNA molecules encoded by the virus. In some embodiments, a retroviral vector is altered so that it does not integrate into the host cell genome.

Illustrative retroviruses (family Retroviridae) include, but are not limited to: (1) genus gammaretrovirus, such as, Moloney murine leukemia virus (M-MuLV or MMLV), Moloney murine sarcoma virus (MoMSV), murine mammary tumor virus (MuMTV), gibbon ape leukemia virus (GaLV), and feline leukemia virus (FLV), (2) genus spumavirus, such as, simian foamy virus, (3) genus lentivirus, such as, human immunodeficiency virus-1 and simian immunodeficiency virus.

As used herein, the term “lentiviral” or “lentivirus” refers to a group (or genus) of complex retroviruses. Illustrative lentiviruses include but are not limited to HIV (human immunodeficiency virus; including HIV type 1, and HIV type 2; visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In some embodiments, the viral vector is an adenoviral vector. The genetic organization of adenovirus includes an approximate 36 kb, linear, double-stranded DNA virus, which allows substitution of large pieces of adenoviral DNA with foreign sequences up to 7 kb (Grunhaus et al., Seminar in Virology 200(2):535-546, 1992)).

In some embodiments, the viral vector is an adeno-associated viral (AVV) vector, such as an AAV vector selected from the group consisting of serotype 1, 2, 3, 4, 5, 6, 7, 8, 9, and 10 or chimeric AAV derived thereof.

In some embodiments, the AAV expression vector is pseudotyped to enhance targeting. A pseudotyping strategy can promote gene transfer and sustain expression in a target cell type. For example, the AAV2 genome can be packaged into the capsid of another AAV serotype such as AAV5, AAV7, or AAV8, producing pseudotyped vectors such as AAV2/5, AAV2/7, and AAV2/8 respectively, as described in Balaji et al. J Surg Res. September; 184(1): 691-698 (2013). In some embodiments, an AAV9 may be used to target expression in myofibroblast-like lineages, as described in Piras et al. Gene Therapy 23:469-478 (2016). In some embodiments, AAV1, AAV6, or AAV9 is used, and in some embodiments, the AAV is engineered, as described in Asokari et al. Hum Gene Ther. November; 24(11): 906-913 (2013); Pozsgai et al. Mol Ther. April 5; 25(4): 855-869 (2017); Kotterman, M. A. and D. V. Schaffer Engineering Adeno-Associated Viruses for Clinical Gene Therapy. Nature Reviews Genetics, 15:445-451 (2014); and US20160340393A1 to Schaffer et al. In some embodiments, the viral vector is AAV engineered to increase target cell infectivity as described in US20180066285A1. In some embodiments, the vector is an AAV9 vector.

In some embodiments, the vector is a non-viral vector. In some embodiments, the non-viral vector is a naked DNA (e.g., a DNA plasmid). In some embodiments, the non-viral vector is a plasmid. In some embodiments, the non-viral vector is a liposome or lipid vector comprising plasmid DNA and a lipid solution.

For example, viral and non-viral vectors and delivery systems are described in Sung & Kim 2019, Biomaterials Research 23:8; Mali, 2013, Indian Journal of Human Genetics, 19(1):3-8; Hardee et al., 2017, Genes 8:65; Bulcha et al., 2020, Signal Transduction and Targeted Therapy; Ghosh et al., 2020, Applied Biosafety: Journal of ABSA International 25(1):7-18, the disclosures of each of which are hereby incorporated by reference herein in their entireties.

In some embodiments, the vectors are recombinant vectors.

In some aspects of the disclosure, a vector is used to deliver the expression cassettes described herein to cardiac cells of a subject, e.g., to treat cardiomyopathy.

In some embodiments, the viral vectors described herein are replication incompetent, in that it cannot independently further replicate and package its genome. For example, when a cardiac cell is targeted with a virion, the transgene is expressed in the targeted cardiac cell, however, due to the fact that the targeted cardiac cell lacks packaging and accessory function genes, the virion is not able to replicate. In some embodiments, the viral vectors described herein are replication-competent.

In some embodiments, the vectors described herein are capable of being delivered to both dividing and non-dividing cells. In some embodiments, the vectors described herein are capable of being delivered to non-dividing cells. In some embodiments, the vectors described herein are capable of being delivered to dividing cells.

In some embodiments, the vectors comprising the expression cassettes described herein lead to cardiac cell-specific expression of the coding sequence(s). In some embodiments, the vectors comprising the expression cassettes described herein lead to cardiomyocyte-specific expression of the coding sequence(s). In some embodiments, the vectors comprising the expression cassettes described herein allow high expression of the coding sequence(s) in a cardiac cell (e.g., a cardiomyocyte) and low or no expression in other cells (e.g., low or no expression in liver cells, low or no expression in muscle cells except for muscle cells of the heart, low or no expression in cardiac fibroblasts). In some embodiments, the vectors comprising the expression cassettes described herein allow high expression of the coding sequence(s) in heart tissue of a subject (e.g., in human heart). In some embodiments, the vectors comprising the expression cassettes described herein allow no or low expression of the coding sequence(s) in tissues of a subject other than the heart (e.g., in liver or in muscles except those of the heart). “High” and “low” can be relative to each other, for example, the expression of a transgene in cardiac cells (e.g., cardiomyocytes) and/or heart tissue can be at least 2 fold, 5 fold, 10 fold, 15 fold, 20 fold, 50 fold, 100 fold, 150 fold, or 200 fold higher than its expression in other cells and tissues (e.g., liver, muscle except for the heart).

In some embodiments, the vector genome has a size of less than 6 kilobases. In some embodiments, the vector genome has a size of less than 5.6 kilobases. In some embodiments, the vector genome has a size of about, at most or less than 4.0 kilobases, 4.5 kilobases, 4.6 kilobases, 4.7 kilobases, 4.8 kilobases, 4.9 kilobases, 5 kilobases, 5.1 kilobases, 5.2 kilobases, 5.3 kilobases, 5.4 kilobases, or 5.5 kilobases. In some embodiments, the vector genome has a size of 4 kilobases to 5.2 kilobases. In some embodiments, the vector genome has a size of 4 kilobases to 5 kilobases. In some embodiments, the vector genome has a size of 4 kilobases to 4.8 kilobases. In some embodiments, the vector genome has a size of equal to or less than 4.9 kilobases. In some embodiments, the vector genome has a size of equal to or less than 4.8 kilobases. In some embodiments, the vector genome has a size of equal to or less than 4.7 kilobases. In some of these embodiments, the vector is an AAV vector, e.g., an AAV9 vector.

In some of these embodiments, the vector is an AAV vector or a variant thereof. In some of these embodiments, the vector is an AAV9 vector or a variant thereof. In some of these embodiments, the vector is an AAV5 vector or a variant thereof. In some of these embodiments, the vector is an AAV2 vector or a variant thereof.

The capsid proteins of AAV largely determine the immunogenicity and tropism of AAV vectors. In some embodiments, the AAV is an AAV subtype 9 (AAV9). In some embodiments, AAV9 is a preferred AAV vector due to its ability to transduce the heart following systemic delivery. While AAV9 can achieve moderate transduction of the heart, the majority of vector traffics to the liver. Moreover, in order to achieve therapeutic levels of transduction in the heart, relatively high systemic doses are required, potentially leading to systemic inflammation and in turn, toxicity.

In some embodiments, the vector is a retroviral vector, e.g., a lentiviral vector. In some embodiments, the disclosure comprises a mouse murine leukemia viral (MMLV) vector to deliver gene products to various tissues and cells, both in vitro and in vivo. encoded by the virus.

In some embodiments, a retroviral vector (e.g., MMLV) comprises a 5′ LTR and/or a 3′ LTR. In some embodiments, the 5′LTR of the retroviral vector comprises a promoter that drives expression of the polynucleotide. In some embodiments, a retroviral (e.g., MMLV) vector comprises psi, also known as a cis-acting sequence. The psi sequence is important for viral genome encapsidation. The psi sequence is known in the art and includes, but is not limited to, the sequence of SEQ ID NO: 41. In some embodiments, a retroviral (e.g., MMLV) vector comprises core protein genes such as gag and pol. The gag gene encodes the capsid and matrix proteins, while the pol gene encodes reverse transcriptase and integrase function. The gag and pol genes are known in the art and may comprise sequences of SEQ ID NO: 42 and 43, respectively. Retroviral core protein and gene sequences are known in the art, and depending on the host/vector system utilized, can be selected by one skilled in the art.

TABLE 11
Exemplary MMLV vector feature sequences

| | | SEQ |
|---|---|---|
| Name | Sequence | ID NO: |
| psi | aagctggccagcaacttatctgtgtctgtccgattgtctagtgtctatgactgattttatgcgcctgcgtc | 41 |
| | ggtactagttagctaactagctctgtatctggcggacccgtggtggaactgacgagttcggaacacc | |
| | cggccgcaaccctgggagacgtcccagggacttcgggggccgtttttgtggcccgacctgagtcc | |
| | aaaaatcccgatcgttttggactctttggtgcacccccctaataggagggatatgtggttctggtagg | |
| | agacgagaacctaaaacagttcccgcctccgtctgaatttttgctttcggtttgggaccgaagccgcg | |
| | ccgcgcgtcttgtctgctg | |
| gag | ggccagactgttaccactcccttaagtttgaccttaggtcactggaaagatgtcgagcggatcgctca | 42 |
| (truncated) | caaccagtcggtagatgtcaagaagagacgttgggttaccttctgctctgcagaatggccaaccttta | |
| | acgtcggatggccgcgagacggcacctttaaccgagacctcatcacccaggttaagatcaaggtct | |
| | tttcacctggcccgcatggacacccagaccaggtcccctacatcgtgacctgggaagccttggcttt | |
| | tgacccccctccctgggtcaagccctttgtacaccctaagcctccgcctcctcttcctccatccgccc | |
| | cgtctctcccccttgaacctcctcgttcgaccccgcctcgatcctccctttatccagccctcactccttc | |
| | tctaggcgcc | |
| pol | ccatatgagatcttatatggggcacccccgccccttgtaaacttccctgaccctgacatgacaagagt | 43 |
| | tactaacagcccctctctccaagctcacttacaggctctctacttagtccagcacgaagtctggagac | |
| | ctctggcggcagcctaccaagaacaactggaccgaccggtggtacctcacccttaccgagtcggc | |
| | gacacagtgtgggtccgccgacaccagactaagaacctagaacctcgctggaaaggaccttacac | |
| | agtcctgctgaccacccccaccgccctcaaagtagacggcatcgcagcttggatacacgccgccc | |
| | acgtgaaggctgccgaccccgggggtggaccatcctctagactg | |

Methods of introducing polynucleotides into a host cell are known in the art, and any known method can be used to introduce the polynucleotides described herein into a cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, microfluidics delivery methods, and the like.

### Recombinant AAV Virions

In some aspects of the disclosure, an rAAV virion is used to deliver the expression cassettes described herein to cardiac cells.

In some embodiments, an adeno-associated virus (AAV) referenced herein is any AAV known in the art or described herein. In some embodiments, an AAV is an AAV selected from the group consisting of serotype 1, 2, 3, 4, 5, 6, 7, 8, 9, 10, 11, 12, rh.10, rh.20, rh.74, or a chimeric or variant AAV derived therefrom. In some embodiments, AAV is AAV1, AAV2, AAV3, AAV4, AAV5, AAV6, AAV7, AAV8, AAV9, AAV10, AAV11, AAV12, AAVrh.10, AAVrh.20, AAVrh.74, or a variant thereof.

In some embodiments, the disclosure provides an rAAV virion comprising an AAV capsid (e.g., AAV9 capsid) and an expression cassette described herein. In some embodiments, the disclosure provides an rAAV virion comprising a modified AAV capsid (e.g., a modified AAV9 capsid comprising one or more substitutions or insertions) and an expression cassette described herein.

The rAAV virions of the disclosure comprise a capsid protein. Capsid proteins are structural proteins that make up the assembled icosahedral packaging of the rAAV virion that contains the expression cassette. Capsid proteins are classified by the serotype. Wild-type capsid serotypes in rAAV virions can be, for example, AAV1, AAV2, AAV3, AAV4, AAV5, AAV6, AAV7, AAV8, AAV9, AAV10, AAV11, AAV12, AAVrh.10, AAVrh.20, or AAVrh.74 (Naso et al. BioDrugs 31:317-334 (2017)). Engineered capsid types include chimeric capsids and mosaic capsids (Choi et al. Curr Gene Ther. 5: 299-310 (2005)). Capsids are selected for rAAV virions based on their ability to transduce specific tissue or cell types (Liu et al. Curr Pharm Des. 21:3248-56 (2015)).

Any capsid protein that can facilitate rAAV virion transduction into cardiac cells for delivery of a transgene, as described herein, can be used. Capsid proteins used in rAAV virions for transgene delivery to cardiac cells that result in high expression can be, for example, AAV4, AAV6, AAV7, AAV8, and AAV9 (Zincarelli et al. Mol. Ther. 16:P1073-1080 (2008)). In some embodiments, the AAV capsid protein described herein is a wild-type AAV capsid protein from AAV serotype 1, 2, 3, 4, 5, 6, 7, 8, 9, 10, 11, 12, rh.10, rh.20, rh.74, or a variant thereof. In some embodiments, the AAV is AAV9 or a variant thereof. In some embodiments, the AAV is AAV5 or a variant thereof. In some embodiments, the AAV is AAV2 or a variant thereof.

Artificial capsids, such as chimeric capsids generated through combinatorial libraries, can also be used for transgene delivery to cardiac cells that results in high expression. Other capsid proteins with various features can also be used in the rAAV virions of the disclosure. AAV vectors and capsids are provided in U.S. Pat. Pub. Nos. U.S. Ser. No. 10/011,640B2; U.S. Pat. No. 7,892,809B2, U.S. Pat. No. 8,632,764B2, U.S. Pat. No. 8,889,641B2, U.S. Pat. No. 9,475,845B2, U.S. Ser. No. 10/889,833B2, U.S. Ser. No. 10/480,011B2, and U.S. Ser. No. 10/894,949B2, the contents of which are herein incorporated by reference; and Int'l Pat. Pub. Nos. WO2020198737A1, WO2019028306A2, WO2016054554A1, WO2018152333A1, WO2017106236A1, WO2008124724A1, WO2017212019A1, WO2020117898A1, WO2017192750A1, WO2020191300A1, and WO2017100671A1, the contents of which are herein incorporated by reference.

In some embodiments, the rAAV virions of the disclosure comprise an AAV9 capsid protein or variant thereof. In some embodiments, the rAAV virions of the disclosure comprise wild-type AAV9 capsid proteins. In some embodiments, the rAAV virions comprise an AAV5 capsid protein or variant thereof.

The wild-type AAV9 VP1 has the amino acid sequence of SEQ ID NO: 46. The wild-type AAV9 VP2 has the amino acid sequence of SEQ ID NO: 47. The wild-type AAV9 VP3 has the amino acid sequence of SEQ ID NO: 48. In some embodiments, the capsid protein described herein comprises a sequence that shares at least about 80%, 85%, 90%, 95%, 96%, 97%, 98%, 99%, 99.5%, or 100% identity to SEQ ID NO: 46. In some embodiments, the capsid protein described herein comprises a sequence that shares at least about 80%, 85%, 90%, 95%, 96%, 97%, 98%, 99%, 99.5%, or 100% identity to SEQ ID NO: 47. In some embodiments, the capsid protein described herein comprises a sequence that shares at least about 80%, 85%, 90%, 95%, 96%, 97%, 98%, 99%, 99.5%, or 100% identity to SEQ ID NO: 48. The N-terminal residue of VP1, VP2, and VP3, as well as the VR sites (VR-IV, VR-V, VR-VII, and VR-VIII), are indicated (in bold, and underlined) in the sequence of full-length VP1 (SEQ ID NO: 46) below.

```
VP1 → (SEQ ID NO: 46)
MAADGYLPDWLEDNLSEGIREWWALKPGAPQPKANQQHQDNARGLVLPGYKYLGPGNGLDKG
EPVNAADAAALEHDKAYDQQLKAGDNPYLKYNHADAEFQERLKEDTSFGGNLGRAVFQAKKR
                          VP2 → (SEQ ID NO: 47)
LLEPLGLVEEAAKTAPGKKRPVEQSPQEPDSSAGIGKSGAQPAKKRLNFGQTGDTESVPDPQ
                                VP3 → (SEQ ID NO: 48)
PIGEPPAAPSGVGSLTMASGGGAPVADNNEGADGVGSSSGNWHCDSQWLGDRVITTSTRTWA
LPTYNNHLYKQISNSTSGGSSNDNAYFGYSTPWGYFDFNRFHCHFSPRDWQRLINNNWGFRP
KRLNFKLFNIQVKEVTDNNGVKTIANNLTSTVQVFTDSDYQLPYVLGSAHEGCLPPFPADVF
MIPQYGYLTLNDGSQAVGRSSFYCLEYFPSQMLRTGNNFQFSYEFENVPFHSSYAHSQSLDR
                                    VR-IV
LMNPLIDQYLYYLSKTINGSGQNQQTLKFSVAGPSNMAVQGRNYIPGPSYRQQRVSTTVTQN
VR-V VR-VII
NNSEFAWPGASSWALNGRNSLMNPGPAMASHKEGEDRFFPLSGSLIFGKQGTGRDNVDADKV
                                            VR-VIII
MITNEEEIKTTNPVATESYGQVATNHQSAQAQAQTGWVQNQGILPGMVWQDRDVYLQGPIWA
KIPHTDGNFHPSPLMGGFGMKHPPPQILIKNTPVPADPPTAFNKDKLNSFITQYSTGQVSVE
IEWELQKENSKRWNPEIQYTSNYYKSNNVEFAVNTEGVYSEPRPIGTRYLTRNL
```

As labeled in AAV9 VP1 (SEQ ID NO: 46) above, the VR-IV site is between amino acids 452 and 458 in the parental sequence (“NGSGQNQ”, SEQ ID NO: 49); the VR-V site is between amino acids 497 and 502 in the parental sequence (“NNSEFA”, SEQ ID NO: 50); the VR-VII site is between amino acids 549 and 553 in the parental sequence (“GRDNV”, SEQ ID NO: 51); the VR-VIII site is between amino acids 581 and 594 in the parental sequence (“ATNHQSAQAQAQTG”, SEQ ID NO: 52). In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 46, excluding the VR-IV, VR-V, VR-VII, and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 46, excluding the VR-IV and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 46, excluding the VR-IV site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 46, excluding the VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 47, excluding the VR-IV, VR-V, VR-VII, and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 47, excluding the VR-IV and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 47, excluding the VR-IV site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 47, excluding the VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 48, excluding the VR-IV, VR-V, VR-VII, and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 48, excluding the VR-IV and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 48, excluding the VR-IV site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 48, excluding the VR-VIII site.

The wild-type AAV5 VP1 has the amino acid sequence of SEQ ID NO: 53. The wild-type AAV9 VP2 has the amino acid sequence of SEQ ID NO: 54. The wild-type AAV9 VP3 has the amino acid sequence of SEQ ID NO: 55. In some embodiments, the capsid protein described herein comprises a sequence that shares at least about 80%, 85%, 90%, 95%, 96%, 97%, 98%, 99%, 99.5%, or 100% identity to SEQ ID NO: 53. In some embodiments, the capsid protein described herein comprises a sequence that shares at least about 80%, 85%, 90%, 95%, 96%, 97%, 98%, 99%, 99.5%, or 100% identity to SEQ ID NO: 54. In some embodiments, the capsid protein described herein comprises a sequence that shares at least about 80%, 85%, 90%, 95%, 96%, 97%, 98%, 99%, 99.5%, or 100% identity to SEQ ID NO: 55.

TABLE 12
Exemplary wild-type AAV capsid protein sequences

| | | SEQ |
|---|---|---|
| Name | DNA Sequence | ID NO: |
| AAV9 VP1 | MAADGYLPDWLEDNLSEGIREWWALKPGAPQPKANQQH | 46 |
| | QDNARGLVLPGYKYLGPGNGLDKGEPVNAADAAALEHD | |
| | KAYDQQLKAGDNPYLKYNHADAEFQERLKEDTSFGGNL | |
| | GRAVFQAKKRLLEPLGLVEEAAKTAPGKKRPVEQSPQEPD | |
| | SSAGIGKSGAQPAKKRLNFGQTGDTESVPDPQPIGEPPAAP | |
| | SGVGSLTMASGGGAPVADNNEGADGVGSSSGNWHCDSQ | |
| | WLGDRVITTSTRTWALPTYNNHLYKQISNSTSGGSSNDNA | |
| | YFGYSTPWGYFDFNRFHCHFSPRDWQRLINNNWGFRPKR | |
| | LNFKLFNIQVKEVTDNNGVKTIANNLTSTVQVFTDSDYQL | |
| | PYVLGSAHEGCLPPFPADVFMIPQYGYLTLNDGSQAVGRS | |
| | SFYCLEYFPSQMLRTGNNFQFSYEFENVPFHSSYAHSQSLD | |
| | RLMNPLIDQYLYYLSKTINGSGQNQQTLKFSVAGPSNMAV | |
| | QGRNYIPGPSYRQQRVSTTVTQNNNSEFAWPGASSWALN | |
| | GRNSLMNPGPAMASHKEGEDRFFPLSGSLIFGKQGTGRDN | |
| | VDADKVMITNEEEIKTTNPVATESYGQVATNHQSAQAQA | |
| | QTGWVQNQGILPGMVWQDRDVYLQGPIWAKIPHTDGNF | |
| | HPSPLMGGFGMKHPPPQILIKNTPVPADPPTAFNKDKLNSF | |
| | ITQYSTGQVSVEIEWELQKENSKRWNPEIQYTSNYYKSNN | |
| | VEFAVNTEGVYSEPRPIGTRYLTRNL | |
| AAV9 VP2 | TAPGKKRPVEQSPQEPDSSAGIGKSGAQPAKKRLNFGQTG | 47 |
| | DTESVPDPQPIGEPPAAPSGVGSLTMASGGGAPVADNNEG | |
| | ADGVGSSSGNWHCDSQWLGDRVITTSTRTWALPTYNNHL | |
| | YKQISNSTSGGSSNDNAYFGYSTPWGYFDFNRFHCHFSPR | |
| | DWQRLINNNWGFRPKRLNFKLFNIQVKEVTDNNGVKTIA | |
| | NNLTSTVQVFTDSDYQLPYVLGSAHEGCLPPFPADVFMIP | |
| | QYGYLTLNDGSQAVGRSSFYCLEYFPSQMLRTGNNFQFSY | |
| | EFENVPFHSSYAHSQSLDRLMNPLIDQYLYYLSKTINGSGQ | |
| | NQQTLKFSVAGPSNMAVQGRNYIPGPSYRQQRVSTTVTQ | |
| | NNNSEFAWPGASSWALNGRNSLMNPGPAMASHKEGEDR | |
| | FFPLSGSLIFGKQGTGRDNVDADKVMITNEEEIKTTNPVAT | |
| | ESYGQVATNHQSAQAQAQTGWVQNQGILPGMVWQDRD | |
| | VYLQGPIWAKIPHTDGNFHPSPLMGGFGMKHPPPQILIKNT | |
| | PVPADPPTAFNKDKLNSFITQYSTGQVSVEIEWELQKENSK | |
| | RWNPEIQYTSNYYKSNNVEFAVNTEGVYSEPRPIGTRYLT | |
| | RNL | |
| AAV9 VP3 | MASGGGAPVADNNEGADGVGSSSGNWHCDSQWLGDRVI | 48 |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSA | |
| | HEGCLPPFPADVFMIPQYGYLTLNDGSQAVGRSSFYCLEY | |
| | FPSQMLRTGNNFQFSYEFENVPFHSSYAHSQSLDRLMNPLI | |
| | DQYLYYLSKTINGSGQNQQTLKFSVAGPSNMAVQGRNYI | |
| | PGPSYRQQRVSTTVTQNNNSEFAWPGASSWALNGRNSLM | |
| | NPGPAMASHKEGEDRFFPLSGSLIFGKQGTGRDNVDADK | |
| | VMITNEEEIKTTNPVATESYGQVATNHQSAQAQAQTGWV | |
| | QNQGILPGMVWQDRDVYLQGPIWAKIPHTDGNFHPSPLM | |
| | GGFGMKHPPPQILIKNTPVPADPPTAFNKDKLNSFITQYST | |
| | GQVSVEIEWELQKENSKRWNPEIQYTSNYYKSNNVEFAV | |
| | NTEGVYSEPRPIGTRYLTRNL | |
| AAV9 VR-IV | NGSGQNQ | 49 |
| AAV9 VR-V | NNSEFA | 50 |
| AAV9 VR-VII | GRDNV | 51 |
| AAV9 VR-VIII | ATNHQSAQAQAQTG | 52 |
| AAV5 VP1 | MSFVDHPPDWLEEVGEGLREFLGLEAGPPKPKPNQQHQD | 53 |
| | QARGLVLPGYNYLGPGNGLDRGEPVNRADEVAREHDISY | |
| | NEQLEAGDNPYLKYNHADAEFQEKLADDTSFGGNLGKA | |
| | VFQAKKRVLEPFGLVEEGAKTAPTGKRIDDHFPKRKKART | |
| | EEDSKPSTSSDAEAGPSGSQQLQIPAQPASSLGADTMSAGG | |
| | GGPLGDNNQGADGVGNASGDWHCDSTWMGDRVVTKST | |
| | RTWVLPSYNNHQYREIKSGSVDGSNANAYFGYSTPWGYF | |
| | DFNRFHSHWSPRDWQRLINNYWGFRPRSLRVKIFNIQVKE | |
| | VTVQDSTTTIANNLTSTVQVFTDDDYQLPYVVGNGTEGCL | |
| | PAFPPQVFTLPQYGYATLNRDNTENPTERSSFFCLEYFPSK | |
| | MLRTGNNFEFTYNFEEVPFHSSFAPSQNLFKLANPLVDQY | |
| | LYRFVSTNNTGGVQFNKNLAGRYANTYKNWFPGPMGRT | |
| | QGWNLGSGVNRASVSAFATTNRMELEGASYQVPPQPNG | |
| | MTNNLQGSNTYALENTMIFNSQPANPGTTATYLEGNMLIT | |
| | SESETQPVNRVAYNVGGQMATNNQSSTTAPATGTYNLQE | |
| | IVPGSVWMERDVYLQGPIWAKIPETGAHFHPSPAMGGFGL | |
| | KHPPPMMLIKNTPVPGNITSFSDVPVSSFITQYSTGQVTVE | |
| | MEWELKKENSKRWNPEIQYTNNYNDPQFVDFAPDSTGEY | |
| | RTTRPIGTRYLTRPL | |
| AAV5 VP2 | TAPTGKRIDDHFPKRKKARTEEDSKPSTSSDAEAGPSGSQQ | 54 |
| | LQIPAQPASSLGADTMSAGGGGPLGDNNQGADGVGNASG | |
| | DWHCDSTWMGDRVVTKSTRTWVLPSYNNHQYREIKSGS | |
| | VDGSNANAYFGYSTPWGYFDFNRFHSHWSPRDWQRLINN | |
| | YWGFRPRSLRVKIFNIQVKEVTVQDSTTTIANNLTSTVQVF | |
| | TDDDYQLPYVVGNGTEGCLPAFPPQVFTLPQYGYATLNR | |
| | DNTENPTERSSFFCLEYFPSKMLRTGNNFEFTYNFEEVPFH | |
| | SSFAPSQNLFKLANPLVDQYLYRFVSTNNTGGVQFNKNLA | |
| | GRYANTYKNWFPGPMGRTQGWNLGSGVNRASVSAFATT | |
| | NRMELEGASYQVPPQPNGMTNNLQGSNTYALENTMIFNS | |
| | QPANPGTTATYLEGNMLITSESETQPVNRVAYNVGGQMA | |
| | TNNQSSTTAPATGTYNLQEIVPGSVWMERDVYLQGPIWA | |
| | KIPETGAHFHPSPAMGGFGLKHPPPMMLIKNTPVPGNITSF | |
| | SDVPVSSFITQYSTGQVTVEMEWELKKENSKRWNPEIQYT | |
| | NNYNDPQFVDFAPDSTGEYRTTRPIGTRYLTRPL | |
| AAV5 VP3 | MSAGGGGPLGDNNQGADGVGNASGDWHCDSTWMGDR | 55 |
| | VVTKSTRTWVLPSYNNHQYREIKSGSVDGSNANAYFGYS | |
| | TPWGYFDFNRFHSHWSPRDWQRLINNYWGFRPRSLRVKI | |
| | FNIQVKEVTVQDSTTTIANNLTSTVQVFTDDDYQLPYVVG | |
| | NGTEGCLPAFPPQVFTLPQYGYATLNRDNTENPTERSSFFC | |
| | LEYFPSKMLRTGNNFEFTYNFEEVPFHSSFAPSQNLFKLAN | |
| | PLVDQYLYRFVSTNNTGGVQFNKNLAGRYANTYKNWFP | |
| | GPMGRTQGWNLGSGVNRASVSAFATTNRMELEGASYQV | |
| | PPQPNGMTNNLQGSNTYALENTMIFNSQPANPGTTATYLE | |
| | GNMLITSESETQPVNRVAYNVGGQMATNNQSSTTAPATG | |
| | TYNLQEIVPGSVWMERDVYLQGPIWAKIPETGAHFHPSPA | |
| | MGGFGLKHPPPMMLIKNTPVPGNITSFSDVPVSSFITQYST | |
| | GQVTVEMEWELKKENSKRWNPEIQYTNNYNDPQFVDFAP | |
| | DSTGEYRTTRPIGTRYLTRPL | |

In some embodiments, the rAAV virions of the disclosure comprise an engineered capsid protein. Engineered capsid proteins can be derived from a parental, e.g., wild-type, capsid and include, for example, variant polypeptide sequence with respect to a parental capsid sequence at one or more sites. For example, variant sites of the parental capsid can occur at the VR-IV site, VR-V site, VR-VIJ site and/or VR-VIII site (see, e.g., Büning and Srivastava. Mol Ther Methods Clin Dev. 12:248-265 (2019)).

In some embodiments, the variant has a substitution or insertion in the VR-IV region of the capsid protein (e.g., of AAV9). In some embodiments, the variant has a substitution or insertion in the VR-V region of the capsid protein (e.g., of AAV9). In some embodiments, the variant has a substitution or insertion in the VR-VII region of the capsid protein (e.g., of AAV9). In some embodiments, the variant has a substitution or insertion in the VR-VIII region of the capsid protein (e.g., of AAV9). In some embodiments, the variant has a substitution or insertion in the VR-IV region and the VR-VIII region of the capsid protein (e.g., of AAV9).

In some embodiments, the capsid protein is an AAV5/AAV9 chimeric capsid protein. In some embodiments, the chimeric capsid protein comprises at least 1, 2, 3, 4, 5 or more polypeptide segments that are derived from AAV5 capsid protein. In some embodiments, the chimeric capsid protein comprises at least 1, 2, 3, 4, 5 or more polypeptide segments that are derived from AAV9 capsid protein. In some embodiments, at least one polypeptide segment is derived from the AAV5 capsid protein and at least one polypeptide segment is derived from the AAV9 capsid protein.

In some embodiments, the capsid protein is a combinatory capsid protein. As used herein, “combinatory capsid protein” refers to a AAV5/AAV9 chimeric capsid protein, which further comprises amino acid variations with respect to the chimeric parental sequence at one or more sites. In some embodiments, the one or more sites of the chimeric parental sequence are selected from those equivalent to the VR-IV site, the VR-V site, the VR-VII site and the VR-VIII site of the AAV9 capsid protein.

In some embodiments, the rAAV virions described herein comprise any capsid protein or variant capsid protein, e.g., any AAV9 variant capsid protein (e.g., comprising one or more substitutions or insertions), described in WO2021/163357A2 and/or U.S. Pat. No. 11,129,908, both of which are incorporated by reference herein in their entirety. In some embodiments, the present disclosure provides a rAAV capsid protein disclosed in WO2021/163357A2 as CR9-01, and this disclosure is specifically incorporated by reference herein in its entirety.

In some embodiments, the rAAV virions described herein comprise any capsid protein or variant capsid protein, e.g., any AAV9 variant capsid protein (e.g., comprising one or more substitutions or insertions), described in WO 2021/216456, which is incorporated by reference herein in its entirety.

In some embodiments, the capsid protein comprises one, two, three, four or more substitutions in the VR-VIII site. In some embodiments, the capsid protein comprises one, two, three, four or more insertions in the VR-VIII site. In some embodiments, the capsid protein comprises, relative to reference SEQ ID NO: 46, one, two, three, four or more substitutions at positions from 584 to 590 in the VR-VIII site, or one, two, three, four or more substitutions at positions from 585 to 590 in the VR-VIII site. In some embodiments, the capsid protein comprises, relative to reference SEQ ID NO: 46, one, two, three, four or more insertions at positions from 584 to 590 in the VR-VIII site, or one, two, three, four or more insertions at positions from 585 to 590 in the VR-VIII site.

In some embodiments, the capsid protein comprises at least two, three, four, five or more substitutions in the VR-VIII site. In some embodiments, the capsid protein comprises at least two, three, four or more insertions in the VR-VIII site. In some embodiments, the capsid protein comprises, relative to reference SEQ ID NO: 46, at least two, three, four, five or more substitutions at positions from 584 to 590 in the VR-VIII site, or at least two, three, four, five or more substitutions at positions from 585 to 590 in the VR-VIII site. In some embodiments, the capsid protein comprises, relative to reference SEQ ID NO: 46, at least two, three, four or more insertions at positions from 584 to 590 in the VR-VIII site, or at least two, three, four or more insertions at positions from 585 to 590 in the VR-VIII site.

In some embodiments, the capsid protein: (i) is cardiotrophic, (ii) exhibits increased transduction efficiency in cardiac cells compared to the parental sequence, (iii) exhibits decreased transduction efficiency in liver cells compared to the parental sequence, and/or (iv) exhibits increased selectivity for the cardiac cells over liver cells compared to the parental sequence. These characteristics may be assessed in cells (e.g., iPSC-derived cardiac cells or cardiomyocytes) in vitro, or in mice or primates in vivo, by any methods known in the art.

In some embodiments, the capsid protein may comprise an amino acid insertion at position 584 (relative to reference sequence SEQ ID NO: 46) comprising one or more of an asparagine (N), a threonine (T), a tyrosine (Y), phenylalanine (F), and an alanine (A).

In some embodiments, the capsid protein may comprise an amino acid insertion at position 585 (relative to reference sequence SEQ ID NO: 46) comprising one or more of a histidine (H) and a methionine (M).

In some embodiments, the capsid protein may comprise an amino acid insertion at position 586 (relative to reference sequence SEQ ID NO: 46) comprising one or more of a histidine (H), a tyrosine (Y), a valine (V), a threonine (T), an alanine (A), an isoleucine (I), a tryptophan (W), a methionine (M), and a leucine (L).

In some embodiments, the capsid protein may comprise an amino acid insertion at position 587 (relative to reference sequence SEQ ID NO: 46) comprising one or more of an isoleucine (I) and a proline (P).

In some embodiments, the capsid protein may comprise an amino acid insertion at position 588 (relative to reference sequence SEQ ID NO: 46) comprising one or more of an isoleucine (I), a threonine (T), and a proline (P).

In some embodiments, the capsid protein may comprise one or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 46).

In some embodiments, the capsid protein may comprise one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D (relative to reference sequence SEQ ID NO: 46).

In some embodiments, a recombinant adeno-associated virus (rAAV) capsid protein shares, or comprises a sequence sharing, at least 80% or 85% amino acid sequence identity to
an AAV9 VP3 reference sequence according to SEQ ID NO: 48, and wherein the capsid protein comprises, relative to reference sequence SEQ ID NO: 46:
an amino acid insertion between position 583 and 584 comprising one or more of an asparagine (N), a threonine (T), a tyrosine (Y), phenylalanine (F), and an alanine (A);
an amino acid insertion between position 584 and 585 comprising one or more of a histidine (H) and a methionine (M);
an amino acid insertion between position 585 and 586 comprising one or more of a histidine (H), a tyrosine (Y), a valine (V), a threonine (T), an alanine (A), an isoleucine (I), a tryptophan (W), a methionine (M), and a leucine (L);
an amino acid insertion between position 586 and 587 comprising one or more of an isoleucine (I) and a proline (P);
an amino acid insertion between position 587 and 588 comprising one or more of an isoleucine (I), a threonine (T), and a proline (P);
an amino acid insertion between position 588 and 589 comprising one or more of a glycine (G) and a glutamine (Q);
one or more amino acid substitutions selected from the group consisting of N452K, N452A, N452V, N452I, G453A, G453N, S454T, S454D, G455N, Q456L, Q456K, N457L, N457V, Q458I, and Q458H; and/or
one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

In some embodiments, the capsid protein may comprise an amino acid insertion at position 584 (relative to reference sequence SEQ ID NO: 46) consisting of a TY, FN, or AT.

In some embodiments, the capsid protein may comprise an amino acid insertion at position 585 (relative to reference sequence SEQ ID NO: 46) consisting of MH.

In some embodiments, the capsid protein may comprise an amino acid insertion at position 586 (relative to reference sequence SEQ ID NO: 46) consisting of HY, VT, AI, WM, or ML.

In some embodiments, the capsid protein may comprise an amino acid insertion at position 587 (relative to reference sequence SEQ ID NO: 46) consisting of PI.

In some embodiments, the capsid protein may comprise an amino acid insertion at position 588 (relative to reference sequence SEQ ID NO: 46) consisting of IT or PT.

In some embodiments, the capsid protein may comprise one or more amino acid substitutions selected from the group consisting of T582D, T582E, N583V, H584Q, S586K, A587P, A587S, Q588G, Q588M, A589S, A591I, G594Q, and G594D (relative to reference sequence SEQ ID NO: 46).

In some embodiments, the capsid protein may comprise one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R (relative to reference sequence SEQ ID NO: 46).

In some embodiments, the capsid protein may comprise one or more amino acid substitutions selected from the group consisting of Q585C, Q585S, and S586I (relative to reference sequence SEQ ID NO: 46).

In some embodiments, the capsid protein may comprise one or more amino acid substitutions selected from the group consisting of Q585C, Q585S, S586I, A587V and A587G (relative to reference sequence SEQ ID NO: 46).

In some embodiments, the capsid protein may comprise one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L (relative to reference sequence SEQ ID NO: 46).

In some embodiments, the capsid protein may comprise one or more amino acid substitutions selected from the group consisting of A587V and A587G (relative to reference sequence SEQ ID NO: 46).

In some embodiments, the capsid protein may comprise two or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 46).

In some embodiments, the capsid protein may comprise the amino acid substitution N452K, N452A, or N452V (relative to reference sequence SEQ ID NO: 46).

In some embodiments, the capsid protein may comprise the amino acid substitution N452K (relative to reference sequence SEQ ID NO: 46).

In some embodiments, the capsid protein may comprise the amino acid substitution G453A or G453N (relative to reference sequence SEQ ID NO: 46).

In some embodiments, the capsid protein may comprise the amino acid substitution S454T or S454D (relative to reference sequence SEQ ID NO: 46).

In some embodiments, the capsid protein may comprise the amino acid substitution G455N (relative to reference sequence SEQ ID NO: 46).

In some embodiments, the capsid protein may comprise the amino acid substitution Q456L or Q456K (relative to reference sequence SEQ ID NO: 46).

In some embodiments, the capsid protein may comprise the amino acid substitution N457L or N457V (relative to reference sequence SEQ ID NO: 46).

In some embodiments, the capsid protein may comprise the amino acid substitution Q458I or Q458H (relative to reference sequence SEQ ID NO: 46).

In some embodiments, the capsid protein comprises relative to reference sequence SEQ ID NO: 46, at position 452 an amino acid selected from the group consisting of: K and N. In some embodiments, the capsid protein comprises, relative to reference sequence SEQ ID NO: 46, an amino acid substitution N452K.

In some embodiments, a recombinant adeno-associated virus (rAAV) capsid protein shares, or comprises a sequence sharing, at least 80% or 85% amino acid sequence identity to an AAV9 VP3 reference sequence according to SEQ ID NO: 48, and wherein the capsid protein comprises, relative to reference sequence SEQ ID NO: 46, amino acid substitution N452K. In some embodiments, N452K is the only substitution in the capsid protein relative to the parental or wild-type AAV9. In some embodiments, N452K is not the only substitution in the capsid protein relative to the parental or wild-type AAV9.

In some embodiments, the capsid protein comprises, relative to reference sequence SEQ ID NO: 46:
at position 585 an amino acid selected from: E, N, G, M, C, V, T and Q;
at position 586 an amino acid selected from: N, T, M, G, D, and S;
at position 587 an amino acid selected from: T, L, I, K, S, N, V and A;
at position 588 an amino acid selected from: V, F, Y, L, T, S, I, R and Q;
at position 589 an amino acid selected from: S, N, L, T, I, R and A; and/or
at position 590 an amino acid selected from: I, S, G, H, R and Q.

In some embodiments, the capsid protein comprises, relative to reference sequence SEQ ID NO: 46:
at position 585 an amino acid selected from: E, N, G, M, C, V, T and Q;
at position 586 an amino acid selected from: N, T, M, G, D, and S;
at position 587 an amino acid selected from: T, L, I, K, S, N, V and A;
at position 588 an amino acid selected from: V, F, Y, L, T, S, I, R and Q;
at position 589 an amino acid selected from: S, N, L, T, I, R and A; and
at position 590 an amino acid selected from: I, S, G, H, R and Q.

In some embodiments, the capsid protein comprises, relative to reference sequence SEQ ID NO: 46:
at position 585 an amino acid selected from: E, N, G, M, C, V and T;
at position 586 an amino acid selected from: N, T, M, G, and D;
at position 587 an amino acid selected from: T, L, I, K, S, N and V;
at position 588 an amino acid selected from: V, F, Y, L, T, S, I and R;
at position 589 an amino acid selected from: S, N, L, T, I and R; and/or
at position 590 an amino acid selected from: I, S, G, H and R.

In some embodiments, the capsid protein comprises, relative to reference sequence SEQ ID NO: 46:
at position 585 an amino acid selected from: E, N, G, M, C, V and T;
at position 586 an amino acid selected from: N, T, M, G, and D;
at position 587 an amino acid selected from: T, L, I, K, S, N and V;
at position 588 an amino acid selected from: V, F, Y, L, T, S, I and R;
at position 589 an amino acid selected from: S, N, L, T, I and R; and
at position 590 an amino acid selected from: I, S, G, H and R.

In some embodiments, the capsid protein comprises, relative to reference sequence SEQ ID NO: 46:
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and/or
at position 590 an amino acid selected from the group consisting of: G, R, S, I, H, N, Y, L, M and Q; and
optionally at position 452 an amino acid selected from the group consisting of: N and K.

In some embodiments, the capsid protein comprises, relative to reference sequence SEQ ID NO: 46:
at position 452 an amino acid selected from the group consisting of: K and N;
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and
at position 590 an amino acid selected from the group consisting of: G, R, S, I, H, N, Y, L, M and Q.

In some embodiments, the capsid protein comprises, relative to reference sequence SEQ ID NO: 46:
at position 584 amino acid R;
at position 585 an amino acid selected from the group consisting of: N, M, C, E, G, S, V, A, T, H and, L;
at position 586 an amino acid selected from the group consisting of: M, D, N, G, A, T, R, and I;
at position 587 an amino acid selected from the group consisting of: T, N, V, L, I, S, R, and P;
at position 588 an amino acid selected from the group consisting of: Y, T, S, I, V, F, L, R, N, D, and G;
at position 589 an amino acid selected from the group consisting of: L, I, R, S, G, N, T, V, Q, F, E, and Y; and/or
at position 590 an amino acid selected from the group consisting of: G, R, S, I, H, N, Y, L, and M; and
optionally at position 452 an amino acid selected from the group consisting of: N and K.

In some embodiments, the capsid protein comprises, relative to reference sequence SEQ ID NO: 46, at least two, three, four, five, six, seven or all eight of any of the following:
(i) at position 452 amino acid K;
(ii) at position 584 amino acid R;
(iii) at position 585 an amino acid selected from the group consisting of: N, M, C, E, G, S, V, A, T, H, and L;
(iv) at position 586 an amino acid selected from the group consisting of: M, D, N, G, A, T, R, and I;
(v) at position 587 an amino acid selected from the group consisting of: T, N, V, L, I, S, R, and P;
(vi) at position 588 an amino acid selected from the group consisting of: Y, T, S, I, V, F, L, R, N, D, and G;
(vii) at position 589 an amino acid selected from the group consisting of: L, I, R, S, G, N, T, V, Q, F, E, and Y; and
(viii) at position 590 an amino acid selected from the group consisting of: G, R, S, I, H, N, Y, L, and M.

In some embodiments, the capsid protein comprises, relative to reference sequence SEQ ID NO: 46:
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and/or
at position 590 an amino acid selected from the group consisting of: I, S, G, H, R and Q; and optionally at position 452 an amino acid selected from the group consisting of: N and K.

In some embodiments, the capsid protein comprises, relative to reference sequence SEQ ID NO: 46:
at position 452 an amino acid selected from the group consisting of: K and N;
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and
at position 590 an amino acid selected from the group consisting of: I, S, G, H, R and Q.

In some embodiments, the capsid protein comprises, relative to reference sequence SEQ ID NO: 46:
at position 585 an amino acid selected from the group consisting of: E, N, G, M, C, V and T;
at position 586 an amino acid selected from the group consisting of: N, T, M, G, and D;
at position 587 an amino acid selected from the group consisting of: T, L, I, K, S, N and V;
at position 588 an amino acid selected from the group consisting of: V, F, Y, L, T, S, I and R;
at position 589 an amino acid selected from the group consisting of: S, N, L, T, I and R; and/or
at position 590 an amino acid selected from the group consisting of: I, S, G, H and R; and optionally at position 452 an amino acid selected from the group consisting of: N and K.

In some embodiments, the capsid protein comprises, relative to reference sequence SEQ ID NO: 46, at least two, three, four, five, six or all seven of any of the following:
(i) at position 452 amino acid K;
(ii) at position 585 an amino acid selected from the group consisting of: E, N, G, M, C, V and T;
(iii) at position 586 an amino acid selected from the group consisting of: N, T, M, G, and D;
(iv) at position 587 an amino acid selected from the group consisting of: T, L, I, K, S, N and V;
(v) at position 588 an amino acid selected from the group consisting of: V, F, Y, L, T, S, I and R;
(vi) at position 589 an amino acid selected from the group consisting of: S, N, L, T, I and R; and
(vii) at position 590 an amino acid selected from the group consisting of: I, S, G, H and R.

In some embodiments, the capsid protein comprises, relative to reference sequence SEQ ID NO: 46:
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and/or
at position 590 an amino acid selected from the group consisting of: I, S, G, R and Q; and
optionally at position 452 an amino acid selected from the group consisting of: N and K.

In some embodiments, the capsid protein comprises, relative to reference sequence SEQ ID NO: 46:
at position 452 an amino acid selected from the group consisting of: K and N;
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and
at position 590 an amino acid selected from the group consisting of: I, S, G, R and Q.

In some embodiments, the capsid protein comprises, relative to reference sequence SEQ ID NO: 46:
at position 585 an amino acid selected from the group consisting of: E, N, M, and C;
at position 586 an amino acid selected from the group consisting of: A, M, G, D, and N;
at position 587 an amino acid selected from the group consisting of: T, N, and V;
at position 588 an amino acid selected from the group consisting of: V, Y, T, S, and I;
at position 589 an amino acid selected from the group consisting of: S, G, L, I and R; and/or at position 590 an amino acid selected from the group consisting of: I, S, G, and R; and
optionally at position 452 an amino acid selected from the group consisting of: N and K.

In some embodiments, the capsid protein comprises, relative to reference sequence SEQ ID NO: 46, at least two, three, four, five, six or all seven of any of the following:
(i) at position 452 amino acid K;
(ii) at position 585 an amino acid selected from the group consisting of: E, N, M, and C;
(iii) at position 586 an amino acid selected from the group consisting of: A, M, G, D, and N;
(iv) at position 587 an amino acid selected from the group consisting of: T, N, and V; (v) at position 588 an amino acid selected from the group consisting of: V, Y, T, S, and I;
(vi) at position 589 an amino acid selected from the group consisting of: S, G, L, I and R; and
(vii) at position 590 an amino acid selected from the group consisting of: I, S, G, and R.

In some embodiments, the capsid protein comprises, relative to reference sequence SEQ ID NO: 46:
at position 452 an amino acid selected from the group consisting of: K and N; and
at position 587 amino acid substitution A587T.

In some embodiments, the capsid protein comprises, relative to reference sequence SEQ ID NO: 46:
at position 452 an amino acid selected from the group consisting of: K and N; and amino acid N or R at one, two or more positions selected from the group consisting of: 584, 585, 586, 588, 589, and 590.

In some embodiments, the capsid protein comprises, relative to reference sequence SEQ ID NO: 46:
at position 452 an amino acid selected from the group consisting of: K and N; and amino acid S at two or more positions selected from the group consisting of: 585, 586, 587, 588, 589 and 590.

In some embodiments, the capsid protein comprises, relative to reference sequence SEQ ID NO: 46:
at position 452 an amino acid selected from the group consisting of: K and N; and at three, four or more positions in the region 585-590 of the VR-VIII site, an amino acid selected from the group consisting of: N, S, T, R and I.

In some embodiments, the capsid protein comprises, relative to reference sequence SEQ ID NO: 46:
at three, four or more positions in the region 585-590 of the VR-VIII site, an amino acid selected from the group consisting of: N, S, T, and R.

In some embodiments, the capsid protein comprises, relative to reference sequence SEQ ID NO: 46:
at position 452 an amino acid selected from the group consisting of: K and N; and at three, four or more positions in the region 585-590 of the VR-VIII site, amino acids selected from the group consisting of: N, S, T, R and I (such as any combination and number of each of these amino acids).

In some embodiments, the capsid protein comprises, relative to reference sequence SEQ ID NO: 46:
at three, four or more positions in the region 585-590 of the VR-VIII site, amino acids selected from the group consisting of: N, S, T, and R (such as any combination and number of each of these amino acids).

In some embodiments, the capsid protein comprises, relative to reference sequence SEQ ID NO: 46:
at position 452 an amino acid selected from the group consisting of: K and N; and at four, five or more positions in the region 585-590 of the VR-VIII site, amino acids selected from the group consisting of: N, S, T, R and I (such as any combination and number of each of these amino acids).

In some embodiments, the capsid protein comprises, relative to reference sequence SEQ ID NO: 46:
at four, five or more positions in the region 585-590 of the VR-VIII site, amino acids selected from the group consisting of: N, S, T, and R (such as any combination and number of each of these amino acids).

In some embodiments, the capsid protein comprises, relative to reference sequence SEQ ID NO: 46, at least two, three, four or more of the amino acid substitutions Q585E, S586N, A587T, Q588V, A589S, Q590I, and/or N452K (or any combination of these substitutions). In some embodiments, the capsid protein comprises, relative to reference sequence SEQ ID NO: 46, amino acid substitutions Q585E, S586N, A587T, Q588V, A589S, Q590I, and N452K.

In some embodiments, the capsid protein comprises, relative to reference sequence SEQ ID NO: 46, at least two, three, four or more of the amino acid substitutions S586T, A587L, Q588F, A589N, Q590S, and/or N452K (or any combination of these substitutions). In some embodiments, the capsid protein comprises, relative to reference sequence SEQ ID NO: 46, amino acid substitutions S586T, A587L, Q588F, A589N, Q590S, and N452K.

In some embodiments, the capsid protein comprises, relative to reference sequence SEQ ID NO: 46, at least two, three, four or more of the amino acid substitutions Q585N, A587T, Q588Y, A589L, Q590G, and/or N452K (or any combination of these substitutions). In some embodiments, the capsid protein comprises, relative to reference sequence SEQ ID NO: 46, amino acid substitutions Q585N, A587T, Q588Y, A589L, Q590G, and N452K.

In some embodiments, the capsid protein comprises, relative to reference sequence SEQ ID NO: 46, at least two, three, four or more of the amino acid substitutions Q585G, A587I, Q588L, A589T, Q590H, and/or 452K (or any combination of these substitutions). In some embodiments, the capsid protein comprises, relative to reference sequence SEQ ID NO: 46, amino acid substitutions Q585G, A587I, Q588L, A589T, Q590H, and N452K.

In some embodiments, the capsid protein comprises, relative to reference sequence SEQ ID NO: 46, at least two, three, four or more of the amino acid substitutions Q585M, S586M, A587T, Q588T, and/or Q590R (or any combination of these substitutions). In some embodiments, the capsid protein comprises, relative to reference sequence SEQ ID NO: 46, amino acid substitutions Q585M, S586M, A587T, Q588T, and Q590R. In some embodiments, the capsid protein comprises, relative to reference sequence SEQ ID NO: 46, amino acid substitutions Q585M, S586M, A587T, Q588T, and Q590R; and amino acid N at position 452.

In some embodiments, the capsid protein comprises, relative to reference sequence SEQ ID NO: 46, at least two, three, four or more of the amino acid substitutions Q585N, A587T, Q588Y, A589L, and/or Q590G (or any combination of these substitutions). In some embodiments, the capsid protein comprises, relative to reference sequence SEQ ID NO: 46, amino acid substitutions Q585N, A587T, Q588Y, A589L, and Q590G. In some embodiments, the capsid protein comprises, relative to reference sequence SEQ ID NO: 46, amino acid substitutions Q585N, A587T, Q588Y, A589L, and Q590G; and amino acid N at position 452.

In some embodiments, the capsid protein comprises, relative to reference sequence SEQ ID NO: 46, at least two, three, four or more of the amino acid substitutions Q585C, A587T, Q588S, A589I, and/or Q590R (or any combination of these substitutions). In some embodiments, the capsid protein comprises, relative to reference sequence SEQ ID NO: 46, amino acid substitutions Q585C, A587T, Q588S, A589I, and Q590R. In some embodiments, the capsid protein comprises, relative to reference sequence SEQ ID NO: 46, amino acid substitutions Q585C, A587T, Q588S, A589I, and Q590R; and amino acid N at position 452.

In some embodiments, the capsid protein comprises, relative to reference sequence SEQ ID NO: 46, at least two, three, four or more of the amino acid substitutions Q585E, S586D, A587N, Q588I, A589R, and/or Q590S (or any combination of these substitutions). In some embodiments, the capsid protein comprises, relative to reference sequence SEQ ID NO: 46, amino acid substitutions Q585E, S586D, A587N, Q588I, A589R, and Q590S. In some embodiments, the capsid protein comprises, relative to reference sequence SEQ ID NO: 46, amino acid substitutions Q585E, S586D, A587N, Q588I, A589R, and Q590S; and amino acid N at position 452.

In some embodiments, the capsid protein comprises, relative to reference sequence SEQ ID NO: 46, at least two, three, four or more of the amino acid substitutions Q585E, S586D, A587N, Q588I, A589R, Q590S, and/or N452K (or any combination of these substitutions). In some embodiments, the capsid protein comprises, relative to reference sequence SEQ ID NO: 46, amino acid substitutions Q585E, S586D, A587N, Q588I, A589R, Q590S, and N452K.

In some embodiments, the capsid protein comprises, relative to reference sequence SEQ ID NO: 46, amino acid S586G and/or Q588Y. In some embodiments, the capsid protein comprises, relative to reference sequence SEQ ID NO: 46, amino acid substitutions S586G and Q588Y; and amino acid N at position 452.

In some embodiments, the capsid protein comprises, relative to reference sequence SEQ ID NO: 46, at least two, three, four or more of the amino acid substitutions S586A, A587N, Q588Y, A589G, and/or N452K (or any combination of these substitutions). In some embodiments, the capsid protein comprises, relative to reference sequence SEQ ID NO: 46, amino acid substitutions S586A, A587N, Q588Y, A589G, and N452K.

In some embodiments, the capsid protein comprises an amino acid sequence of X₁DVQX₂X₃PGFX₄X₅X₆X₇X₈ (SEQ ID NO: 56) at the VR-VIII site (e.g., of an AAV9 capsid protein or a variant thereof), wherein each of X₁, X₂, X₃, X₄, X₅, X₆, X₇, and X₈ is any amino acid.

In some embodiments, X₁ is alanine (A).

In some embodiments, X₂ is glutamine (Q).

In some embodiments, X₇ is threonine (T).

In some embodiments, X₅ is alanine (A) or proline (P).

In some embodiments, X₆ is glutamine (Q) or glutamic acid (E).

In some embodiments, X₄ is glutamine (Q), glycine (G), arginine (R), asparagine (N), histidine (H), methionine (M), proline (P), or serine (S).

In some embodiments, X₅ is glutamic acid (E), methionine (M), glutamine (Q), aspartic acid (D), leucine (L), alanine (A), cysteine (C), histidine (H), phenylalanine (F), tyrosine (Y), threonine (T), valine (V), isoleucine (I), serine (S), or asparagine (N). In some embodiments, X₅ is glutamic acid (E).

In some embodiments, X₃ is leucine (L), histidine (H), valine (V), cysteine (C), glutamine (Q), glycine (G), isoleucine (I), methionine (M), phenylalanine (F), proline (P), threonine (T), or tyrosine (Y).

In some embodiments, X₁ is A, X₂ is Q, X₇ is T, and/or the capsid protein comprises in the VR-VIII site an amino acid sequence of ADVQQX₃PGFX₄X₅X₆TX₈ (SEQ ID NO: 57), wherein each of X₃, X₄, X₅, X₆, and X₈ is any amino acid.

In some embodiments, X₅ is A or P, and X₆ is Q or E, and/or the capsid protein comprises in the VR-VIII site an amino acid sequence of X₁DVQX₂X₃PGFX₄AQX₇X₈ (SEQ ID NO: 58), X₁DVQX₂X₃PGFX₄AEX₇X₈ (SEQ ID NO: 59), X₁DVQX₂X₃PGFX₄PQX₇X₈ (SEQ ID NO: 60), X₁DVQX₂X₃PGFX₄PEX₇X₈ (SEQ ID NO: 61), ADVQQX₃PGFX₄AQTX₈ (SEQ ID NO: 62), ADVQQX₃PGFX₄AETX₈ (SEQ ID NO: 63), ADVQQX₃PGFX₄PQTX₈ (SEQ ID NO: 64), or ADVQQX₃PGFX₄PETX₈ (SEQ ID NO: 65), wherein each of X₁, X₂, X₃, X₄, X₇, and X₈ is any amino acid.

In some embodiments, X₄ is Q, X₅ is A, and X₆ is Q, and/or the capsid protein comprises in the VR-VIII site an amino acid sequence of X₁DVQX₂X₃PGFQAQX₇X₈ (SEQ ID NO: 66) or ADVQQX₃PGFQAQTX₈ (SEQ ID NO: 67), wherein each of X₁, X₂, X₃, X₇, and X₈ is any amino acid.

In some embodiments, X₃ is L, and X₈ is E, and/or the capsid protein comprises in the VR-VIII site an amino acid sequence of X₁DVQX₂LPGFX₄X₅X₆X₇E (SEQ ID NO: 68) or ADVQQLPGFX₄X₅X₆TE (SEQ ID NO: 69), wherein each of X₁, X₂, X₄, X₅, X₆, and X₇ is any amino acid.

In some embodiments, X₄ is Q, and/or the capsid protein comprises in the VR-VIII site an amino acid sequence of X₁DVQX₂X₃PGFQX₅X₆X₇X₈ (SEQ ID NO: 70) or ADVQQX₃PGFQX₅X₆TX₈ (SEQ ID NO: 71), wherein each of X₁, X₂, X₃, X₅, X₆, X₇, and X₈ is any amino acid.

In some embodiments, the capsid protein comprises, consists essentially of, or consists of a sequence having at least about 80% (e.g., at least 80%, 85%, 90%, 95%, 96%, 97%, 98%, 99%, 99.5%, or 100%) identity to any one of the following sequences at the VR-VIII site (positions 581-594 relative to reference sequence SEQ ID NO: 46), with up to 1, 2, or 3 substitutions: ADVQQLPGFQAQTE (SEQ ID NO: 72), ADVQQHPGFQAQTE (SEQ ID NO: 73), ADVQQVPGFQAQTM (SEQ ID NO: 74), ADVQQVPGFQAQTQ (SEQ ID NO: 75), ADVQQLPGFGAQTE (SEQ ID NO: 76), ADVQQLPGFRPETE (SEQ ID NO: 77), and ADVQQLPGFNAQTE (SEQ ID NO: 78).

In some embodiments, the capsid protein comprises any substitution and/or insertion motif described herein. In some embodiments, the capsid protein comprises a substitution motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any substitution motif described herein. In some embodiments, the capsid protein comprises an insertion motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any insertion motif described herein.

It should be noted that the above modified VR-VIII motifs are described in the context of AAV9 capsid proteins for illustrative purposes only and are not meant to be limited to AAV9 capsid proteins. Instead, any modified VR-VIII motif described herein can be applied to other AAV capsid proteins of a different serotype (e.g., AAV5, AAVrh.10, or AAVrh.74), for example, by replacing the wild-type sequence at the VR-VIII site of the corresponding capsid protein (e.g., amino acid positions 570 to 583 of wild-type AAV5 VP1 capsid protein sequence according to SEQ ID NO: 53, amino acid positions 583 to 596 of wild-type AAVrh.10 VP1 capsid protein sequence, or amino acid positions 583 to 596 of wild-type AAVrh.74 VP1 capsid protein sequence) with any of the modified VR-VIII motifs described herein to generate a variant of the capsid protein of a particular serotype. In some embodiments, the capsid protein is a variant of an AAV5, AAV9, AAVth.10, or AAVrh.74 capsid protein.

In some embodiments, the capsid protein comprises an insertion polypeptide or insertion motif compared to the wild-type or parental capsid protein. In some embodiments, the capsid protein additionally comprises one or more amino acid substitutions in the amino acid sequence of the wild-type or parental capsid protein sequence from which it is derived. In some embodiments, the insertion motif is inserted at a surface loop region of the capsid protein, for example, at a VR-IV, VR-V, VR-VII and/or VR-VIII site, as described.

In some embodiments, the insertion motif comprises or consists of an amino acid sequence of “RGDAARL” (SEQ ID NO: 79); and/or the capsid protein comprises an amino acid sequence of “RGDAARL” (SEQ ID NO: 79).

In some embodiments, the insertion motif comprises or consists of an amino acid sequence of “SHVRGDL” (SEQ ID NO: 80); and/or the capsid protein comprises an amino acid sequence of “SHVRGDL” (SEQ ID NO: 80).

In some embodiments, the insertion motif comprises or consists of an amino acid sequence of “VVSSGAR” (SEQ ID NO: 81); and/or the capsid protein comprises an amino acid sequence of “VVSSGAR” (SEQ ID NO: 81).

In some embodiments, the insertion motif comprises or consists of an amino acid sequence of “VRGD” (SEQ ID NO: 82); and/or the capsid protein comprises an amino acid sequence of “VRGD” (SEQ ID NO: 82).

The insertion motif can occur (e.g., be inserted) at any position of the capsid protein, for example, at a surface or an exposed region of the capsid protein. In some embodiments, the engineered AAV capsid protein comprises an insertion motif as described herein inserted at a surface loop region of the capsid protein, e.g., the VR-I, VR-II, VR-IV, VR-V, VR-VII, and/or VR-VIII site of the capsid protein. In some embodiments, the engineered AAV capsid protein comprises an insertion motif as described inserted at the VR-IV and/or the VR-VIII site of the capsid protein. In certain of these embodiments, the engineered capsid protein additionally comprises one or more amino acid substitutions in the same VR site as the insertion or at a different location from the insertion.

In some embodiments, the capsid protein is a variant AAV9 capsid protein that comprises an insertion polypeptide or insertion motif at the VR-VIII site, e.g., between amino acids 588 (glutamine (Q)) and 589 (alanine (A)) within the VR-VIII site in reference to the wild-type full-length AAV9 capsid protein of SEQ ID NO: 46. In some embodiments, the variant AAV9 capsid protein further comprises one or more amino acid substitutions within the VR-VIII site, including, for example, at one or more of amino acid positions 587-590 in reference to the wild-type full-length AAV9 capsid protein of SEQ ID NO: 46. In certain of these embodiments, the insertion motif comprises an amino acid sequence of RGDAARL (SEQ ID NO: 79), RTDLKGL (SEQ ID NO: 83), YPSTGSG (SEQ ID NO: 84), FAGSLTRA (SEQ ID NO: 85), DRTLTTR (SEQ ID NO: 86), RIAGRDV (SEQ ID NO: 87), or SLGSGVR (SEQ ID NO: 88).

In some embodiments, the capsid protein is an engineered AAV9 capsid protein that comprises an insertion polypeptide or insertion motif at the VR-IV site, e.g., between amino acids 453 (glycine (G)) and 454 (serine (S)), and/or between amino acids 456 (glutamine (Q)) and 457 (asparagine (N)), within the VR-IV site in reference to the wild-type full-length AAV9 capsid protein of SEQ ID NO: 46. In certain of these embodiments, the insertion motif comprises an amino acid sequence of SHVRGDL (SEQ ID NO: 80), VVSSGAR (SEQ ID NO: 81), PQYGRGG (SEQ ID NO: 89), LQVSRVS (SEQ ID NO: 90), VRSYSSN (SEQ ID NO: 91), “TMRVGSL” (SEQ ID NO: 92), GAYSRGV (SEQ ID NO: 93), LRGGSLG (SEQ ID NO: 94), or “VYGTGVR” (SEQ ID NO: 95).

In some embodiments, the capsid protein is an engineered AAV5 capsid protein that comprises an insertion polypeptide or insertion motif at the VR-VIII site, e.g., between amino acids 574 (glutamine (Q)) and 575 (serine (S)) within the VR-VIII site in reference to the wild-type full-length AAV5 capsid protein of SEQ ID NO: 53. In certain of these embodiments, the insertion motif comprises an amino acid sequence of DKLIIVS (SEQ ID NO: 96), AEDRTKL (SEQ ID NO: 97), LSASASL (SEQ ID NO: 98), LADQTKL (SEQ ID NO: 99), LLLKLQE (SEQ ID NO: 100), ELPVKTG (SEQ ID NO: 101), LDLKVVG (SEQ ID NO: 102), or RDAVL (SEQ ID NO: 103).

In some embodiments, the capsid protein comprises the amino acid sequence of SEQ ID NO: 104, or an amino acid sequence having at least 80%, 85%, 90%, 95% or 98% sequence identity to SEQ ID NO: 104. In some embodiments, the capsid protein is that of ZC621 described herein, or an amino acid sequence having at least 80%, 85%, 90%, 95% or 98% sequence identity to the capsid protein of ZC621 described herein.

In some embodiments, the capsid protein comprises the amino acid sequence of SEQ ID NO: 105, or an amino acid sequence having at least 80%, 85%, 90%, 95% or 98% sequence identity to SEQ ID NO: 105. In some embodiments, the capsid protein is that of ZC623 described herein, or an amino acid sequence having at least 80%, 85%, 90%, 95% or 98% sequence identity to the capsid protein of ZC623 described herein.

In some embodiments, the capsid protein comprises the amino acid sequence of SEQ ID NO: 106, or an amino acid sequence having at least 80%, 85%, 90%, 95% or 98% sequence identity to SEQ ID NO: 106. In some embodiments, the capsid protein is that of ZC625 described herein, or an amino acid sequence having at least 80%, 85%, 90%, 95% or 98% sequence identity to the capsid protein of ZC625 described herein.

In some embodiments, the capsid protein comprises the amino acid sequence of SEQ ID NO: 107, or an amino acid sequence having at least 80%, 85%, 90%, 95% or 98% sequence identity to SEQ ID NO: 107. In some embodiments, the capsid protein is that of ZC626 described herein, or an amino acid sequence having at least 80%, 85%, 90%, 95% or 98% sequence identity to the capsid protein of ZC626 described herein.

In some embodiments, the capsid protein comprises the amino acid sequence of SEQ ID NO: 108, or an amino acid sequence having at least 80%, 85%, 90%, 95% or 98% sequence identity to SEQ ID NO: 108. In some embodiments, the capsid protein is that of ZC630 described herein, or an amino acid sequence having at least 80%, 85%, 90%, 95% or 98% sequence identity to the capsid protein of ZC630 described herein.

In some embodiments, the capsid protein comprises the amino acid sequence of SEQ ID NO: 109, or an amino acid sequence having at least 80%, 85%, 90%, 95% or 98% sequence identity to SEQ ID NO: 109. In some embodiments, the capsid protein is that of ZC631 described herein, or an amino acid sequence having at least 80%, 85%, 90%, 95% or 98% sequence identity to the capsid protein of ZC631 described herein.

In some embodiments, the capsid protein comprises the amino acid sequence of SEQ ID NO: 110, or an amino acid sequence having at least 80%, 85%, 90%, 95% or 98% sequence identity to SEQ ID NO: 110. In some embodiments, the capsid protein is that of ZC632 described herein, or an amino acid sequence having at least 80%, 85%, 90%, 95% or 98% sequence identity to the capsid protein of ZC632 described herein.

In some embodiments, the capsid protein comprises the amino acid sequence of SEQ ID NO: 111, or an amino acid sequence having at least 80%, 85%, 90%, 95% or 98% sequence identity to SEQ ID NO: 111. In some embodiments, the capsid protein is that of ZC633 described herein, or an amino acid sequence having at least 80%, 85%, 90%, 95% or 98% sequence identity to the capsid protein of ZC633 described herein.

In some embodiments, the capsid protein comprises the amino acid sequence of SEQ ID NO: 112, or an amino acid sequence having at least 80%, 85%, 90%, 95% or 98% sequence identity to SEQ ID NO: 112. In some embodiments, the capsid protein is that of ZC634 described herein, or an amino acid sequence having at least 80%, 85%, 90%, 95% or 98% sequence identity to the capsid protein of ZC634 described herein.

In some embodiments, the capsid protein comprises the amino acid sequence of SEQ ID NO: 113, or an amino acid sequence having at least 80%, 85%, 90%, 95% or 98% sequence identity to SEQ ID NO: 113. In some embodiments, the capsid protein is that of ZC635 described herein, or an amino acid sequence having at least 80%, 85%, 90%, 95% or 98% sequence identity to the capsid protein of ZC635 described herein.

In some embodiments, the capsid protein comprises the amino acid sequence of SEQ ID NO: 114, or an amino acid sequence having at least 80%, 85%, 90%, 95% or 98% sequence identity to SEQ ID NO: 114. In some embodiments, the capsid protein is that of CR9-01 described herein, or an amino acid sequence having at least 80%, 85%, 90%, 95% or 98% sequence identity to the capsid protein of CR9-01 described herein.

TABLE 13
Exemplary engineered AAV capsid protein sequences

| | | SEQ |
|---|---|---|
| Name | Sequence | ID NO: |
| ZC621 | MAADGYLPDWLEDNLSEGIREWWALKPGAPQPKANQQHQDNAR | 104 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VADVQQLPGFQAQTEWVQNQGILPGMVWQDRDVYLQGPIWAKI | |
| | PHTDGNFHPSPLMGGFGMKHPPPQILIKNTPVPADPPTAFNKDKLN | |
| | SFITQYSTGQVSVEIEWELQKENSKRWNPEIQYTSNYYKSNNVEFA | |
| | VNTEGVYSEPRPIGTRYLTRNL | |
| ZC623 | MAADGYLPDWLEDNLSEGIREWWALKPGAPQPKANQQHQDNAR | 105 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VADVQQHPGFQAQTEWVQNQGILPGMVWQDRDVYLQGPIWAKI | |
| | PHTDGNFHPSPLMGGFGMKHPPPQILIKNTPVPADPPTAFNKDKLN | |
| | SFITQYSTGQVSVEIEWELQKENSKRWNPEIQYTSNYYKSNNVEFA | |
| | VNTEGVYSEPRPIGTRYLTRNL | |
| ZC625 | MAADGYLPDWLEDNLSEGIREWWALKPGAPQPKANQQHQDNAR | 106 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSHVRGDLSGQNQQTLKFSVAGPSNMAVQGRNYIPGPS | |
| | YRQQRVSTTVTQNNNSEFAWPGASSWALNGRNSLMNPGPAMAS | |
| | HKEGEDRFFPLSGSLIFGKQGTGRDNVDADKVMITNEEEIKTTNPV | |
| | ATESYGQVATNHQSAQAQAQTGWVQNQGILPGMVWQDRDVYL | |
| | QGPIWAKIPHTDGNFHPSPLMGGFGMKHPPPQILIKNTPVPADPPT | |
| | AFNKDKLNSFITQYSTGQVSVEIEWELQKENSKRWNPEIQYTSNY | |
| | YKSNNVEFAVNTEGVYSEPRPIGTRYLTRNL | |
| ZC626 | MAADGYLPDWLEDNLSEGIREWWALKPGAPQPKANQQHQDNAR | 107 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGVVSSGARSGQNQQTLKFSVAGPSNMAVQGRNYIPGPS | |
| | YRQQRVSTTVTQNNNSEFAWPGASSWALNGRNSLMNPGPAMAS | |
| | HKEGEDRFFPLSGSLIFGKQGTGRDNVDADKVMITNEEEIKTTNPV | |
| | ATESYGQVATNHQSAQAQAQTGWVQNQGILPGMVWQDRDVYL | |
| | QGPIWAKIPHTDGNFHPSPLMGGFGMKHPPPQILIKNTPVPADPPT | |
| | AFNKDKLNSFITQYSTGQVSVEIEWELQKENSKRWNPEIQYTSNY | |
| | YKSNNVEFAVNTEGVYSEPRPIGTRYLTRNL | |
| ZC630 | MAADGYLPDWLEDNLSEGIREWWALKPGAPQPKANQQHQDNAR | 108 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VATNHQSSVRGDAARLAGAQTGWVQNQGILPGMVWQDRDVYL | |
| | QGPIWAKIPHTDGNFHPSPLMGGFGMKHPPPQILIKNTPVPADPPT | |
| | AFNKDKLNSFITQYSTGQVSVEIEWELQKENSKRWNPEIQYTSNY | |
| | YKSNNVEFAVNTEGVYSEPRPIGTRYLTRNL | |
| ZC631 | MAADGYLPDWLEDNLSEGIREWWALKPGAPQPKANQQHQDNAR | 109 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VADVQQVPGFQAQTMWVQNQGILPGMVWQDRDVYLQGPIWAKI | |
| | PHTDGNFHPSPLMGGFGMKHPPPQILIKNTPVPADPPTAFNKDKLN | |
| | SFITQYSTGQVSVEIEWELQKENSKRWNPEIQYTSNYYKSNNVEFA | |
| | VNTEGVYSEPRPIGTRYLTRNL | |
| ZC632 | MAADGYLPDWLEDNLSEGIREWWALKPGAPQPKANQQHQDNAR | 110 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VADVQQVPGFQAQTQWVQNQGILPGMVWQDRDVYLQGPIWAKI | |
| | PHTDGNFHPSPLMGGFGMKHPPPQILIKNTPVPADPPTAFNKDKLN | |
| | SFITQYSTGQVSVEIEWELQKENSKRWNPEIQYTSNYYKSNNVEFA | |
| | VNTEGVYSEPRPIGTRYLTRNL | |
| ZC633 | MAADGYLPDWLEDNLSEGIREWWALKPGAPQPKANQQHQDNAR | 111 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VADVQQLPGFGAQTEWVQNQGILPGMVWQDRDVYLQGPIWAKI | |
| | PHTDGNFHPSPLMGGFGMKHPPPQILIKNTPVPADPPTAFNKDKLN | |
| | SFITQYSTGQVSVEIEWELQKENSKRWNPEIQYTSNYYKSNNVEFA | |
| | VNTEGVYSEPRPIGTRYLTRNL | |
| ZC634 | MAADGYLPDWLEDNLSEGIREWWALKPGAPQPKANQQHQDNAR | 112 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VADVQQLPGFRPETEWVQNQGILPGMVWQDRDVYLQGPIWAKIP | |
| | HTDGNFHPSPLMGGFGMKHPPPQILIKNTPVPADPPTAFNKDKLNS | |
| | FITQYSTGQVSVEIEWELQKENSKRWNPEIQYTSNYYKSNNVEFA | |
| | VNTEGVYSEPRPIGTRYLTRNL | |
| ZC635 | MAADGYLPDWLEDNLSEGIREWWALKPGAPQPKANQQHQDNAR | 113 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VADVQQLPGFNAQTEWVQNQGILPGMVWQDRDVYLQGPIWAKI | |
| | PHTDGNFHPSPLMGGFGMKHPPPQILIKNTPVPADPPTAFNKDKLN | |
| | SFITQYSTGQVSVEIEWELQKENSKRWNPEIQYTSNYYKSNNVEFA | |
| | VNTEGVYSEPRPIGTRYLTRNL | |
| CR9-01 | MAADGYLPDWLEDNLSEGIREWWALKPGAPQPKANQQHQDNAR | 114 |
| | GLVLPGYKYLGPGNGLDKGEPVNAADAAALEHDKAYDQQLKAG | |
| | DNPYLKYNHADAEFQERLKEDTSFGGNLGRAVFQAKKRLLEPLG | |
| | LVEEAAKTAPGKKRPVEQSPQEPDSSAGIGKSGAQPAKKRLNFGQ | |
| | TGDTESVPDPQPIGEPPAAPSGVGSLTMASGGGAPVADNNEGADG | |
| | VGSSSGNWHCDSQWLGDRVITTSTRTWALPTYNNHLYKQISNSTS | |
| | GGSSNDNAYFGYSTPWGYFDFNRFHCHFSPRDWQRLINNNWGFR | |
| | PKRLNFKLFNIQVKEVTDNNGVKTIANNLTSTVQVFTDSDYQLPY | |
| | VLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLY | |
| | YLSKTISQVNGRPRDLKFSVAGPSNMAVQGRNYIPGPSYRQQRVS | |
| | TTVTQNNNSEFAWPGASSWALNGRNSLMNPGPAMASHKEGEDR | |
| | FFPLSGSLIFGKQGTGRDNVDADKVMITNEEEIKTTNPVATESYGQ | |
| | VATNHQSAQAQAQTGWVQNQGILPGMVWQDRDVYLQGPIWAKI | |
| | PHTDGNFHPSPLMGGFGMKHPPPQILIKNTPVPADPPTAFNKDKLN | |
| | SFITQYSTGQVSVEIEWELQKENSKRWNPEIQYTSNYYKSNNVEFA | |
| | VNTEGVYSEPRPIGTRYLTRNL | |

In some embodiments, the capsid is not a chimeric capsid and/or not a combinatory capsid.

In some embodiments, a recombinant adeno-associated virus (rAAV) virion comprises a capsid protein (such as any described herein) and a vector genome. The vector genome may comprise an expression cassette flanked by inverted terminal repeats (ITRs), wherein the expression cassette is any one described herein for expression of a Cas endonuclease and/or a guide RNA targeting a mutant PLN (e.g., PLN-R14Del).

In some embodiments, the rAAV virion specifically transduces heart cells.

In some embodiments, the rAAV virion specifically transduces cardiomyocytes.

In some embodiments, the rAAV virion traffics to the heart.

In some embodiments, the rAAV virion traffics to at least one organ other than the liver.

In some embodiments, the rAAV virion exhibits a higher heart transduction efficiency than an rAAV virion having an AAV9 VP1 capsid protein according to SEQ ID NO: 46.

In some embodiments, administration of the rAAV virion to a subject leads to a lower liver viral load than administration of an rAAV virion having an AAV9 VP1 capsid protein according to SEQ ID NO: 46. In some embodiments, administration of the rAAV virion to a subject leads to a lower liver viral load in a primate or as assessed in a primate, than administration of an rAAV virion having an AAV9 VP1 capsid protein according to SEQ ID NO: 46. In some embodiments, administration of the rAAV virion to a subject leads to at least 2, 3, 4, 5, 6, 7, 8, 9 or 10 times lower liver viral load than administration of an rAAV virion having an AAV9 VP1 capsid protein according to SEQ ID NO: 46 (e.g., in a primate or as assessed in a primate).

In some embodiments, the rAAV virion exhibits a higher heart-to-liver transduction ratio than an rAAV virion having an AAV9 VP1 capsid protein according to SEQ ID NO: 46.

In some embodiments, the rAAV virion exhibits a heart-to-liver transduction ratio which is at least 2, 3, 4, 5, 6, 7, 8, 9 or 10 times higher than an rAAV virion having an AAV9 VP1 capsid protein according to SEQ ID NO: 46.

In some embodiments, the rAAV virion exhibits a higher transduction efficiency than an rAAV virion having an AAV9 VP1 capsid protein according to SEQ ID NO: 46, assessed in a primate.

In some embodiments, the rAAV virion exhibits a higher heart transduction efficiency than an rAAV virion having an AAV9 VP1 capsid protein according to SEQ ID NO: 46 (e.g., as assessed in a primate).

In some embodiments, the rAAV virion exhibits a higher heart-to-liver transduction ratio than an rAAV virion having an AAV9 VP1 capsid protein according to SEQ ID NO: 46, assessed in a primate.

In some embodiments, the rAAV virion exhibits at least 2, 3, 4, 5, 6, 7, 8, 9, or 10 times higher heart-to-liver transduction ratio than an rAAV virion having an AAV9 VP1 capsid protein according to SEQ ID NO: 46 (e.g., as assessed in a primate).

In some embodiments, the rAAV virions comprise an AAVrh.74 capsid protein or a variant thereof. AAVrh.74 capsid proteins are known in the art. In some embodiments, the rAAV virions comprise an AAVrh.10 capsid protein or a variant thereof. AAVrh.10 capsid proteins are known in the art. In some embodiments, the rAAV virions comprise an AAV-SLB101 capsid protein or a variant thereof as known in the art or described in, e.g., WO 2021/072197, which is incorporated by reference herein in its entirety. In some embodiments, the rAAV virions comprise an AAVmod capsid protein or a variant thereof as known in the art or described in, e.g., WO 2022/173847 or in Olivieri et al. (2021) 24^{th} Annual Meeting of the American Society of Gene & Cell Therapy available at https://www.affiniatx.com/pdf/asgct_2021_olivieri.pdf, both of which are incorporated by reference herein in their entirety. In some embodiments, the rAAV virions comprise the AAV^{mutldec1}, AAV^{deco1}, and/or AAV^{mut1} capsid protein or a variant thereof as known in the art or described in, e.g., WO 2022/173847. In some embodiments, the rAAV virions comprise an AAVcc.47 capsid protein or a variant thereof as known in the art or described in, e.g., Gonzalez et al. Nature Communications 13:5947 (2022), which is incorporated by reference herein in its entirety. In some embodiments, the rAAV virions comprise an AAVHSC16 capsid protein or a variant thereof as known in the art or described in, e.g., Smith et al. Molecular Therapy Methods & Clinical Development 26:224-238 (2022), which is incorporated by reference herein in its entirety. In some embodiments, the rAAV virions comprise a MyoAAV capsid protein or variant thereof as known in the art or described in, e.g., Tabebordbar et al. Cell 184(19):4919-4938. (2021), which is incorporated by reference herein in its entirety. In some embodiments, the rAAV virions comprise the MyoAAV-4E, MyoAAV-3F, MyoAAV-4A, or MyoAAV-4D capsid protein or variant thereof as known in the art or described in, e.g., Tabebordbar et al. In some embodiments, the rAAV virions comprise the 4D-C102 or C102 capsid protein or a variant thereof as known in the art or described in, e.g., US2021/0380643. Exemplary sequences of some of these capsid proteins are provided below.

TABLE 14
Exemplary alternative AAV capsid protein sequences

| | | SEQ |
|---|---|---|
| Name | Sequence | ID NO: |
| AAV-SLB101 | MAADGYLPDWLEDNLSEGIREWWALKPGAPQPKANQQH | 115 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSAQRGDLGLSAQA | |
| | QTGWVQNQGILPGMVWQDRDVYLQGPIWAKIPHTDGNFH | |
| | PSPLMGGFGMKHPPPQILIKNTPVPADPPTAFNKDKLNSFIT | |
| | QYSTGQVSVEIEWELQKENSKRWNPEIQYTSNYYKSNNVE | |
| | FAVNTEGVYSEPRPIGTRYLTRNL | |
| AAVmut1dec1 | MAADGYLPDWLEDNLSEGIREWWALKPGAPQPKANQQH | 116 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGASTNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSAQRGDLLLSAQA | |
| | QTGWVQNQGILPGMVWQDRDVYLQGPIWAKIPHTDGNFH | |
| | PSPLMGGFGMKHPPPQILIKNTPVPADPPTAFNKDKLNSFIT | |
| | QYSTGQVSVEIEWELQKENSKRWNPEIQYTSNYYKSNNVE | |
| | FAVNTEGVYSEPRPIGTRYLTRNL | |
| AAVdeco1 | MAADGYLPDWLEDNLSEGIREWWALKPGAPQPKANQQH | 117 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSAQRGDLLLSAQA | |
| | QTGWVQNQGILPGMVWQDRDVYLQGPIWAKIPHTDGNFH | |
| | PSPLMGGFGMKHPPPQILIKNTPVPADPPTAFNKDKLNSFIT | |
| | QYSTGQVSVEIEWELQKENSKRWNPEIQYTSNYYKSNNVE | |
| | FAVNTEGVYSEPRPIGTRYLTRNL | |
| AAVmut1 | MAADGYLPDWLEDNLSEGIREWWALKPGAPQPKANQQH | 118 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGASTNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSAQAQAQTGWVQ | |
| | NQGILPGMVWQDRDVYLQGPIWAKIPHTDGNFHPSPLMGG | |
| | FGMKHPPPQILIKNTPVPADPPTAFNKDKLNSFITQYSTGQV | |
| | SVEIEWELQKENSKRWNPEIQYTSNYYKSNNVEFAVNTEG | |
| | VYSEPRPIGTRYLTRNL | |
| AAVcc.47 | MAADGYLPDWLEDNLSEGIREWWALKPGAPQPKANQQH | 119 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTIGVSLGGGQTLKFSVAGPSNMAVQGRNYI | |
| | PGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSAQAQAQTGWVQ | |
| | NQGILPGMVWQDRDVYLQGPIWAKIPHTDGNFHPSPLMGG | |
| | FGMKHPPPQILIKNTPVPADPPTAFNKDKLNSFITQYSTGQV | |
| | SVEIEWELQKENSKRWNPEIQYTSNYYKSNNVEFAVNTEG | |
| | VYSEPRPIGTRYLTRNL | |
| AAVHSC16 | MAADGYLPDWLEDNLSEGIREWWALKPGAPQPKANQQH | 120 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEIAWPRASSWALNGRNSLM | |
| | NPGPAMASHKEGEDRFFPLSGSLIFGKQGTGRDNVDADKV | |
| | MITNEEEIKTTNPVATESYGQVATNHQSAQAQAQTGWVQ | |
| | NQGILPGMVWQDRDVYLQGPIWAKIPHTDGNFHPSPLMGG | |
| | FGMKHPPPQILIKNTPVPADPPTAFNKDKLNSFITQYSTGQV | |
| | SVEIEWELQKENSKRWNPEIQYTSNYCKSNNVEFAVNTEG | |
| | VYSEPRPIGTRYLTRNL | |
| MyoAAV-4E | MAADGYLPDWLEDNLSEGIREWWALKPGAPQPKANQQH | 121 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQENRRGDFNNTAQA | |
| | QTGWVQNQGILPGMVWQDRDVYLQGPIWAKIPHTDGNFH | |
| | PSPLMGGFGMKHPPPQILIKNTPVPADPPTAFNKDKLNSFIT | |
| | QYSTGQVSVEIEWELQKENSKRWNPEIQYTSNYYKSNNVE | |
| | FAVNTEGVYSEPRPIGTRYLTRNL | |
| MyoAAV-3F | MAADGYLPDWLEDNLSEGIREWWALKPGAPQPKANQQH | 122 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSAQRGDHASWAQA | |
| | QTGWVQNQGILPGMVWQDRDVYLQGPIWAKIPHTDGNFH | |
| | PSPLMGGFGMKHPPPQILIKNTPVPADPPTAFNKDKLNSFIT | |
| | QYSTGQVSVEIEWELQKENSKRWNPEIQYTSNYYKSNNVE | |
| | FAVNTEGVYSEPRPIGTRYLTRNL | |
| MyoAAV-4A | MAADGYLPDWLEDNLSEGIREWWALKPGAPQPKANQQH | 123 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQSNSRGDYNSLAQA | |
| | QTGWVQNQGILPGMVWQDRDVYLQGPIWAKIPHTDGNFH | |
| | PSPLMGGFGMKHPPPQILIKNTPVPADPPTAFNKDKLNSFIT | |
| | QYSTGQVSVEIEWELQKENSKRWNPEIQYTSNYYKSNNVE | |
| | FAVNTEGVYSEPRPIGTRYLTRNL | |
| MyoAAV-4D | MAADGYLPDWLEDNLSEGIREWWALKPGAPQPKANQQH | 124 |
| | QDNARGLVLPGYKYLGPGNGLDKGEPVNAADAAALEHDK | |
| | AYDQQLKAGDNPYLKYNHADAEFQERLKEDTSFGGNLGR | |
| | AVFQAKKRLLEPLGLVEEAAKTAPGKKRPVEQSPQEPDSS | |
| | AGIGKSGAQPAKKRLNFGQTGDTESVPDPQPIGEPPAAPSG | |
| | VGSLTMASGGGAPVADNNEGADGVGSSSGNWHCDSQWL | |
| | GDRVITTSTRTWALPTYNNHLYKQISNSTSGGSSNDNAYFG | |
| | YSTPWGYFDFNRFHCHFSPRDWQRLINNNWGFRPKRLNFK | |
| | LFNIQVKEVTDNNGVKTIANNLTSTVQVFTDSDYQLPYVL | |
| | GSAHEGCLPPFPADVFMIPQYGYLTLNDGSQAVGRSSFYCL | |
| | EYFPSQMLRTGNNFQFSYEFENVPFHSSYAHSQSLDRLMNP | |
| | LIDQYLYYLSKTINGSGQNQQTLKFSVAGPSNMAVQGRNY | |
| | IPGPSYRQQRVSTTVTQNNNSEFAWPGASSWALNGRNSLM | |
| | NPGPAMASHKEGEDRFFPLSGSLIFGKQGTGRDNVDADKV | |
| | MITNEEEIKTTNPVATESYGQVATNHQASTRGDHGVLAQA | |
| | QTGWVQNQGILPGMVWQDRDVYLQGPIWAKIPHTDGNFH | |
| | PSPLMGGFGMKHPPPQILIKNTPVPADPPTAFNKDKLNSFIT | |
| | QYSTGQVSVEIEWELQKENSKRWNPEIQYTSNYYKSNNVE | |
| | FAVNTEGVYSEPRPIGTRYLTRNL | |
| 4D-C102 | MAADGYLPDWLEDTLSEGIRQWWKLKPGPPPPKPAERHK | 125 |
| variant | DDSRGLVLPGYKYLGPFNGLDKGEPVNEADAAALEHDKA | |
| sequence 1 | YDRQLDSGDNPYLKYNHADAEFQERLKEDTSFGGNLGRA | |
| | VFQAKKRVLEPLGLVEEPVKTAPGKKRPVEHSPVEPDSSSG | |
| | TGKAGQQPARKRLNFGQTGDADSVPDPQPLGQPPAAPSGL | |
| | GTNTMATGSGAPMADNNEGADGVGNSSGNWHCDSTWM | |
| | GDRVITTSTRTWALPTYNNHLYKQISSQSGASNDNHYFGYS | |
| | TPWGYFDFNRFHCHFSPRDWQRLINNNWGFRPKRLNFKLF | |
| | NIQVKEVTQNDGTTTIANNLTSTVQVFTDSEYQLPYVLGSA | |
| | HQGCLPPFPADVFMVPQYGYLTLNNGSQAVGRSSFYCLEY | |
| | FPSQMLRTGNNFTFSYTFEDVPFHSSYAHSQSLDRLMNPLI | |
| | DQYLYYLSRTNTPSGTTTQSRLQFSQAGASDIRDQSRNWLP | |
| | GPCYRQQRVSKTSADNNNSEYSWTGATKYHLNGRDSLVN | |
| | PGPAMASHKDDEEKFFPQSGVLIFGKQGSEKTNVDIEKVMI | |
| | TDEEEIRTTNPVATEQYGSVSTNLQRGNLANKTTNKDARQ | |
| | AATADVNTQGVLPGMVWQDRDVYLQGPIWAKIPHTDGHF | |
| | HPSPLMGGFGLKHPPPQILIKNTPVPANPSTTFSAAKFASFIT | |
| | QYSTGQVSVEIEWELQKENSKRWNPEIQYTSNYNKSINVDF | |
| | TVDTNGVYSEPRPIGTRYLTRNL | |
| 4D-C102 | MAADGYLPDWLEDTLSEGIRQWWKLKPGPPPPKPAERHK | 126 |
| variant | DDSRGLVLPGYKYLGPFNGLDKGEPVNEADAAALEHDKA | |
| sequence 2 | YDRQLDSGDNPYLKYNHADAEFQERLKEDTSFGGNLGRA | |
| | VFQAKKRVLEPLGLVEEPVKTAPGKKRPVEHSPVEPDSSSG | |
| | TGKAGQQPARKRLNFGQTGDADSVPDPQPLGQPPAAPSGL | |
| | GTNTMATGSGAPMADNNEGADGVGNSSGNWHCDSTWM | |
| | GDRVITTSTRTWALPTYNNHLYKQISSQSGASNDNHYFGYS | |
| | TPWGYFDFNRFHCHFSPRDWQRLINNNWGFRPKRLNFKLF | |
| | NIQVKEVTQNDGTTTIANNLTSTVQVFTDSEYQLPYVLGSA | |
| | HQGCLPPFPADVFMVPQYGYLTLNNGSQAVGRSSFYCLEY | |
| | FPSQMLRTGNNFTFSYTFEDVPFHSSYAHSQSLDRLMNPLI | |
| | DQYLYYLSRTNTPSGTTTQSRLQFSQAGASDIRDQSRNWLP | |
| | GPCYRQQRVSKTSADNNNSEYSWTGATKYHLNGRDSLVN | |
| | PGPAMASHKDDEEKFFPQSGVLIFGKQGSEKTNVDIEKVMI | |
| | TDEEEIRTTNPVATEQYGSVSTNLQRGNLANKIQRTDARQA | |
| | ATADVNTQGVLPGMVWQDRDVYLQGPIWAKIPHTDGHFH | |
| | PSPLMGGFGLKHPPPQILIKNTPVPANPSTTFSAAKFASFITQ | |
| | YSTGQVSVEIEWELQKENSKRWNPEIQYTSNYNKSINVDFT | |
| | VDTNGVYSEPRPIGTRYLTRNL | |

In some embodiments, the rAAV is replication defective, in that the rAAV virion cannot independently further replicate and package its genome. For example, when a cardiac cell is targeted with rAAV virions, the transgene is expressed in the targeted cardiac cell, however, due to the fact that the targeted cardiac cell lacks AAV rep and cap genes and accessory function genes, the rAAV is not able to replicate.

In some embodiments, rAAV virions of the present disclosure encapsulating the expression cassettes as described herein, can be produced using helper-free production. rAAVs are replication-deficient viruses and normally require components from a live helper virus, such as adenovirus, in a host cell for packaging of infectious rAAV virions. rAAV helper-free production systems allow the production of infectious rAAV virions without the use of a live helper virus. In the helper-free system, a host packaging cell line is co-transfected with three plasmids. A first plasmid may contain adenovirus gene products (e.g. E2A, E4, and VA RNA genes) needed for the packaging of rAAV virions. A second plasmid may contain required AAV genes (e.g., REP and CAP genes). A third plasmid contains the polynucleotide sequence encoding the transgene of interest and a promoter flanked by ITRs. A host packaging cell line can be, for example, AAV-293 host cells. Suitable host cells contain additional components required for packaging infectious rAAV virions that are not supplied by the plasmids. In some embodiments, the CAP genes can encode, for example, AAV capsid proteins as described herein.

### Pharmaceutical Compositions

Also provided herein are pharmaceutical compositions comprising at least one vector described herein.

The present disclosure provides pharmaceutical compositions for treating and/or preventing heart disease associated with a PLN mutation such as PLN-R14Del.

In some embodiments, the present disclosure provides pharmaceutical compositions comprising a vector (e.g., a viral vector) or a virion (e.g., an rAAV) described herein, and one or more pharmaceutically acceptable carriers, diluents or excipients for parenteral delivery. In some embodiments, the vector comprises polynucleotides encoding a Cas protein and a guide RNA, as described herein.

In some embodiments, the present disclosure provides pharmaceutical compositions comprising a guide RNA or a polynucleotide encoding a guide RNA, as described herein, and one or more pharmaceutically acceptable carriers, diluents or excipients for parenteral delivery.

In various embodiments, the pharmaceutical compositions described herein contain one or more pharmaceutically acceptable excipients. Pharmaceutically acceptable excipients can include vehicles (e.g., carriers, diluents and excipients) that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Illustrative pharmaceutical forms suitable for injectable use include, e.g., sterile aqueous solutions or dispersions; formulations including sesame oil, peanut oil or aqueous propylene glycol; and sterile powders for the extemporaneous preparation of sterile injectable solutions or dispersions.

In some embodiments, the pharmaceutical compositions of the disclosure comprise about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, about 1×10¹⁴ GC/mL, or about 5×10¹⁴ GC/mL of the viral vector (e.g. rAAV virion).

In some embodiments, the pharmaceutical compositions of the disclosure comprise about 1×10⁸ viral genomes per milliliter (vg/mL), about 5×10⁸ vg/mL, about 1×10⁹ vg/mL, about 5×10⁹ vg/mL, about 1×10¹⁰ vg/mL, about 5×10¹⁰ vg/mL, about 1×10¹¹ vg/mL, about 5×10¹¹ vg/mL, about 1×10¹² vg/mL, about 5×10¹² vg/mL, about 5×10¹³ vg/mL, about 1×10¹⁴ vg/mL, or about 5×10¹⁴ vg/mL of the viral vector (e.g. rAAV virion).

In some embodiments, the pharmaceutical compositions of the disclosure comprise less than about 1×10¹⁵ viral genomes per milliliter (vg/mL), less than about 5×10¹⁴ vg/mL, less than about 1×10¹⁴ vg/mL, less than about 5×10¹³ vg/mL, less than about 1×10¹³ vg/mL, less than about 5×10¹² vg/mL, less than about 1×10¹² vg/mL, less than about 5×10¹¹ vg/mL, or less than about 1×10¹¹ vg/mL of the viral vector (e.g. rAAV virion).

In some embodiments, the pharmaceutical compositions of the disclosure comprise less than about 1×10¹⁴ viral genomes per milliliter (vg/mL) or less than about 1×10¹³ vg/mL of the viral vector (e.g., rAAV virion).

In some embodiments, the pharmaceutical compositions of the disclosure comprise from about 1×10¹¹ viral genomes per milliliter (vg/mL) to about 1×10¹⁵ vg/mL of the viral vector (e.g. rAAV virion). In some embodiments, the pharmaceutical compositions of the disclosure comprise from about 1×10¹¹ viral genomes per milliliter (vg/mL) to about 1×10¹⁴ vg/mL of the viral vector (e.g., rAAV virion). In some embodiments, the pharmaceutical compositions of the disclosure comprise from about 1×10¹² viral genomes per milliliter (vg/mL) to about 1×10¹⁴ vg/mL of the viral vector (e.g., rAAV virion). In some embodiments, the pharmaceutical compositions of the disclosure comprise from about 1×10¹² viral genomes per milliliter (vg/mL) to about 1×10¹³ vg/mL of the viral vector (e.g., rAAV virion). In some embodiments, the pharmaceutical compositions of the disclosure comprise from about 1×10¹² viral genomes per milliliter (vg/mL) to about 6×10¹³ vg/mL of the viral vector (e.g., rAAV virion).

In some embodiments, the pharmaceutical compositions of the disclosure comprise any amount or concentration range of the viral vectors or rAAV virions of the disclosure between the values referenced herein.

In some embodiments, the pharmaceutical compositions of the disclosure are administered in a total volume of about 1 mL, 5 mL, 10 mL, about 20 mL, about 25 mL, about 30 mL, about 35 mL, about 40 mL, about 45 mL, about 50 mL, about 55 mL, about 60 mL, 65 mL, about 70 mL, about 75 mL, about 80 mL, about 85 mL, about 90 mL, about 95 mL, about 100 mL, about 105 mL, about 110 mL, about 115 mL, about 120 mL, about 125 mL, about 130 mL, about 135 mL, about 140 mL, about 145 mL, about 150 mL, about 155 mL, about 160 mL, about 165 mL, about 170 mL, about 175 mL, about 180 mL, about 185 mL, about 190 mL, about 200 mL, about 205 mL, about 210 mL, about 215 mL, or about 220 mL.

### Kits

In some embodiments, the present disclosure provides a kit comprising a container housing a pharmaceutical composition as described herein.

The kit can include any of compositions described herein, either mixed together or individually packaged, and in dry or hydrated form. The rAAV virions and/or other agents described herein can be packaged separately into discrete vials, bottles or other containers. Alternatively, any of the rAAV virions and/or agents described herein can be packaged together as a single composition, or as two or more compositions that can be used together or separately. The compounds and/or agents described herein can be packaged in appropriate ratios and/or amounts to facilitate conversion of selected cells across differentiation boundaries to form cardiac progenitor cells and/or cardiomyocytes.

The kit can include instructions for administering those compositions, compounds and/or agents. Such instructions can provide the information described throughout this application. The rAAV virion or pharmaceutical composition can be provided within any of the kits in the form of a delivery device. Alternatively, a delivery device can be separately included in the kits, and the instructions can describe how to assemble the delivery device prior to administration to a subject.

Any of the kits can also include syringes, catheters, scalpels, sterile containers for sample or cell collection, diluents, pharmaceutically acceptable carriers, and the like. The kits can provide other factors such as any of the supplementary factors or drugs described herein for the compositions in the preceding section or other parts of the application.

### Cells

Also provided herein is an isolated cell or population of cells comprising any vector described herein. Also provided herein is an isolated cell or population of cells comprising two or more vectors described herein.

In some embodiments, the cell is a cardiac cell.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

In some embodiments, the cell is a cardiomyocyte.

In some embodiments, the cell is an induced pluripotent stem cell (iPSC). In some embodiments, a cell is an iPSC-derived cardiomyocyte.

The disclosure provides methods comprising contacting the cell with any vector or virion (e.g., rAAV virion) described herein. In some embodiments, the cell is a cardiac cell. In some embodiments, the cell is a cardiomyocyte. In some embodiments, the contacting is in vitro. In some embodiments, the contacting is in vivo.

The disclosure provides methods comprising contacting the tissue with any vector or virion (e.g., rAAV virion) described herein. In some embodiments, the tissue is cardiac tissue. In some embodiments, the contacting is in vitro. In some embodiments, the contacting is in vivo.

The disclosure provides methods comprising contacting the organ with any vector or virion (e.g., rAAV virion) described herein. In some embodiments, the organ is a heart. In some embodiments, the heart is diseased or at risk of disease. In some embodiments, the heart has borderline or reduced ejection fraction. In some embodiments, the heart has a normal ejection fraction. In some embodiments, the heart comprises a genetic mutation in PLN associated with a heart disease. In some embodiments, the genetic mutation is a deleterious or dominant negative mutation in PLN. In some embodiments, the genetic mutation is a PLN-R14Del mutation. In some embodiments, the contacting is in vitro. In some embodiments, the contacting is in vivo.

Also provided herein is a cell therapy composition comprising any cell described herein.

The disclosure provides methods for expressing a polynucleotide a cell. The method may comprise, for example, transducing a target cell with the rAAV virions, rAAV vector genomes, or expression cassettes described herein. A target cell can be, for example and without limitation, a cardiac cell, a muscle cell, an induced pluripotent stem cell-derived cardiomyocyte (iPSC-CM), a cardiomyocyte, a PLN mutant cell.

In some embodiments, the disclosure provides methods for expressing a polynucleotide in a target cell. The method may comprise, for example, transducing a target cell with the rAAV virions, rAAV vector genomes, or expression cassettes described herein. In some embodiments, the virions, rAAV vector genomes, or expression cassettes encode a Cas protein and a gRNA specific for PLN-R14Del. A target cell can be, for example and without limitation, a cardiac cell, a muscle cell, an induced pluripotent stem cell-derived cardiomyocyte (iPSC-CM) comprising a PLN-R14Del, a cardiomyocyte comprising a PLN-R14Del, or an iPSC comprising a PLN-R14Del. In some embodiments, a method of expressing a Cas protein and/or a gRNA specific for PLN-R14Del in a cell comprises transducing a target cell or population of target cells with an rAAV virion or rAAV vector genomes described herein.

In one embodiment, the cell is a PLN-R14Del cell. In one embodiment, the cell comprises a mutation of the endogenous PLN gene. In one embodiment, the mutation in the PLN gene causes or is known to cause cardiomyopathy, e.g., DCM or arrhythmogenic right ventricular cardiomyopathy. See Eijgenraam et. al, Scientific Reports. 2020; 10:9819.

In some embodiments, reprogramming factors may be capable of converting a cardiac fibroblast comprising a PLN-R14Del mutation to a cardiac myocyte, either directly or through an intermediate cell type. In particular, direct reprogramming is possible, or reprogramming by first converting the fibroblast to a pluripotent or totipotent stem cell. Such a pluripotent stem cell is termed an induced pluripotent stem (iPS) cell. An iPS cell that is subsequently converted to a cardiac myocyte (CM) cell is termed an iPS-CM cell. In the examples, iPS-CM derived in vitro from cardiac fibroblasts are used in vivo to select capsid proteins of interest. The disclosure also envisions using the capsid proteins disclosure to in turn generate iPS-CM cells in vitro but, particular, in vivo, as part of a therapeutic gene therapy regimen. Induced cardiomyocyte-like (iCM) cells refer to cells directly reprogrammed into cardiomyocytes. Thus, the methods of the disclosure allow for transducing an IPS cell with an rAAV virion or rAAV vector genome comprising a PLN-R14Del mutation with a Cas protein and a gRNA specific for PLN-R14Del, followed by conversion to an IPS-CM cell or an iCM cell.

In some embodiments, the administration of the guide RNA or vectors described herein to a cell with a PLN gene comprising a deletion of Arg14 results in editing efficiency of, or efficiency of indel formation in, the PLN gene of at least 20%, 25%, 30%, 35%, 40%, 45%, 50%, 55%, 60% or 70%. In some embodiments, the administration of the guide RNA or vectors described herein to a cell with a PLN gene comprising a deletion of Arg14 results in editing efficiency of, or efficiency of indel formation in, the PLN gene of at least or about 25%. In some embodiments, the administration of the guide RNA or vectors described herein to a cell with a PLN gene comprising a deletion of Arg14 results in editing efficiency of, or efficiency of indel formation in, the PLN gene of at least or about 30%. In some embodiments, the administration of the guide RNA or vectors described herein to a cell with a PLN gene comprising a deletion of Arg14 results in editing efficiency of, or efficiency of indel formation in, the PLN gene of at least or about 35%. In some embodiments, the administration of the guide RNA or vectors described herein to a cell with a PLN gene comprising a deletion of Arg14 results in editing efficiency of, or efficiency of indel formation in, the PLN gene of at least or about 40%. In some embodiments, the administration of the guide RNA or vectors described herein to a cell with a PLN gene comprising a deletion of Arg14 results in editing efficiency of, or efficiency of indel formation in, the PLN gene of at least or about 45%. In some embodiments, the administration of the guide RNA or vectors described herein to a cell with a PLN gene comprising a deletion of Arg14 results in editing efficiency of, or efficiency of indel formation in, the PLN gene of at least or about 50%. In some embodiments, the administration of the guide RNA or vectors described herein to a cell with a PLN gene comprising a deletion of Arg14 results in editing efficiency of, or efficiency of indel formation in, the PLN gene of at least or about 55%. In some embodiments, the administration of the guide RNA or vectors described herein to a cell with a PLN gene comprising a deletion of Arg14 results in editing efficiency of, or efficiency of indel formation in, the PLN gene of at least or about 60%. In some embodiments, the administration of the guide RNA or vectors described herein to a cell with a PLN gene comprising a deletion of Arg14 results in editing efficiency of, or efficiency of indel formation in, the PLN gene of at least or about 65%. In some embodiments, the administration of the guide RNA or vectors described herein to a cell with a PLN gene comprising a deletion of Arg14 results in editing efficiency of, or efficiency of indel formation in, the PLN gene of at least or about 70%. In some embodiments, the administration of the guide RNA or vectors described herein to a cell with a PLN gene comprising a deletion of Arg14 results in editing efficiency of, or efficiency of indel formation in, the PLN gene of at least or about 75%. In some embodiments, the administration of the guide RNA or vectors described herein to a cell with a PLN gene comprising a deletion of Arg14 results in editing efficiency of, or efficiency of indel formation in, the PLN gene of at least or about 80%. In some embodiments, the administration of the guide RNA or vectors described herein to a cell with a PLN gene comprising a deletion of Arg14 results in editing efficiency of, or efficiency of indel formation in, the PLN gene of at least or about 85%. In some embodiments, the administration of the guide RNA or vectors described herein to a cell with a PLN gene comprising a deletion of Arg14 results in editing efficiency of, or efficiency of indel formation in, the PLN gene of at least or about 90%. In some embodiments, the administration of the guide RNA or vectors described herein to a cell with a PLN gene comprising a deletion of Arg14 results in editing efficiency of, or efficiency of indel formation in, the PLN gene of at least or about 95%.

In some embodiments, the administration of the guide RNA or vectors described herein to a cell with a PLN gene comprising a deletion of Arg14 does not, or substantially does not, edit or form indels in the wild-type PLN gene.

### Patient Populations

Subjects who are suitable for treatment using the compositions and methods of the present disclosure include individuals (e.g., mammalian subjects, such as humans, non-human primates, domestic mammals, experimental non-human mammalian subjects such as mice, rats, etc.) having a mutation in PLN gene.

In some embodiments, the subject to be treated in accordance with the methods described herein is a mammal. In some embodiments, the subject to be treated in accordance with the methods described herein is a human. In some embodiments, the subject to be treated in accordance with the methods described herein is a female.

In some embodiments, the subject to be treated has a mutation in PLN gene. In some embodiments, the subject to be treated has any mutation in PLN known in the art or described herein. In some embodiments, the subject to be treated has PLN-R14del mutation.

The p.(Arg14del) pathogenic variant of the PLN gene (PLN-R14Del) is a prevalent genetic deviation and has been identified in 14% of Dutch dilated cardiomyopathy and arrhythmogenic right ventricular cardiomyopathy patients. See Eijgenraam et. al, Scientific Reports. 2020; 10:9819. PLN-R14Del-related cardiomyopathy is characterized by high prevalence of malignant ventricular arrhythmias and end-stage heart failure (HF), with poor prognosis and high mortality from adolescence. Without wishing to be bound by theory, targeting the PLN-R14Del allele, as described herein, is protective against PLN-R14Del-related cardiomyopathy (e.g., dilated cardiomyopathy, hypertrophic cardiomyopathy and arrhythmogenic right ventricular cardiomyopathy).

In some embodiments, the subject to be treated has a deleterious mutation in PLN gene. In some embodiments, the subject to be treated has a PLN-R14Del mutation in PLN gene.

In some embodiments, the subject to be treated has or is at risk of a disease or condition associated with a mutation in a PLN gene (e.g., PLN-R14Del).

In some embodiments, the subject to be treated has or is at risk of a heart disease, e.g., a heart disease associated with a mutation in a PLN gene (e.g., PLN-R14Del).

In some embodiments, the subject to be treated has or is at risk of a cardiomyopathy, e.g., a cardiomyopathy associated with a mutation in a PLN gene (e.g., PLN-R14Del). In some embodiments, the cardiomyopathy is DCM, HCM, ACM or ARVC.

In some embodiments, the subject to be treated has or is at risk of heart failure, e.g., a heart failure associated with a mutation in a PLN gene (e.g., PLN-R14Del).

In some embodiments, the subject to be treated has or is at risk of arrhythmia, e.g., arrhythmia associated with a mutation in a PLN gene (e.g., PLN-R14Del).

In some embodiments, the compositions and methods disclosed herein can be used for the prevention and/or treatment of cardiomyopathies in a subject. In some embodiments, the compositions and methods described herein can be used to treat cardiomyopathies associated with mutations in PLN gene, such as dilated cardiomyopathy, and other heart diseases associated with mutations in the PLN gene.

### Methods of Treating Heart Disease

### Heart Diseases to be Treated

In some embodiments, the heart disease to be treated or prevented in accordance with the methods described herein is a heart disease associated with a deleterious mutation in a PLN gene. In some embodiments, the heart disease to be treated or prevented in accordance with the methods described herein is a heart disease associated with PLN-R14Del mutation.

In some embodiments, the heart disease to be treated or prevented in accordance with the methods described herein is cardiomyopathy, e.g., DCM. In some embodiments, the heart disease to be treated or prevented in accordance with the methods described herein is an idiopathic DCM. In some embodiments, the heart disease to be treated or prevented in accordance with the methods described herein is HCM. In some embodiments, the heart disease to be treated or prevented in accordance with the methods described herein is ACM. In some embodiments, the heart disease to be treated or prevented in accordance with the methods described herein is ARVC.

In some embodiments, the heart disease to be treated or prevented in accordance with the methods described herein is PLN mutation-associated cardiomyopathy, e.g., PLN mutation-associated DCM, PLN mutation-associated HCM, PLN mutation-associated ACM, or PLN-mutation associated ARVC. In some embodiments, the heart disease to be treated or prevented in accordance with the methods described herein is PLN mutation-associated idiopathic DCM.

In some embodiments, the heart disease to be treated or prevented in accordance with the methods described herein is a heart failure. In some embodiments, the heart disease to be treated or prevented in accordance with the methods described herein is a PLN mutation-associated heart failure.

In some embodiments, the heart disease to be treated or prevented in accordance with the methods described herein is a heart failure with reduced ejection fraction. In some embodiments, the heart disease to be treated or prevented in accordance with the methods described herein is a PLN mutation-associated heart failure with reduced ejection fraction.

In some embodiments, the heart disease to be treated or prevented in accordance with the methods described herein is an ischemic heart failure. In some embodiments, the heart disease to be treated or prevented in accordance with the methods described herein is a PLN mutation-associated ischemic heart failure.

In some embodiments, the heart disease to be treated or prevented in accordance with the methods described herein is an arrhythmia, e.g., atrial and/or ventricular arrhythmia, and/or malignant ventricular arrhythmia. In some embodiments, the heart disease to be treated or prevented in accordance with the methods described herein is a PLN mutation-associated arrhythmia, e.g., atrial and/or ventricular arrhythmia, and/or malignant ventricular arrhythmia.

The cardiomyopathy treated or prevented by the compositions and methods described herein can also include cardiomyopathies associated with a pulmonary embolus, a venous thrombosis, a myocardial infarction, a transient ischemic attack, a peripheral vascular disorder, atherosclerosis, ischemic cardiac disease and/or other myocardial injury or vascular disease. In certain embodiments, the cardiomyopathies treated by the compositions and methods described herein can include cardiac diseases associated with myocardial tissue hypercontractility, such as heart failure related to left ventricular hypercontractility.

### Methods of Treating Heart Disease

The compositions that are described herein can be employed in a method of treating a subject with a cardiac disease or condition. “Treating” or “treatment of a condition or subject in need thereof” refers to (1) taking steps to obtain beneficial or desired results, including clinical results such as the reduction of symptoms; (2) inhibiting the disease, for example, arresting or reducing the development of the disease or its clinical symptoms; (3) relieving the disease, for example, causing regression of the disease or its clinical symptoms; and/or (4) delaying the disease.

In some embodiments, the compositions and methods described herein can induce detectable expression of a therapeutic protein or nucleic acid (e.g., Cas9 protein or guide RNA) in a subject in need thereof.

### Routes and Modes of Administration

The vectors and virions (e.g., AAV) of the present disclosure can be administered to a subject in need thereof by systemic application (such as parenteral application), e.g., by intravenous, intra-arterial or intraperitoneal delivery of a vector in analogy to what has been shown in animal models (Katz et al., 2012, Gene Ther. 19:659-669). In some embodiments, the vectors and virions (e.g., AAV) of the present disclosure are to be administered parenterally. In some embodiments, the vectors and virions (e.g., AAV) of the present disclosure are to be administered intravenously (e.g., by IV infusion).

In some embodiments, the vectors and virions (e.g., AAV) of the present disclosure can be delivered by direct administration to the heart tissue.

In some embodiments, the vectors and virions (e.g., AAV) of the present disclosure can be delivered by intracoronary administration. In some embodiments, the administration is by antegrade epicardial coronary artery infusion, e.g., a single infusion over a 10-minute period in a cardiac catheterization laboratory after angiography (percutaneous intracoronary delivery without vessel balloon occlusion) with the use of standard 5F or 6F guide or diagnostic catheters (Jaski et al., 2009, J Card Fail. 15: 171-181).

In some embodiments, the vectors and virions (e.g., AAV) of the present disclosure can be delivered by direct injection into the heart or cardiac catheterization. In some embodiments, the vectors and virions (e.g., AAV) of the present disclosure can be delivered by intracardiac catheter delivery via retrograde coronary sinus infusion (RCSI).

When direct injection is used, it may be performed either by open-heart surgery or by minimally invasive surgery. In some cases, the vectors and virions (e.g., AAV) can be delivered to the pericardial space by injection or infusion.

In some embodiments, the amount, concentration, and volume of the composition that modulates contractile function in myocardial tissue administered to a subject can be controlled and/or optimized to substantially improve the functional parameters of the heart while mitigating adverse side effects.

The amount of the composition that modulates contractile function administered to myocardial tissue can also be an amount required to result in the detectable expression of a therapeutic protein or nucleic acid (e.g., Cas9 or guide RNA) in the heart; preserve and/or improve contractile function; delay the emergence of cardiomyopathy or reverse the pathological course of the disease; increase myocyte viability; improve myofilament function; inhibit left ventricular hypertrophy; cardiac hypertrophy regression, normalize systolic and diastolic function in heart; and restore normal cross-bridge behavior at the myofilament level.

In some embodiments, the compositions and methods disclosed herein result in restoration of function of PLN gene and gene product in a cardiac cell or tissue (such as heart) of the subject being treated.

In some embodiments, the methods of the disclosure comprise administering an rAAV virion at a dose of about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, about 1×10¹⁴ GC/mL, or about 5×10¹⁴ GC/mL of the rAAV virion.

In some embodiments, the methods of the disclosure comprise intravenously administering an rAAV virion at a dose of about 3×10¹² GC/mL, about 3×10¹³ GC/mL, about 1×10¹⁴ GC/mL, or about 3×10¹⁴ GC/mL of the rAAV virion.

In some embodiments, the methods of the disclosure comprise administering, by localized delivery to the heart, an rAAV virion at a dose of about 3×10¹¹ GC/mL, about 3×10¹² GC/mL, about 1×10¹³ GC/mL, or about 3×10¹³ GC/mL of the rAAV virion.

In some embodiments, the methods of the disclosure comprise administering an rAAV virion at a dose of about 1×10⁸ viral genomes per milliliter (vg/mL), about 5×10⁸ vg/mL, about 1×10⁹ vg/mL, about 5×10⁹ vg/mL, about 1×10¹⁰ vg/mL, about 5×10¹⁰ vg/mL, about 1×10¹¹ vg/mL, about 5×10¹¹ vg/mL, about 1×10¹² vg/mL, about 5×10¹² vg/mL, about 5×10¹³ vg/mL, about 1×10¹⁴ vg/mL, or about 5×10¹⁴ vg/mL of the rAAV virion.

In some embodiments, the methods of the disclosure comprise administering an rAAV virion at a dose of less than about 1×10¹⁵ viral genomes per milliliter (vg/mL), less than about 5×10¹⁴ vg/mL, less than about 1×10¹⁴ vg/mL, less than about 5×10¹³ vg/mL, less than about 1×10¹³ vg/mL, less than about 5×10¹² vg/mL, less than about 1×10¹² vg/mL, less than about 5×10¹¹ vg/mL, or less than about 1×10¹¹ vg/mL of the viral vector (e.g. rAAV virion).

In some embodiments, the methods of the disclosure comprise administering an rAAV virion at a dose of less than about 1×10¹⁴ viral genomes per milliliter (vg/mL) or less than about 1×10¹³ vg/mL of the viral vector (e.g. rAAV virion).

In some embodiments, the methods of the disclosure comprise administering an rAAV virion at a dose from about 1×10¹¹ viral genomes per milliliter (vg/mL) to about 1×10¹⁵ vg/mL of the viral vector (e.g. rAAV virion). In some embodiments, the methods of the disclosure comprise administering an rAAV virion at a dose from about 1×10¹¹ viral genomes per milliliter (vg/mL) to about 1×10¹⁴ vg/mL of the viral vector (e.g. rAAV virion). In some embodiments, the methods of the disclosure comprise administering an rAAV virion at a dose from about 1×10¹² viral genomes per milliliter (vg/mL) to about 1×10¹⁴ vg/mL of the viral vector (e.g. rAAV virion). In some embodiments, the methods of the disclosure comprise administering an rAAV virion at a dose from about 1×10¹² viral genomes per milliliter (vg/mL) to about 1×10¹³ vg/mL of the viral vector (e.g., rAAV virion). In some embodiments, the methods of the disclosure comprise administering an rAAV virion at a dose from about 1×10¹² viral genomes per milliliter (vg/mL) to about 6×10¹³ vg/mL of the viral vector (e.g., rAAV virion).

In some embodiments, the methods of the disclosure comprise administering an rAAV virion at any dose or dose range of the disclosure between the values referenced herein.

In some embodiments, the methods of the disclosure comprise intravenously administering an rAAV virion at a dose of about 1×10¹² vg/mL, about 3×10¹² vg/mL, about 6×10¹² vg/mL, or about 9×10¹² vg/mL of the rAAV virion.

In some embodiments, the methods of the disclosure comprise administering, by localized delivery to the heart, an rAAV virion at a dose of about 1×10¹² vg/mL, about 3×10¹² vg/mL, about 6×10¹² vg/mL, or about 9×10¹² vg/mL of the rAAV virion.

Genome copies per milliliter can be determined by quantitative polymerase change reaction (qPCR) using a standard curve generated with a reference sample having a known concentration of the polynucleotide genome of the virus. For AAV, the reference sample used is often the transfer plasmid used in generation of the rAAV virion but other reference samples may be used.

Alternatively or in addition, the concentration of a viral vector can be determined by measuring the titer of the vector on a cell line. Viral titer is typically expressed as viral particles (vp) per unit volume (e.g., vp/mL). In various embodiments, the pharmaceutical compositions of the disclosure comprise about 1×10⁸ viral particles per milliliter (vp/mL), about 5×10⁸ vp/mL, about 1×10⁹ vp/mL, about 5×10⁹ vp/mL, about 1×10¹⁰ vp/mL, about 5×10¹⁰ vp/mL, about 1×10¹¹ vp/mL, about 5×10¹¹ vp/mL, about 1×10¹² vp/mL, about 5×10¹² vp/mL, about 5×10¹³ vp/mL, or about 1×10¹⁴ vp/mL, or about 5×10¹⁴ of the viral vector (e.g., rAAV virion).

The viral vector administered to the subject can be traced by a variety of methods. For example, recombinant viruses labeled with or expressing a marker (such as green fluorescent protein, or beta-galactosidase) can readily be detected. The recombinant viruses may be engineered to cause the target cell to express a marker protein, such as a surface-expressed protein or a fluorescent protein. Alternatively, the infection of target cells with recombinant viruses can be detected by their expression of a cell marker that is not expressed by the animal employed for testing (for example, a human-specific antigen when injecting cells into an experimental animal). The presence and phenotype of the target cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

### Effect of Administration of Therapies

In some embodiments, the administration of the therapies described herein to a subject results in editing efficiency of, or efficiency of indel formation in, the PLN gene comprising a deletion of Arg14 of at least 20%, 25%, 30%, 35%, 40%, 45%, 50%, 55%, 60% or 70% (such as in vivo editing efficiency). In some embodiments, the administration of the therapies described herein to a subject results in editing efficiency of, or efficiency of indel formation in, the PLN gene comprising a deletion of Arg14 of at least or about 25%. In some embodiments, the administration of the therapies described herein to a subject results in editing efficiency of, or efficiency of indel formation in, the PLN gene comprising a deletion of Arg14 of at least or about 30%. In some embodiments, the administration of the therapies described herein to a subject results in editing efficiency of, or efficiency of indel formation in, the PLN gene comprising a deletion of Arg14 of at least or about 35%. In some embodiments, the administration of the therapies described herein to a subject results in editing efficiency of, or efficiency of indel formation in, the PLN gene comprising a deletion of Arg14 of at least or about 40%. In some embodiments, the administration of the therapies described herein to a subject results in editing efficiency of, or efficiency of indel formation in, the PLN gene comprising a deletion of Arg14 of at least or about 45%. In some embodiments, the administration of the therapies described herein to a subject results in editing efficiency of, or efficiency of indel formation in, the PLN gene comprising a deletion of Arg14 of at least or about 50%. In some embodiments, the administration of the therapies described herein to a subject results in editing efficiency of, or efficiency of indel formation in, the PLN gene comprising a deletion of Arg14 of at least or about 55%. In some embodiments, the administration of the therapies described herein to a subject results in editing efficiency of, or efficiency of indel formation in, the PLN gene comprising a deletion of Arg14 of at least or about 60%. In some embodiments, the administration of the therapies described herein to a subject results in editing efficiency of, or efficiency of indel formation in, the PLN gene comprising a deletion of Arg14 of at least or about 65%. In some embodiments, the administration of the therapies described herein to a subject results in editing efficiency of, or efficiency of indel formation in, the PLN gene comprising a deletion of Arg14 of at least or about 70%. In some embodiments, the administration of the therapies described herein to a subject results in editing efficiency of, or efficiency of indel formation in, the PLN gene comprising a deletion of Arg14 of at least or about 75%. In some embodiments, the administration of the therapies described herein to a subject results in editing efficiency of, or efficiency of indel formation in, the PLN gene comprising a deletion of Arg14 of at least or about 80%. In some embodiments, the administration of the therapies described herein to a subject results in editing efficiency of, or efficiency of indel formation in, the PLN gene comprising a deletion of Arg14 of at least or about 85%. In some embodiments, the administration of the therapies described herein to a subject results in editing efficiency of, or efficiency of indel formation in, the PLN gene comprising a deletion of Arg14 of at least or about 90%. In some embodiments, the administration of the therapies described herein to a subject results in editing efficiency of, or efficiency of indel formation in, the PLN gene comprising a deletion of Arg14 of at least or about 95%.

In some embodiments, the administration of the therapies described herein to a subject does not, or substantially does not, edit or form indels in the wild-type PLN gene (in vivo).

In some embodiments, the administration of the therapies described herein to a subject restores or improves cardiac function in the subject. In some embodiments, the administration of the therapies described herein to a subject restores or improves contractile function of the heart in the subject.

In some embodiments, the administration of the therapies described herein to a subject improves ejection fraction in the subject. In some embodiments, the administration of the therapies described herein to a subject increases ejection fraction in the subject.

In some embodiments, the administration of the therapies described herein to a subject reduces left ventricular hypertrophy, left ventricular mass and/or left ventricular wall thickness in the subject. In some embodiments, the administration of the therapies described herein to a subject improves left ventricular relaxation and/or left ventricular filling pressure in the subject.

### Ejection Fraction

A decrease in ejection fraction (%) is associated with the PLN-R14Del mutation.

In some embodiments, the methods of the disclosure increase or prevent a decrease in the ejection fraction (%) in a subject with a PLN-R14Del mutation. In some embodiments, the methods of the disclosure increase the ejection fraction by at least at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, at least 150%, or at least 200% in a subject with a PLN-R14Del mutation.

### Left Ventricle Internal Dimension (LVID)

An increase in LVID is associated with the PLN-R14Del mutation.

In some embodiments, the methods of the disclosure decrease or prevent an increase in LVID (mm) in a subject with a PLN-R14Del mutation. In some embodiments, the methods of the disclosure decrease or prevent an increase in LVID (mm) by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% in a subject with a PLN-R14Del mutation.

### Left Ventricle (LV) Mass

An increase in LV mass is associated with the PLN-R14Del mutation.

In some embodiments, the methods of the disclosure decrease or prevent an increase in LV mass (as measured in mg/g of body weight) in a subject with a PLN-R14Del mutation. In some embodiments, the methods of the disclosure decrease or prevent an increase in LV mass (as measured in mg/g of body weight) by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% in a subject with a PLN-R14Del mutation.

### Stroke Volume

A decrease in stroke volume is associated with the PLN-R14Del mutation.

In some embodiments, the methods of the disclosure increase or prevent a decrease in stroke volume in a subject with a PLN-R14Del mutation. In some embodiments, the methods of the disclosure increase or prevent a decrease in stroke volume by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% in a subject with a PLN-R14Del mutation.

### R Amplitude

A decrease in R Amplitude (mV) is associated with the PLN-R14Del mutation.

In some embodiments, the methods of the disclosure increase or prevent a decrease in R Amplitude (mV) in a subject with a PLN-R14Del mutation. In some embodiments, the methods of the disclosure increase or prevent a decrease in R Amplitude (mV) by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% in a subject with a PLN-R14Del mutation.

### O Wave, R Wave, and S Wave (ORS) Interval

An increase in the QRS interval is associated with the PLN-R14Del mutation.

In some embodiments, the methods of the disclosure decrease or prevent an increase in the QRS interval in a subject with a PLN-R14Del mutation. In some embodiments, the methods of the disclosure decrease or prevent an increase in the QRS interval by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% in a subject with a PLN-R14Del mutation.

### Mortality

A increase in mortality over time is associated with the PLN-R14Del mutation.

In some embodiments, the methods of the disclosure increase life span or prevent an increase mortality over time in a subject with a PLN-R14Del mutation. In some embodiments, the methods of the disclosure increase life span or prevents an increase in mortality in a subject with a PLN-R14Del mutation.

### EXAMPLES

### Example 1: Novel Expression Cassettes

The purpose of this study was to conduct an in vitro and in vivo experiment to identify a gRNA that specifically modifies the PLN R14del pathogenic allele and not the wild-type (WT) PLN allele. The goal was to provide evidence that a gene therapy approach using CRISPR-Cas9 technology could be used to correct the mutation in the pathogenic allele while leaving the WT allele unaltered. This approach could potentially treat or cure diseases associated with the PLN R14del mutation while minimizing the risk of unintended side effects.

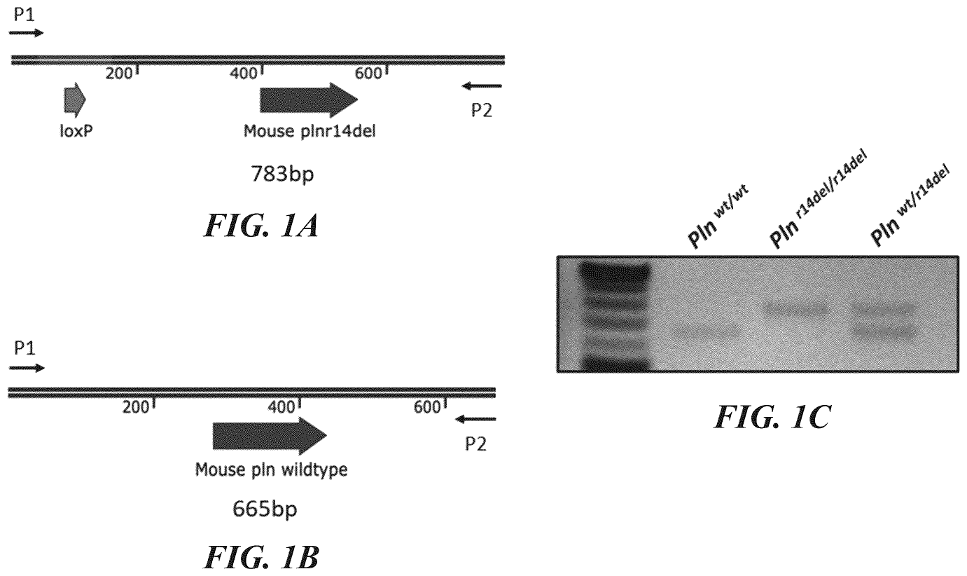

A PCR assay was developed to distinguish between the PLN R14del allele and the WT allele based on detection of different-sized PCR products (WT allele=665 bp; R14del allele=783 bp). FIG. 1A and FIG. 1B show the methods used to identify the different genotypes. To demonstrate the utility of the assay, PCR amplification was performed on DNA samples from mice with different genotypes. FIG. 1C shows an agarose gel that displays the results of the PCR amplification for WT/WT, WT/R14del, and R14del/R14del genotypes. This image shows that the PCR assay was able to correctly identify the genotypes of the mice based on the size of the PCR products generated by the PLN alleles.

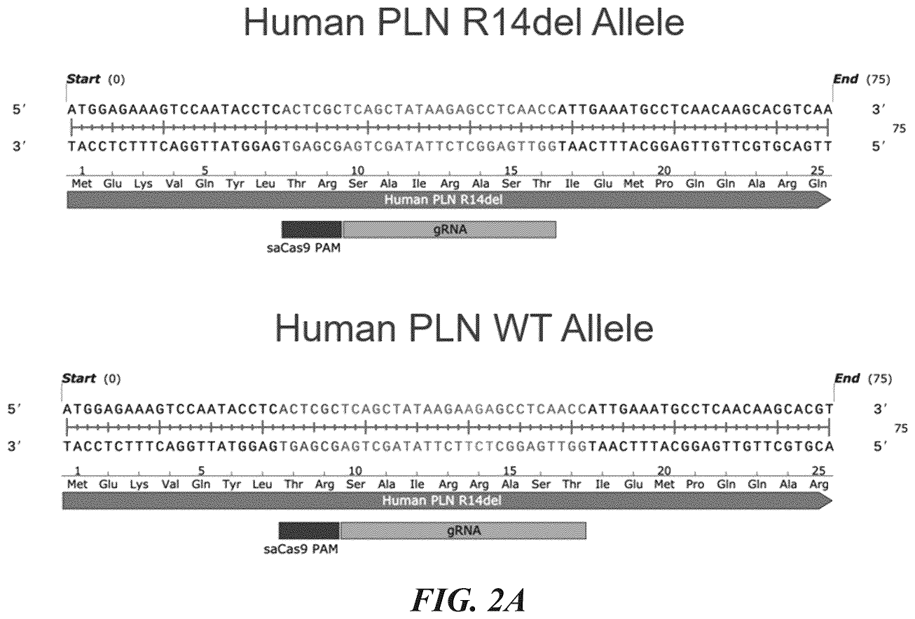

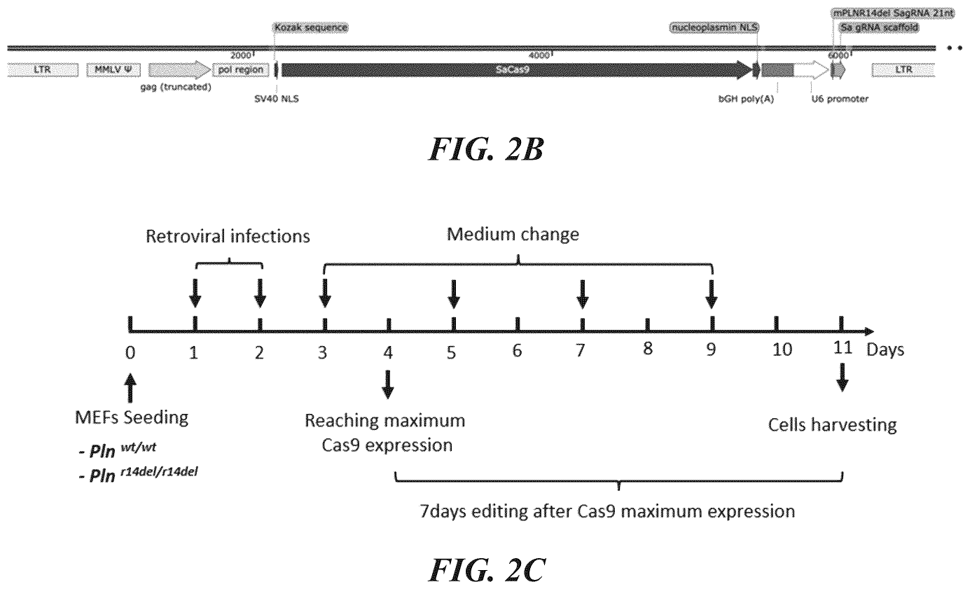

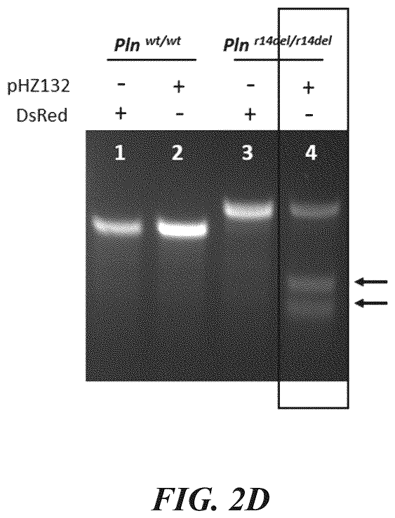

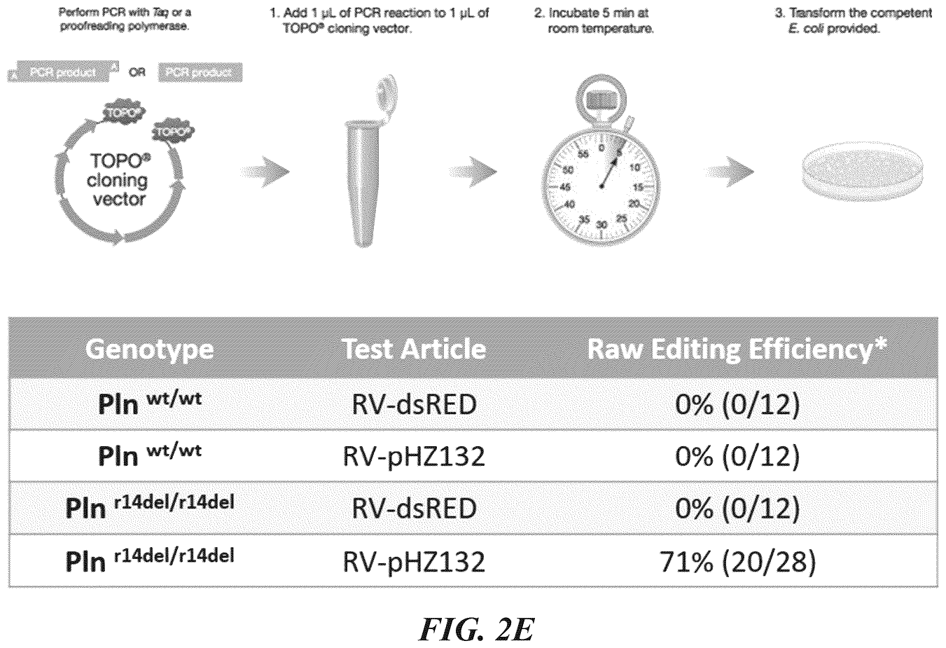

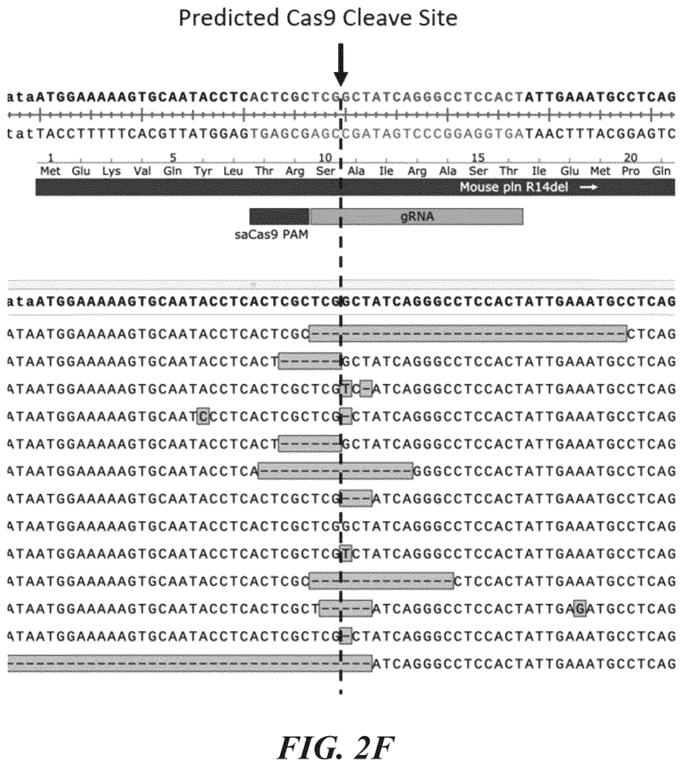

A guide RNA (gRNA) was designed to specifically target the PLN R14del allele. FIG. 2A shows a comparison of gRNA sequences for the WT and PLN R14del alleles. The gRNA was then cloned into a retroviral expression cassette that contained Cas9 from Staphylococcus aureus (SaCas9), which was named pHZ132 (FIG. 2B; FIG. 14; SEQ ID NO: 37). To test the efficiency and specificity of the gRNA for the PLN R14del allele, mouse embryonic fibroblasts (MEFs) with either the WT or R14del/R14del genotype were seeded and transduced with the pHZ132 retrovirus (RV-pHZ132) or a DsRed control retrovirus (RV-DsRed) on days 1 and 2 post-seeding. On day 11, the cells were harvested for subsequent analysis (FIG. 2C). DNA was harvested from both cell populations, and a PCR assay was performed to amplify the PLN locus, as described above. T7 Endonuclease I (T7E1) was then used to cleave non-perfectly matched DNA in a qualitative assay to determine the gene editing outcome. As shown in FIG. 2D, only R14del/R14del MEFs treated with RV-pHZ132 yielded lower bands, suggesting that non-perfectly matched DNA was generated. TA cloning and sequencing of the PCR products showed that the R14del/R14del MEFs treated with RV-pHZ132 exhibited 71% editing, whereas the other treatment groups did not yield any editing (FIG. 2E). Sequencing alignments of the RV-pHZ312-treated R14del/R14del MEFs revealed indel modifications in the PLN R14del allele (FIG. 2F). Overall, these results indicate that the gRNA designed to specifically target the PLN R14del allele was effective and specific in inducing gene editing in the R14del/R14del MEFs.

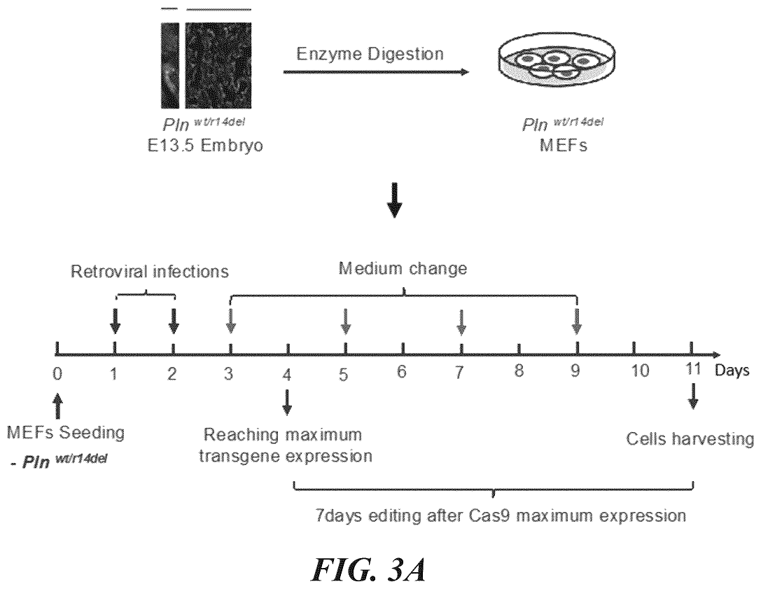

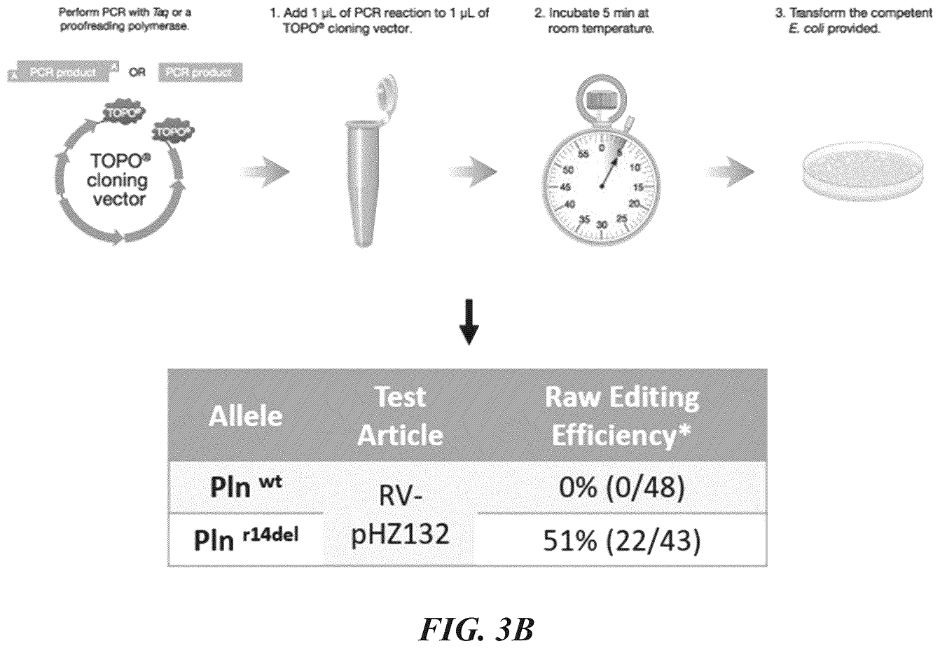

A similar experiment was conducted using WT/R14del MEFs (FIG. 3A). The WT allele did not yield any edited DNA whereas the R14del allele was edited in 51% of clones (FIG. 3B), demonstrating that the PLN R14del allele can be specifically edited in heterozygous cells.

Following this successful demonstration of in vitro proof-of-concept using MEFs, an in vivo experiment was performed to further validate the approach using AAV in PLN WT/R14del mice (FIGS. 4A-4C).

To create an AAV gene editing therapy that specifically targets the PLN R14del allele, the above gRNA was cloned into an AAV saCas9 cassette. In this cassette, saCas9 was placed under a human cardiac troponin T promoter (cTnT promoter) with the bovine growth hormone polyadenylation (bGH-pA) signal, while the gRNA was placed under a human U6 promoter. Both saCas9 and gRNA were arranged in a head-to-tail orientation, just like in the pHZ132 retroviral vector. The resulting AAV cassette was named pHZ131 (FIG. 4A; FIG. 13; SEQ ID NO: 36).

To investigate the efficiency and specificity of AAV9-pHZ131 in editing the PLN WT/R14del allele in mice, three different doses of the vector were administered via retro-orbital injection to five-month-old PLN WT/R14del mice, and their hearts were harvested after four weeks (FIG. 4B). Following DNA extraction, the PCR assay of FIGS. 1A-1C and TA cloning and sequencing methods of FIGS. 2A-2C were performed. The results show that the AAV9-pHZ131 vector was able to edit the PLN R14del allele in a dose-dependent manner, with the highest concentration of AAV resulting in 24% of sequenced clones being edited (FIG. 4C). Due to the use of a cardiac-specific promoter to drive saCas9 expression, only cardiomyocytes (CMs) were edited, which account for approximately 50% of total nucleus numbers in the heart. CM editing efficiency was adjusted by doubling the total editing percentage, resulting in approximately 50% of CMs being edited for the highest dose of AAV9-pHZ131. Consistent with the in vitro experiments shown in FIGS. 2A-2C and FIGS. 3A-3B, the PLN WT allele was not edited. These results demonstrate that AAV9-pHZ131 can efficiently and specifically edit the PLN R14del allele while leaving the PLN WT allele unedited.

### Example 2: Crispr Gene Therapy of PLN-R14Del in a PLN-R14^{Δ/Δ} Model

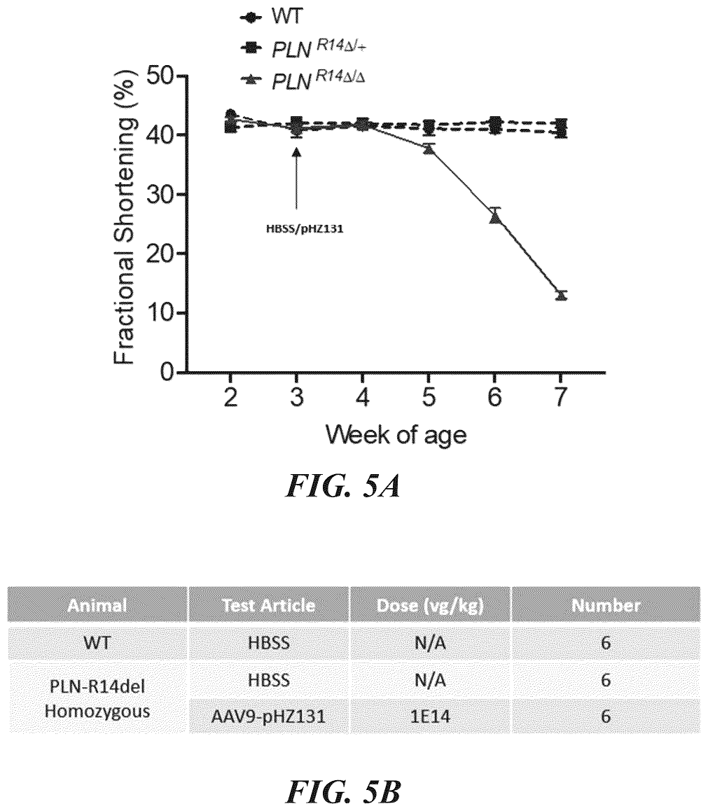

The purpose of this study was to evaluate the ability of AAV9-pHZ131 gene therapy to ameliorate the symptoms of cardiomyopathy in mice with the homozygous PLN R14del pathogenic allele. This allele has been linked to cardiomyopathy in mice, as well as to similar histopathological abnormalities and premature mortality in humans. As shown in FIG. 5A, fractional shortening (a marker of cardiac function) exhibited a rapid decline between 4 to 7 weeks of age in the PLNR14del homozygous mouse model.

In this study, three-week-old homozygous PLNR14del mice were injected via retro-orbital injection with either a vehicle control (HBSS) or AAV9-pHZ131 at a dose of 1E14 vg/kg. Six mice were included in each group (FIG. 5B). Cardiac function was assessed by echocardiography.

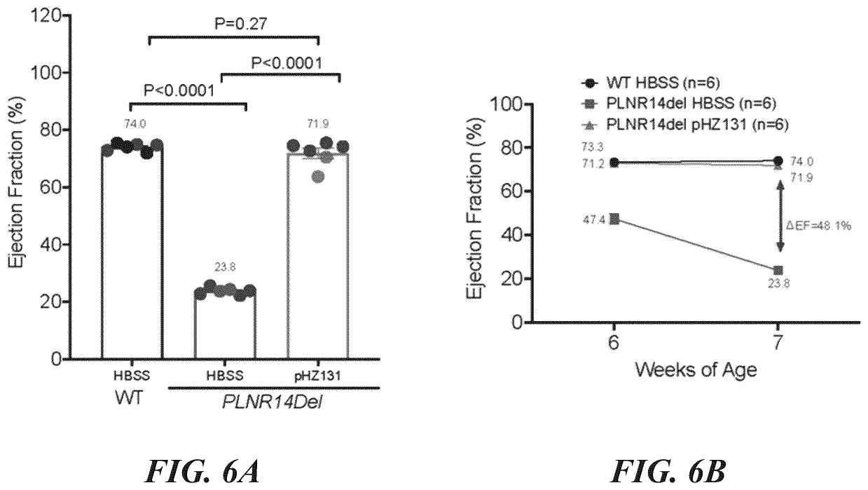

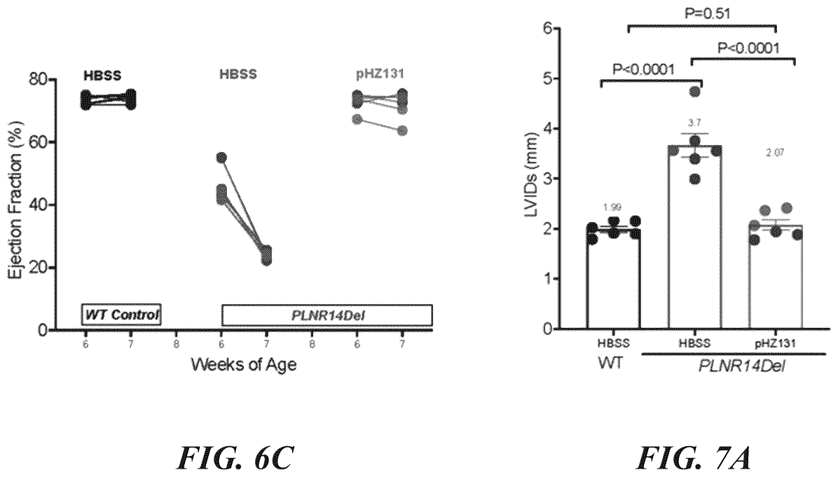

PLNR14del-associated cardiomyopathy is characterized by reduced cardiac contractility. To evaluate the efficacy of AAV9-pHZ131 in improving heart contractility in homozygous PLNR14del mice, ejection fractions were measured in mice aged 6 or 7 weeks, three or four weeks after AAV administration. Ejection fraction was significantly improved among PLNR14del mice treated with AAV9-pHZ131, compared to those treated with vehicle control (p<0.0001, FIG. 6A). Treatment of PLRN14del mice with AAV9-pHZ131 increased ejection fraction to a level that was not significantly different from that of wildtype mice (p=0.27, FIG. 6A). Ejection fraction was higher at both 6 and 7 weeks in PLNR14delHBSS mice treated with AAV9-pHZ131 compared to those treated with vector control (FIGS. 6B-6C); at 7 weeks, ejection fraction was 48.1% higher in the AAV9-pHZ131-treated group (FIG. 6B).

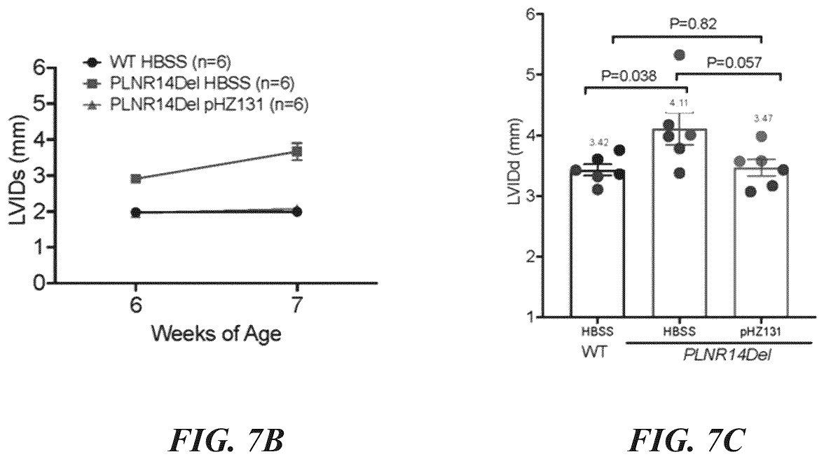

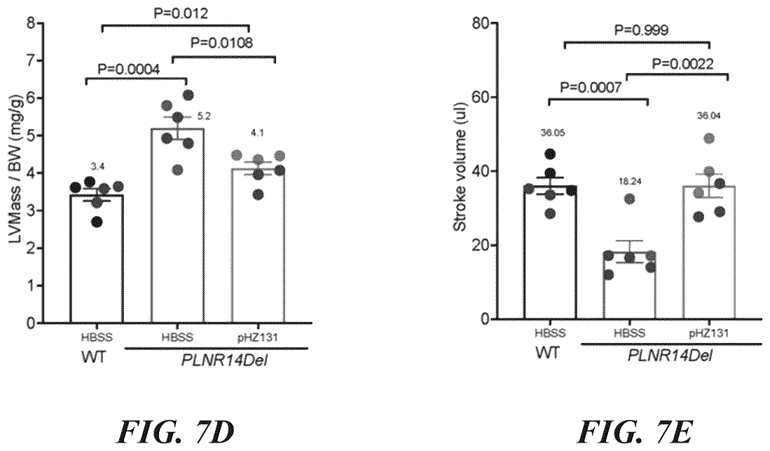

In addition to a reduction in ejection fraction, PLNR14del-associated cardiomyopathy is characterized by several other notable features, including an increase in left ventricular internal diameter end diastole (LVIDd), left ventricular internal diameter end systole (LVIDs), and left ventricular mass (LVMass), as well as a decrease in stroke volume. In PLRN14del mice treated with AAV9-pHZ131, LVIDs and LVMass were significantly reduced (p<0.001, FIG. 7A and p=0.0108, FIG. 7D, respectively) compared with PLRN14del mice treated with vector control. LVIDs was lower at both 6 and 7 weeks in PLNR14delHBSS mice treated with AAV9-pHZ131, compared to those treated with vector control (FIG. 7B). LVIDd showed a trend toward a decrease in AAV9-pHZ131-treated mice (p=0.057, FIG. 7C), and stroke volume was increased (p=0.0022, FIG. 7E) compared with PLRN14del mice treated with vector control. Treatment of PLRN14del mice with AAV9-pHZ131 improved LVIDs, LVMass, and stroke volume to a level that was not significantly different from that of wildtype mice (p=0.51, FIG. 7A; p=0.82, FIG. 7D; and p=0.999, FIG. 7E, respectively).

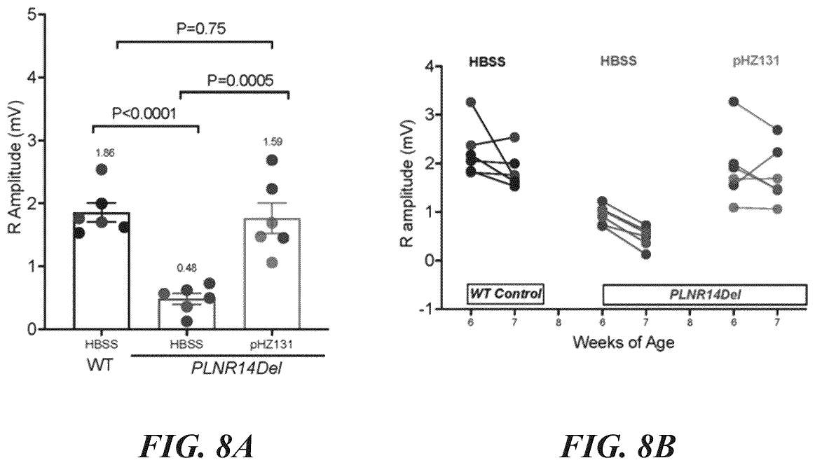

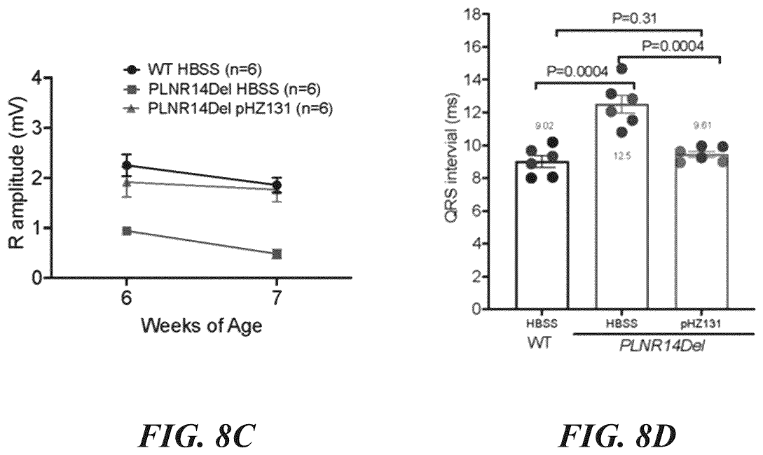

To assess the ability of AAV9-pHZ131 to preserve ECG parameters, the R amplitude and QRS interval were measured. The results showed a significant increase in R amplitude (p=0.0005, FIG. 8A) and a significant decrease in QRS interval (p=0.0004, FIG. 8D) in PLNR14del mice treated with AAV9-pHZ131 versus those treated with vector control. Among the AAV9-pHZ131-treated mice, both R amplitude and QRS interval were restored to levels comparable to those of wildtype mice. R amplitude was higher at both 6 to 7 weeks in PLNR14delHBSS mice treated with AAV9-pHZ131, compared with those treated with vector control (FIGS. 8B-8C).

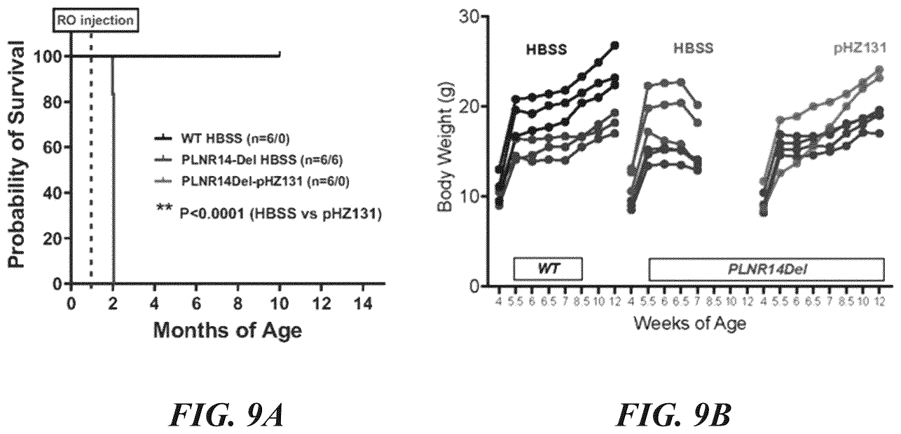

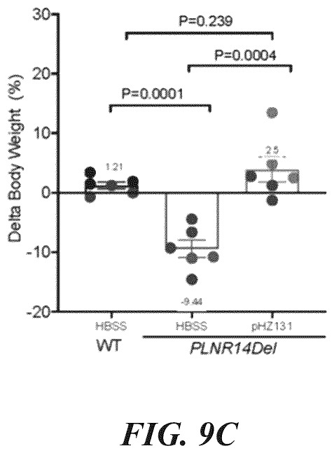

In order to investigate the impact of AAV9-pHZ131 on mortality and body weight, the survival and body weight of the study subjects were assessed over a period of 8 months. All WT mice and all PLN R14del mice treated with pHZ131 survived for the entire duration of the study, with survival rates of 6/6 and 6/6, respectively. Conversely, the survival rate of PLN R14del mice treated with HBSS significantly declined by 100% within two months, with a survival rate of 6/0 (FIG. 9A). Body weight was monitored between weeks 4 and 7 (FIG. 9B). The change in body weight between weeks 6.5 and 7 was not statistically significant between the WT controls and pHZ131-treated PLN R14del mice (p=0.239). However, the body weight of pHZ131-treated PLN R14del mice was found to be significantly higher than that of HBSS-treated controls (p=0.0004, FIG. 9C).

These findings are surprising and suggest that AAV9-pHZ131 is effective in enhancing heart function and survival in PLN R14del mice. The observation of this effect in mice provides evidence that this gene therapy can effectively treat existing cardiomyopathy, as well as prevent its development.

### Example 3: Dose Ranging Study Using PLN-R144A Model for Crispr Gene Therapy of PLN-R14Del

The purpose of this study was to determine the most optimal dose of AAV9-pHZ131 gene therapy for improving symptoms of cardiomyopathy in mice with the homozygous PLN R14del pathogenic allele.

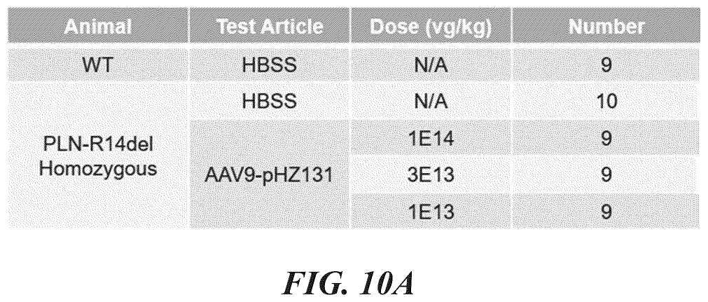

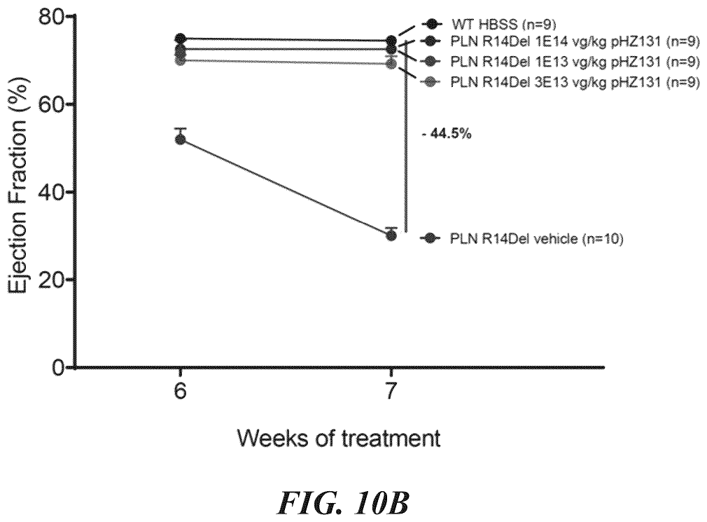

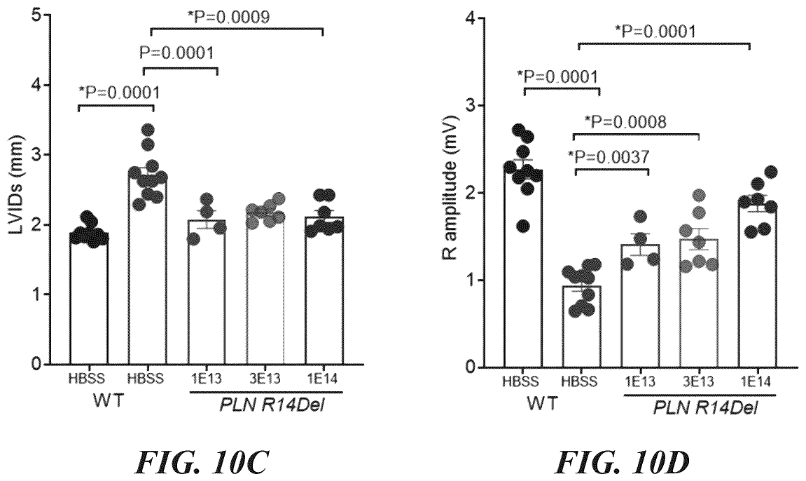

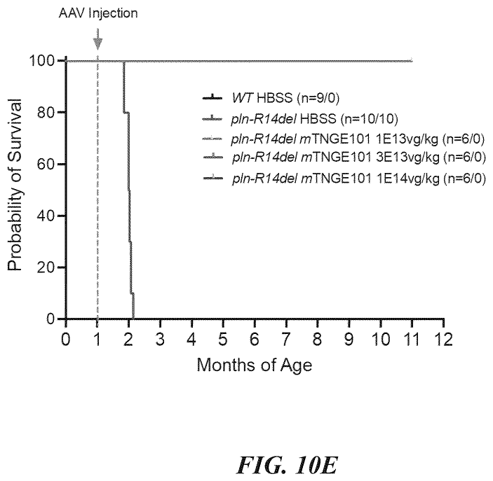

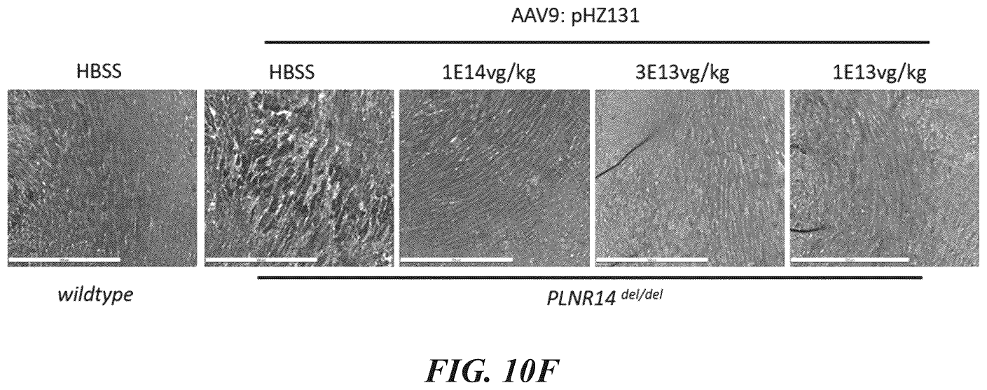

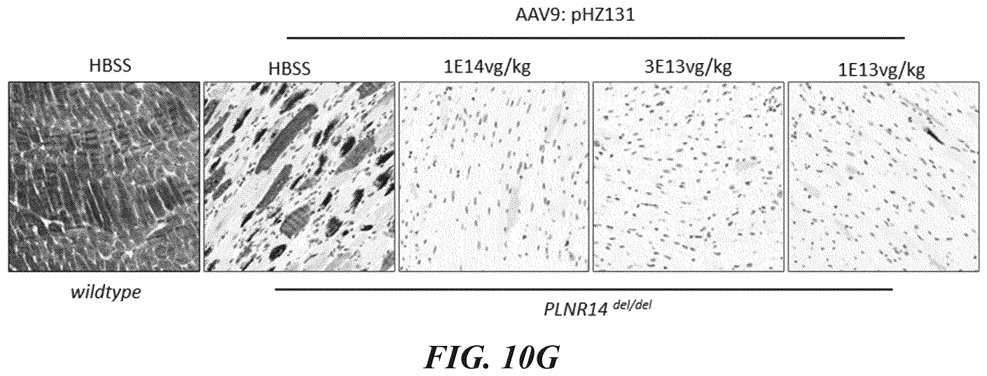

Groups of nine to ten three-week-old homozygous PLN R14del mice were administered retro-orbital injections with either a vehicle control (HBSS) or AAV9-pHZ131 at three different doses: 1E14 vg/kg, 3E13 vg/kg, and 1E13 vg/kg. Additionally, a control group of nine healthy wildtype mice was included (FIG. 10A). Cardiac function was evaluated in mice aged 6 to 7 weeks, approximately three to four weeks after AAV administration. Following this, a small group of mice (N=2-3) were selected for expression and histology analysis.

To assess the effectiveness of varying doses of AAV9-pHZ131 in enhancing heart contractility in homozygous PLNR14del mice, Ejection Fractions were measured in mice aged 6 to 7 weeks, approximately three to four weeks after AAV administration. Strikingly, the lowest dose (1E13 vg/kg) of AAV9-pHZ131 improved the ejection fraction in AAV9-pHZ131 treated PLN R14del mice by 44.5% compared to vector control (FIG. 10B). Ejection fraction among mice receiving this dose of AAV9-pHZ131 was improved to a level comparable to that of wildtype mice.

Left ventricular internal diameter end diastole (LVIDd) was also significantly improved in PLN R14del mice treated with 1E13 vg/kg of AAV9-pHZ131 compared to vector control (p=0.0001, FIG. 10C). LVIDd among mice receiving this dose of AAV9-pHZ131 was decreased almost to the level of wildtype mice. The lowest dose (1E13 vg/kg) of AAV9-pHZ131 also resulted in a significant increase in R amplitude, indicating the preservation of ECG parameters (p=0.0037, FIG. 10D).

To examine the effect of various doses of AAV9-pHZ131 on mortality, the survival of the study subjects (N=9 or 10 per group) was evaluated over a period of 11 months. All PLN R14del mice treated with different doses of pHZ131 survived throughout the study. In contrast, the survival rate of PLN R14del mice treated with HBSS significantly declined by 100% within two months (FIG. 10E).

PLNR14del-associated cardiomyopathy is distinguished by cardiac fibrosis and PLN protein aggregation. To investigate this, Trichrome and DAP staining were performed on heart tissue from 2-month-old mice, 5 weeks post-viral administration. The results showed that all PLN R14del mice treated with different doses of pHZ131 had a significant reduction in both cardiac fibrosis (FIG. 10F) and PLN protein aggregation (FIG. 10G) compared to vehicle control (HBSS).

These findings are surprising and suggest that AAV9-pHZ131 is effective in improving heart function and survival in PLN R14del mice even at a very low dose (1E13 vg/kg).

### Example 4: Gene Editing Human PLN-R14^{Δ/Δ} IPSC-CMS

Due to the difference in DNA sequence between the PLNR14del locus in mice and humans (FIG. 11A), the gRNA used for the human PLNR14del locus is different from the gRNA used in mice, as described in Examples 1-3. It is crucial to test the efficiency of the gene therapy vector with the human gRNA and compare it to the gene therapy vector with the mouse gRNA.

The purpose of this study was to develop an in vitro model using iPSC-derived cardiomyocytes (iPS-CMs) and evaluate the efficacy of gene therapy vector with the human gRNA for editing PLNR14del.

Two isogenic cell lines were generated from a wildtype human iPSC line using CRISPR editing: a PLNR14del homozygous iPSC line and a PLN knockout iPSC line (FIG. 11B). Using an in-house established protocol, PLNR14del homozygote iPSCs were successfully differentiated into cardiomyocytes (PLNR14del iPSC-CMs) (FIG. 11C).

The mouse PLNR14del gRNA in the pHZ131 cassette was replaced with the human gRNA to create a new cassette, pHZ128 (FIG. 11D; FIG. 12; SEQ ID NO: 35).

To evaluate the editing efficiency of pHZ128 for the human PLNR14del locus, pHZ128 was packaged with AAVCR9-01 capsid, which preferentially infects iPSC-CMs. AAVCR9-1-pHZ128 virus was used to infect wildtype and PLNR14del iPSC-CMs one week after seeding, at two multiplicities of infection (MOIs, 100K and 300K). Cells were harvested after 3 weeks of culture (two weeks after infection with AAVCR9-1-pHZ128), which is seven days after peak Cas9 expression is reached (data not shown). Only the PLNR14del locus was edited by AAVCR9-1-pHZ128 (˜65% editing), whereas the PLN wildtype group did not show any editing (FIG. 11E).

These results show that AAV-pHZ128 can efficiently and specifically edit the PLN R14del allele while leaving the PLN WT allele unedited.

### Example 5: Illustrative AAV and MMLV Vectors

Vectors comprising expression cassettes were constructed for experiments in mice and for clinical use. A plasmid map of pHZ128_cTnT-saCas9-U6-hPLNR14del SagRNA21nt, which comprises the human hPLNR14del SagRNA 21nt and SaCas9 in an AAV expression cassette, is shown in FIG. 12. A plasmid map of pHZ131_cTnT-saCas9-U6-hPLNR14del SagRNA21nt, which comprises the mouse hPLNR14del SagRNA 21nt and SaCas9 in an AAV expression cassette, is shown in FIG. 13. A plasmid map of pHZ132_cTnT-saCas9-U6-hPLNR14del SagRNA21nt, which comprises the mouse hPLNR14del SagRNA 21nt and SaCas9 in an MMLV expression cassette, is shown in FIG. 14.

### Example 6: Self-Inactivating Cas Vectors for Crispr Gene Therapy of PLN-R14Del

The purpose of this study was to generate a construct comprising a self-inactivating Cas and a gRNA that specifically targets the PLN R14del pathogenic allele to be used in CRISPR-Cas9 gene therapies. Long-term or constitutive expression of Cas proteins as part of a gene therapy raises concerns about off-target effects, potential immune responses, and unintended genomic modifications, as Cas9 is a prokaryotic protein and it will be highly immunogenic in human. Thus, limiting Cas expression after the editing of the target gene is complete may offer several advantages, including increased safety profile, reduced immunogenicity by Cas proteins, and/or reduced off-target effects.

Protospacer adjacent motifs, or PAM sequences, are pivotal in determining the efficiency of CRISPR-Cas genome editing. Optimal PAMs allow for a swifter initiation of the editing process compared to non-optimal PAMs. However, non-optimal PAMs, though still compatible with a given Cas enzyme, can impede the editing process. This is because the Cas enzyme often struggles to recognize and bind to the target DNA with non-optimal PAMs. We leveraged these distinctions between optimal and non-optimal PAMs to devise a Cas9 self-inactivation strategy. The aim was to eliminate Cas protein expression while preserving the efficacy and efficiency of human gene editing. The schematic representation of this design is shown in FIG. 15A. As an illustration, we utilized saCas9 and selected a gRNA target site with the optimal PAM (NNGRRT) in the human genome, where N is any nucleotide, and R is guanine or adenine. We then incorporated a self-inactivation site with less optimal PAMs (NNGRRC, NNGRRG, or NNGRRA). Because optimal PAMs enable a quicker initiation of the editing process compared to non-optimal PAMs, the human genome undergoes editing before Cas self-inactivation occurs. This design should ensure that both the efficacy and efficiency of human editing are maintained.

To generate a self-inactivating Cas construct for AAV-based gene editing therapy, the expression cassette described in Examples 1 and 4 (pHZ131 for mouse and pHZ128 for human)—the one that includes a saCas9 driven by a human cTnT promoter, and a gRNA specifically targeting the PLN R14del allele driven by a human U6 promoter, arranged in a head-to-tail orientation—was further modified to include a self-inactivation site in placed within the 5′ end of the saCas9-coding sequence, after the start codon “ATG” (FIG. 15A;

FIG. 16). The self-inactivation site contained a less optimal PAM sequence of saCas9 (e.g., NNGRRC, NNGRRG, or NNGRRA) and a copy of the same gRNA sequence as the gRNA used in the construct that is specific to PLN R14del. Because the genomic PLN R14del target site of the gRNA is designed to be adjacent to an optimal PAM sequence (e.g., in the case of saCas9, it is NNGRRT), after the saCas9 is expressed, the cleaving of the genomic PLN R14del target site by the saCas9 would be more effective and would thus take place first. The saCas9 will also cut the self-inactivation site directed by the same gRNA, but it would be less effective and take place later due to the less optimal PAM sequence (FIGS. 15A-15B). Accordingly, the self-inactivating Cas construct was designed to achieve the sequential editing of (1) the genomic target site, thereby to edit the target gene; and (2) the Cas expression cassette, thereby to terminate the expression of the Cas protein after the genomic gene editing is complete.

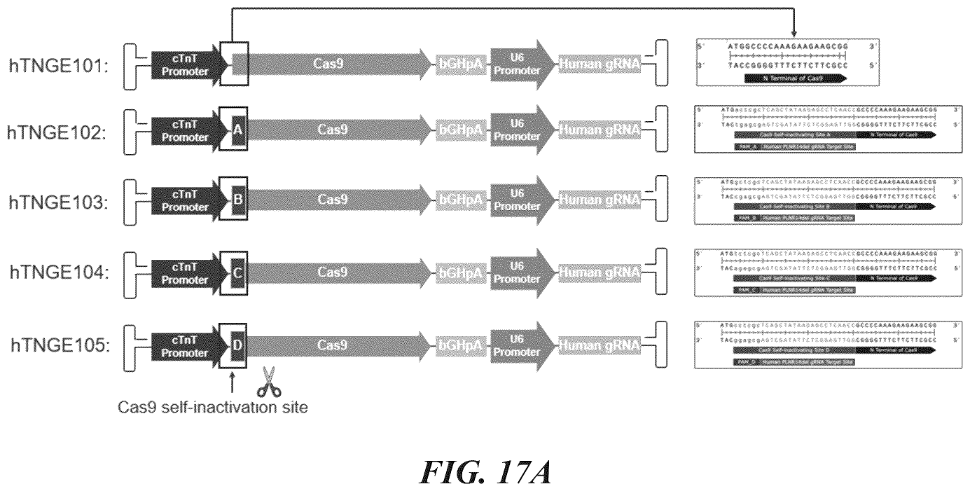

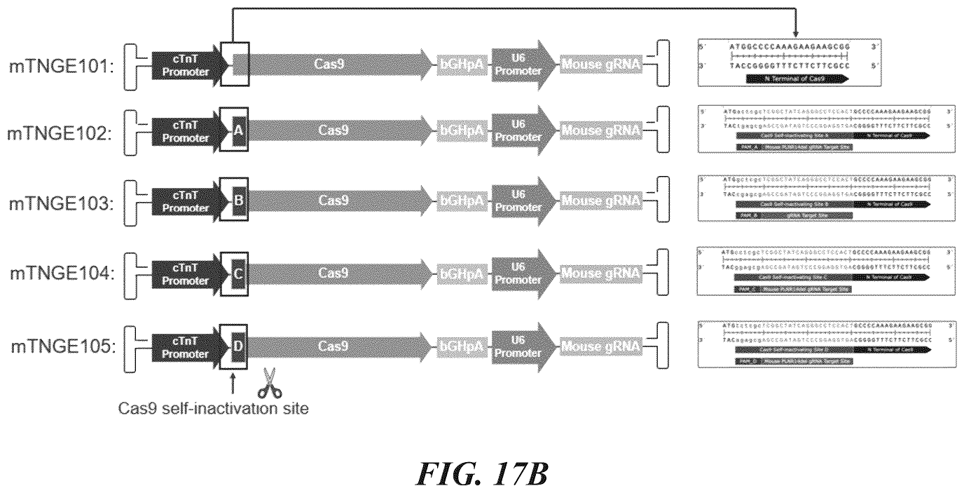

To test this design, a set of constructs were designed for the human (h) and mouse (m) PLN gene, respectively (FIGS. 17A-17B). Within each set, there are a control, constitutive saCas9 construct (TNGE101) and a series of self-inactivating saCas9 constructs (TNGE102-TNGE105) containing a self-inactivation site with optimal (TNGE102, gcgagt) and different less optimal (TNGE103, gcgagc; TNGE104, gcgaga; TNGE105, gcgagg) PAM sequences for saCas9. The mouse version of the TNGE101 construct (mTNGE101) is equivalent and identical to pHZ131 (FIG. 13; SEQ ID NO: 36) described in Example 1; the human version of the TNGE101 construct (hTNGE101) is equivalent and identical to pHZ128 (FIG. 12; SEQ ID NO: 35) described in Example 4. The sequences of the human expression cassettes of hTNGE102-hTNGE105 (from the 5′ ITR to the 3′ ITR, exclusive of the ITRs) are shown in SEQ ID NOs: 128-131, respectively. The sequences of the mouse expression cassettes of mTNGE102-mTNGE105 (from the 5′ ITR to the 3′ ITR, exclusive of the ITRs) are shown in SEQ ID NOs: 132-135, respectively. The constitutive Cas construct was predicted to have long-lasting expression of the Cas protein after introduction into the organism, while the self-inactivating constructs were predicted to have transient Cas protein expression.

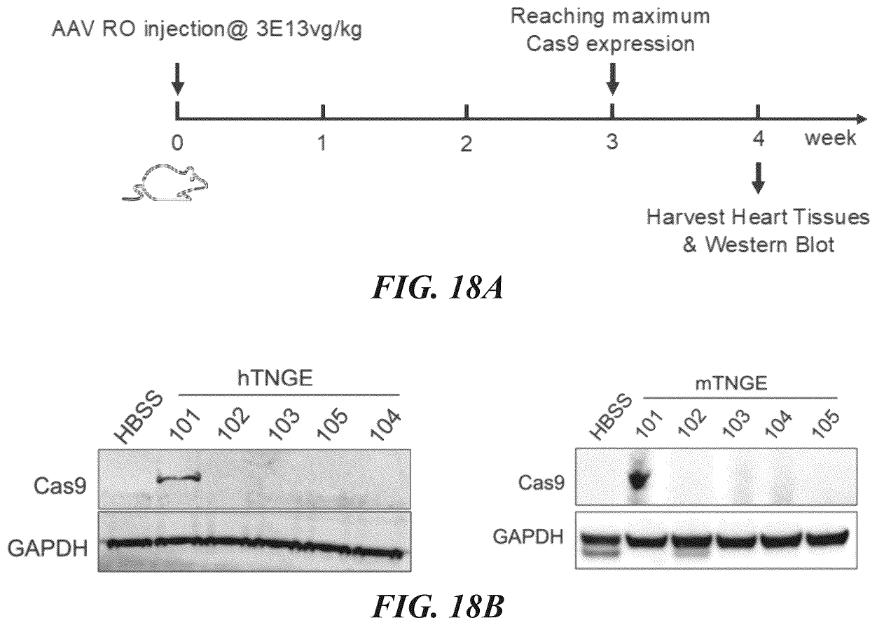

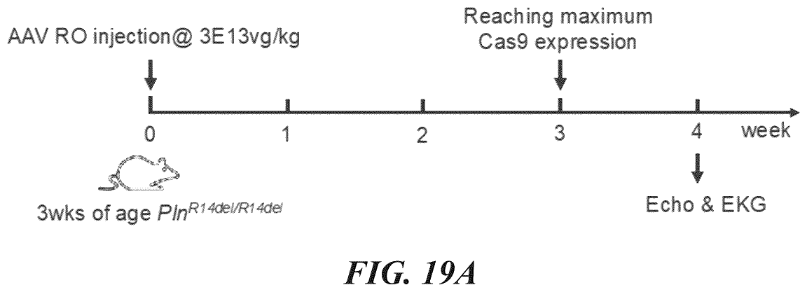

As shown in FIGS. 18A-18B, within four weeks after injection of the AAV vectors into mice, all four self-inactivating designs successfully reduced Cas9 expression, alleviating safety concerns related to long-term expression of Cas9, while the constitutive design had prolonged Cas9 expression.

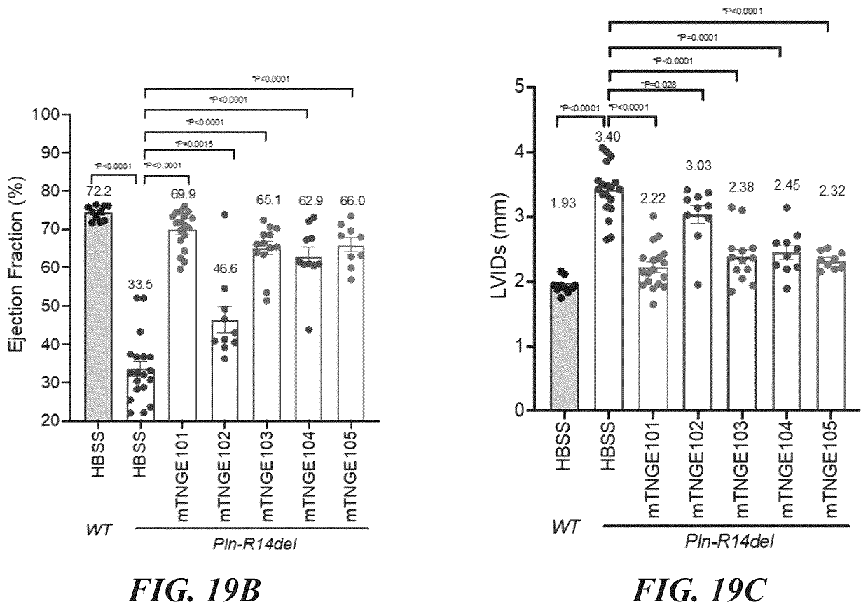

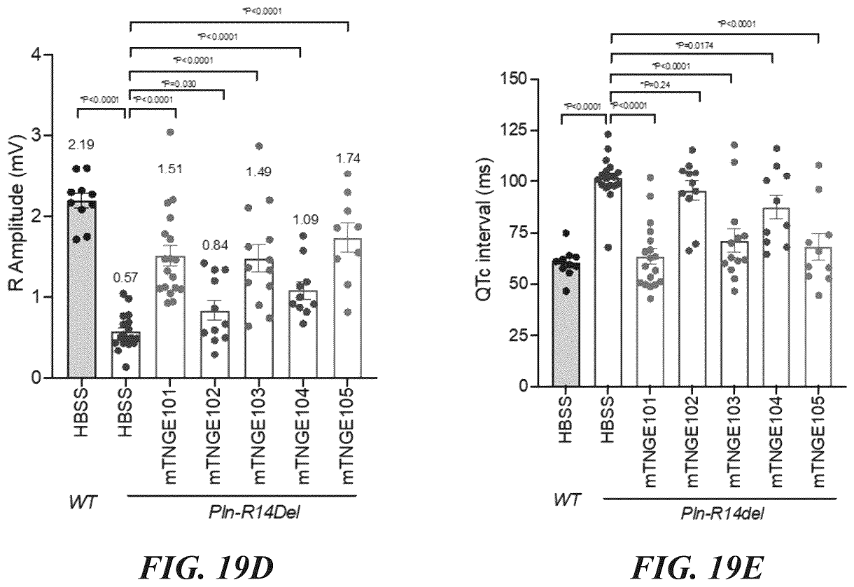

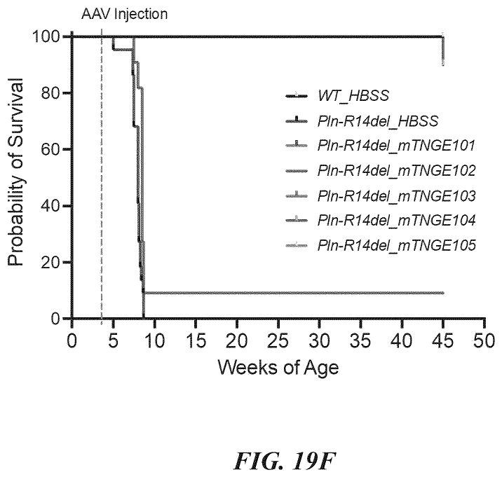

Next, the murine constructs were tested for their gene editing efficacy in the PLNR14del mouse model as described in Example 2. Three-week-old PLN R14del/R14del homozygous mice were injected via retro-orbital injection with either a vehicle control (HBSS), the constitutive Cas construct (mTNGE101), or each of the self-inactivating Cas constructs (mTNGE102-mTNGE105) at a dose of 3E13 vg/kg. Four weeks post injection, cardiac function was assessed by echocardiography and electrocardiogram. As shown in FIGS. 19B-19E, Cas expression from the constitutive construct significant improved PLNR14del-associated cardiomyopathy symptoms in homozygous PLNR14del mice, to a level similar to that of wildtype mice, compared to control-treated mice as measured by ejection fraction (p<0.0001, FIG. 19B), LVIDs (p<0.0001, FIG. 19C), R amplitude (p<0.0001, FIG. 19D), and QTc interval (p<0.0001, FIG. 19E), consistent with previous observations. Similarly, self-inactivating Cas constructs with less optimal PAM sequences (mTNGE103-mTNGE105) also significantly rescued PLNR14del-associated phenotypes in those measurements, mostly comparable to the effects of the constitutive Cas construct despite the transient Cas expression, suggesting that a lower level of transiently expressed Cas9 can be efficacious (FIGS. 18B, 19B-19E). On the other hand, the self-inactivating Cas construct with the optimal PAM sequence (mTNGE102), potentially due to its cleaving and self-activating of the Cas construct in competition with its genomic gene editing, resulted in less effective (yet in most measurements still significant compared to control treatment) reversal of the cardiomyopathy phenotypes as measured by ejection fraction (p<0.05, FIG. 19B), LVIDs (p<0.05, FIG. 19C), R amplitude (p<0.05, FIG. 19D), and QTc interval (p=0.24, FIG. 19E).

Finally, the self-inactivating vectors with transient expression of Cas9 that showed good improvement in heart function were also tested in the survival assay as described in Example 2. As expected, the self-inactivating vectors, with the exception of mTNGE102 (which contains the optimal PAM sequence), resulted in high rates of survival throughout the duration of the study (with the most recent time point being 45 weeks post AAV injection) similar to constitutively expressed Cas9. Like mTNGE101, mTNGE103 and mTNGE105 exhibited 100% survival at 45 weeks post injection. mTNGE104 exhibited 90% survival at 45 weeks post injection (FIG. 19F). Conversely, the survival rate of control-treated PLN R14del mice declined by 100% within two months, and the less-effective mTNGE102 construct only prolonged survival by approximately 1 week compared to the HBSS control group (FIG. 19F). Although further monitoring might be necessary to fully comprehend the long-term effects and safety profile of these treatments, the findings here suggest that transient expression of Cas9 can be therapeutically beneficial and will likely lead to a safer product due to its reduction of immune response against transduced cells, risks of off-target gene editing, and the need for permanent immunosuppression.

### INCORPORATION BY REFERENCE

Various references such as patents, patent applications, and publications are cited herein, the disclosures of which are hereby incorporated herein by reference in their entireties. Also, all references mentioned herein are specifically incorporated by reference to disclose and describe the methods and/or materials in connection with which the publications are cited.

What is claimed is:

1. A vector for specifically targeting a phospholamban (PLN) gene comprising a deletion of Arg14 comprising:
(i) a first polynucleotide encoding a Cas endonuclease protein operably linked to a human troponin T promoter; and
(ii) a second polynucleotide encoding a guide RNA (gRNA) operably linked to an RNA expression-driving promoter, wherein the gRNA is complementary to a sequence of the PLN gene comprising a deletion of Arg14,
wherein the first polynucleotide and the second polynucleotide have a head-to-tail orientation.

2. The vector of claim 1, wherein the gRNA comprises at least 16 nucleotides with up to 3 mismatches of the nucleotide sequence ggttgaggctcttatagctga (SEQ ID NO: 1), optionally wherein the mismatch(es) is not in the sequence of ttatagctga (SEQ ID NO: 127) within SEQ ID NO: 1.

3. The vector of claim 2, wherein the gRNA comprises at least 16, 17, 18 or 19 nucleotides of the nucleotide sequence ggttgaggctcttatagctga (SEQ ID NO: 1).

4. The vector of claim 2, wherein the gRNA comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), and tggttgaggctcttatagctga (SEQ ID NO: 4).

5. The vector of claim 2, wherein the gRNA comprises the nucleotide sequence of ggttgaggctcttatagctga (SEQ ID NO: 1).

6. The vector of claim 2, wherein the gRNA comprises the nucleotide sequence of SEQ ID NO: 5, SEQ ID NO: 6, SEQ ID NO: 7, or SEQ ID NO: 8.

7. The vector of any one of claims 1-6, wherein the RNA expression-driving promoter is a U6 promoter.

8. The vector of claim 7, wherein the U6 promoter is a human U6 promoter having the nucleotide sequence of SEQ ID NO: 22.

9. The vector of any one of claims 1-8, wherein the Cas endonuclease protein is saCas9.

10. The vector of claim 9, wherein the saCas9 comprises a nucleotide sequence having at least 70%, 85%, 90%, 95% or 98% identity to SEQ ID NO: 10 and/or an amino acid sequence having at least 70%, 85%, 90%, 95% or 98% identity to SEQ ID NO: 11.

11. The vector of claim 9, wherein the saCas9 comprises the nucleotide sequence of SEQ ID NO: 10 and/or the amino acid sequence of SEQ ID NO: 11.

12. The vector of any one of claims 1-11, wherein the human troponin T promoter has a nucleotide sequence having at least 70%, 80%, 85%, 90%, 95% or 98% identity to SEQ ID NO: 12.

13. The vector of any one of claims 1-11, wherein the human troponin T promoter has the nucleotide sequence of SEQ ID NO: 12.

14. The vector of any one of claims 1-11, wherein the human troponin T promoter has a nucleotide sequence having at least 70%, 80%, 85%, 90%, 95% or 98% identity to SEQ ID NO: 13.

15. The vector of any one of claims 1-11, wherein the human troponin T promoter has the nucleotide sequence of SEQ ID NO: 13.

16. The vector of any one of claims 1-11, wherein the human troponin T promoter has a nucleotide sequence having at least 70%, 80%, 85%, 90%, 95%, 98% or 100% identity to SEQ ID NO: 14.

17. The vector of any one of claims 1-16, wherein the vector comprises the following 5′ to 3′ arrangement of elements: 5′-the human troponin T promoter-the first polynucleotide encoding the Cas endonuclease protein-the RNA expression-driving promoter-the second polynucleotide encoding the gRNA-3′.

18. The vector of claim 1-17, wherein the vector comprises the following 5′ to 3′ arrangement of elements: 5′-the human troponin T promoter-the first polynucleotide encoding saCas9-human U6 promoter-the second polynucleotide encoding the gRNA-3′.

19. The vector of any one of claims 1-18, wherein the vector further comprises a polyadenylation sequence.

20. The vector of claim 19, wherein the polyadenylation sequence is selected from a BGH polyadenylation sequence and a SV40 polyadenylation sequence.

21. The vector of claim 20, wherein the BGH polyadenylation sequence has at least 90%, 95%, 98% or 100% identity to SEQ ID NO: 29, and/or the SV40 polyadenylation sequence has at least 90%, 95%, 98% or 100% identity to SEQ ID NO: 30.

22. The vector of any one of claims 19-21, wherein the polyadenylation sequence is between the first polynucleotide encoding the Cas endonuclease protein and the RNA expression-driving promoter, or wherein the vector comprises the following 5′ to 3′ arrangement of elements: 5′-the human troponin T promoter-the first polynucleotide encoding the Cas endonuclease protein-the polyadenylation sequence-the RNA expression-driving promoter-the second polynucleotide encoding the gRNA-3′.

23. The vector of any one of claims 1-22, wherein the vector is a DNA-based vector, an mRNA-based vector, an adeno-associated virus-based vector, a retrovirus-based vector, or a lentivirus-based vector.

24. The vector of any one of claims 1-22, wherein the vector is an adeno-associated virus vector, a retroviral vector, or a lentiviral vector.

25. The vector of any one of claims 1-22, wherein the vector is an AAV9 vector.

26. The vector of any one of claims 1-22, wherein the vector is a modified adeno-associated virus vector.

27. The vector of claim 26, wherein the modified adeno-associated virus comprises any capsid protein described herein, optionally wherein the capsid protein is wild-type AAV9 capsid protein or a variant thereof.

28. The vector of any one of claims 1-27, which, when introduced into iPSC-derived cardiomyocytes, has an at least 20%, 25%, 30%, 35%, 40%, 45%, 50%, 55%, 60% or 70% editing efficiency of, or efficiency of indel formation in, the PLN gene comprising a deletion of Arg14.

29. The vector of claim 28, which, when introduced into iPSC-derived cardiomyocytes, has an at least 50% or at least 70% editing efficiency of, or efficiency of indel formation in, the PLN gene comprising a deletion of Arg14.

30. The vector of any one of claims 1-29, which, when introduced into iPSC-derived cardiomyocytes, does not, or substantially does not, edit or form indels in the wild-type PLN gene.

31. A guide RNA (gRNA) for specifically targeting a phospholamban (PLN) gene comprising a deletion of Arg14, wherein the gRNA comprising the nucleotide sequence of SEQ ID NO: 5.

32. A guide RNA (gRNA) for specifically targeting a phospholamban (PLN) gene comprising a deletion of Arg14, wherein the gRNA comprising the nucleotide sequence of SEQ ID NO: 6.

33. A guide RNA (gRNA) for specifically targeting a phospholamban (PLN) gene comprising a deletion of Arg14, wherein the gRNA comprising the nucleotide sequence of SEQ ID NO: 7.

34. A guide RNA (gRNA) for specifically targeting a phospholamban (PLN) gene comprising a deletion of Arg14, wherein the gRNA comprising the nucleotide sequence of SEQ ID NO: 8.

35. A gene editing composition or kit comprising (i) the gRNA of any one of claims 31-34, and (ii) a saCas9 endonuclease protein or a polynucleotide encoding the same, optionally wherein the polynucleotide encoding the saCas9 comprises the nucleotide sequence of SEQ ID NO: 10, and/or the saCas9 comprises the amino acid sequence of SEQ ID NO: 11.

36. A vector for specifically targeting a phospholamban (PLN) gene comprising a deletion of Arg14 comprising:
(i) a polynucleotide encoding a Cas endonuclease protein, operably linked to a protein expression-driving promoter; and
(ii) a polynucleotide encoding the guide RNA of any one of claims 31-34, operably linked to an RNA expression-driving promoter.

37. The vector of claim 36, wherein the Cas endonuclease protein is saCas9, the protein expression-driving promoter is a human troponin T promoter, and the RNA expression-driving promoter is a human U6 promoter.

38. The vector of claim 36 or 37, which, when introduced into iPSC-derived cardiomyocytes, has an at least 20%, 25%, 30%, 35%, 40%, 45%, 50%, 55%, 60% or 70% editing efficiency of, or efficiency of indel formation in, the PLN gene comprising a deletion of Arg14.

39. The vector of claim 38, which, when introduced into iPSC-derived cardiomyocytes, has an at least 50% or at least 70% editing efficiency of, or efficiency of indel formation in, the PLN gene comprising a deletion of Arg14.

40. The vector of any one of claims 36-39, which, when introduced into iPSC-derived cardiomyocytes, does not, or substantially does not, edit or form indels in the wild-type PLN gene.

41. A pharmaceutical composition comprising the vector of any one of claims 1-30 and 36-40.

42. A method of treating or preventing a disease or condition in a human with a deletion of Arg14 in a phospholamban (PLN) gene, comprising administering to the human a therapeutically effective amount of a composition or vector comprising a guide RNA (gRNA), wherein the gRNA comprises at least 16 nucleotides with up to 3 mismatches of the nucleotide sequence ggttgaggctcttatagctga (SEQ ID NO: 1), optionally wherein the mismatch(es) is not in the sequence of ttatagctga (SEQ ID NO: 127) within SEQ ID NO: 1.

43. The method of claim 42, wherein the gRNA comprises at least 16, 17, 18 or 19 nucleotides of the nucleotide sequence ggttgaggctcttatagctga (SEQ ID NO: 1).

44. The method of claim 42, wherein the gRNA comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), and tggttgaggctcttatagctga (SEQ ID NO: 4).

45. The method of claim 42, wherein the gRNA comprises the nucleotide sequence of ggttgaggctcttatagctga (SEQ ID NO: 1).

46. The method of claim 42, wherein the gRNA comprises the nucleotide sequence of SEQ ID NO: 5, SEQ ID NO: 6, SEQ ID NO: 7, or SEQ ID NO: 8.

47. The method of claim 42, wherein the gRNA comprises the nucleotide sequence of SEQ ID NO: 5.

48. The method of any one of claims 42-47, the method comprises administering the gRNA in the vector, and wherein the vector comprises:
(i) a first polynucleotide encoding a Cas endonuclease protein, operably linked to a protein expression-driving promoter; and
(ii) a second polynucleotide encoding the gRNA, operably linked to an RNA expression-driving promoter.

49. The method of claim 48, wherein the Cas endonuclease protein is saCas9.

50. The method of claim 49, wherein the saCas9 comprises a nucleotide sequence having at least 70%, 85%, 90%, 95% or 98% identity to SEQ ID NO: 10 and/or an amino acid sequence having at least 70%, 85%, 90%, 95% or 98% identity to SEQ ID NO: 11.

51. The method of claim 49, wherein the saCas9 comprises the nucleotide sequence of SEQ ID NO: 10 and/or the amino acid sequence of SEQ ID NO: 11.

52. The method of any one of claims 48-51, wherein the protein expression-driving promoter is a cardiac cell-specific or a cardiomyocyte-specific promoter.

53. The method of any one of claims 48-51, wherein the protein expression-driving promoter is a human troponin T promoter.

54. The method of claim 53, wherein the human troponin T promoter has a nucleotide sequence having at least 70%, 80%, 90%, 95% or 98% identity to SEQ ID NO: 12.

55. The method of claim 53, wherein the human troponin T promoter has the nucleotide sequence of SEQ ID NO: 12.

56. The method of claim 53, wherein the human troponin T promoter has a nucleotide sequence having at least 70%, 80%, 90%, 95% or 98% identity to SEQ ID NO: 13.

57. The method of claim 53, wherein the human troponin T promoter has the nucleotide sequence of SEQ ID NO: 13.

58. The method of claim 53, wherein the human troponin T promoter has a nucleotide sequence having at least 70%, 80%, 90%, 95%, 98% or 100% identity to the nucleotide sequence of SEQ ID NO: 14.

59. The method of any one of claims 48-58, wherein the RNA expression-driving promoter is a U6 promoter.

60. The method of claim 59, wherein the U6 promoter is a human U6 promoter having the nucleotide sequence of SEQ ID NO: 22.

61. The method of claim 48, wherein the Cas endonuclease protein is saCas9, the protein expression-driving promoter is a human troponin T promoter, and the RNA expression-driving promoter is a human U6 promoter.

62. The method of any one of claims 42-61, wherein the vector is a DNA-based vector, an mRNA-based vector, an adeno-associated virus-based vector, a retrovirus-based vector, or a lentivirus-based vector.

63. The method of any one of claims 42-61, wherein the vector is an adeno-associated virus vector, a retroviral vector, or a lentiviral vector.

64. The method of any one of claims 42-61, wherein the vector is an AAV9 vector.

65. The method of any one of claims 42-61, wherein the vector is a modified adeno-associated virus vector.

66. The method of claim 65, wherein the modified adeno-associated virus comprises any capsid protein described herein, optionally wherein the capsid protein is wild-type AAV9 capsid protein or a variant thereof.

67. A method of treating or preventing a disease or condition in a human with a deletion of Arg14 in a phospholamban (PLN) gene, comprising administering to the human the vector of any one of claims 1-30 and 36-40.

68. The method of any one of claims 42-67, wherein the disease or condition is a cardiac disease or condition.

69. The method of claim 68, wherein the cardiac disease or condition is cardiomyopathy.

70. The method of claim 69, wherein the cardiomyopathy is dilated cardiomyopathy, hypertrophic cardiomyopathy, arrhythmogenic cardiomyopathy, and/or arrhythmogenic right ventricular cardiomyopathy.

71. The method of claim 68, wherein the cardiac disease or condition is heart failure.

72. The method of claim 68, wherein the cardiac disease or condition is malignant ventricular tachycardia.

73. The method of claim 68, wherein the cardiac disease or condition is arrhythmia.

74. The method of any one of claims 42-73, wherein the method improves one or more measures of cardiac function.

75. The method of any one of claims 42-74, wherein the method increases ejection fraction.

76. The method of any one of claims 42-75, wherein the method reduces left ventricular internal dimension and/or left ventricular mass.

77. The method of any one of claims 42-76, wherein the human has a heterozygous deletion of Arg14 in a phospholamban (PLN) gene.

78. The method of any one of claims 42-77, wherein the administering is systemic administration or local administration to the heart.

79. The method of claim 78, wherein the systemic administration is intravenous administration.

80. The method of claim 78, wherein the local administration is by direct injection into the heart or cardiac tissue, intracoronary administration or retrograde coronary sinus infusion.

81. The method of any one of claims 42-80, wherein the administering results in an at least 20%, 25%, 30%, 35%, 40%, 45%, 50%, 55%, 60% or 70% editing efficiency of, or efficiency of indel formation in, the PLN gene comprising a deletion of Arg14.

82. The method of claim 81, wherein the administering results in an at least 50% or at least 70% editing efficiency of, or efficiency of indel formation in, the PLN gene comprising a deletion of Arg14.

83. The method of any one of claims 42-82, wherein the composition or vector does not, or substantially does not, edit or form indels in the wild-type PLN gene.

84. The method of any one of claims 42-83, wherein the vector is administered at a dose in the range of about 1×10¹² vector genomes (vg) per kg to about 1×10¹⁴ vector genomes (vg) per kg.

85. The method of any one of claims 42-83, wherein the vector is administered at a dose of less than about 1×10¹⁴ vector genomes (vg) per kg, or less than about 1×10¹³ vector genomes (vg) per kg.

86. A self-inactivating vector for specifically targeting a phospholamban (PLN) gene comprising a deletion of Arg14 comprising:
(i) a first polynucleotide encoding a Cas endonuclease protein operably linked to a human troponin T promoter;
(ii) a second polynucleotide encoding a guide RNA (gRNA) operably linked to an RNA expression-driving promoter, wherein the gRNA is complementary to a sequence of the PLN gene comprising a deletion of Arg14; and
(iii) a self-inactivation site,
wherein the first polynucleotide and the second polynucleotide have a head-to-tail orientation.

87. The vector of claim 86, wherein the Cas endonuclease protein is saCas9.

88. The vector of claim 87, wherein the saCas9 comprises a nucleotide sequence having at least 70%, 85%, 90%, 95% or 98% identity to SEQ ID NO: 10 and/or an amino acid sequence having at least 70%, 85%, 90%, 95% or 98% identity to SEQ ID NO: 11.

89. The vector of claim 87 or 88, wherein the saCas9 comprises the nucleotide sequence of SEQ ID NO: 10 and/or the amino acid sequence of SEQ ID NO: 11.

90. The vector of any one of claims 86-89, wherein the human troponin T promoter has a nucleotide sequence having at least 70%, 80%, 85%, 90%, 95% or 98% identity to SEQ ID NO: 12, SEQ ID NO: 13, or SEQ ID NO: 14.

91. The vector of any one of claims 86-90, wherein the human troponin T promoter has the nucleotide sequence of SEQ ID NO: 12, SEQ ID NO: 13, or SEQ ID NO: 14.

92. The vector of any one of claims 86-91, wherein the gRNA comprises at least 16 nucleotides with up to 3 mismatches of the nucleotide sequence ggttgaggctcttatagctga (SEQ ID NO: 1), optionally wherein the mismatch(es) is not in the sequence of ttatagctga (SEQ ID NO: 127) within SEQ ID NO: 1.

93. The vector of claim 92, wherein the gRNA comprises at least 16, 17, 18 or 19 nucleotides of the nucleotide sequence ggttgaggctcttatagctga (SEQ ID NO: 1).

94. The vector of claim 92, wherein the gRNA comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), and tggttgaggctcttatagctga (SEQ ID NO: 4).

95. The vector of claim 92, wherein the gRNA comprises the nucleotide sequence of ggttgaggctcttatagctga (SEQ ID NO: 1).

96. The vector of claim 92, wherein the gRNA comprises the nucleotide sequence of SEQ ID NO: 5, SEQ ID NO: 6, SEQ ID NO: 7, or SEQ ID NO: 8.

97. The vector of any one of claims 86-96, wherein the RNA expression-driving promoter is a U6 promoter.

98. The vector of claim 97, wherein the U6 promoter is a human U6 promoter having the nucleotide sequence of SEQ ID NO: 22.

99. The vector of any one of claims 86-98, wherein the self-inactivation site is 5′ to the human troponin T promoter, within the human troponin T promoter, between the human troponin T promoter and the first polynucleotide encoding the Cas endonuclease protein, within the first polynucleotide encoding the Cas endonuclease protein, or 3′ to the first polynucleotide encoding the Cas endonuclease protein.

100. The vector of claim 99, wherein the self-inactivation site is within the first polynucleotide encoding the Cas endonuclease protein, optionally near the 5′ end of the first polynucleotide encoding the Cas endonuclease protein, further optionally after the start codon “ATG”.

101. The vector of any one of claims 86-100, wherein the self-inactivation site comprises a gRNA target region.

102. The vector of claim 101, wherein the gRNA target region is recognized by the gRNA encoded by the second polynucleotide.

103. The vector of claim 102, wherein the gRNA target region comprises at least 16 nucleotides with up to 3 mismatches of the nucleotide sequence ggttgaggctcttatagctga (SEQ ID NO: 1), or a nucleotide sequence reverse complement to at least 16 nucleotides with up to 3 mismatches of the nucleotide sequence ggttgaggctcttatagctga (SEQ ID NO: 1), optionally wherein the mismatch(es) is not in the sequence of ttatagctga (SEQ ID NO: 127) within SEQ ID NO: 1.

104. The vector of claim 102, wherein the gRNA target region comprises at least 16, 17, 18 or 19 nucleotides of the nucleotide sequence ggttgaggctcttatagctga (SEQ ID NO: 1), or a nucleotide sequence reverse complement to at least 16, 17, 18 or 19 nucleotides of the nucleotide sequence ggttgaggctcttatagctga (SEQ ID NO: 1).

105. The vector of claim 102, wherein the gRNA target region comprises the nucleotide sequence selected from the group consisting of: ggttgaggctcttatagctga (SEQ ID NO: 1), ttgaggctcttatagctga (SEQ ID NO: 2), gttgaggctcttatagctga (SEQ ID NO: 3), tggttgaggctcttatagctga (SEQ ID NO: 4), and a nucleotide sequence reverse complement to any one of the foregoing.

106. The vector of claim 102, wherein the gRNA target region comprises the nucleotide sequence of ggttgaggctcttatagctga (SEQ ID NO: 1), or a nucleotide sequence reverse complement to ggttgaggctcttatagctga (SEQ ID NO: 1).

107. The vector of any one of claims 86-106, wherein the self-inactivation site further comprises a less optimal PAM sequence of the Cas endonuclease protein encoded by the first polynucleotide.

108. The vector of claim 107, wherein the Cas endonuclease protein is saCas9, and the less optimal PAM sequence is NNGRRC, NNGRRG, or NNGRRA, wherein N is any nucleotide, and R is guanine or adenine.

109. The vector of claim 108, wherein the less optimal PAM sequence is gcgagc, gcgaga, or gcgagg.

110. The vector of any one of claims 86-109, wherein the vector comprises the following 5′ to 3′ arrangement of elements: 5′-the human troponin T promoter-the first polynucleotide encoding the Cas endonuclease protein-the RNA expression-driving promoter-the second polynucleotide encoding the gRNA-3′, wherein the self-inactivation site is within the first polynucleotide encoding the Cas endonuclease protein, optionally near the 5′ end of the first polynucleotide encoding the Cas endonuclease protein, further optionally after the start codon “ATG”.

111. The vector of any one of claims 86-110, wherein the vector further comprises a polyadenylation sequence.

112. The vector of claim 111, wherein the polyadenylation sequence is selected from a BGH polyadenylation sequence and a SV40 polyadenylation sequence.

113. The vector of claim 112, wherein the BGH polyadenylation sequence has at least 90%, 95%, 98% or 100% identity to SEQ ID NO: 29, and/or the SV40 polyadenylation sequence has at least 90%, 95%, 98% or 100% identity to SEQ ID NO: 30.

114. The vector of any one of claims 111-113, wherein the polyadenylation sequence is between the first polynucleotide encoding the Cas endonuclease protein and the RNA expression-driving promoter, or wherein the vector comprises the following 5′ to 3′ arrangement of elements: 5′-the human troponin T promoter-the first polynucleotide encoding the Cas endonuclease protein-the polyadenylation sequence-the RNA expression-driving promoter-the second polynucleotide encoding the gRNA-3′.

115. The vector of any one of claims 86-114, wherein the vector is a DNA-based vector, an mRNA-based vector, an adeno-associated virus-based vector, a retrovirus-based vector, or a lentivirus-based vector.

116. The vector of any one of claims 86-114, wherein the vector is an adeno-associated virus vector, a retroviral vector, or a lentiviral vector.

117. The vector of any one of claims 86-114, wherein the vector is an AAV9 vector.

118. The vector of any one of claims 86-114, wherein the vector is a modified adeno-associated virus vector.

119. The vector of claim 118, wherein the modified adeno-associated virus comprises any capsid protein described herein, optionally wherein the capsid protein is wild-type AAV9 capsid protein or a variant thereof.

120. The vector of any one of claims 86-119, which, when introduced into iPSC-derived cardiomyocytes, has an at least 20%, 25%, 30%, 35%, 40%, 45%, 50%, 55%, 60% or 70% editing efficiency of, or efficiency of indel formation in, the PLN gene comprising a deletion of Arg14.

121. The vector of claim 120, which, when introduced into iPSC-derived cardiomyocytes, has an at least 50% or at least 70% editing efficiency of, or efficiency of indel formation in, the PLN gene comprising a deletion of Arg14.

122. The vector of any one of claims 86-121, which, when introduced into iPSC-derived cardiomyocytes, does not, or substantially does not, edit or form indels in the wild-type PLN gene.

123. The vector of any one of claims 86-122, wherein expression of the Cas endonuclease protein is terminated within about 4-8 weeks after introduced into iPSC-derived cardiomyocytes.

124. A pharmaceutical composition comprising the vector of any one of claims 86-123.

125. A method of treating or preventing a disease or condition in a human with a deletion of Arg14 in a phospholamban (PLN) gene, comprising administering to the human the vector of any one of claims 86-123 or the pharmaceutical composition of claim 124.

126. The method of any one of claim 125, wherein the disease or condition is a cardiac disease or condition.

127. The method of claim 126, wherein the cardiac disease or condition is cardiomyopathy.

128. The method of claim 127, wherein the cardiomyopathy is dilated cardiomyopathy, hypertrophic cardiomyopathy, arrhythmogenic cardiomyopathy, and/or
arrhythmogenic right ventricular cardiomyopathy.

129. The method of claim 126, wherein the cardiac disease or condition is heart failure.

130. The method of claim 126, wherein the cardiac disease or condition is malignant ventricular tachycardia.

131. The method of claim 126, wherein the cardiac disease or condition is arrhythmia.

132. The method of any one of claims 125-131, wherein the method improves one or more measures of cardiac function.

133. The method of any one of claims 125-132, wherein the method increases ejection fraction.

134. The method of any one of claims 125-133, wherein the method reduces left ventricular internal dimension and/or left ventricular mass.

135. The method of any one of claims 125-134, wherein the human has a heterozygous deletion of Arg14 in a phospholamban (PLN) gene.

136. The method of any one of claims 125-135, wherein the administering is systemic administration or local administration to the heart.

137. The method of claim 136, wherein the systemic administration is intravenous administration.

138. The method of claim 136, wherein the local administration is by direct injection into the heart or cardiac tissue, intracoronary administration or retrograde coronary sinus infusion.

139. The method of any one of claims 125-138, wherein the administering results in an at least 20%, 25%, 30%, 35%, 40%, 45%, 50%, 55%, 60% or 70% editing efficiency of, or efficiency of indel formation in, the PLN gene comprising a deletion of Arg14.

140. The method of claim 139, wherein the administering results in an at least 50% or at least 70% editing efficiency of, or efficiency of indel formation in, the PLN gene comprising a deletion of Arg14.

141. The method of any one of claims 125-140, wherein the vector or pharmaceutical composition does not, or substantially does not, edit or form indels in the wild-type PLN gene.

142. The method of any one of claims 125-141, wherein expression of the Cas endonuclease protein is terminated within about 4-8 weeks after the administering to the human.

143. The method of any one of claims 125-142, wherein the vector is administered at a dose in the range of about 1×10¹² vector genomes (vg) per kg to about 1×10¹⁴ vector genomes (vg) per kg.

144. The method of any one of claims 125-142, wherein the vector is administered at a dose of less than about 1×10¹⁴ vector genomes (vg) per kg, or less than about 1×10¹³ vector genomes (vg) per kg.

145. A self-inactivating vector for specifically targeting a gene of interest comprising:
(i) a first polynucleotide encoding a Cas endonuclease protein operably linked to a protein expression-driving promoter;
(ii) a second polynucleotide encoding a guide RNA (gRNA) operably linked to an RNA expression-driving promoter, wherein the gRNA is complementary to a sequence of the gene of interest; and
(iii) a self-inactivation site,
optionally wherein the first polynucleotide and the second polynucleotide have a head-to-tail orientation.

146. The vector of claim 145, wherein the Cas endonuclease protein is saCas9.

147. The vector of claim 146, wherein the saCas9 comprises a nucleotide sequence having at least 70%, 85%, 90%, 95% or 98% identity to SEQ ID NO: 10 and/or an amino acid sequence having at least 70%, 85%, 90%, 95% or 98% identity to SEQ ID NO: 11.

148. The vector of claim 146 or 147, wherein the saCas9 comprises the nucleotide sequence of SEQ ID NO: 10 and/or the amino acid sequence of SEQ ID NO: 11.

149. The vector of any one of claims 145-148, wherein the RNA expression-driving promoter is a U6 promoter.

150. The vector of claim 149, wherein the U6 promoter is a human U6 promoter having the nucleotide sequence of SEQ ID NO: 22.

151. The vector of any one of claims 145-150, wherein the self-inactivation site is 5′ to the protein expression-driving promoter, within the protein expression-driving promoter, between the protein expression-driving promoter and the first polynucleotide encoding the Cas endonuclease protein, within the first polynucleotide encoding the Cas endonuclease protein, or 3′ to the first polynucleotide encoding the Cas endonuclease protein.

152. The vector of claim 151, wherein the self-inactivation site is within the first polynucleotide encoding the Cas endonuclease protein, optionally near the 5′ end of the first polynucleotide encoding the Cas endonuclease protein, further optionally after the start codon “ATG”.

153. The vector of any one of claims 145-152, wherein the self-inactivation site comprises a gRNA target region.

154. The vector of claim 153, wherein the gRNA target region is recognized by the gRNA encoded by the second polynucleotide.

155. The vector of claim 102, wherein the gRNA target region comprises the gRNA sequence or a portion thereof, or a nucleotide sequence reverse complement to the gRNA sequence or a portion thereof.

156. The vector of any one of claims 145-155, wherein the self-inactivation site further comprises a less optimal PAM sequence of the Cas endonuclease protein encoded by the first polynucleotide.

157. The vector of claim 156, wherein the Cas endonuclease protein is saCas9, and the less optimal PAM sequence is NNGRRC, NNGRRG, or NNGRRA, wherein N is any nucleotide, and R is guanine or adenine.

158. The vector of claim 157, wherein the less optimal PAM sequence is gcgagc, gcgaga, or gcgagg.

159. The vector of any one of claims 145-158, wherein the vector comprises the following 5′ to 3′ arrangement of elements: 5′-the protein expression-driving promoter-the first polynucleotide encoding the Cas endonuclease protein-the RNA expression-driving promoter-the second polynucleotide encoding the gRNA-3′, wherein the self-inactivation site is within the first polynucleotide encoding the Cas endonuclease protein, optionally near the 5′ end of the first polynucleotide encoding the Cas endonuclease protein, further optionally after the start codon “ATG”.

160. The vector of any one of claims 145-159, wherein the vector is a DNA-based vector, an mRNA-based vector, an adeno-associated virus-based vector, a retrovirus-based vector, or a lentivirus-based vector.

161. The vector of any one of claims 145-159, wherein the vector is an adeno-associated virus vector, a retroviral vector, or a lentiviral vector.

162. The vector of any one of claims 145-159, wherein the vector is an AAV9 vector.

163. The vector of any one of claims 145-159, wherein the vector is a modified adeno-associated virus vector.

164. The vector of claim 163, wherein the modified adeno-associated virus comprises any capsid protein described herein, optionally wherein the capsid protein is wild-type AAV9 capsid protein or a variant thereof.

165. The vector of any one of claims 145-164, wherein expression of the Cas endonuclease protein is terminated within about 4-8 weeks after the vector is introduced into a cell.

166. The vector of any one of claims 145-165, wherein the gene of interest is selected from the group consisting of phospholamban (PLN), cardiac troponin T (TNNT2), BAG family molecular chaperone regulator 3 (BAG3), myosin heavy chain (MYH7), tropomyosin 1 (TPM1), myosin binding protein C (MYBPC3), 5′-AMP-activated protein kinase subunit gamma-2 (PRKAG2), troponin I type 3 (TNNI3), titin (TTN), myosin light chain 2 (MYL2), actin, alpha cardiac muscle 1 (ACTC1), potassium voltage-gated channel, KQT-like subfamily, member 1 (KCNQ1), myocyte enhancer factor 2c (MEF2C), cardiac LIM protein (CSRP3), DWORF, junctophilin (JPH2), alpha-crystallin B chain (CRYAB), LMNA (Lamin A and Lamin C isoforms), troponin I type 3 (TNNI3), lysosomal-associated membrane protein 2 (LAMP2, including LAMP2a, LAMP2b and LAMP2c isoforms), desmoplakin (DSP, including DPI and DPII isoforms), desmoglein 2 (DSG2), junction plakoglobin (JUP), plakophilin-2 (PKP2), matrix metallopeptidase 11 (MMP11), synaptopodin 2 like (SYNPO2L, including SYNPO2LA and SYNPO2LB), RNA binding motif protein 20 (RBM20), metastasis suppressor protein 1 (MTSS1), proprotein convertase subtilisin/kexin type 9 (PCSK9), acid alpha-glucosidase (GAA), and frataxin (FXN).

167. A pharmaceutical composition comprising the vector of any one of claims 145-166.

168. A method of editing a gene of interest in a cell, comprising introducing to the cell the vector of any one of claims 145-166.

169. The method of claim 168, wherein expression of the Cas endonuclease protein is terminated within about 4-8 weeks after introducing the vector to the cell.
